SEQUENCE LISTING (138 sequences)

SEQ ID NO: 1 - protein, 117 aa, synthetic construct
  source 1..117: note = 101A6 VH amino acid sequence
EVKLVESGGGLVQPGGSLSLSCAASGFTFTDYYLSWVRQPPGKALEWLALIRNKANGYTTEYSASVKGRFTISRDSSQSILYLQMDALRAEDSATYYCARDTLAAYWGQGTSVTVSS

SEQ ID NO: 2 - protein, 107 aa, synthetic construct
  source 1..107: note = 101A6 VL amino acid sequence
DIVMTQSHKFMSTSVGDRVSFTCKASQDVGSAVIWYQQKPGQSPKLLIYWASTRHTGVPDRFTGSGSGTDFTLTISNVQSEDLADYFCQQYSSYPLTFGAGTKLEIK

SEQ ID NO: 3 - protein, 120 aa, synthetic construct
  source 1..120: note = 64A10 VH amino acid sequence
QVQLKESGPVLVAPSQSLSITCTVSGFSLTSYGVYWVRQPPGKGPEWLGVIWTSGNTNYNSALMSRLSISKDNSKSQVFLKMNSLQTDDTAMYYCVKHDGGNYVGVMNYWGQGTSVTVSS

SEQ ID NO: 4 - protein, 107 aa, synthetic construct
  source 1..107: note = 64A10 VL amino acid sequence
DIQMTQSPATLSVTPGDRVSLSCRASQSISDYLHWYQQKSQESPRLLIKYASQSISGIPSRFSGSGSGSDFTLSINSVEPEDVGVYYCQNGHSFPPTFGGGTKLEIK

SEQ ID NO: 5 - protein, 113 aa, synthetic construct
  source 1..113: note = 4E12 VH amino acid sequence
QVQLQQSGAELMRPGVSVKISCKGSGYTFTDYAMHWVKQNHAKSLEWIGVISTYYGNVSYNQKFKGKATMTVDKSSSTAYMELARLTSEDSAIYYCARGFAYWGQGTLVTVSA

SEQ ID NO: 6 - protein, 112 aa, synthetic construct
  source 1..112: note = 4E12 VL amino acid sequence
DIVITQDELSNLVTSGESVSISCRSSKSLQKKDGKTYLNWFLQRPGQSPQLLIYLMSTRASGVSDRFSGSGSGTDFTLEISRVKAEDVGVYYCQQLVEYPFTFGSGTKLEIK

SEQ ID NO: 7 - protein, 120 aa, synthetic construct
  source 1..120: note = 19C2 VH amino acid sequence
QVTLKESGPGILKPSQTLSLTCSFSGFSLTTSGMGINWIRQPSGKGLEWLALIWWDDDKYYNPSLKNRLTVSKDPSRNQVFLTITSVDTTDSATYYCARSLITTAPSDYWGQGTSVTVSS

SEQ ID NO: 8 - protein, 107 aa, synthetic construct
  source 1..107: note = 19C2 VL amino acid sequence
DIVMTQSQKFMSTSVGDRVSITCKASQNVGTAITWYQQRPGQSPQLLIYSASNRYTGVPDRFTGSGSGTDFTLTISNLQSEDLADYFCLQYSDYPLTFGAGTNLELK

SEQ ID NO: 9 - protein, 120 aa, synthetic construct
  source 1..120: note = 4B12 VH amino acid sequence
QVTLKESGPGILKPAQTLSLTCSFSGFSLSTSGMGVGWIRQPSGKGLEWLAHIWWDDDKHYNPSLKSRLTISKDASRNQVFLRISSVDTADTATYYCVRSNYGYDWFAYWGQGTLVTVSA

SEQ ID NO: 10 - protein, 107 aa, synthetic construct
  source 1..107: note = 4B12 VL amino acid sequence
DIKMTQSPSSMYVSLGERVTITCKASQDINTYLGWFQQKPGKSPKTLIYRANRLVDGVPSRFSGSGSGQDYSLTISSLEYEDMEIYYCLQYDEFPLTFGAGTKLEIK

SEQ ID NO: 11 - protein, 118 aa, synthetic construct
  source 1..118: note = 57F4 VH amino acid sequence
EVQLVESGGGLVKPGGSRKLSCAASGFTFSDYGMHWVRQAPEKGLEWVAYISSGSSALYYADTVKGRFTISRDNVKNTLFLQMTSLRSEDTAMYYCARRSYYSYFAYWGQGTLVTVSA

SEQ ID NO: 12 - protein, 108 aa, synthetic construct
  source 1..108: note = 57F4 VL amino acid sequence
QIVLTQSPALMAASPGEKVTITCRVSSSISSSTLHWYQQKSETSPKPWIYGTSNLASGVPVRFSGSGSGTSYSLTISSMEAEDAATYYCQQWSSFPFTFGSGTKLEIK

SEQ ID NO: 13 - protein, 117 aa, synthetic construct
  source 1..117: note = 8H3 VH amino acid sequence
EVKLVESGGGLVQPGGSLSLSCAASGFTFTDYYMSWVRQPPGKALEWLALIRNKANGYTTEYSASVKGRFTISRDNSQSILYLQMNALRAEDSATYYCARDSFEVYWGQGTLVTVSA

SEQ ID NO: 14 - protein, 107 aa, synthetic construct
  source 1..107: note = 8H3 VL amino acid sequence
DIVLTQSHKFMSTSVGDRVNITCKASQDVGTAVAWYQQKPGQSPKLLIYWASTRHTGVPDRFTGSGSGTDFTLTIRNVQSEDLADYFCQQFSSFPFTFGGGTKLEIK

SEQ ID NO: 15 - protein, 120 aa, synthetic construct
  source 1..120: note = 64A10HZ VH amino acid sequence
QVQLQESGPGLVKPSETLSLTCTVSGFSLTSYGVYWVRQPPGKGPEWLGVIWTSGNTNYNPSLKSRLTISKDNSKNQVSLKLSSVTAADTAVYYCVKHDGGNYVGVMNYWGQGTLVTVSS

SEQ ID NO: 16 - protein, 107 aa, synthetic construct
  source 1..107: note = 64A10HZ VL amino acid sequence
EIVMTQSPDFQSVTPKEKVTITCRASQSISDYLHWYQQKPDQSPKLLIKYASQSISGVPSRFSGSGSGSDFTLTINSLEAEDAATYYCQNGHSFPPTFGQGTKLEIK

SEQ ID NO: 17 - protein, 120 aa, synthetic construct
  source 1..120: note = 64A10HZ05 VH amino acid sequence
QVQLQESGPGLVKPSETLSLTCTVSGFSLTSYGVYWVRQPPGKGLEWLGVIWTSGNTNYNPSLKSRLTISKDNSKNQVSLKLSSVTAADTAVYYCVKHDGGNYVGVMNYWGQGTLVTVSS

SEQ ID NO: 18 - protein, 107 aa, synthetic construct
  source 1..107: note = 64A10HZ05 VL amino acid sequence
DIQMTQSPSSLSASVGDRVTITCRASQSISDYLHWYQQKPGKAPKLLIKYASQSISGVPSRFSGSGSGSDFTLTISSLQPEDFATYYCQNGHSFPPTFGGGTKVEIK

SEQ ID NO: 19 - protein, 117 aa, synthetic construct
  source 1..117: note = 101A6HZ VH amino acid sequence
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSS

SEQ ID NO: 20 - protein, 107 aa, synthetic construct
  source 1..107: note = 101A6HZ VL amino acid sequence
DIVMTQSPDSLAVSLGERATINCKASQDVGSAVIWYQQKPGQSPKLLIYWASTRHTGVPDRFSGSGSGTDFTLTISSLQAEDVAVYYCQQYSSYPLTFGQGTKLEIK

SEQ ID NO: 21 - protein, 113 aa, synthetic construct
  source 1..113: note = 4E12HZ VH amino acid sequence
QVQLVQSGAEVKRPGASVKVSCKTSGYTFTDYAMHWVRQAPGKGLEWIGVISTYYGNVSYNQKFKGRVTMTRDTSTSTVYMELSSLTSEDTAIYYCARGFAYWGQGTLVTVSS

SEQ ID NO: 22 - protein, 112 aa, synthetic construct
  source 1..112: note = 4E12HZ VL amino acid sequence
DIVITQTELSLPVTPGERASISCRSSKSLQKKDGKTYLNWFLQKPGQSPQLLIYLMSTRASGVSDRFSGSGSGTDFTLKISRVEAEDVGVYYCQQLVEYPFTFGQGTKLEIK

SEQ ID NO: 23 - protein, 120 aa, synthetic construct
  source 1..120: note = 19C2HZ VH amino acid sequence
QVTLKESGPTLVKPTQTLTLTCTFSGFSLSTSGMGINWIRQPPGKALEWLALIWWDDDKYYNPSLKNRLTITKDTSKNQVVLTMTNMDPVDTATYYCARSLITTAPSDYWGQGTLVTVSS

SEQ ID NO: 24 - protein, 107 aa, synthetic construct
  source 1..107: note = 19C2HZ VL amino acid sequence
DIQMTQSPSSLSASVGDRVTITCKASQNVGTAITWYQQRPGKAPKLLIYSASNRYTGVPSRFSGSGSGTDFTLTISNLQPEDLATYFCLQYSDYPLTFGQGTRLEIK

SEQ ID NO: 25 - protein, 330 aa, synthetic construct [sequence omitted]

SEQ ID NO: 26 - protein, 107 aa, synthetic construct [sequence omitted]

SEQ ID NO: 27 - protein, 7 aa, synthetic construct
  source 1..7: note = 101A6/101A6HZ/8H3 Chothia CDR H1
GFTFTDY

SEQ ID NO: 28 - protein, 8 aa, synthetic construct
  source 1..8: note = 101A6/101A6HZ/8H3 Chothia CDR H2
RNKANGYT

SEQ ID NO: 29 - protein, 6 aa, synthetic construct
  source 1..6: note = 101A6/101A6HZ Chothia/kabat/AbM CDR H3
DTLAAY

SEQ ID NO: 30 - protein, 11 aa, synthetic construct
  source 1..11: note = 101A6/101A6HZ Chothia/Kabat/AbM CDR L1
KASQDVGSAVI

SEQ ID NO: 31 - protein, 7 aa, synthetic construct
  source 1..7: note = 101A6/101A6HZ/8H3 Chothia/Kabat/AbM CDR L2
WASTRHT

SEQ ID NO: 32 - protein, 9 aa, synthetic construct
  source 1..9: note = 101A6/101A6HZ Chothia/Kabat/IMGT/AbM CDR L3
QQYSSYPLT

SEQ ID NO: 33 - protein, 5 aa, synthetic construct
  source 1..5: note = 101A6/101A6HZ Kabat CDR H1
DYYLS

SEQ ID NO: 34 - protein, 19 aa, synthetic construct
  source 1..19: note = 101A6HZ Kabat CDR H2
LIRNKANGYTTEYAASVKG

SEQ ID NO: 35 - protein, 19 aa, synthetic construct
  source 1..19: note = 101A6/8H3 Kabat CDR H2
LIRNKANGYTTEYSASVKG

SEQ ID NO: 36 - protein, 8 aa, synthetic construct
  source 1..8: note = 101A6/101A6HZ/8H3 IMGT CDR H1
GFTFTDYY

SEQ ID NO: 37 - protein, 10 aa, synthetic construct
  source 1..10: note = 101A6/101A6HZ/8H3 IMGT CDR H2
IRNKANGYTT

SEQ ID NO: 38 - protein, 8 aa, synthetic construct
  source 1..8: note = 101A6/101A6HZ IMGT CDR H3
ARDTLAAY

SEQ ID NO: 39 - protein, 6 aa, synthetic construct
  source 1..6: note = 101A6/101A6HZ IMGT CDR L1
QDVGSA

SEQ ID NO: 40 - skipped

SEQ ID NO: 41 - protein, 7 aa, synthetic construct
  source 1..7: note = 64A10/64A10HZ/64A10HZ05 Chothia CDR H1
GFSLTSY

SEQ ID NO: 42 - protein, 5 aa, synthetic construct
  source 1..5: note = 64A10/64A10HZ/64A10HZ05 Chothia CDR H2
WTSGN

SEQ ID NO: 43 - protein, 12 aa, synthetic construct
  source 1..12: note = 64A10/64A10HZ/64A10HZ05 Chothia/Kabat/AbM CDR H3
HDGGNYVGVMNY

SEQ ID NO: 44 - protein, 11 aa, synthetic construct
  source 1..11: note = 64A10/64A10HZ/64A10HZ05 Chothia/Kabat/AbM CDR L1
RASQSISDYLH

SEQ ID NO: 45 - protein, 7 aa, synthetic construct
  source 1..7: note = 64A10/64A10HZ/64A10HZ05 Chothia/Kabat/AbM CDR L2
YASQSIS

SEQ ID NO: 46 - protein, 9 aa, synthetic construct
  source 1..9: note = 64A10/64A10HZ/64A10HZ05 Chothia/Kabat/IMGT/AbM CDR L3
QNGHSFPPT

SEQ ID NO: 47 - protein, 5 aa, synthetic construct
  source 1..5: note = 64A10/64A10HZ/64A10HZ05 Kabat CDR H1
SYGVY

SEQ ID NO: 48 - protein, 16 aa, synthetic construct
  source 1..16: note = 64A10HZ/64A10HZ05 Kabat CDR H2
VIWTSGNTNYNPSLKS

SEQ ID NO: 49 - protein, 16 aa, synthetic construct
  source 1..16: note = 64A10 Kabat CDR H2
VIWTSGNTNYNSALMS

SEQ ID NO: 50 - protein, 8 aa, synthetic construct
  source 1..8: note = 64A10/64A10HZ/64A10HZ05 IMGT CDR H1
GFSLTSYG

SEQ ID NO: 51 - protein, 7 aa, synthetic construct
  source 1..7: note = 64A10/64A10HZ/64A10HZ05 IMGT CDR H2
IWTSGNT

SEQ ID NO: 52 - protein, 14 aa, synthetic construct
  source 1..14: note = 64A10/64A10HZ/64A10HZ05 IMGT CDR H3
VKHDGGNYVGVMNY

SEQ ID NO: 53 - protein, 6 aa, synthetic construct
  source 1..6: note = 64A10/64A10HZ/64A10HZ05 IMGT CDR L1
QSISDY

SEQ ID NO: 54 - skipped

SEQ ID NO: 55 - protein, 7 aa, synthetic construct
  source 1..7: note = 4E12/4E12HZ Chothia CDR H1
GYTFTDY

SEQ ID NO: 56 - protein, 6 aa, synthetic construct
  source 1..6: note = 4E12/4E12HZ Chothia CDR H2
STYYGN

SEQ ID NO: 57 - protein, 4 aa, synthetic construct
  source 1..4: note = 4E12/4E12HZ Chothia/Kabat/AbM CDR H3
GFAY

SEQ ID NO: 58 - protein, 16 aa, synthetic construct
  source 1..16: note = 4E12/4E12HZ Chothia/Kabat/AbM CDR L1
RSSKSLQKKDGKTYLN

SEQ ID NO: 59 - protein, 7 aa, synthetic construct
  source 1..7: note = 4E12/4E12HZ Chothia/Kabat/AbM CDR L2
LMSTRAS

SEQ ID NO: 60 - protein, 9 aa, synthetic construct
  source 1..9: note = 4E12/4E12HZ Chothia/Kabat/IMGT/AbM CDR L3
QQLVEYPFT

SEQ ID NO: 61 - protein, 5 aa, synthetic construct
  source 1..5: note = 4E12/4E12HZ Kabat CDR H1
DYAMH

SEQ ID NO: 62 - protein, 17 aa, synthetic construct
  source 1..17: note = 4E12/4E12HZ Kabat CDR H2
VISTYYGNVSYNQKFKG

SEQ ID NO: 63 - protein, 8 aa, synthetic construct
  source 1..8: note = 4E12/4E12HZ IMGT CDR H1
GYTFTDYA

SEQ ID NO: 64 - protein, 8 aa, synthetic construct
  source 1..8: note = 4E12/4E12HZ IMGT CDR H2
ISTYYGNV

SEQ ID NO: 65 - protein, 6 aa, synthetic construct
  source 1..6: note = 4E12/4E12HZ IMGT CDR H3
ARGFAY

SEQ ID NO: 66 - protein, 11 aa, synthetic construct
  source 1..11: note = 4E12/4E12HZ IMGT CDR L1
KSLQKKDGKTY

SEQ ID NO: 67 - skipped

SEQ ID NO: 68 - protein, 9 aa, synthetic construct
  source 1..9: note = 19C2 Chothia CDR H1
GFSLTTSGM

SEQ ID NO: 69 - protein, 5 aa, synthetic construct
  source 1..5: note = 19C2/19C2HZ/4B12 Chothia CDR H2
WWDDD

SEQ ID NO: 70 - protein, 10 aa, synthetic construct
  source 1..10: note = 19C2/19C2HZ Chothia/Kabat/AbM CDR H3
SLITTAPSDY

SEQ ID NO: 71 - protein, 11 aa, synthetic construct
  source 1..11: note = 19C2/19C2HZ Chothia/Kabat/AbM CDR L1
KASQNVGTAIT

SEQ ID NO: 72 - protein, 7 aa, synthetic construct
  source 1..7: note = 19C2/19C2HZ Chothia/Kabat/AbM CDR L2
SASNRYT

SEQ ID NO: 73 - protein, 9 aa, synthetic construct
  source 1..9: note = 19C2/19C2HZ Chothia/Kabat/IMGT/AbM CDR L3
LQYSDYPLT

SEQ ID NO: 74 - protein, 9 aa, synthetic construct
  source 1..9: note = 19C2HZ/4B12 Chothia CDR H1
GFSLSTSGM

SEQ ID NO: 75 - protein, 7 aa, synthetic construct
  source 1..7: note = 19C2/19C2HZ Kabat CDR H1
TSGMGIN

SEQ ID NO: 76 - protein, 16 aa, synthetic construct
  source 1..16: note = 19C2/19C2HZ Kabat CDR H2
LIWWDDDKYYNPSLKN

SEQ ID NO: 77 - protein, 10 aa, synthetic construct
  source 1..10: note = 19C2 IMGT CDR H1
GFSLTTSGMG

SEQ ID NO: 78 - protein, 7 aa, synthetic construct
  source 1..7: note = 19C2/19C2HZ/4B12 IMGT CDR H2
IWWDDDK

SEQ ID NO: 79 - protein, 12 aa, synthetic construct
  source 1..12: note = 19C2/19C2HZ IMGT CDR H3
ARSLITTAPSDY

SEQ ID NO: 80 - protein, 10 aa, synthetic construct
  source 1..10: note = 19C2HZ/4B12 IMGT CDR H1
GFSLSTSGMG

SEQ ID NO: 81 - protein, 6 aa, synthetic construct
  source 1..6: note = 19C2/19C2HZ IMGT CDR L1
QNVGTA

SEQ ID NO: 82 - skipped

SEQ ID NO: 83 - protein, 10 aa, synthetic construct
  source 1..10: note = 4B12 Chothia/Kabat/AbM CDR H3
SNYGYDWFAY

SEQ ID NO: 84 - protein, 11 aa, synthetic construct
  source 1..11: note = 4B12 Chothia/Kabat/AbM CDR L1
KASQDINTYLG

SEQ ID NO: 85 - protein, 7 aa, synthetic construct
  source 1..7: note = 4B12 Chothia/Kabat/AbM CDR L2
RANRLVD

SEQ ID NO: 86 - protein, 9 aa, synthetic construct
  source 1..9: note = 4B12 Chothia/Kabat/IMGT/AbM CDR L3
LQYDEFPLT

SEQ ID NO: 87 - protein, 7 aa, synthetic construct
  source 1..7: note = 4B12 Kabat CDR H1
TSGMGVG

SEQ ID NO: 88 - protein, 16 aa, synthetic construct
  source 1..16: note = 4B12 Kabat CDR H2
HIWWDDDKHYNPSLKS

SEQ ID NO: 89 - protein, 12 aa, synthetic construct
  source 1..12: note = 4B12 IMGT CDR H3
VRSNYGYDWFAY

SEQ ID NO: 90 - protein, 6 aa, synthetic construct
  source 1..6: note = 4B12 IMGT CDR L1
QDINTY

SEQ ID NO: 91 - skipped

SEQ ID NO: 92 - protein, 7 aa, synthetic construct
  source 1..7: note = 57F4 Chothia CDR H1
GFTFSDY

SEQ ID NO: 93 - protein, 6 aa, synthetic construct
  source 1..6: note = 57F4 Chothia CDR H2
SSGSSA

SEQ ID NO: 94 - protein, 9 aa, synthetic construct
  source 1..9: note = 57F4 Chothia/Kabat/AbM CDR H3
RSYYSYFAY

SEQ ID NO: 95 - protein, 12 aa, synthetic construct
  source 1..12: note = 57F4 Chothia/Kabat/AbM CDR L1
RVSSSISSSTLH

SEQ ID NO: 96 - protein, 7 aa, synthetic construct
  source 1..7: note = 57F4 Chothia/Kabat/AbM CDR L2
GTSNLAS

SEQ ID NO: 97 - protein, 9 aa, synthetic construct
  source 1..9: note = 57F4 Chothia/Kabat/IMGT/AbM CDR L3
QQWSSFPFT

SEQ ID NO: 98 - protein, 5 aa, synthetic construct
  source 1..5: note = 57F4 Kabat CDR H1
DYGMH

SEQ ID NO: 99 - protein, 17 aa, synthetic construct
  source 1..17: note = 57F4 Kabat CDR H2
YISSGSSALYYADTVKG

SEQ ID NO: 100 - protein, 8 aa, synthetic construct
  source 1..8: note = 57F4 IMGT CDR H1
GFTFSDYG

SEQ ID NO: 101 - protein, 8 aa, synthetic construct
  source 1..8: note = 57F4 IMGT CDR H2
ISSGSSAL

SEQ ID NO: 102 - protein, 11 aa, synthetic construct
  source 1..11: note = 57F4 IMGT CDR H3
ARRSYYSYFAY

SEQ ID NO: 103 - protein, 7 aa, synthetic construct
  source 1..7: note = 57F4 IMGT CDR L1
SSISSST

SEQ ID NO: 104 - skipped

SEQ ID NO: 105 - protein, 6 aa, synthetic construct
  source 1..6: note = 8H3 Chothia/Kabat/AbM CDR H3
DSFEVY

SEQ ID NO: 106 - protein, 11 aa, synthetic construct
  source 1..11: note = 8H3 Chothia/Kabat/AbM CDR L1
KASQDVGTAVA

SEQ ID NO: 107 - protein, 9 aa, synthetic construct
  source 1..9: note = 8H3 Chothia/Kabat/IMGT/AbM CDR L3
QQFSSFPFT

SEQ ID NO: 108 - protein, 5 aa, synthetic construct
  source 1..5: note = 8H3 Kabat CDR H1
DYYMS

SEQ ID NO: 109 - protein, 8 aa, synthetic construct
  source 1..8: note = 8H3 IMGT CDR H3
ARDSFEVY

SEQ ID NO: 110 - protein, 6 aa, synthetic construct
  source 1..6: note = 8H3 IMGT CDR L1
QDVGTA

SEQ ID NO: 111 - protein, 447 aa, synthetic construct
  source 1..447: note = 101A6HZ heavy chain amino acid sequence
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 214 aa, synthetic construct
  source 1..214: note = 101A6HZ light chain amino acid sequence
DIVMTQSPDSLAVSLGERATINCKASQDVGSAVIWYQQKPGQSPKLLIYWASTRHTGVPDRFSGSGSGTDFTLTISSLQAEDVAVYYCQQYSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 10 aa, synthetic construct
  source 1..10: note = 101A6/101A6HZ AbM CDR H1
GFTFTDYYLS

SEQ ID NO: 114 - protein, 12 aa, synthetic construct
  source 1..12: note = 101A6/101A6HZ/8H3 AbM CDR H2
LIRNKANGYTTE

SEQ ID NO: 115 - protein, 10 aa, synthetic construct
  source 1..10: note = 64A10/64A10HZ/64A10HZ05 AbM CDR H1
GFSLTSYGVY

SEQ ID NO: 116 - protein, 9 aa, synthetic construct
  source 1..9: note = 64A10/64A10HZ/64A10HZ05 AbM CDR H2
VIWTSGNTN

SEQ ID NO: 117 - protein, 10 aa, synthetic construct
  source 1..10: note = 4E12/4E12HZ AbM CDR H1
GYTFTDYAMH

SEQ ID NO: 118 - protein, 10 aa, synthetic construct
  source 1..10: note = 4E12/4E12HZ AbM CDR H2
VISTYYGNVS

SEQ ID NO: 119 - protein, 12 aa, synthetic construct
  source 1..12: note = 19C2 AbM CDR H1
GFSLTTSGMGIN

SEQ ID NO: 120 - protein, 9 aa, synthetic construct
  source 1..9: note = 19C2/19C2HZ AbM CDR H2
LIWWDDDKY

SEQ ID NO: 121 - protein, 12 aa, synthetic construct
  source 1..12: note = 19C2HZ AbM CDR H1
GFSLSTSGMGIN

SEQ ID NO: 122 - protein, 12 aa, synthetic construct
  source 1..12: note = 4B12 AbM CDR H1
GFSLSTSGMGVG

SEQ ID NO: 123 - protein, 9 aa, synthetic construct
  source 1..9: note = 4B12 AbM CDR H2
HIWWDDDKH

SEQ ID NO: 124 - protein, 10 aa, synthetic construct
  source 1..10: note = 57F4 AbM CDR H1
GFTFSDYGMH

SEQ ID NO: 125 - protein, 10 aa, synthetic construct
  source 1..10: note = 57F4 AbM CDR H2
YISSGSSALY

SEQ ID NO: 126 - protein, 10 aa, synthetic construct
  source 1..10: note = 8H3 AbM CDR H1
GFTFTDYYMS

SEQ ID NO: 127 - DNA, 351 bp, synthetic construct
  source 1..351: mol_type = other DNA; note = 101A6HZ VH nucleotide sequence
gaggtgcagctggtggaatccggaggaggcctggtccaacccggcggctccctgagactgtcttgtgccgcctccggctttacattcaccgactactacctgtcttgggtgcggcaggcccctggcaagggcctggaatggctggctctgatccggaacaaggccaacggctacacaaccgagtacgccgcttctgtgaagggcagattcaccatctctcgggattcctctaagaactcgctgtacctgcagatgaactctctgaagaccgaggacacagccgtgtactactgcgccagagataccctggccgcctactggggccagggcaccctggtgaccgtgtcctcc

SEQ ID NO: 128 - DNA, 321 bp, synthetic construct
  source 1..321: mol_type = other DNA; note = 101A6HZ VL nucleotide sequence
gacatcgtgatgacccagtctcctgattccctggccgtgagcctgggcgagagagccaccatcaactgcaaggcttctcaggatgtgggatctgccgtgatctggtaccagcagaagcctggccagagccccaagctgctgatctactgggcctccaccaggcataccggcgtgcctgaccggttcagcggctctggctccggcacagacttcaccctgacaatctccagtctgcaggccgaggacgtcgctgtgtactactgccagcagtactcctcctatcctctgacctttggacaaggtaccaagctggagatcaag

SEQ ID NO: 129 - DNA, 1341 bp, synthetic construct
  source 1..1341: mol_type = other DNA; note = 101A6HZ heavy chain nucleotide sequence
gaagtgcaactagtggaaagtggtggtggtctcgtgcagcctggcggaagcctgagactgagctgtgctgcttctggcttcaccttcaccgactactatctgtcttgggtccgccaggcccccggcaagggcctggaatggctggctctgatccggaacaaggccaacggctacaccacagagtacgccgcttccgtgaaaggcagatttaccatctcccgggactcctccaagaactccctgtacctgcagatgaactccctgaagaccgaggacaccgccgtgtactactgcgccagagatacactggccgcctactggggccaaggcaccctggtgaccgtgtcctctgcctctacaaagggcccctccgtgtttccactggctccctcctctaagagcacctctggcggcacagccgctctgggctgtctggtgaaggattacttcccagagcccgtgacagtgagctggaactctggcgccctgacctctggagtgcacacatttccagctgtgctgcagtccagcggcctgtactccctgtcttccgtggtgaccgtgcccagctcttccctgggcacccagacatatatctgcaacgtgaaccacaagccatccaatacaaaggtggacaagaaggtggagcccaagtcctgtgataagacccatacatgccccccttgtcctgctccagagctgctgggaggaccttccgtgttcctgtttccacccaaacctaaggacaccctgatgatcagcaggacccccgaggtgacatgcgtggtggtggacgtgtctcacgaggatcccgaggtgaagttcaactggtatgtcgatggcgtggaggtgcataatgctaagaccaagcctcgggaggagcagtacaactccacctatagagtggtgagcgtgctgacagtgctgcatcaggactggctgaacggcaaggagtataagtgcaaggtgtccaataaggccctgcccgctcccatcgagaagaccatcagcaaggccaagggccagcctagggagccacaggtgtacacactgcctccatcccgggacgagctgaccaagaaccaggtgagcctgacatgtctggtgaagggcttctatcctagcgatatcgctgtggagtgggagtccaatggccagccagagaacaattacaagaccacaccccctgtgctggactccgatggcagcttctttctgtattctaagctgaccgtggataagtcccggtggcagcagggcaacgtgttctcctgtagcgtgatgcacgaagccctgcataatcactatactcagaaaagtctgtcactgtcacctggtaaa

SEQ ID NO: 130 - DNA, 642 bp, synthetic construct
  source 1..642: mol_type = other DNA; note = 101A6HZ light chain nucleotide sequence
gacatcgtgatgacccagtctcctgattccctggccgtgagcctgggcgagagagccaccatcaactgcaaggcttctcaggatgtgggatctgccgtgatctggtaccagcagaagcctggccagagccccaagctgctgatctactgggcctccaccaggcataccggcgtgcctgaccggttcagcggctctggctccggcacagacttcaccctgacaatctccagtctgcaggccgaggacgtcgctgtgtactactgccagcagtactcctcctatcctctgacctttggacaaggtaccaagctggagatcaagcggaccgtggctgctccttccgtgttcatcttccctccatctgatgaacagctgaagtctggcaccgcttctgtggtgtgcctgctgaacaacttctaccccagagaagccaaggtccagtggaaggtggacaacgccctgcagtccggcaatagccaagagtctgtgaccgagcaggactccaaggactctacctactccctgtcctctaccctgacactcagcaaggccgactacgagaagcacaaagtgtacgcctgcgaggttacccaccagggcctgtcctctcctgtgaccaaatccttcaacagaggcgaatgt

SEQ ID NO: 131 - protein, 330 aa, synthetic construct
  source 1..330: note = Mutant human heavy chain constant region IgG1m
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - DNA, 1341 bp, synthetic construct
  source 1..1341: mol_type = other DNA; note = 101A6HZm heavy chain nucleotide sequence
gaggtgcagctggtggaatctggcggaggactcgtgcagcctggcggatctctgagactgtcttgcgctgcctctggctttaccttcaccgactactacctgtcttgggtgcggcaggccccaggcaagggcctggaatggctggcactgatccggaacaaggccaacggctacaccaccgagtacgccgcctccgtcaaaggcagattcaccatctcccgcgactccagcaagaactctctgtacctgcagatgaacagcctgaagaccgaggacaccgccgtgtactactgcgccagagataccctggccgcttactggggccagggcacactggtgaccgtctcctctgcttctaccaagggcccttctgtgtttcctctggccccttctagcaagtctacctctggcggcaccgctgccctgggatgtcttgtgaaggattacttccccgaacctgtgaccgtgtcctggaactccggcgccctgacatctggagtgcatacctttcccgctgtgctgcagtctagcggcctgtactccctgtcctccgtcgttaccgtgccttccagcagcctgggcacccagacctacatctgcaacgtgaaccacaagccctccaacaccaaagtggataagaaggtggaacccaagtcctgcgacaagacccacacctgtcctccttgccctgccccagaggccgctggcgctccatcagtgttcctgttccctcctaaacctaaggacaccctgatgatctctcggacacctgaggtgacctgcgtggtggtcgacgtgtctcacgaggaccctgaagtgaagttcaactggtacgtggatggcgtggaagtgcacaacgccaagaccaagcctagagaggaacagtacaactccacctatagagtggtgtccgtactgacagtgctgcatcaggactggctgaacggcaaagagtacaagtgcaaggtgtccaacaaagctctgcctgctcctatcgagaagaccatctccaaggccaagggccagcctcgggagcctcaagtgtacaccctgccccctagcagggacgagctgaccaagaatcaggtgtccctgacttgtctggtcaaaggcttctacccctccgacatcgccgtggagtgggagtccaatggccaacccgagaacaactacaaaaccacacctccagtgctggactctgacggctccttcttcctgtattccaagctgaccgtggacaagtccagatggcagcagggcaacgtgttctcctgctccgtgatgcacgaggctctgcacaatcactacacacagaagtccctgagcttgtctcctggcaag

SEQ ID NO: 133 - protein, 5 aa, synthetic construct
  source 1..5: note = Linker
GGGGS

SEQ ID NO: 134 - protein, 20 aa, synthetic construct
  source 1..20: note = Linker
GGGGSGGGGSGGGGSGGGGS

SEQ ID NO: 135 - protein, 447 aa, synthetic construct
  source 1..447: note = 101A6HZm heavy chain amino acid sequence
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 446 aa, synthetic construct
  source 1..446: note = 101A6HZm heavy chain amino acid sequence (Pyroglutamate at N-terminus; no C-terminal lysine)
  MOD_RES 1: note = pyroglutamate or pyroglutamic acid
XVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 446 aa, synthetic construct
  source 1..446: note = 101A6HZm heavy chain amino acid sequence (no C-terminal lysine)
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 447 aa, synthetic construct
  source 1..447: note = 101A6HZm heavy chain amino acid sequence (Pyroglutamate at N-terminus)
  MOD_RES 1: note = pyroglutamate or pyroglutamic acid
XVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0250420 A1
(43) Pub. Date: Aug. 27, 2026

(54) PTK7-BINDING PROTEIN AND USE THEREOF

(71) Applicant: SICHUAN KELUN-BIOTECH BIOPHARMACEUTICAL CO., LTD., Chengdu, Sichuan (CN)

(72) Inventors: Hu Long, Chengdu, Sichuan (CN); Jiangjiang Hu, Chengdu, Sichuan (CN); Mei Chen, Chengdu, Sichuan (CN); Hanjie Liu, Chengdu, Sichuan (CN); Hongjiao Jiang, Chengdu, Sichuan (CN); Junyou Ge, Chengdu, Sichuan (CN); Xiangyang Tan, Chengdu, Sichuan (CN); Miao Tan, Chengdu, Sichuan (CN)

(73) Assignee: SICHUAN KELUN-BIOTECH BIOPHARMACEUTICAL CO., LTD., Chengdu, Sichuan (CN)

(21) Appl. No.: 18/995,560

(22) PCT Filed: Aug. 18, 2023
(86) PCT No.: PCT/CN2023/113721

§ 371 Date: Jan. 16, 2025

(30) Foreign Application Priority Data
Aug. 19, 2022 (CN) 202210998273.4
Oct. 14, 2022 (CN) 202211260998.X

(51) Int. Cl. C07K 16/40

(52) U.S. Cl. CPC C07K 16/40; C07K 2317/24; C07K 2317/33; C07K 2317/565; C07K 2317/732; C07K 2317/92

(57) ABSTRACT

The present invention relates to the field of disease treatment, and particularly relates to an anti-PTK7 antibody or antigen-binding fragment thereof, nucleic acid molecules encoding the same, and a method for preparing the same. The anti-PTK7 antibody or antigen-binding fragment thereof of the present invention has high affinity binding and endocytosis activity to PTK7, and meanwhile has good hydrophilicity. Therefore, the present invention further relates to use of the antibody or antigen-binding fragment thereof in disease treatment and diagnosis.

### CROSS REFERENCE TO RELATED APPLICATIONS

This application claims the benefit of Chinese Application No. 202210998273.4, filed Aug. 19, 2022 and Chinese Application No. 202211260998.X, filed Oct. 14, 2022, the disclosures each of which are incorporated herein by reference in their entireties.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14463-063-228_SEQ_LISTING.xml”, was created on Aug. 15, 2023, and is 152,208 bytes in size.

### BACKGROUND OF THE INVENTION

### (1) Technical Field

The present invention belongs to the field of therapeutic monoclonal antibodies, and more specifically relates to an antibody for PTK7 and use of the antibody in treating and diagnosing the diseases.

### (2) Description of Related Art

PTK7 (protein tyrosine kinase 7) belongs to receptor tyrosine kinase family, and due to the mutation of kinase domain can lack kinase activity. As being originally identified in colon cancer cells, so PTK7 is also called as CCK4 (colon carcinoma kinase-4). PTK7 is composed of 7 extracellular immunoglobulin domains, a transmembrane domain and an intracellular tyrosine kinase domain, and its ligand is unknown. The extracellular segment of PTK7 can be cleaved at the second position by ADAM protease and MT1-MMP. Hydrolysis of protease enables PTK7 to generate two soluble fragments. PTK7 is an evolutionary conservative transmembrane receptor, its function includes multiple processes from embryonic morphogenesis to epidermal wound repair, and it is also a multifunctional synergistic receptor and plays a role of a molecular switch in Wnt, sema-plexin (semaphorin, axon guidance factor, plexin protein) and VEGF signal pathways.

PTK7 has a certain expression in normal tissues such as endometrium, ovary and placenta, but it is highly expressed in various solid tumors: 47.4% in NSCLC, with H-score mean value of 117.4; 45.10% in ovarian cancer, with H-score mean value of 151; and 28.6% in TNBC, with H-score mean value of 159.2. It is reported that in esophageal cancer cases, PTK7 was expressed by more than 80%, 60% were positive for 2⁺ or 3⁺ IHC staining, normal esophageal tissues were negative for PTK7 staining, and the high expression of PTK7 was related to poor prognosis. Cofetuzumab Pelidotin, an ADC drug targeting PTK7 and developed by Pfizer, has shown good safety and initial efficacy in clinical stage I. According to the high expression of PTK7 in various tumors and clinical verification of the safety and effectiveness of the target drug, PTK7 is an ideal therapeutic target.

At present, there are no antibody drugs targeting PTK7 on the market. Therefore, it is urgent and necessary to develop antibodies targeting PTK7 with higher specificity, lower toxic and side effects, better clinical therapeutic effect and more convenient administration mode, which will provide patients with more drug choices.

### BRIEF SUMMARY OF THE INVENTION

In this application, the inventor has developed an anti-human PTK7 humanized antibody having good hydrophilicity and stability, which can specifically recognize/bind human PTK7 with high affinity, can enter PTK7 expression cells with high efficiency in an endocytic way, and can be used in corresponding treatment and diagnosis fields. Thus, the following present invention was realized.

### Antibody of the Present Invention

In one aspect, the present invention provides a specific PTK7-binding antibody or antigen-binding fragment thereof, and the antibody or antigen-binding fragment thereof comprises the following complementarity determining regions (CDRs):
(a) CDR-H1, CDR-H2 and CDR-H3 contained in a heavy chain variable region (VH) shown as SEQ ID NO: 19; and/or CDR-L1, CDR-L2 and CDR-L3 contained in a light chain variable region (VL) shown as SEQ ID NO: 20;
(b) CDR-H1, CDR-H2 and CDR-H3 contained in a heavy chain variable region (VH) shown as SEQ ID NO: 1; and/or CDR-L1, CDR-L2 and CDR-L3 contained in a light chain variable region (VL) shown as SEQ ID NO: 2;
(c) CDR-H1, CDR-H2 and CDR-H3 contained in a heavy chain variable region (VH) shown as SEQ ID NO: 3; and/or CDR-L1, CDR-L2 and CDR-L3 contained in a light chain variable region (VL) shown as SEQ ID NO: 4;
(d) CDR-H1, CDR-H2 and CDR-H3 contained in a heavy chain variable region (VH) shown as SEQ ID NO: 5; and/or CDR-L1, CDR-L2 and CDR-L3 contained in a light chain variable region (VL) shown as SEQ ID NO: 6;
(e) CDR-H1, CDR-H2 and CDR-H3 contained in a heavy chain variable region (VH) shown as SEQ ID NO: 7; and/or CDR-L1, CDR-L2 and CDR-L3 contained in a light chain variable region (VL) shown as SEQ ID NO: 8;
(f) CDR-H1, CDR-H2 and CDR-H3 contained in a heavy chain variable region (VH) shown as SEQ ID NO: 9; and/or CDR-L1, CDR-L2 and CDR-L3 contained in a light chain variable region (VL) shown as SEQ ID NO: 10;
(g) CDR-H1, CDR-H2 and CDR-H3 contained in a heavy chain variable region (VH) shown as SEQ ID NO: 11; and/or CDR-L1, CDR-L2 and CDR-L3 contained in a light chain variable region (VL) shown as SEQ ID NO: 12;
(h) CDR-H1, CDR-H2 and CDR-H3 contained in a heavy chain variable region (VH) shown as SEQ ID NO: 13; and/or CDR-L1, CDR-L2 and CDR-L3 contained in a light chain variable region (VL) shown as SEQ ID NO: 14;
(i) CDR-H1, CDR-H2 and CDR-H3 contained in a heavy chain variable region (VH) shown as SEQ ID NO: 15; and/or CDR-L1, CDR-L2 and CDR-L3 contained in a light chain variable region (VL) shown as SEQ ID NO: 16;
(j) CDR-H1, CDR-H2 and CDR-H3 contained in a heavy chain variable region (VH) shown as SEQ ID NO: 17; and/or CDR-L1, CDR-L2 and CDR-L3 contained in a light chain variable region (VL) shown as SEQ ID NO: 18;
(k) CDR-H1, CDR-H2 and CDR-H3 contained in a heavy chain variable region (VH) shown as SEQ ID NO: 21; and/or CDR-L1, CDR-L2 and CDR-L3 contained in a light chain variable region (VL) shown as SEQ ID NO: 22;
(l) CDR-H1, CDR-H2 and CDR-H3 contained in a heavy chain variable region (VH) shown as SEQ ID NO: 23; and/or CDR-L1, CDR-L2 and CDR-L3 contained in a light chain variable region (VL) shown as SEQ ID NO: 24; or
(m) CDR-H1, CDR-H2 and CDR-H3 contained in the following heavy chain variable region (VH), and/or CDR-L1, CDR-L2 and CDR-L3 contained in the following light chain variable region (VL); at least one CDR of the heavy chain variable region (VH) and/or the light chain variable region (VL) contains a mutation as compared to any one of the heavy chain variable regions (VH) and/or the light chain variable regions (VL) in (a) to (1), and the mutation refers to replacement, deletion or addition of one or several amino acids (for example, replacement, deletion or addition of 1, 2 or 3 amino acids); preferably, the replacement is conservative replacement.

In certain embodiments, the replacement is conservative replacement.

In certain embodiments, the CDR is defined according to an IMGT, Kabat, Chothia or AbM numbering system.

In certain embodiments, the PTK7 includes human PTK7 and/or monkey PTK7. In certain embodiments, the monkey is Macaca mulatta.

In certain embodiments, the antibody or antigen-binding fragment thereof of the present invention comprises the heavy chain variable region (VH) and/or the light chain variable region (VL), and the CDR is defined according to the Chothia numbering system as follows:
(1a) a heavy chain variable region (VH) comprising the following 3 CDRs: CDR-H1 having a sequence shown as SEQ ID NO: 27 or a variant thereof; CDR-H2 having a sequence shown as SEQ ID NO: 28 or a variant thereof; and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(1b) a heavy chain variable region (VH) comprising the following 3 CDRs: CDR-H1 having a sequence shown as SEQ ID NO: 41 or a variant thereof; CDR-H2 having a sequence shown as SEQ ID NO: 42 or a variant thereof; and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(1c) a heavy chain variable region (VH) comprising the following 3 CDRs: CDR-H1 having a sequence shown as SEQ ID NO: 55 or a variant thereof; CDR-H2 having a sequence shown as SEQ ID NO: 56 or a variant thereof; and CDR-H3 having a sequence shown as SEQ ID NO: 57 or a variant thereof; and/or, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof;
(1d) a heavy chain variable region (VH) comprising the following 3 CDRs: CDR-H1 having a sequence shown as SEQ ID NO: 68 or a variant thereof; CDR-H2 having a sequence shown as SEQ ID NO: 69 or a variant thereof; and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof;
(1e) a heavy chain variable region (VH) comprising the following 3 CDRs: CDR-H1 having a sequence shown as SEQ ID NO: 74 or a variant thereof; CDR-H2 having a sequence shown as SEQ ID NO: 69 or a variant thereof; and CDR-H3 having a sequence shown as SEQ ID NO: 83 or a variant thereof; and/or, a light chain variable region (VL) comprising the following 3 CDRs: CDR-L1 having a sequence shown as SEQ ID NO: 84 or a variant thereof; CDR-L2 having a sequence shown as SEQ ID NO: 85 or a variant thereof; and CDR-L3 having a sequence shown as SEQ ID NO: 86 or a variant thereof;
(1f) a heavy chain variable region (VH) comprising the following 3 CDRs: CDR-H1 having a sequence shown as SEQ ID NO: 92 or a variant thereof; CDR-H2 having a sequence shown as SEQ ID NO: 93 or a variant thereof; and CDR-H3 having a sequence shown as SEQ ID NO: 94 or a variant thereof; and/or, a light chain variable region (VL) comprising the following 3 CDRs: CDR-L1 having a sequence shown as SEQ ID NO: 95 or a variant thereof; CDR-L2 having a sequence shown as SEQ ID NO: 96 or a variant thereof; and CDR-L3 having a sequence shown as SEQ ID NO: 97 or a variant thereof;
(1g) a heavy chain variable region (VH) comprising the following 3 CDRs: CDR-H1 having a sequence shown as SEQ ID NO: 27 or a variant thereof; CDR-H2 having a sequence shown as SEQ ID NO: 28 or a variant thereof; and CDR-H3 having a sequence shown as SEQ ID NO: 105 or a variant thereof; and/or, a light chain variable region (VL) comprising the following 3 CDRs: CDR-L1 having a sequence shown as SEQ ID NO: 106 or a variant thereof; CDR-L2 having a sequence shown as SEQ ID NO: 31 or a variant thereof; and CDR-L3 having a sequence shown as SEQ ID NO: 107 or a variant thereof; (1h) a heavy chain variable region (VH) comprising the following 3 CDRs: CDR-H1 having a sequence shown as SEQ ID NO: 41 or a variant thereof; CDR-H2 having a sequence shown as SEQ ID NO: 42 or a variant thereof; and CDR-H3 having a sequence shown as SEQ ID NO: 43 or a variant thereof; and, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(1i) a heavy chain variable region (VH) comprising the following 3 CDRs: CDR-H1 having a sequence shown as SEQ ID NO: 41 or a variant thereof; CDR-H2 having a sequence shown as SEQ ID NO: 42 or a variant thereof; and CDR-H3 having a sequence shown as SEQ ID NO: 43 or a variant thereof; or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(1j) a heavy chain variable region (VH) comprising the following 3 CDRs: CDR-H1 having a sequence shown as SEQ ID NO: 27 or a variant thereof; CDR-H2 having a sequence shown as SEQ ID NO: 28 or a variant thereof; and CDR-H3 having a sequence shown as SEQ ID NO: 29 or a variant thereof; and, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(1k) a heavy chain variable region (VH) comprising the following 3 CDRs: CDR-H1 having a sequence shown as SEQ ID NO: 55 or a variant thereof; CDR-H2 having a sequence shown as SEQ ID NO: 56 or a variant thereof; and CDR-H3 having a sequence shown as SEQ ID NO: 57 or a variant thereof; and, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof; or
(1l) a heavy chain variable region (VH) comprising the following 3 CDRs: CDR-H1 having a sequence shown as SEQ ID NO: 74 or a variant thereof; CDR-H2 having a sequence shown as SEQ ID NO: 69 or a variant thereof; and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof; and
the variant in any one of (1a)-(1l) is subjected to replacement, deletion or addition of one or several amino acids (such as replacement, deletion or addition of 1, 2 or 3 amino acids) as compared to the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR is defined according to the Kabat numbering system as follows:
(2a) heavy chain variable region (VH) comprising the following 3 CDRs: CDR-H1 having a sequence shown as SEQ ID NO: 33 or a variant thereof; CDR-H2 having a sequence shown as SEQ ID NO: 34 or a variant thereof; and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(2b) a heavy chain variable region (VH) comprising the following 3 CDRs: CDR-H1 having a sequence shown as SEQ ID NO: 33 or a variant thereof; CDR-H2 having a sequence shown as SEQ ID NO: 35 or a variant thereof; and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(2c) a heavy chain variable region (VH) comprising the following 3 CDRs: CDR-H1 having a sequence shown as SEQ ID NO: 47 or a variant thereof; CDR-H2 having a sequence shown as SEQ ID NO: 49 or a variant thereof; and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(2d) a heavy chain variable region (VH) comprising the following 3 CDRs: CDR-H1 having a sequence shown as SEQ ID NO: 61 or a variant thereof; CDR-H2 having a sequence shown as SEQ ID NO: 62 or a variant thereof; and CDR-H3 having a sequence shown as SEQ ID NO: 57 or a variant thereof; and/or, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof;
(2e) a heavy chain variable region (VH) comprising the following 3 CDRs: CDR-H1 having a sequence shown as SEQ ID NO: 75 or a variant thereof; CDR-H2 having a sequence shown as SEQ ID NO: 76 or a variant thereof; and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof;
(2f) a heavy chain variable region (VH) comprising the following 3 CDRs: CDR-H1 having a sequence shown as SEQ ID NO: 87 or a variant thereof; CDR-H2 having a sequence shown as SEQ ID NO: 88 or a variant thereof; and CDR-H3 having a sequence shown as SEQ ID NO: 83 or a variant thereof; and/or, a light chain variable region (VL) comprising the following 3 CDRs: CDR-L1 having a sequence shown as SEQ ID NO: 84 or a variant thereof; CDR-L2 having a sequence shown as SEQ ID NO: 85 or a variant thereof; and CDR-L3 having a sequence shown as SEQ ID NO: 86 or a variant thereof;
(2g) a heavy chain variable region (VH) comprising the following 3 CDRs: CDR-H1 having a sequence shown as SEQ ID NO: 98 or a variant thereof; CDR-H2 having a sequence shown as SEQ ID NO: 99 or a variant thereof; and CDR-H3 having a sequence shown as SEQ ID NO: 94 or a variant thereof; and/or, a light chain variable region (VL) comprising the following 3 CDRs: CDR-L1 having a sequence shown as SEQ ID NO: 95 or a variant thereof; CDR-L2 having a sequence shown as SEQ ID NO: 96 or a variant thereof; and CDR-L3 having a sequence shown as SEQ ID NO: 97 or a variant thereof;
(2h) a heavy chain variable region (VH) comprising the following 3 CDRs: CDR-H1 having a sequence shown as SEQ ID NO: 108 or a variant thereof; CDR-H2 having a sequence shown as SEQ ID NO: 35 or a variant thereof; and CDR-H3 having a sequence shown as SEQ ID NO: 105 or a variant thereof; and/or, a light chain variable region (VL) comprising the following 3 CDRs: CDR-L1 having a sequence shown as SEQ ID NO: 106 or a variant thereof; CDR-L2 having a sequence shown as SEQ ID NO: 31 or a variant thereof; and CDR-L3 having a sequence shown as SEQ ID NO: 107 or a variant thereof;
(2i) a heavy chain variable region (VH) comprising the following 3 CDRs: CDR-H1 having a sequence shown as SEQ ID NO: 47 or a variant thereof; CDR-H2 having a sequence shown as SEQ ID NO: 48 or a variant thereof; and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(2j) a heavy chain variable region (VH) comprising the following 3 CDRs: CDR-H1 having a sequence shown as SEQ ID NO: 47 or a variant thereof; CDR-H2 having a sequence shown as SEQ ID NO: 48 or a variant thereof; and CDR-H3 having a sequence shown as SEQ ID NO: 43 or a variant thereof; and, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(2k) a heavy chain variable region (VH) comprising the following 3 CDRs: CDR-H1 having a sequence shown as SEQ ID NO: 61 or a variant thereof; CDR-H2 having a sequence shown as SEQ ID NO: 62 or a variant thereof; and CDR-H3 having a sequence shown as SEQ ID NO: 57 or a variant thereof; and, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof; or
(2l) a heavy chain variable region (VH) comprising the following 3 CDRs: CDR-H1 having a sequence shown as SEQ ID NO: 75 or a variant thereof; CDR-H2 having a sequence shown as SEQ ID NO: 76 or a variant thereof; and CDR-H3 having a sequence shown as SEQ ID NO: 70 or a variant thereof; and, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof;
the variant in any one of (2a)-(21) is subjected to replacement, deletion or addition of one or several amino acids (such as replacement, deletion or addition of 1, 2 or 3 amino acids) compared with the sequence from which the variant is derived; and in certain embodiments, the replacement is conservative replacement.

In certain embodiments, the antibody or antigen-binding fragment thereof according to the present invention comprises the heavy chain variable region (VH) and/or the light chain variable region (VL), and the CDR is defined according to the IMGT numbering system as follows:
(3a) a heavy chain variable region (VH) comprising the following 3 CDRs: CDR-H1 having a sequence shown as SEQ ID NO: 36 or a variant thereof; CDR-H2 having a sequence shown as SEQ ID NO: 37 or a variant thereof; and CDR-H3 having a sequence shown as SEQ ID NO: 38 or a variant thereof; and/or, a light chain variable region (VL) comprising the following 3 CDRs: CDR-L1 having a sequence shown as SEQ ID NO: 39 or a variant thereof; CDR-L2 having a sequence shown as SEQ ID NO: 40 or a variant thereof; and CDR-L3 having a sequence shown as SEQ ID NO: 32 or a variant thereof;
(3b) a heavy chain variable region (VH) comprising the following 3 CDRs: CDR-H1 having a sequence shown as SEQ ID NO: 50 or a variant thereof; CDR-H2 having a sequence shown as SEQ ID NO: 51 or a variant thereof; and CDR-H3 having a sequence shown as SEQ ID NO: 52 or a variant thereof; and/or, a light chain variable region (VL) comprising the following 3 CDRs: CDR-L1 having a sequence shown as SEQ ID NO: 53 or a variant thereof; CDR-L2 having a sequence shown as SEQ ID NO: 54 or a variant thereof; and CDR-L3 having a sequence shown as SEQ ID NO: 46 or a variant thereof;
(3c) a heavy chain variable region (VH) comprising the following 3 CDRs: CDR-H1 having a sequence shown as SEQ ID NO: 63 or a variant thereof; CDR-H2 having a sequence shown as SEQ ID NO: 64 or a variant thereof; and CDR-H3 having a sequence shown as SEQ ID NO: 65 or a variant thereof; and/or, a light chain variable region (VL) comprising the following 3 CDRs: CDR-L1 having a sequence shown as SEQ ID NO: 66 or a variant thereof; CDR-L2 having a sequence shown as SEQ ID NO: 67 or a variant thereof; and CDR-L3 having a sequence shown as SEQ ID NO: 60 or a variant thereof;
(3d) a heavy chain variable region (VH) comprising the following 3 CDRs: CDR-H1 having a sequence shown as SEQ ID NO: 77 or a variant thereof; CDR-H2 having a sequence shown as SEQ ID NO: 78 or a variant thereof; and CDR-H3 having a sequence shown as SEQ ID NO: 79 or a variant thereof; and/or, a light chain variable region (VL) comprising the following 3 CDRs: CDR-L1 having a sequence shown as SEQ ID NO: 81 or a variant thereof; CDR-L2 having a sequence shown as SEQ ID NO: 82 or a variant thereof; and CDR-L3 having a sequence shown as SEQ ID NO: 73 or a variant thereof;
(3e) a heavy chain variable region (VH) comprising the following 3 CDRs: CDR-H1 having a sequence shown as SEQ ID NO: 80 or a variant thereof; CDR-H2 having a sequence shown as SEQ ID NO: 78 or a variant thereof; and CDR-H3 having a sequence shown as SEQ ID NO: 89 or a variant thereof; and/or, a light chain variable region (VL) comprising the following 3 CDRs: CDR-L1 having a sequence shown as SEQ ID NO: 90 or a variant thereof; CDR-L2 having a sequence shown as SEQ ID NO: 91 or a variant thereof; and CDR-L3 having a sequence shown as SEQ ID NO: 86 or a variant thereof;
(3f) a heavy chain variable region (VH) comprising the following 3 CDRs: CDR-H1 having a sequence shown as SEQ ID NO: 100 or a variant thereof; CDR-H2 having a sequence shown as SEQ ID NO: 101 or a variant thereof; and CDR-H3 having a sequence shown as SEQ ID NO: 102 or a variant thereof; and/or, a light chain variable region (VL) comprising the following 3 CDRs: CDR-L1 having a sequence shown as SEQ ID NO: 103 or a variant thereof; CDR-L2 having a sequence shown as SEQ ID NO: 104 or a variant thereof; and CDR-L3 having a sequence shown as SEQ ID NO: 97 or a variant thereof;
(3g) a heavy chain variable region (VH) comprising the following 3 CDRs: CDR-H1 having a sequence shown as SEQ ID NO: 36 or a variant thereof; CDR-H2 having a sequence shown as SEQ ID NO: 37 or a variant thereof; and CDR-H3 having a sequence shown as SEQ ID NO: 109 or a variant thereof; and/or, a light chain variable region (VL) comprising the following 3 CDRs: CDR-L1 having a sequence shown as SEQ ID NO: 110 or a variant thereof; CDR-L2 having a sequence shown as SEQ ID NO: 40 or a variant thereof; and CDR-L3 having a sequence shown as SEQ ID NO: 107 or a variant thereof;
(3h) a heavy chain variable region (VH) comprising the following 3 CDRs: CDR-H1 having a sequence shown as SEQ ID NO: 50 or a variant thereof; CDR-H2 having a sequence shown as SEQ ID NO: 51 or a variant thereof; and CDR-H3 having a sequence shown as SEQ ID NO: 52 or a variant thereof; and, a light chain variable region (VL) comprising the following 3 CDRs: CDR-L1 having a sequence shown as SEQ ID NO: 53 or a variant thereof; CDR-L2 having a sequence shown as SEQ ID NO: 54 or a variant thereof; and CDR-L3 having a sequence shown as SEQ ID NO: 46 or a variant thereof;
(3i) a heavy chain variable region (VH) comprising the following 3 CDRs: CDR-H1 having a sequence shown as SEQ ID NO: 50 or a variant thereof; CDR-H2 having a sequence shown as SEQ ID NO: 51 or a variant thereof; and CDR-H3 having a sequence shown as SEQ ID NO: 52 or a variant thereof; or, a light chain variable region (VL) comprising the following 3 CDRs: CDR-L1 having a sequence shown as SEQ ID NO: 53 or a variant thereof; CDR-L2 having a sequence shown as SEQ ID NO: 54 or a variant thereof; and CDR-L3 having a sequence shown as SEQ ID NO: 46 or a variant thereof;
(3j) a heavy chain variable region (VH) comprising the following 3 CDRs: CDR-H1 having a sequence shown as SEQ ID NO: 36 or a variant thereof; CDR-H2 having a sequence shown as SEQ ID NO: 37 or a variant thereof; and CDR-H3 having a sequence shown as SEQ ID NO: 38 or a variant thereof; and, a light chain variable region (VL) comprising the following 3 CDRs: CDR-L1 having a sequence shown as SEQ ID NO: 39 or a variant thereof; CDR-L2 having a sequence shown as SEQ ID NO: 40 or a variant thereof; and CDR-L3 having a sequence shown as SEQ ID NO: 32 or a variant thereof;
(3k) a heavy chain variable region (VH) comprising the following 3 CDRs: CDR-H1 having a sequence shown as SEQ ID NO: 63 or a variant thereof; CDR-H2 having a sequence shown as SEQ ID NO: 64 or a variant thereof; and CDR-H3 having a sequence shown as SEQ ID NO: 65 or a variant thereof; and, a light chain variable region (VL) comprising the following 3 CDRs: CDR-L1 having a sequence shown as SEQ ID NO: 66 or a variant thereof; CDR-L2 having a sequence shown as SEQ ID NO: 67 or a variant thereof; and CDR-L3 having a sequence shown as SEQ ID NO: 60 or a variant thereof; or
(3l) a heavy chain variable region (VH) comprising the following 3 CDRs: CDR-H1 having a sequence shown as SEQ ID NO: 80 or a variant thereof; CDR-H2 having a sequence shown as SEQ ID NO: 78 or a variant thereof; and CDR-H3 having a sequence shown as SEQ ID NO: 79 or a variant thereof; and/or, a light chain variable region (VL) comprising the following 3 CDRs: CDR-L1 having a sequence shown as SEQ ID NO: 81 or a variant thereof; CDR-L2 having a sequence shown as SEQ ID NO: 82 or a variant thereof; and CDR-L3 having a sequence shown as SEQ ID NO: 73 or a variant thereof;
the variant in any one of (3a)-(3l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR is defined according to the AbM numbering system as follows:
(4a) a heavy chain variable region (VH) comprising the following 3 CDRs: CDR-H1 having a sequence shown as SEQ ID NO: 113 or a variant thereof; CDR-H2 having a sequence shown as SEQ ID NO: 114 or a variant thereof; and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(4b) a heavy chain variable region (VH) comprising the following 3 CDRs: CDR-H1 having a sequence shown as SEQ ID NO: 115 or a variant thereof; CDR-H2 having a sequence shown as SEQ ID NO: 116 or a variant thereof; and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(4c) a heavy chain variable region (VH) comprising the following 3 CDRs: CDR-H1 having a sequence shown as SEQ ID NO: 117 or a variant thereof; CDR-H2 having a sequence shown as SEQ ID NO: 118 or a variant thereof; and CDR-H3 having a sequence shown as SEQ ID NO: 57 or a variant thereof; and/or, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof;
(4d) a heavy chain variable region (VH) comprising the following 3 CDRs: CDR-H1 having a sequence shown as SEQ ID NO: 119 or a variant thereof; CDR-H2 having a sequence shown as SEQ ID NO: 120 or a variant thereof; and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof;
(4e) a heavy chain variable region (VH) comprising the following 3 CDRs: CDR-H1 having a sequence shown as SEQ ID NO: 122 or a variant thereof; CDR-H2 having a sequence shown as SEQ ID NO: 123 or a variant thereof; and CDR-H3 having a sequence shown as SEQ ID NO: 83 or a variant thereof; and/or, a light chain variable region (VL) comprising the following 3 CDRs: CDR-L1 having a sequence shown as SEQ ID NO: 84 or a variant thereof; CDR-L2 having a sequence shown as SEQ ID NO: 85 or a variant thereof; and CDR-L3 having a sequence shown as SEQ ID NO: 86 or a variant thereof;
(4f) a heavy chain variable region (VH) comprising the following 3 CDRs: CDR-H1 having a sequence shown as SEQ ID NO: 124 or a variant thereof; CDR-H2 having a sequence shown as SEQ ID NO: 125 or a variant thereof; and CDR-H3 having a sequence shown as SEQ ID NO: 94 or a variant thereof; and/or, a light chain variable region (VL) comprising the following 3 CDRs: CDR-L1 having a sequence shown as SEQ ID NO: 95 or a variant thereof; CDR-L2 having a sequence shown as SEQ ID NO: 96 or a variant thereof; and CDR-L3 having a sequence shown as SEQ ID NO: 97 or a variant thereof;
(4g) a heavy chain variable region (VH) comprising the following 3 CDRs: CDR-H1 having a sequence shown as SEQ ID NO: 126 or a variant thereof; CDR-H2 having a sequence shown as SEQ ID NO: 114 or a variant thereof; and CDR-H3 having a sequence shown as SEQ ID NO: 105 or a variant thereof; and/or, a light chain variable region (VL) comprising the following 3 CDRs: CDR-L1 having a sequence shown as SEQ ID NO: 106 or a variant thereof; CDR-L2 having a sequence shown as SEQ ID NO: 31 or a variant thereof; and CDR-L3 having a sequence shown as SEQ ID NO: 107 or a variant thereof;
(4h) a heavy chain variable region (VH) comprising the following 3 CDRs: CDR-H1 having a sequence shown as SEQ ID NO: 115 or a variant thereof; CDR-H2 having a sequence shown as SEQ ID NO: 116 or a variant thereof; and CDR-H3 having a sequence shown as SEQ ID NO: 43 or a variant thereof; and, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(4i) a heavy chain variable region (VH) comprising the following 3 CDRs: CDR-H1 having a sequence shown as SEQ ID NO: 115 or a variant thereof; CDR-H2 having a sequence shown as SEQ ID NO: 116 or a variant thereof; and CDR-H3 having a sequence shown as SEQ ID NO: 43 or a variant thereof; or, a light chain variable region (VL) comprising the following 3 CDRs: CDR-L1 having a sequence shown as SEQ ID NO: 44 or a variant thereof; CDR-L2 having a sequence shown as SEQ ID NO: 45 or a variant thereof; and CDR-L3 having a sequence shown as SEQ ID NO: 46 or a variant thereof;
(4j) a heavy chain variable region (VH) comprising the following 3 CDRs: CDR-H1 having a sequence shown as SEQ ID NO: 113 or a variant thereof; CDR-H2 having a sequence shown as SEQ ID NO: 114 or a variant thereof; and CDR-H3 having a sequence shown as SEQ ID NO: 29 or a variant thereof; and, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(4k) a heavy chain variable region (VH) comprising the following 3 CDRs: CDR-H1 having a sequence shown as SEQ ID NO: 117 or a variant thereof; CDR-H2 having a sequence shown as SEQ ID NO: 118 or a variant thereof; and CDR-H3 having a sequence shown as SEQ ID NO: 57 or a variant thereof; and, a light chain variable region (VL) comprising the following 3 CDRs: CDR-L1 having a sequence shown as SEQ ID NO: 58 or a variant thereof; CDR-L2 having a sequence shown as SEQ ID NO: 59 or a variant thereof; and CDR-L3 having a sequence shown as SEQ ID NO: 60 or a variant thereof; or
(4l) a heavy chain variable region (VH) comprising the following 3 CDRs: CDR-H1 having a sequence shown as SEQ ID NO: 121 or a variant thereof; CDR-H2 having a sequence shown as SEQ ID NO: 120 or a variant thereof; and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof; CDR-L2 having a sequence shown as SEQ ID NO: 72 or a variant thereof; and CDR-L3 having a sequence shown as SEQ ID NO: 73 or a variant thereof;
the variant in any one of (4a)-(4l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof includes framework regions (FRs) from human immunoglobulin.

In certain embodiments, the antibody or antigen-binding fragment thereof according to the present invention comprises:
(a) a VH having a sequence shown as SEQ ID NO: 19 or a variant thereof and/or a VL having a sequence shown as SEQ ID NO: 20 or a variant thereof;
(b) a VH having a sequence shown as SEQ ID NO: 1 or a variant thereof and/or a VL having a sequence shown as SEQ ID NO: 2 or a variant thereof;
(c) a VH having a sequence shown as SEQ ID NO: 3 or a variant thereof and/or a VL having a sequence shown as SEQ ID NO: 4 or a variant thereof;
(d) a VH having a sequence shown as SEQ ID NO: 5 or a variant thereof and/or a VL having a sequence shown as SEQ ID NO: 6 or a variant thereof;
(e) a VH having a sequence shown as SEQ ID NO: 7 or a variant thereof and/or a VL having a sequence shown as SEQ ID NO: 8 or a variant thereof;
(f) a VH having a sequence shown as SEQ ID NO: 9 or a variant thereof and/or a VL having a sequence shown as SEQ ID NO: 10 or a variant thereof;
(g) a VH having a sequence shown as SEQ ID NO: 11 or a variant thereof and/or a VL having a sequence shown as SEQ ID NO: 12 or a variant thereof;
(h) a VH having a sequence shown as SEQ ID NO: 13 or a variant thereof and/or a VL having a sequence shown as SEQ ID NO: 14 or a variant thereof;
(i) a VH having a sequence shown as SEQ ID NO: 15 or a variant thereof and/or a VL having a sequence shown as SEQ ID NO: 16 or a variant thereof;
(j) a VH having a sequence shown as SEQ ID NO: 17 or a variant thereof and/or a VL having a sequence shown as SEQ ID NO: 18 or a variant thereof;
(k) a VH having a sequence shown as SEQ ID NO: 21 or a variant thereof and/or a VL having a sequence shown as SEQ ID NO: 22 or a variant thereof; or
(l) a VH having a sequence shown as SEQ ID NO: 23 or a variant thereof and/or a VL having a sequence shown as SEQ ID NO: 24 or a variant thereof;
the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement.

In certain embodiments, the antibody or antigen-binding fragment according to any one of the above embodiments further has characteristics selected from the following:
(1) binding PTK7 (such as human or monkey PTK7) with EC₅₀ of less than about 50 ng/mL, such as less than about 40 ng/mL, 30 ng/mL, 25 ng/mL, 24 ng/mL, 23 ng/mL, 22 ng/mL, 21 ng/mL, 20 ng/mL, 19 ng/mL, 18 ng/mL, 17 ng/mL, 16 ng/mL, 15 ng/mL, 14 ng/mL, 13 ng/mL, 12 ng/mL, 11 ng/mL, 10 ng/mL, 9 ng/mL, 8 ng/mL, 7 ng/mL, 6 ng/mL or less; preferably, the EC₅₀ is measured by ELISA;
(2) binding PTK7 (such as human or monkey PTK7) with KD of less than about 100 nM, such as less than about 90 nM, 80 nM, 70 nM, 60 nM, 50 nM, 40 nM, 30 nM, 20 nM, 15 nM, 10 nM, 5 nM, 4 nM, 3 nM, 2 nM, 1 nM or less; preferably, the KD is measured by bio-layer interferometry (BLI) (such as ForteBio Octet®);
(3) having ADCC and CDC activities, such as inducing killing of cells (like tumor cells) expressing PTK7 by ADCC and CDC; and in certain embodiments, the antibody or antigen-binding fragment comprises a wild-type Fc region. In certain embodiments, the antibody or antigen-binding fragment comprises the heavy chain constant region (CH) shown as SEQ ID NO: 25.
or, not having ADCC and CDC activities; in certain embodiments, the antibody or antigen-binding fragment comprises a mutation-containing or chemically modified Fc region. In certain embodiments, the antibody or antigen-binding fragment comprises the heavy chain constant region (CH) shown as SEQ ID NO: 131.
(4) inducing PTK7 internalization, such as, measuring by flow cytometry;
(5) inhibiting cell proliferation; and/or
(6) inhibiting tumor growth.

In certain embodiments, the antibody or antigen-binding fragment according to any one of the above embodiments may comprise a constant region from or deviated from human immunoglobulin.

In certain embodiments, a heavy chain of the antibody or antigen-binding fragment thereof comprises a heavy chain constant region from or derived from human immunoglobulin (such as IgG1, IgG2, IgG3 or IgG4). In certain embodiments, the antibody or antigen-binding fragment thereof comprises a wild-type Fc region, or comprises a mutation-containing or chemically modified Fc region which has a changed effector function (such as improved ADCC activity) as compared to the wild-type Fc region. In certain embodiments, the heavy chain of the antibody or antigen-binding fragment thereof comprises the sequence shown as SEQ ID NO: 25 or a variant thereof, and the variant is subjected to replacement of up to 20 amino acids (such as replacement of up to 15, up to 10 or up to 5 amino acids, like replacement of 1, 2, 3, 4 or 5 amino acids) as compared to SEQ ID NO: 25. In certain embodiments, the replacement is conservative replacement.

In certain embodiments, the variant is subjected to replacement of 3 amino acids as compared to SEQ ID NO: 25. In certain embodiments, the variant is subjected to replacement of amino acids at positions corresponding to positions 117, 118 and 120 in SEQ ID NO: 25. In certain embodiments, the variant is subjected to replacement of alanine at positions corresponding to positions 117, 118 and 120 in SEQ ID NO: 25. In certain embodiments, the variant has the heavy chain constant region (CH) shown as SEQ ID NO: 131.

In certain embodiments, a light chain of the antibody or antigen-binding fragment thereof comprises a light chain constant region from or derived from human immunoglobulin (such as κ or λ). In certain embodiments, the light chain of the antibody or antigen-binding fragment thereof comprises a sequence shown as SEQ ID NO: 26 and a variant thereof, and the variant is subjected to replacement of up to 20 amino acids (such as replacement of up to 15, up to 10 or up to 5 amino acids, like replacement of 1, 2, 3, 4 or 5 amino acids) as compared to SEQ ID NO: 26. In certain embodiments, the replacement is conservative replacement.

In certain embodiments, the antibody or an antigen-binding fragment thereof comprises the heavy chain constant region (CH) shown as SEQ ID NO: 25 and the light chain constant region (CL) shown as SEQ ID NO: 26.

In certain embodiments, the antibody or an antigen-binding fragment thereof comprises the heavy chain constant region (CH) shown as SEQ ID NO: 131 and the light chain constant region (CL) shown as SEQ ID NO: 26.

In certain embodiments, the antibody or antigen-binding fragment thereof having the heavy chain constant region is not bound to an Fc receptor (like CD16a protein, CD32a protein, CD32b protein and CD64 protein). Therefore, in such embodiments, the antibody or antigen-binding fragment thereof may reduce Fc receptor-mediated nonspecific cytotoxicity and improve the safety of the antibody or antigen-binding fragment thereof in the subject. In certain embodiments, the antibody or antigen-binding fragment thereof having the heavy chain constant region may be bound to FcRn. Therefore, in such embodiments, the antibody or antigen-binding fragment thereof has a long half-life in the subject. In certain embodiments, the antibody or antigen-binding fragment thereof having the heavy chain constant region may induce PTK7-mediated endocytosis. Therefore, in such embodiments, the antibody or antigen-binding fragment thereof has the potential as a vector for targeted delivery of drugs, toxins, enzymes or DNA for treatment.

In certain embodiments, the antibody or antigen-binding fragment thereof according to the present invention comprises:
(a) a heavy chain comprising the VH shown as SEQ ID NO: 19 and the heavy chain constant region (CH) shown as SEQ ID NO: 131, and, a light chain comprising VL having the sequence shown as SEQ ID NO: 20 and the light chain constant region (CL) shown as SEQ ID NO: 26;
(b) a heavy chain comprising the VH shown as SEQ ID NO: 19 and the heavy chain constant region (CH) shown as SEQ ID NO: 25, and, a light chain comprising VL having the sequence shown as SEQ ID NO: 20 and the light chain constant region (CL) shown as SEQ ID NO: 26;
(c) a heavy chain comprising the VH shown as SEQ ID NO: 1 and the heavy chain constant region (CH) shown as SEQ ID NO: 25, and, a light chain comprising VL having the sequence shown as SEQ ID NO: 2 and the light chain constant region (CL) shown as SEQ ID NO: 26;
(d) a heavy chain comprising the VH shown as SEQ ID NO: 1 and the heavy chain constant region (CH) shown as SEQ ID NO: 131, and, a light chain comprising VL having the sequence shown as SEQ ID NO: 2 and the light chain constant region (CL) shown as SEQ ID NO: 26;
(e) a heavy chain comprising the VH shown as SEQ ID NO: 3 and the heavy chain constant region (CH) shown as SEQ ID NO: 25, and, a light chain comprising VL having the sequence shown as SEQ ID NO: 4 and the light chain constant region (CL) shown as SEQ ID NO: 26;
(f) a heavy chain comprising the VH shown as SEQ ID NO: 5 and the heavy chain constant region (CH) shown as SEQ ID NO: 25, and, a light chain comprising VL having the sequence shown as SEQ ID NO: 6 and the light chain constant region (CL) shown as SEQ ID NO: 26;
(g) a heavy chain comprising the VH shown as SEQ ID NO: 7 and the heavy chain constant region (CH) shown as SEQ ID NO: 25, and, a light chain comprising VL having the sequence shown as SEQ ID NO: 8 and the light chain constant region (CL) shown as SEQ ID NO: 26;
(h) a heavy chain comprising the VH shown as SEQ ID NO: 9 and the heavy chain constant region (CH) shown as SEQ ID NO: 25, and, a light chain comprising VL having the sequence shown as SEQ ID NO: 10 and the light chain constant region (CL) shown as SEQ ID NO: 26;
(i) a heavy chain comprising the VH shown as SEQ ID NO: 11 and the heavy chain constant region (CH) shown as SEQ ID NO: 25, and, a light chain comprising VL having the sequence shown as SEQ ID NO: 12 and the light chain constant region (CL) shown as SEQ ID NO: 26;
(j) a heavy chain comprising the VH shown as SEQ ID NO: 13 and the heavy chain constant region (CH) shown as SEQ ID NO: 25, and, a light chain comprising VL having the sequence shown as SEQ ID NO: 14 and the light chain constant region (CL) shown as SEQ ID NO: 26;
(k) a heavy chain comprising the VH shown as SEQ ID NO: 15 and the heavy chain constant region (CH) shown as SEQ ID NO: 25, and, a light chain comprising VL having the sequence shown as SEQ ID NO: 16 and the light chain constant region (CL) shown as SEQ ID NO: 26;
(l) a heavy chain comprising the VH shown as SEQ ID NO: 17 and the heavy chain constant region (CH) shown as SEQ ID NO: 25, and, a light chain comprising VL having the sequence shown as SEQ ID NO: 18 and the light chain constant region (CL) shown as SEQ ID NO: 26;
(m) a heavy chain comprising the VH shown as SEQ ID NO: 21 and the heavy chain constant region (CH) shown as SEQ ID NO: 25, and, a light chain comprising VL having the sequence shown as SEQ ID NO: 22 and the light chain constant region (CL) shown as SEQ ID NO: 26;
(n) a heavy chain comprising the VH shown as SEQ ID NO: 23 and the heavy chain constant region (CH) shown as SEQ ID NO: 25, and, a light chain comprising VL having the sequence shown as SEQ ID NO: 24 and the light chain constant region (CL) shown as SEQ ID NO: 26;
(o) a heavy chain comprising the VH shown as SEQ ID NO: 3 and the heavy chain constant region (CH) shown as SEQ ID NO: 131, and, a light chain comprising VL having the sequence shown as SEQ ID NO: 4 and the light chain constant region (CL) shown as SEQ ID NO: 26;
(p) a heavy chain comprising the VH shown as SEQ ID NO: 5 and the heavy chain constant region (CH) shown as SEQ ID NO: 131, and, a light chain comprising VL having the sequence shown as SEQ ID NO: 6 and the light chain constant region (CL) shown as SEQ ID NO: 26;
(q) a heavy chain comprising the VH shown as SEQ ID NO: 7 and the heavy chain constant region (CH) shown as SEQ ID NO: 131, and, a light chain comprising VL having the sequence shown as SEQ ID NO: 8 and the light chain constant region (CL) shown as SEQ ID NO: 26;
(r) a heavy chain comprising the VH shown as SEQ ID NO: 9 and the heavy chain constant region (CH) shown as SEQ ID NO: 131, and, a light chain comprising VL having the sequence shown as SEQ ID NO: 10 and the light chain constant region (CL) shown as SEQ ID NO: 26;
(s) a heavy chain comprising the VH shown as SEQ ID NO: 11 and the heavy chain constant region (CH) shown as SEQ ID NO: 131, and, a light chain comprising VL having the sequence shown as SEQ ID NO: 12 and the light chain constant region (CL) shown as SEQ ID NO: 26;
(t) a heavy chain comprising the VH shown as SEQ ID NO: 13 and the heavy chain constant region (CH) shown as SEQ ID NO: 131, and, a light chain comprising VL having the sequence shown as SEQ ID NO: 14 and the light chain constant region (CL) shown as SEQ ID NO: 26;
(u) a heavy chain comprising the VH shown as SEQ ID NO: 15 and the heavy chain constant region (CH) shown as SEQ ID NO: 131, and, a light chain comprising VL having the sequence shown as SEQ ID NO: 16 and the light chain constant region (CL) shown as SEQ ID NO: 26;
(v) a heavy chain comprising the VH shown as SEQ ID NO: 17 and the heavy chain constant region (CH) shown as SEQ ID NO: 131, and, a light chain comprising VL having the sequence shown as SEQ ID NO: 18 and the light chain constant region (CL) shown as SEQ ID NO: 26;
(w) a heavy chain comprising the VH shown as SEQ ID NO: 21 and the heavy chain constant region (CH) shown as SEQ ID NO: 131, and, a light chain comprising VL having the sequence shown as SEQ ID NO: 22 and the light chain constant region (CL) shown as SEQ ID NO: 26; or
(x) a heavy chain comprising the VH shown as SEQ ID NO: 23 and the heavy chain constant region (CH) shown as SEQ ID NO: 131, and, a light chain comprising VL having the sequence shown as SEQ ID NO: 24 and the light chain constant region (CL) shown as SEQ ID NO: 26.

In certain embodiments, the antibody or antigen-binding fragment thereof according to the present invention is a murine antibody, a chimeric antibody, a humanized antibody or a fully humanized antibody.

In certain embodiments, the antibody or antigen-binding fragment thereof according to any one of the above embodiments is selected from scFv, Fab, Fab′, (Fab′)2, Fab′-SH, an Fv fragment, disulfide bond linked Fv (dsFv), a diabody, a bispecific antibody and a multispecific antibody.

### Derived Antibody

The antibody or the antigen-binding fragment thereof according to the present invention may be derivatized, e.g., the antibody or antigen-binding fragment thereof may be linked to another molecule (e.g., another polypeptide or protein). Typically, derivatization (like marking) of the antibody or antigen-binding fragment thereof will not adversely affect its binding to PTK7 (particularly human PTK7). Therefore, the antibody or antigen-binding fragment thereof according to the present invention are also contemplated to include such derivatized forms. For example, the antibody or antigen-binding fragment thereof according to the present invention may be functionally linked (by chemical coupling, gene fusion, non-covalent linkage, or otherwise) to one or more other molecule groups, e.g., another antibody (like forming a bispecific antibody), a detection reagent, a pharmaceutical reagent, and/or a protein or polypeptide (like an avidin or a polyhistidine tag) capable of mediating binding of an antibody or antigen-binding fragment to another molecule.

As one of derivatives of the antibody, a conjugate according to the present invention includes the antibody or antigen-binding fragment thereof and the coupling part according to the present invention.

In certain embodiments, the coupling part is selected from a detectable marker. The detectable marker according to the present invention may be any substance which can be detected by means of fluorescence, spectrum, photochemistry, biochemistry, immunology, electricity, optics or chemistry. Such marker is well-known in the art, and the examples of the marker include, but are not limited to, enzymes (like horseradish peroxidase, alkaline phosphatase, β-galactosidase, urease, glucose oxidase), radionuclides (like 3H, 1251, 35S, 14C or 32P), fluorochrome (like fluorescein isothiocyanate (FITC), fluorescein, tetramethylrhodamine isothiocyanate (TRITC), phycoerythrin (PE), texas red, rhodamine, quantum dots or cyanine dye derivatives (like Cy7 and Alexa 750)), acridinium ester compounds, magnetic beads (like Dynabeads®), thermal measurement markers such as colloidal gold or colored glass or plastic (like polystyrene, polypropylene and latex) beads, and biotin for binding the above marker-modified avidins (like streptavidin). In certain embodiments, such marker can be used for immunological detection (like enzyme linked immunoassay, radioimmunoassay, fluorescence immunoassay and chemiluminescence immunoassay). In certain embodiments, the detectable marker is selected from radioisotopes, fluorescent substances, luminescent substances, colored substances or enzymes. In certain embodiments, the detectable marker described above may be linked to the antibody or antigen-binding fragment thereof according to the present invention by linkers of different lengths so as to reduce potential steric hindrance.

In certain embodiments, the coupling part is selected from a therapeutic reagent. In certain embodiments, the therapeutic reagent is preferably an anti-tumor reagent, such as a cytotoxic reagent, cytokine, toxin or radionuclide.

In certain embodiments, the coupling part is selected from a substance which can improve the biological properties of the antibody (e.g., increasing serum half-life), such as a chemical group like polyethylene glycol (PEG), methyl or ethyl, or a glycosyl group.

As one of the derivatives of the antibody, a multispecific antibody according to the present invention comprises the antibody or antigen-binding fragment thereof according to the present invention.

In certain embodiments, the multispecific antibody comprises the antibody or antigen-binding fragment thereof according to the present invention as a first antigen-binding domain, and further comprises at least one second antigen-binding domain for other targets.

In certain embodiments, each antigen-binding domain of the multispecific antibody retains a respective original binding specificity.

In certain embodiments, the multispecific antibody is a bispecific antibody or a trispecific antibody or a tetraspecific antibody.

### Preparation of Antibody

The antibody according to the present invention may be prepared by various methods known in the art, such as by genetic engineering recombination technology. For example, DNA molecules encoding the heavy chain and light chain genes of the antibody according to the present invention may be obtained by chemical synthesis or PCR amplification. The obtained DNA molecules are inserted into an expression vector and then transfected into a host cell. Then, the transfected host cell is cultured under specific conditions and then the antibody according to the present invention is expressed.

The antigen-binding fragments according to the present invention may be obtained by hydrolyzing complete antibody molecules (see Morimoto et al., J. Biochem. Biophys. Methods 24:107-117 (1992) and Brennan et al., Science 229:81 (1985)). In addition, these antigen-binding fragments may also be directly generated by recombinant host cells (reviewed in Hudson, Curr. Opin. Immunol. 11: 548-557 (1999); Little et al., Immunol. Today, 21: 364-370 (2000)). For example, a Fab′ fragment may be directly obtained from the host cell; the Fab′ fragment may be chemically coupled to form an F(ab′)₂ fragment (Carter et al., Bio/Technology, 10: 163-167 (1992)). In addition, Fv, Fab or F(ab′)₂ fragments may also be directly separated from a recombinant host cell culture solution. Ordinary technicians in the art are fully aware of other techniques for preparing these antigen-binding fragments.

Therefore, in another aspect, the present invention provides isolated nucleic acid molecules comprising a nucleotide sequence encoding the antibody or antigen-binding fragment thereof according to the present invention, or the heavy chain variable region and/or light chain variable region thereof. In certain embodiments, the nucleotide sequence is substitutable according to the codon degeneracy in the art. In certain embodiments, the nucleotide sequence is codon-optimized.

In certain embodiments, the isolated nucleic acid molecules comprise nucleic acid molecules encoding the heavy chain variable region of the antibody, and/or nucleic acid molecules encoding the light chain variable region of the antibody;
the nucleic acid molecules encoding the heavy chain variable region of the antibody comprise: (i) a nucleotide sequence shown as SEQ ID NO: 127, (ii) a sequence substantially identical to SEQ ID NO: 127 (e.g., a sequence having at least about 85%, 90%, 95%, 99% or higher sequence identity, or a sequence subjected to substitution of one or more nucleotides, as compared to SEQ ID NO: 127), or (iii) a degenerate sequence of (i) or (ii); and/or,
the nucleic acid molecules encoding the light chain variable region of the antibody comprise: (iv) a nucleotide sequence shown as SEQ ID NO: 128, (v) a sequence substantially identical to SEQ ID NO: 128 (e.g., a sequence having at least about 85%, 90%, 95%, 99% or higher sequence identity, or a sequence subjected to substitution of one or more nucleotides, as compared to SEQ ID NO: 128), or (vi) a degenerate sequence of (iv) or (v).

In another aspect, the present invention provides a vector (like a cloning vector or an expression vector) comprising the isolated nucleic acid molecules according to the present invention. In certain embodiments, the vector according to the present invention is, for example, a plasmid, a cosmid, a bacteriophage and a lentivirus. In certain embodiments, the vector is capable of expressing the antibody or antigen-binding fragment thereof according to the present invention in a subject (e.g., mammal like human).

In certain embodiments, the vector comprises a first nucleotide sequence encoding the heavy chain or heavy chain variable region of the antibody or antigen-binding fragment thereof according to the present invention and a second nucleotide sequence encoding the light chain or light chain variable region thereof; the first nucleotide sequence and the second nucleotide sequence are in the same or different vectors. When the first nucleotide sequence and the second nucleotide sequence are in different vectors, the vector according to the present invention comprises a first vector having the first nucleotide sequence and a second vector having the second nucleotide sequence.

In certain embodiments, the antibody or antigen-binding fragment thereof according to the present invention can be used for constructing a chimeric antigen receptor (CAR) comprising an extracellular antigen-binding domain (like scFv) that specifically binds PTK7, a transmembrane domain, and one or more intracellular T cell signaling domains. In such embodiments, the isolated nucleic acid molecules according to the present invention may comprise a nucleotide sequence encoding the chimeric antigen receptor, and the nucleotide sequence encoding the chimeric antigen receptor further comprises a nucleotide sequence encoding the antibody or antigen-binding fragment thereof (like scFv) according to the present invention. In certain embodiments, the isolated nucleic acid molecules according to the present invention encodes chimeric antigen receptor comprising the antigen-binding fragment of the antibody according to the present invention (like scFv).

In certain embodiments, the antibody or antigen-binding fragment thereof according to the present invention can be used for constructing chimeric antigen receptor modified immune cells, and the chimeric antigen receptor modified immune cells comprise the CAR and immune cells (like T lymphocytes and NK cells).

In another aspect, the present invention provides a host cell comprising the isolated nucleic acid molecules according to the present invention or the vector according to the present invention. The host cell may be eukaryotic cell (like mammalian cell, insect cell or yeast cell) or prokaryotic cell (like Escherichia coli). Suitable eukaryotic cells include but are not limited to NS0 cell, Vero cell, Hela cell, COS cell, CHO cell, ExpiCHO cell, HEK293 cell, Expi293 cell, BHK cell and MDCKII cell. Suitable insect cells include but are not limited to Sf9 cell. In certain embodiments, the host cell according to the present invention is mammalian cell, such as CHO (like CHO-K1, CHO-S, CHO DXB11, ExpiCHO and CHO DG44).

In certain embodiments, the host cell according to the present invention may be chimeric antigen receptor T cell (CAR-T). In such embodiments, the isolated nucleic acid molecules in the host cell may comprise a nucleotide sequence encoding the chimeric antigen receptor, and the nucleotide sequence encoding the chimeric antigen receptor further comprises a nucleotide sequence encoding the antibody or antigen-binding fragment thereof (like scFv) according to the present invention. In certain embodiments, the isolated nucleic acid molecules in the host cell encode the chimeric antigen receptor comprising the antigen-binding fragment (like scFv) of the antibody according to the present invention.

In another aspect, the present invention provides a method for preparing the antibody or antigen-binding fragment thereof according to the present invention, and the method includes: culturing the host cell according to the present invention under a condition that the expression of the antibody or antigen-binding fragment thereof is allowed, and recovering the antibody or antigen-binding fragment thereof from the cultured host cell culture.

### Therapeutic Use

In another aspect, the present invention provides a pharmaceutical composition comprising the antibody or antigen-binding fragment thereof, the nucleic acid molecules, the vector, the host cell, the conjugate, or the multispecific antibody according to the present invention, and a pharmaceutically acceptable carrier and/or excipient.

In certain embodiments, the pharmaceutical composition according to the present invention comprises the antibody or antigen-binding fragment thereof, and the pharmaceutically acceptable carrier and/or excipient according to the present invention.

In certain embodiments, the pharmaceutical composition may also comprise an additional pharmaceutical active agent. In certain embodiments, the additional pharmaceutical active agent is a drug having anti-tumor activity. In certain embodiments, the additional pharmaceutical active agent is selected from an EGFR inhibitor, a BCR-ABL, FLT3, KIT or RET inhibitor, an HER2 inhibitor, an HER3 inhibitor, an HER4 inhibitor, an IGFR-1 inhibitor, an mTOR inhibitor, a PI3 kinase inhibitor, a c-met or VEGF inhibitor, a PARP inhibitor, a chemotherapeutic drug or any combination thereof. In certain embodiments, the antibody or antigen-binding fragment thereof and the additional pharmaceutical active agent according to the present invention are provided as independent components or mixed components. Therefore, the antibody or antigen-binding fragment thereof according to the present invention may be simultaneously, separately or sequentially administrated with the additional pharmaceutical active agent.

In certain embodiments, the antibody or antigen-binding fragment thereof, the nucleic acid molecules, the vector, the host cell, the conjugate, or the multispecific antibody in the pharmaceutical composition according to the present invention are sufficient to (e.g., in the subject):
(a) inhibit cell proliferation;
(b) inhibit tumor growth;
(c) induce and/or improve antibody-dependent cytotoxic activity;
(d) inhibit PTK7-mediated signaling;
(e) prevent and/or treat PTK7-mediated disease/disorder; or
(f) any combination of (a)-(e).

In certain embodiments, the PTK7-mediated disease/disorder is tumor, such as tumor expressing PTK7. In certain embodiments, the tumor is selected from uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

In another aspect, the present invention provides use of the antibody or antigen-binding fragment thereof, the nucleic acid molecules, the vector, the host cell, the conjugate, the multispecific antibody or the pharmaceutical composition according to the present invention in preparation of drugs. The drugs are used for inhibiting cell proliferation, or preventing and/or treating and/or assisting in tumor treatment.

In certain embodiments, the drugs are used for inhibiting the proliferation of cell (such as tumor cell) expressing the PTK7.

In another aspect, the present invention provides a method for inhibiting cell proliferation, and the method includes: making the cell in contact with the antibody or antigen-binding fragment thereof, the nucleic acid molecules, the vector, the host cell, the conjugate, the multispecific antibody or the pharmaceutical composition according to the present invention. In certain embodiments, the cell is the cell expressing PTK7, such as tumor cell.

In another, the present invention provides a method for preventing and/or treating/or assisting in tumor treatment in a subject, and the method includes: administrating an effective amount of the antibody or antigen-binding fragment thereof, the nucleic acid molecules, the vector, the host cell, the conjugate, the multispecific antibody or the pharmaceutical composition according to the present invention to the subject in need thereof.

In certain embodiments, the method further includes: performing a second therapy to the subject, the second therapy being selected from surgery, chemotherapy, radiotherapy, immunotherapy, gene therapy, DNA therapy, RNA therapy, nano therapy, virus therapy, adjuvant therapy, and any combination thereof. In certain embodiments, the second therapy can be applied simultaneously, separately, or sequentially to the above method.

In any one of the above embodiments, the tumor involved in the antibody or antigen-binding fragment thereof, the nucleic acid molecules, the vector, the host cell, the conjugate, the multispecific antibody or the pharmaceutical composition according to the present invention may be of any tumor form. In certain embodiments, the tumor involved in the antibody or antigen-binding fragment thereof, the nucleic acid molecules, the vector, the host cell, the conjugate, the multispecific antibody or the pharmaceutical composition according to the present invention is PTK7 positive tumor. In certain embodiments, the tumor involved in the antibody or antigen-binding fragment thereof, the nucleic acid molecules, the vector, the host cell, the conjugate, the multispecific antibody or the pharmaceutical composition according to the present invention is selected from uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

The antibody or the antigen-binding fragment thereof according to the present invention and the pharmaceutical composition according to the present invention may be prepared into any dosage form known in the medical field, such as tablets, pills, suspensions, emulsions, solutions, gels, capsules, powder, granules, elixirs, lozenges, suppositories, injections (including injections, sterile powder for injection and concentrated solutions for injection), inhalers, sprays and the like. The preferable dosage form depends on the expected administration mode and treatment use. The pharmaceutical composition according to the present invention is to be sterile and is to be stable under production and storage conditions. One preferred dosage form is the injection. Such injection may be a sterile injection solution. For example, the sterile injection solution may be prepared by the following method: doping an essential dose of the antibody according to the present invention in a suitable solvent, and optionally simultaneously doping other desirable ingredients (including, but not limited to, a pH adjuster, a surfactant, an adjuvant, an ionic strength enhancer, an isotonic reagent, a preservative, a diluent, or any combination thereof), and then performing filtration sterilization. In addition, the sterile injection solution may be prepared as sterile lyophilized powder (e.g., by vacuum drying or freeze drying) facilitating storage and use. Such sterile lyophilized powder may be dispersed in a suitable vector, e.g., sterile pyrogen-free water, prior to use.

In addition, the antibody or antigen-binding fragment thereof according to the present invention may be present in the pharmaceutical composition in a unit dose form for ease of administration.

The antibody or antigen-binding fragment thereof, and the pharmaceutical composition according to the present invention may be administered by any suitable method known in the art, including, but not limited to, oral, buccal, sublingual, eyeball, local, parenteral, rectal, intrathecal, intraalveolar, inguinal, intravesical, topical (e.g., powder, ointment, or drops), or nasal administration. However, for many therapeutic uses, the preferred route/manner of administration is parenteral administration (e.g., intravenous injection, subcutaneous injection, intraperitoneal injection, intramuscular injection). The technician needs to understand that the route and/or manner of administration will vary according to the intended purpose. In a preferred embodiment, the antibody or antigen-binding fragment thereof, and the pharmaceutical compositions according to the present invention are administered by intravenous infusion or injection.

The pharmaceutical composition according to the present invention may include a “therapeutically effective amount” or a “prophylactically effective amount” of the antibody or antigen-binding fragment thereof according to the present invention. The “prophylactically effective amount” refers to the amount sufficient to prevent, or delay the occurrence of a disease. The “therapeutically effective amount” refers to the amount sufficient to cure or at least partially prevent a disease and complications thereof in a patient suffered the disease. The therapeutically effective amount of the antibody or antigen-binding fragment thereof according to the present invention may vary according to the following factors: the severity of the disease to be treated, the overall state of the immune system of the patient, the general conditions of the patient such as age, weight, and gender, the manner of administration of the drug, and other treatments performed simultaneously, and the like.

According to the present invention, the dosing regimen may be adjusted to achieve an optimal response for purposes (e.g., treatment or prevention response). For example, the drug may be administrated by a single dose, or the drug may be administrated by multiple doses over a period of time, or the dose may be reduced or increased in proportion to the degree of urgency of the treatment.

According to the present invention, the subject may be mammal, such as human.

### Detection Use

The antibody or the antigen-binding fragment according to the present invention is capable of being specifically bound to PTK7 so as to detect the presence or level of the PTK7 in the sample.

Therefore, in another aspect, the present invention provides a kit comprising the antibody or antigen-binding fragment according to the present invention. In certain embodiments, the antibody or antigen-binding fragment according to the present invention has a detectable marker. In a preferred embodiment, the kit further includes a secondary antibody for specifically recognizing the antibody or antigen-binding fragment thereof according to the present invention. Preferably, the secondary antibody further includes a detectable marker.

According to the present invention, the detectable marker may be any substance which can be detected by means of fluorescence, spectrum, photochemistry, biochemistry, immunology, electricity, optics or chemistry. Particularly preferably, such marker can be used for immunological detection (like enzyme linked immunoassay, radioimmunoassay, fluorescence immunoassay and chemiluminescence immunoassay). Such marker is well-known in the art, and the examples of the marker include, but are not limited to, enzymes (like horseradish peroxidase, alkaline phosphatase, β-galactosidase, urease, glucose oxidase), radionuclides (like 3H, 125I, 35S, 14C or 32P), fluorochrome (like fluorescein isothiocyanate (FITC), fluorescein, tetramethylrhodamine isothiocyanate (TRITC), phycoerythrin (PE), texas red, rhodamine, quantum dots or cyanine dye derivatives (like Cy7 and Alexa 750)), acridinium ester compounds, magnetic beads (like Dynabeads®), thermal measurement markers such as colloidal gold or colored glass or plastic (like polystyrene, polypropylene, latex) beads, and biotin for binding to the above marker-modified avidins (like streptavidin). In certain embodiments, the detectable marker described above may be linked to the antibody according to the present invention by linkers of different lengths so as to reduce potential steric hindrance.

In another aspect, the present invention provides a method for detecting the presence or level of PTK7 in a sample, and the method includes a step of using the antibody or antigen-binding fragment thereof according to the present invention. In a preferred embodiment, the antibody or antigen-binding fragment thereof according to the present invention further has a detectable marker. In another preferred embodiment, the method further includes: detecting the antibody or antigen-binding fragment thereof according to the present invention by a reagent having the detectable marker. The method may be suitable for diagnosis or non-diagnostic purposes (for example, the sample is a cell sample, rather than a sample from the patient).

In certain embodiments, the method includes: making the sample in contact with the antibody or antigen-binding fragment thereof according to the present invention under a condition that a complex is allowed to be formed between the antibody or antigen-binding fragment thereof and PTK7, and detecting the formation of the complex.

Because of low expression or not expression of PTK7 in normal tissue, as well as expression or high expression in some cancers, the tumor may be diagnosed by detecting the presence or level of the PTK7 in the sample. Therefore, in certain embodiments, the method is used for diagnosing the tumor, such as PTK7 positive tumor, like uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

In certain embodiments, the method includes: detecting the expression level of the PTK7 in a to-be-detected sample from the subject, and comparing the expression level with a reference value (such as health control); and the increase in the expression level compared with the reference value is an indication of the tumor.

In another aspect, the present invention provides use of the antibody or antigen-binding fragment thereof according to the present invention; and the kit is used for detecting the presence or level of the PTK7 in the sample, and/or diagnosing tumor.

In another, the present invention provides a diagnostic or therapeutic kit comprising the antibody or antigen-binding fragment thereof, the nucleic acid molecules, the vector, the host cell, the conjugate or the multispecific antibody according to the present invention, and an instruction for use.

The antibody according to the invention has high binding affinity with PTK7, can induce efficient endocytosis after being bound to cell expressing PTK7, and has good hydrophilicity, and may have advantages of being better in quality and in vivo metabolic properties, and suitable for coupling. Therefore, the antibody according to the present invention has a potential for preventing and/or treating tumors. In addition, the antibody according to the present invention is the humanized antibody, which can be safely administered to human subjects, and can reduce the risk of causing immunogenic reaction. Therefore, the antibody according to the invention has great clinical value.

### BRIEF DESCRIPTION OF THE DRAWINGS

FIG. 1A: Binding of an ELISA detection murine antibody to human PTK7 protein.

FIG. 1B: ELISA detection of binding of a murine antibody to monkey PTK7 protein.

FIG. 2A: Determination of binding of an anti-human PTK7 chimeric and humanized antibody to H1299 cell.

FIG. 2B: Determination of binding activity of an anti-human PTK7 humanized antibody to human lung adenocarcinoma H1975.

FIG. 2C: Determination of binding activity of an anti-human PTK7 humanized antibody to human breast cancer cell T47D.

FIG. 2D: Determination of binding activity of an anti-human PTK7 humanized antibody to CHO-hPTK7 overexpression cell.

FIG. 3: Detection of ADCC activity of an anti-human PTK7 humanized antibody.

FIG. 4A: Endocytosis detection of an anti-human PTK7 humanized antibody to H1299 cell.

FIG. 4B: Endocytosis detection of an anti-human PTK7 humanized antibody to H1975 cell.

FIG. 4C: Endocytosis detection of an anti-human PTK7 chimeric and humanized antibody to T47D cell.

FIG. 4D: Endocytosis detection of an anti-human PTK7 humanized antibody to human pharyngeal squamous cancer cell (FADU).

FIG. 5: ELISA detection of epitope competitive binding of an anti-human PTK7 humanized antibody.

FIG. 6A: Flow cytometry of epitope competitive binding of an anti-human PTK7 humanized antibody (marked as Hu24, 64A10HZ and 4E12HZ).

FIG. 6B: Flow cytometry of epitope competitive binding of an anti-human PTK7 humanized antibody (marked as 101A6HZ and 19CT2HZ).

### DETAILED DESCRIPTION

### Definitions

In the present invention, unless otherwise specified, scientific and technical terms used herein have the meanings commonly understood by those skilled in the art. Moreover, the operation steps such as cell culture, biochemistry, nucleic acid chemistry and immunology laboratory used in the present invention are all routine steps widely used in the corresponding fields. Meanwhile, in order to better understand the present invention, definitions and explanations of related terms are provided below.

As used herein, the term “antibody” is used in the most extensive sense, it includes various antibody structures, including but not limited to monoclonal antibodies, polyclonal antibodies, multispecific antibodies (like bispecific antibodies), and antibody fragments, as long as they show required antigen-binding activity. For example, an immunoglobulin molecule can be composed of two pairs of polypeptide chains (each pair having one light chain (LC) and one heavy chain (HC). The light chain of the antibody can be classified as kappa (κ) and lambda (λ) light chains. The heavy chain can be classified as μ, δ, γ, α or ε, and the isotypes of the antibody are defined as IgM, IgD, IgG, IgA, and IgE, respectively. In the light chain and heavy chain, the variable regions and the constant regions are linked by a “J” region having about 12 or more amino acids, and the heavy chain further comprises a “D” region having about 3 or more amino acids. Each heavy chain comprises the heavy chain variable region (VH) and the heavy chain constant region (CH). The heavy chain constant region is composed of 3 domains (CH1, CH2, and CH3). Each light chain comprises the light chain variable region (VL) and the light chain constant region (CL). The light chain constant region is composed of a domain CL. The constant domains do not directly participate in the binding between the antibody and the antigen, but show a variety of effector functions, such as mediating the binding of immunoglobulins to host tissues or factors, including various cells of the immune system (like effector cells) and a first component (C1q) of a classical complement system. The VH and VL regions may also be subdivided into regions with high variability (referred to as complementarity determining regions (CDRs)), with more conservative regions interspersed therebetween, referred to as framework regions (FRs). Each VH and each VL are composed of 3 CDRs and 4 FRs arranged in the following order: FR1, CDR1, FR2, CDR2, FR3, CDR3 and FR4 from the amino terminus to the carboxy terminus. The variable regions (VH and VL) of each heavy chain/light chain pair form antigen-binding parts, respectively. The allocation of amino acids in each region or domain may follow the definition of Kabat, Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883.

In the present invention, unless otherwise specified, when referring to the term “antibody”, it includes not only the complete antibody, but also the antigen-binding fragment of the antibody.

As used herein, the term “complementarity determining region” or “CDR” refers to amino acid residues for antigen-binding in the variable region of the antibody. The exact boundaries of these amino acid residues can be defined according to various numbering systems known in the art, e.g., as defined according to the AbM numbering system (Martin A C R, Cheetham J C, Rees A R (1989) Modelling antibody hypervariable loops: A combined algorithm. Proc Natl Acad Sci USA 86:9268-9272) or the IMGT numbering system (Lefranc et al., Dev. Comparat. Immunol. 27:55-77, 2003). For a given antibody, those skilled in the art will easily identify the CDR defined according to each numbering system. Moreover, the correspondence between different numbering systems is well-known to those skilled in the art (for example, see Lefranc et al., Dev. Comparat. Immunol. 27:55-77, 2003).

In the present invention, the CDR in the antibody or antigen-binding fragment thereof according to the present invention can be determined according to various numbering systems known in the art. In certain embodiments, the CDR in the antibody or antigen-binding fragment thereof according to the present invention is preferably determined according to the IMGT, Kabat, Chouiia, or AbM numbering system.

The following general rules disclosed in www.bioinf.org.uk: Prof. Andrew C.R. Martin's Group and reproduced below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26-- | H30--H35B | H26--H35B |
| | (Kabat Numbering)³ | | H32 . . . 34 | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire amino acid sequence of the V_{H} is commonly numbered according to Kabat while the three CDRs within the variable region may be defined according to any one of the aforementioned numbering schemes. In particular embodiments, the numbering of the amino acid positions in the V_{H} may be sequential beginning with amino acid position 1 and continuing sequentially to the end of the sequence or according to Kabat. Unless specified otherwise, the amino acid positions in the V_{H} and V_{L} herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

As used herein, the term “framework region” or “FR” residues refers to those amino acid residues in the variable region of the antibody other than the CDR residues as defined above.

The term “antibody” is not limited by any specific antibody production method. For example, it includes a recombinant antibody, a monoclonal antibody, and a polyclonal antibody. The antibodies can be of different isotypes, e.g., IgG (like IgG1, IgG2, IgG3, or IgG4 subtype), IgA1, IgA2, IgD, IgE, or IgM antibodies.

As used herein, the “antigen-binding fragment” of the term antibody refers to the molecules other than the complete antibody, and it includes a part of the complete antibody, which is bound to the antigen to which the complete antibody is bound. For example, the antigen-binding fragment may be polypeptide of a fragment of a full-length antibody, which retains the capability of specifically binding the same antigen to which the full-length antibody is bound, and/or competes for specific binding to the antigen with the full-length antibody, which is also referred to as the “antigen-binding fragment”. Generally, the full text of Fundamental Immunology, Ch. 7 (Paul, W., ed., Version 2, Raven Press, N.Y. (1989) is merged into the present invention with as reference for all purposes. The antigen-binding fragment of the antibody may be produced by a recombinant DNA technology or by enzymatic or chemical cleavage of the complete antibody. Non-limiting examples of the antigen-binding fragment include Fab, Fab′, Fab′-SH, F(ab′)₂, Fd, Fv, dAb and complementarity determining region (CDR) fragments, single chain antibodies (like scFv), chimeric antibodies, diabodies, linear antibodies, nano-antibodies (technology based on Domantis), domain antibodies (technology based on Ablynx), and similar polypeptides, which comprise at least a part of the antibody sufficient to enable antigen-specific binding ability to the polypeptide. Engineered variants of the antibodies are summarized in Holliger et al., 2005; Nat Biotechnol, 23: 1126-1136.

As used herein, the term “full-length antibody” refers to an antibody composed of two “full-length heavy chains” or “heavy chains” and two “full-length light chains” or “light chains”. The “full-length heavy chains” or “heavy chains” refer to polypeptide chains composed of the heavy chain variable region (VH), the heavy chain constant region CH1 domain, a hinge region (HR), a heavy chain constant region CH2 domain, a heavy chain constant region CH3 domain in the N- to C-terminal direction; and moreover, the full-length antibody optionally further comprises a heavy chain constant region CH4 domain when the full-length antibody is of the IgE isotype. Preferably, the “full-length heavy chains” refer to polypeptide chains composed of VH, CH1, HR, CH2, and CH3 in the N- to C-terminal direction. The “full-length light chains” or “light chains” refer to polypeptide chains composed of the light chain variable region (VL) and the light chain constant region (CL) in the N- to C-terminal direction. The two pairs of full-length antibody chains are linked together by a disulfide bond between CL and CH1 and a disulfide bond between the HRs of the two full-length heavy chains. The full-length antibodies according to the present invention may be from a single species, e.g., human; and may also be the chimeric or humanized antibodies. The full-length antibodies according to the present invention comprise two antigen-binding parts formed by VH and VL pairs, respectively, and the two antigen-binding parts specifically recognize/bind the same antigen.

As used herein, the term “Fd fragment” refers to an antibody fragment composed of VH and CH1 domains; the term “dAb fragment” refers to an antibody fragment composed of VH domains (Ward et al., Nature 341:544 546 (1989)); the term “Fab fragment” refers to an antibody fragment composed of VL, VH, CL, and CH1 domains; the term “F(ab′)₂ fragment” refers to an antibody fragment comprising two Fab fragments linked by a disulfide bridge on the hinge region; the term “Fab′ fragment” refers to a fragment obtained after reducing the disulfide bond linking the two heavy chain fragments in the F(ab′)₂ fragment, and the obtained fragment is composed of a complete Fd fragment (composed of VH and CH1 domains) of the light chain and the heavy chain.

As used herein, the term “Fv fragment” refers to an antibody fragment composed of VL and VH domains of a single arm of the antibody. The Fv fragment is generally considered to be a minimal antibody fragment that forms a complete antigen-binding site. It is generally considered that six CDRs enable the antigen-binding specificity to the antibody. However, one variable region (e.g., an Fd fragment, which contains only three CDRs specific to the antigen) also can be used for recognizing and being bound to the antigen, although its affinity may be lower than the complete binding site.

As used herein, the term “Fc fragment” refers to an antibody fragment formed by binding second and third constant regions of the first heavy chain of the antibody to second and third constant regions of the second heavy chain through disulfide bonds. The Fc fragment of the antibody has a variety of different functions, but does not participate in antigen-binding.

As used herein, the term “scFv” refers to a single polypeptide chain comprising VL and VH domains, and the VL and VH are linked by the linker (refer to, for example, Bird et al., Science 242:423-426 (1988); Huston et al., Proc. Natl. Acad. Sci. USA 85:5879-5883 (1988); and Pluckthun, The Pharmacology of Monoclonal Antibodies, Volume 113, edited by Roseburg and Moore, Springer-Verlag, New York, Pages 269-315 (1994)). Such scFv molecules may have general structure: NH2-VL-linker-VH—COOH or NH2-VH-linker-VL-COOH. Suitable linker in the prior art comprises repeated GGGGS (SEQ ID NO:133) amino acid sequences or variants thereof. For example, a linker having the amino acid sequence (GGGGS)4 (SEQ ID NO:134) can be used, and the variants thereof can also be used (Holliger, et al. (1993), Proc. Natl. Acad. Sci. USA 90: 6444-6448). Other linkers applicable to the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, there may be a disulfide bond between VL and VH of scFv.

As used herein, the term “diabody” means that its VH and VL domains are expressed on a single polypeptide chain, but too short linkers are used so as not to allow pairing between two domains of the same chain, thereby forcing the domains to pair with the complementary domains of the other chain and producing two antigen-binding parts (refer to, for example, Holliger P. et al, Proc. Natl. Acad. Sci. USA 90:6444-6448 (1993), and Poljak R. J. et al., Structure 2:1121-1123 (1994)).

Each of the above antibody fragments retains the capability of specifically binding the same antigen to which the full-length antibody is bound, and/or competes for specific binding to the antigen with the full-length antibody. In the present invention, those skilled in the art can obtain the antigen-binding fragment (like the above antibody fragment) of the antibody from the given antibody (like the antibody according to the present invention) with the known conventional techniques, and specifically screen the antigen-binding fragment of antibody in the same manner as for the complete antibody.

As used herein, the term “multispecific antibody” refers to an antibody with various different antigen-binding specificities, including, for example, a bispecific antibody, a trispecific antibody and a tetraspecific antibody. The “bispecific antibody” refer to an antibody with two different antigen-binding specificities, and the first antibody (or the fragment thereof) and the secondary antibody (or the fragment thereof) or the antibody analogs form the conjugate through a coupling arm using the coupling manner including but not limited to chemical reaction, gene fusion and enzymatic promotion. The “multispecific antibody” includes, for example, the trispecific antibody and the tetraspecific antibody, the trispecific antibody is an antibody with three different antigen-binding specificities, and the tetraspecific antibody is an antibody with four different antigen-binding specificities.

As used herein, the terms “monoclonal antibody”, “McAb” and “mAb” have the same meaning and are interchangeable for use, which refers to one antibody or one fragment of the antibody from a population of highly homologous antibody molecules, i.e., a population of identical antibody molecules in addition to natural mutations that may occur spontaneously. The McAb has high specificity for a single epitope on an antigen. The polyclonal antibody corresponds to the monoclonal antibody and is generally composed of at least two or more different antibodies, and these different antibodies generally recognize different epitopes on the antigen. In addition, the modifier “monoclonal” indicates only that the characteristic of the antibody is obtained from a highly homologous antibody population, cannot be understood as requiring any specific method for preparing the antibody.

As used herein, the term “Chimeric antibody” refers to an antibody of which a part of the light chain or/and the heavy chain is derived from one antibody (which may be derived from a particular species or belong to a particular antibody class or subclass), and of which the other part of the light chain or/and the heavy chain is derived from another antibody (which may be derived from the same or different species or belong to the same or different antibody class or subclass), but the binding activity to a target antibody is still retained. For example, the term “Chimeric antibody” may include an antibody of which the heavy variable chain region and the light variable chain region are derived from the first antibody (like humanized), and the heavy variable constant region and the light variable constant region are derived from the secondary antibody (like murine). For example, the antibody produced by immunizing a human transgenic mouse may be referred to as the chimeric antibody composed of a human variable region and a murine constant region.

As used herein, the term “Humanized antibody” refers to an antibody that can be prepared by substituting a part of a human antibody with a part of a non-human antibody that is prepared by immunizing mammal other than human. Specifically, it is known that is can be prepared by constructing a chimera having genes encoding a human antibody constant region (Proc. Natl. Acad. Sci. (USA) (1987), vol. 84, p. 3439)-3443, Journal of Immunology (1987), Volume 139, Issue 1, Page 3521). The DNA sequence of the human constant region has been described in the prior art, and the constant region gene may be easily obtained from known clones. The DNA sequence encoding the antibody variable region can then be fused to the human constant region. The isoforms of the human constant region can be selected based on the desired effective function or antibody-dependent cytotoxic activity. Suitable isoforms are IgG1, IgG3, and IgG4. The constant region of human light chains, κ chain, and λ chain can all be used. Such humanized chimeric antibody may be expressed by conventional methods.

As used herein, the term “Fully humanized antibody (Human antibody)” means that it can be prepared by using a mouse (XenoMouse (Chemical Biology (2000), vol. 7, issue 8, p. R185-6), HuMAb-Mouse (Infection and Immunity (2002), vol. 70, issue 2, p. 612-9), TC mouse (Biotechnology and Genetics Engineering Review (2002), vol. 19, p. 73-82) and a KM mouse (Cloning Stem Cells (2002), vol. 4, issue 1, p. 91-102)), in which the constant region gene of human immunoglobulin is transferred, and the target antibody can be mass-produced by isolating antibody-producing lymphocytes from the mice to hybridomas. It can be prepared by phage display (FEBS Letter (1998), vol. 441, p. 20-24). In this method, a phage integrating human antibody gene into circular single-stranded DNA is used, and the fully humanized antibody can be expressed on the surface of the phage in a form of fusion with the coat protein of the phage.

As used herein, the term “variant”, in the context of polypeptide (including polypeptide), also refers to polypeptide or peptide comprising an amino acid sequence that has been changed by introducing an amino acid residue for replacement, deletion, or addition. In some cases, the term “variant” also refers to polypeptide or peptide that have been modified (i.e., by covalently linking any type of molecules to the polypeptide or peptide). For example, but not by way of limitation, the polypeptide may be modified, e.g., by glycosylation, acetylation, pegylation, phosphorylation, amidation, derivatization by known protecting/blocking groups, proteolytic cleavage, linking to a cell ligand or other protein. The derived polypeptide or peptide can be produced by chemical modification using techniques known to those skilled in the art, including but not limited to specific chemical cleavage, acetylation, formylation, metabolic synthesis of chlamydomycin, and the like. Furthermore, the variant has similar, identical, or improved functionality with the polypeptide or peptide from which it is derived.

As used herein, the term “specific binding” refers to a non-random binding reaction between two molecules, such as the reaction between the antibody and the related antigen. The strength or affinity of the specific binding interaction may be shown as the equilibrium dissociation constant (K_{D}) or the half maximum effect concentration (EC₅₀) of the interaction.

The specific binding properties between two molecules can be determined using methods known in the art. One method involves measuring antigen-binding site/antigen complex formation and dissociation speed. Both the “binding rate constant” (kₐ or kₒₙ) and the “dissociation rate constant” (k_{dis} or k_{off}) can be calculated through the concentration and the actual association and dissociation rate (see Malmqvist M, Nature, 1993, 361:186-187). The ratio of k_{dis}/kₒₙ is equal to the dissociation constant K_{D} (see Davies et al, Annual Rev Biochem, 1990; 59:439-473). K_{D}, kₒₙ and k_{dis} can be measured using any efficient method. In certain embodiments, the dissociation constant may be measured using bioluminescence interferometry (like ForteBio Octet method). In addition, the dissociation constant can be measured using surface plasmon resonance techniques (like Biacore) or Kinexa.

As used herein, the term “vector” refers to a nucleic acid delivery tool into which the polynucleotide can be inserted. When the vector enables the expression of protein encoded by the inserted polynucleotide, the vector is referred to as the expression vector. The vector may be introduced into the host cell by transformation, transduction, or transfection to enable expression of carried genetic material elements in the host cell. The vector is well-known to those skilled in the art, including but not limited to: plasmid, phagemid, coxs plasmid, and artificial chromosome such as yeast artificial chromosomes (YAC), bacterial artificial chromosomes (BAC), or P1-derived artificial chromosomes (PAC), and bacteriophage such as λ bacteriophage or M13 bacteriophage, and animal virus. The animal virus that can be used as the vector includes but is not limited to: reverse transcriptase virus (including lentivirus), adenovirus, adeno-associated virus, herpes virus (like herpes simplex virus), poxvirus, baculovirus, papilloma virus, and papilloma vacuoles virus (like SV40). One vector may comprise a variety of elements that control expression, including but not limited to: a promoter sequence, a transcription initiation sequence, an enhancer sequence, a selection element, and a reporter gene. In addition, the vector can also contain origin positions of replication.

Expression and cloning vectors have a nucleic acid sequence that enables replication of the vector in one or more selected host cells. Typically, this sequence in the cloning vector enables replication of the vector independently of host chromosomal DNA and includes an origin of replication or an autonomously replicating sequence. The term “expression vector” used herein refers to a vector comprising recombinant polynucleotide, and this vector comprises an expression regulatory sequence effectively linked to the nucleotide sequence to be expressed. The expression vector comprises sufficient cis-acting elements for expression; and other elements for expression may be provided by the host cell or by an in vitro expression system. The expression vector includes those known in the art, such as cosmid, plasmid (like plasmid exposed or contained in liposome) and virus (like lentivirus, retrovirus, adenovirus and adeno-associated virus).

As used herein, the term “host cell” refers to the cell useful for introducing the vector, including but not limited to, prokaryotic cell such as E. Coli or Bacillus subtilis, fungal cell such as yeast cell or Aspergillus, insect cell such as S2 Drosophila cell or Sf9, or animal cell such as fibroblast, NS0 cell, Vero cell, Hela cell, COS cell, CHO cell (like CHO-K1, CHO-S, CHO DXB11, ExpiCHO, CHO DG44 cell), ExpiCHO cell, HEK293 cell, Expi293 cell, BHK cell and MDCKII cell.

As used herein, the term “identity” refers to the matching of sequences between two polypeptides or between two nucleic acids. When a position in two to-be-compared sequences is occupied by a same base or amino acid monomeric subunit (e.g., a position in each of two DNA molecules is occupied by adenine, or a position in each of the two polypeptides is occupied by lysine), the molecules are the same at that position. The “percentage identity” between the two sequences refers to a function obtained by the formula: the number of matched positions common to the two sequences/the number of to-be-compared positions×100. For example, if there are 6 matches in the 10 positions of the two sequences, the two sequences have 60% identity. For example, DNA sequences CTGACT and CAGGTT have 50% identity in total (3 matches in the total of 6 positions). Typically, comparison is performed when two sequences are aligned to achieve the maximum identity. Such alignment can be achieved by using, for example, a method proposed by Needleman et al. (1970) J. Mol. Biol. 48: 443-453 and conveniently performed by a computer program, such as an Align program (DNAstar, Inc.). The percentage identity between two amino acid sequences can also be determined through E. Meyers and W. Miller (Comput. Appl Biosci., 4:11-17 (1988)) algorithms that are integrated into an ALIGN program (Version 2.0) by using a PAM120 weight residue table, a notch length penalty of 12, and a notch penalty of 4. In addition, the percentage identity between two amino acid sequences can be determined through Needleman and Wunsch (J MoI Biol. 48:444-453 (1970)) algorithms in a GAP program that is integrated into a GCG software package (www.gcg.com) by using a Blossum 62 matrix or PAM250 matrix and notch weight of 16, 14, 12, 10, 8, 6 or 4 and length weights of 1, 2, 3, 4, 5 or 6.

As used herein, the term “conservative replacement” means amino acid replacement that does not adversely affect or change the intended properties of proteins/polypeptides comprising the amino acid sequence. For example, the conservative replacement can be introduced by standard techniques known in the art, such as site-directed mutagenesis and PCR-mediated mutagenesis. Conservative amino acid replacement includes replacement of amino acid residues with amino acid residues having similar side chains, e.g., replacement with residues that are physically or functionally similar to the corresponding amino acid residues (e.g., having similar size, shape, charge, chemical properties, including the capability of forming covalent or hydrogen bonds). Families of the amino acid residues having similar side chains have been defined in the art. These families include amino acids having basic side chains (like lysine, arginine, and histidine), acidic side chains (like aspartic acid, and glutamic acid), uncharged polar side chains (like glycine, asparagine, glutamine, serine, threonine, tyrosine, cysteine, and tryptophan), non-polar side chains (like alanine, valine, leucine, isoleucine, proline, phenylalanine, and methionine), β-branched side chains (like threonine, valine, and isoleucine), and aromatic side chains (like tyrosine, phenylalanine, tryptophan, and histidine). Thus, the corresponding amino acid residue is preferably substituted with another amino acid residue from the same side chain family. Methods for identifying amino acid conservative replacements are well-known in the art (refer to, for example, Brummell et al., Biochem. 32:1180-1187 (1993); Kobayashi et al. Protein Eng. 12(10):879-884 (1999); and Burks et al. Proc. Natl Acad. Set USA 94:412-417 (1997), which is incorporated herein by reference).

The compilation of the twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, the terms “polypeptide” and “protein” have the same meaning and are interchangeable for use. And in the present invention, the amino acid is generally shown as a single letter or three letter abbreviation known in the art. For example, alanine may be shown as A or Ala.

As used herein, the term “pharmaceutically acceptable carrier and/or excipient” refers to a vector and/or excipient that is pharmacologically and/or physiologically compatible with the subject and active ingredients, which is well-known in the art (refer to, for example, Remington's Pharmaceutical Sciences. Edited by Gennaro A R, 19th ed. Pennsylvania: Mack Publishing Company, 1995) and includes, but is not limited to, pH modulators, surfactants, adjuvants, ionic strength enhancers, diluents, osmotic pressure maintaining reagents, absorption delaying reagents, preservatives. For example, the pH modulators include, but are not limited to, phosphate buffers. The surfactants include, but are not limited to, cationic, anionic, or nonionic surfactants, such as Tween-80. The ionic strength enhancers include, but are not limited to, sodium chloride. The preservatives include, but are not limited to, various antibacterial and antifungal reagents, such as paraben, trichlorotert-butyl alcohol, phenol, and sorbic acid. The osmotic pressure maintaining reagents include, but are not limited to, sugars, NaCl, and analogs thereof. The absorption delaying reagents include, but are not limited to, monostearate and gelatin. The diluents include, but are not limited to, water, aqueous buffers (like buffered saline), alcohols and polyols (like glycerol), etc. The preservatives include, but are not limited to, various antibacterial and antifungal reagents, such as thiomersalate, 2-phenoxyethanol, paraben, trichlorotert-butyl alcohol, phenol, and sorbic acid. The stabilizers have the meaning normally understood by those skilled in the art, and are capable of stabilizing the desired activity of active ingredients in drugs, including but not limited to, sodium glutamate, gelatin, SPGA, sugars (like sorbitol, mannitol, starch, sucrose, lactose, glucan, or glucose), amino acids (like glutamic acid, and glycine), proteins (like dried whey, albumin, or casein), or degradation products thereof (like lactalbumin hydrolysates), etc.

As used herein, the term “prevention” refers to a method implemented to prevent or delay the occurrence of a disease or disorder or symptom (like a tumor) in the subject. As used herein, the term “treatment” refers to a method implemented to obtain a beneficial or desired clinical outcome in a subject (human or animal individual) that is exhibiting a disease symptom or diagnosed as having a disease (“in need thereof”). For the purpose of the present invention, the beneficial or desired clinical outcomes include, but are not limited to, alleviating symptoms, narrowing the scope of a disease, stabilizing (i.e., no longer deteriorating) the state of a disease, delaying or slowing the development of a disease, ameliorating or alleviating the state of a disease, and alleviating (partially or totally), whether detectable or undetectable. In addition, the “treatment” may also refer to prolonging the survival as compared to a desired survival (if not treated).

As used herein, the term “subject” refers to mammal, such as primate mammal like human. In certain embodiments, the subject (like human) suffers from a tumor, or has a risk of suffering from the above disease.

As used herein, the term “effective amount” refers to the amount sufficient to achieve or at least partially achieve the desired effect. For example, prophylactically effective amount for disease (like tumor) refers to the amount sufficient to prevent, or delay the occurrence of a disease (like tumor); effective amount for disease treatment refers to the amount sufficient to cure or at least partially prevent a disease and complications thereof in a patient suffered a disease. Determination of such effective amounts is entirely within the range of abilities of those skilled in the art. For example, the amount effective for therapeutic use will depend on the severity of the disease to be treated, the overall state of the immune system of the patient, the general situation of the patient, such as age, weight and gender, and the manner in which the drug is administered, and other treatments administered simultaneously.

As used herein, the term “effector function” refers to those attributable to the biological activity of the antibody Fc region (a native sequence Fc region or an amino acid sequence variant Fc region) and which change with antibody isotype. Examples of the effector function of the antibody include, but are not limited to, Fc receptor binding affinity, antibody-dependent cell-mediated cytotoxicity (ADCC), complement dependent Cytotoxicity (CDC), antibody dependent cell phagocytosis (ADCP), down-regulation of cell surface receptors (like B cell receptors), B cell activation, cytokine secretion, half-life/clearance of antibodies and antigen-antibody complexes, and the like. Methods for changing the effector function of the antibody are known in the art, e.g., by introducing mutations in the Fc region.

As used herein, the term “antibody-dependent cell-mediated cytotoxicity (ADCC)” refers to a cytotoxic form in which Ig binds to the Fc receptors (FcRs) in cytotoxic cells (like natural killer (NK) cells, neutrophils, or macrophages) to specifically bind these cytotoxic effector cells to antigen-attached target cells, followed by the secretion of cytotoxin to kill the target cells.

As used herein, the term “antibody-mediated internalization” refers to a phenomenon that the antibody traverses the cell membrane after binding to cell surface antigen. Internalization includes antibody-mediated receptor (e.g., PTK7) internalization.

In the present invention, combination therapies include the use of anti-PTK7 antibodies or antigen-binding fragments thereof according to the present invention in combination with one or more additional active therapeutic reagents (like chemotherapeutic reagents) of a second therapy or other prevention or treatment modes (like radiotherapy).

In such combination therapies, various active agents often have different complementary mechanisms of action, and the combination therapies may result in a synergistic effect. The combination therapies comprise therapeutic reagents that affect immune response (like enhancing or activating response) and therapeutic reagents that affect (like inhibit or kill) tumor/cancer cells. The combination therapies may reduce the likelihood of drug-resistant cancer cell development. The combination therapies may allow for a reduction in the dose of one or more of the reagents so as to reduce or eliminate adverse effects associated with one or more of the reagents. Such combination therapies may have a synergistic treatment or prevention effect on a potential disease, disorder, or symptom.

In the present invention, “combination” includes therapies that may be administered separately, such as therapies for separately formulating for single dosing (e.g., may be provided in a kit), and administrating together as a single formulation (i.e., “co-formulation”). In certain embodiments, the anti-PTK7 antibody or the antigen-binding fragment thereof according to the present invention can be administered sequentially. In other embodiments, the anti-PTK7 antibody or the antigen-binding fragment thereof can be administered simultaneously. The antibody or the antigen-binding fragment thereof according to the present invention may be used in any combination with at least one other (active) reagent.

In the present invention, PTK7-positive results from immunohistochemical and staining intensity evaluation by professional clinicians.

The terms “cancer” and “tumor” can be used interchangeably, which is a major class of diseases characterized by uncontrolled growth of abnormal cells in vivo. Uncontrolled cell division may lead to malignant tumors or formation of cells that invade adjacent tissues, and might be transferred to a distal site of the body through the lymphatic system or blood flow. Cancer includes benign and malignant cancers as well as dormant tumors or micro-metastases. Cancer also includes hematological malignancies.

The term “hematological malignancies” include lymphomas, leukemias, myeloma or lymphoid malignancies, and spleen and lymph node cancers. Exemplary lymphomas include B-cell lymphomas and T-cell lymphomas. B-cell lymphomas include, for example, Hodgkin lymphomas. T-cell lymphomas include, for example, cutaneous T-cell lymphoma. Hematological malignancies also include leukemias, such as secondary leukemias or acute lymphocytic leukemias. Hematological malignancies also include myeloma (like multiple myeloma) and other hematological and/or B-cell or T-cell related cancers.

As used herein, the term “about” or “approximately” when used in conjunction with a numerical variable generally means that the value of the variable is within experimental error (e.g., within a 95% confidence interval for the mean) or within ±10% or wider range.

### Antibody

In one aspect, the present application provides a specific PTK7-binding antibody or antigen-binding fragment thereof, and the antibody or antigen-binding fragment thereof comprises the following complementarity determining regions (CDRs):
(a) CDR-H1, CDR-H2 and CDR-H3 contained in a heavy chain variable region (VH) having the amino acid sequence set forth in SEQ ID NO: 19; and/or CDR-L1, CDR-L2 and CDR-L3 contained in a light chain variable region (VL) having the amino acid sequence set forth in SEQ ID NO: 20;
(b) CDR-H1, CDR-H2 and CDR-H3 contained in a heavy chain variable region (VH) having the amino acid sequence set forth in SEQ ID NO: 1; and/or CDR-L1, CDR-L2 and CDR-L3 contained in a light chain variable region (VL) having the amino acid sequence set forth in SEQ ID NO: 2;
(c) CDR-H1, CDR-H2 and CDR-H3 contained in a heavy chain variable region (VH) having the amino acid sequence set forth in SEQ ID NO: 3; and/or CDR-L1, CDR-L2 and CDR-L3 contained in a light chain variable region (VL) having the amino acid sequence set forth in SEQ ID NO: 4;
(d) CDR-H1, CDR-H2 and CDR-H3 contained in a heavy chain variable region (VH) having the amino acid sequence set forth in SEQ ID NO: 5; and/or CDR-L1, CDR-L2 and CDR-L3 contained in a light chain variable region (VL) having the amino acid sequence set forth in SEQ ID NO: 6;
(e) CDR-H1, CDR-H2 and CDR-H3 contained in a heavy chain variable region (VH) having the amino acid sequence set forth in SEQ ID NO: 7; and/or CDR-L1, CDR-L2 and CDR-L3 contained in a light chain variable region (VL) having the amino acid sequence set forth in SEQ ID NO: 8;
(f) CDR-H1, CDR-H2 and CDR-H3 contained in a heavy chain variable region (VH) having the amino acid sequence set forth in SEQ ID NO: 9; and/or CDR-L1, CDR-L2 and CDR-L3 contained in a light chain variable region (VL) having the amino acid sequence set forth in SEQ ID NO: 10;
(g) CDR-H1, CDR-H2 and CDR-H3 contained in a heavy chain variable region (VH) having the amino acid sequence set forth in SEQ ID NO: 11; and/or CDR-L1, CDR-L2 and CDR-L3 contained in a light chain variable region (VL) having the amino acid sequence set forth in SEQ ID NO: 12;
(h) CDR-H1, CDR-H2 and CDR-H3 contained in a heavy chain variable region (VH) having the amino acid sequence set forth in SEQ ID NO: 13; and/or CDR-L1, CDR-L2 and CDR-L3 contained in a light chain variable region (VL) having the amino acid sequence set forth in SEQ ID NO: 14;
(i) CDR-H1, CDR-H2 and CDR-H3 contained in a heavy chain variable region (VH) having the amino acid sequence set forth in SEQ ID NO: 15; and/or CDR-L1, CDR-L2 and CDR-L3 contained in a light chain variable region (VL) having the amino acid sequence set forth in SEQ ID NO: 16;
(j) CDR-H1, CDR-H2 and CDR-H3 contained in a heavy chain variable region (VH) having the amino acid sequence set forth in SEQ ID NO: 17; and/or CDR-L1, CDR-L2 and CDR-L3 contained in a light chain variable region (VL) having the amino acid sequence set forth in SEQ ID NO: 18;
(k) CDR-H1, CDR-H2 and CDR-H3 contained in a heavy chain variable region (VH) having the amino acid sequence set forth in SEQ ID NO: 21; and/or CDR-L1, CDR-L2 and CDR-L3 contained in a light chain variable region (VL) having the amino acid sequence set forth in SEQ ID NO: 22;
(l) CDR-H1, CDR-H2 and CDR-H3 contained in a heavy chain variable region (VH) having the amino acid sequence set forth in SEQ ID NO: 23; and/or CDR-L1, CDR-L2 and CDR-L3 contained in a light chain variable region (VL) having the amino acid sequence set forth in SEQ ID NO: 24; or
(m) CDR-H1, CDR-H2 and CDR-H3 contained in the heavy chain variable region (VH) of (a) to (1), and/or CDR-L1, CDR-L2 and CDR-L3 contained in the light chain variable region (VL) of (a) to (1); at least one CDR of the heavy chain variable region (VH) and/or the light chain variable region (VL) contains a mutation as compared to any one of the heavy chain variable regions (VH) and/or the light chain variable regions (VL) in (a) to (1), and the mutation refers to replacement, deletion or addition of one or several amino acids (for example, replacement, deletion or addition of 1, 2 or 3 amino acids); preferably, the replacement is conservative replacement.

In one aspect, the present application provides a specific PTK7-binding antibody or antigen-binding fragment thereof, and the antibody or antigen-binding fragment thereof comprises the following complementarity determining regions (CDRs): (a) CDR-H1, CDR-H2 and CDR-H3 contained in a heavy chain variable region (VH) having the amino acid sequence set forth in SEQ ID NO: 19; and/or CDR-L1, CDR-L2 and CDR-L3 contained in a light chain variable region (VL) having the amino acid sequence set forth in SEQ ID NO: 20.

In one aspect, the present application provides a specific PTK7-binding antibody or antigen-binding fragment thereof, and the antibody or antigen-binding fragment thereof comprises the following complementarity determining regions (CDRs): (b) CDR-H1, CDR-H2 and CDR-H3 contained in a heavy chain variable region (VH) having the amino acid sequence set forth in SEQ ID NO: 1; and/or CDR-L1, CDR-L2 and CDR-L3 contained in a light chain variable region (VL) having the amino acid sequence set forth in SEQ ID NO: 2.

In one aspect, the present application provides a specific PTK7-binding antibody or antigen-binding fragment thereof, and the antibody or antigen-binding fragment thereof comprises the following complementarity determining regions (CDRs): (c) CDR-H1, CDR-H2 and CDR-H3 contained in a heavy chain variable region (VH) having the amino acid sequence set forth in SEQ ID NO: 3; and/or CDR-L1, CDR-L2 and CDR-L3 contained in a light chain variable region (VL) having the amino acid sequence set forth in SEQ ID NO: 4.

In one aspect, the present application provides a specific PTK7-binding antibody or antigen-binding fragment thereof, and the antibody or antigen-binding fragment thereof comprises the following complementarity determining regions (CDRs): (d) CDR-H1, CDR-H2 and CDR-H3 contained in a heavy chain variable region (VH) having the amino acid sequence set forth in SEQ ID NO: 5; and/or CDR-L1, CDR-L2 and CDR-L3 contained in a light chain variable region (VL) having the amino acid sequence set forth in SEQ ID NO: 6.

In one aspect, the present application provides a specific PTK7-binding antibody or antigen-binding fragment thereof, and the antibody or antigen-binding fragment thereof comprises the following complementarity determining regions (CDRs): (e) CDR-H1, CDR-H2 and CDR-H3 contained in a heavy chain variable region (VH) having the amino acid sequence set forth in SEQ ID NO: 7; and/or CDR-L1, CDR-L2 and CDR-L3 contained in a light chain variable region (VL) having the amino acid sequence set forth in SEQ ID NO: 8.

In one aspect, the present application provides a specific PTK7-binding antibody or antigen-binding fragment thereof, and the antibody or antigen-binding fragment thereof comprises the following complementarity determining regions (CDRs): (f) CDR-H1, CDR-H2 and CDR-H3 contained in a heavy chain variable region (VH) having the amino acid sequence set forth in SEQ ID NO: 9; and/or CDR-L1, CDR-L2 and CDR-L3 contained in a light chain variable region (VL) having the amino acid sequence set forth in SEQ ID NO: 10.

In one aspect, the present application provides a specific PTK7-binding antibody or antigen-binding fragment thereof, and the antibody or antigen-binding fragment thereof comprises the following complementarity determining regions (CDRs): (g) CDR-H1, CDR-H2 and CDR-H3 contained in a heavy chain variable region (VH) having the amino acid sequence set forth in SEQ ID NO: 11; and/or CDR-L1, CDR-L2 and CDR-L3 contained in a light chain variable region (VL) having the amino acid sequence set forth in SEQ ID NO: 12.

In one aspect, the present application provides a specific PTK7-binding antibody or antigen-binding fragment thereof, and the antibody or antigen-binding fragment thereof comprises the following complementarity determining regions (CDRs): (h) CDR-H1, CDR-H2 and CDR-H3 contained in a heavy chain variable region (VH) having the amino acid sequence set forth in SEQ ID NO: 13; and/or CDR-L1, CDR-L2 and CDR-L3 contained in a light chain variable region (VL) having the amino acid sequence set forth in SEQ ID NO: 14.

In one aspect, the present application provides a specific PTK7-binding antibody or antigen-binding fragment thereof, and the antibody or antigen-binding fragment thereof comprises the following complementarity determining regions (CDRs): (i) CDR-H1, CDR-H2 and CDR-H3 contained in a heavy chain variable region (VH) having the amino acid sequence set forth in SEQ ID NO: 15; and/or CDR-L1, CDR-L2 and CDR-L3 contained in a light chain variable region (VL) having the amino acid sequence set forth in SEQ ID NO: 16.

In one aspect, the present application provides a specific PTK7-binding antibody or antigen-binding fragment thereof, and the antibody or antigen-binding fragment thereof comprises the following complementarity determining regions (CDRs): (j) CDR-H1, CDR-H2 and CDR-H3 contained in a heavy chain variable region (VH) having the amino acid sequence set forth in SEQ ID NO: 17; and/or CDR-L1, CDR-L2 and CDR-L3 contained in a light chain variable region (VL) having the amino acid sequence set forth in SEQ ID NO: 18.

In one aspect, the present application provides a specific PTK7-binding antibody or antigen-binding fragment thereof, and the antibody or antigen-binding fragment thereof comprises the following complementarity determining regions (CDRs): (k) CDR-H1, CDR-H2 and CDR-H3 contained in a heavy chain variable region (VH) having the amino acid sequence set forth in SEQ ID NO: 21; and/or CDR-L1, CDR-L2 and CDR-L3 contained in a light chain variable region (VL) having the amino acid sequence set forth in SEQ ID NO: 22.

In one aspect, the present application provides a specific PTK7-binding antibody or antigen-binding fragment thereof, and the antibody or antigen-binding fragment thereof comprises the following complementarity determining regions (CDRs): (1) CDR-H1, CDR-H2 and CDR-H3 contained in a heavy chain variable region (VH) having the amino acid sequence set forth in SEQ ID NO: 23; and/or CDR-L1, CDR-L2 and CDR-L3 contained in a light chain variable region (VL) having the amino acid sequence set forth in SEQ ID NO: 24.

In one aspect, the present application provides a specific PTK7-binding antibody or antigen-binding fragment thereof, and the antibody or antigen-binding fragment thereof comprises the following complementarity determining regions (CDRs): (m) CDR-HT, CDR-H2 and CDR-H3 contained in the heavy chain variable region (VH) set forth in one of (a) to (1), and/or CDR-LT, CDR-L2 and CDR-L3 contained in the above light chain variable region (VL) set forth in (a) to (1); at least one CDR of the heavy chain variable region (VH) and/or the light chain variable region (VL) contains a mutation as compared to any one of the heavy chain variable regions (VH) and/or the light chain variable regions (VL) in (a) to (1), and the mutation refers to replacement, deletion or addition of one or several amino acids (for example, replacement, deletion or addition of 1, 2 or 3 amino acids); preferably, the replacement is conservative replacement.

In certain embodiments, the replacement is conservative replacement.

In certain embodiments, the CDR is defined according to an IMGT, Kabat, Chothia or AbM numbering system.

In certain embodiments, the PTK7 includes human PTK7 and/or monkey PTK7. In certain embodiments, the monkey is Macaca mulatta.

In certain embodiments, the antibody or antigen-binding fragment thereof of the present invention comprises the heavy chain variable region (VH) and/or the light chain variable region (VL), and the CDR is defined according to the Chothia numbering system as follows:
(1a) a heavy chain variable region (VH) comprising the following 3 CDRs: CDR-H1 having the amino acid sequence set forth in SEQ ID NO: 27 or a variant thereof; CDR-H2 having the amino acid sequence set forth in SEQ ID NO: 28 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(1b) a heavy chain variable region (VH) comprising the following 3 CDRs: CDR-H1 having the amino acid sequence set forth in SEQ ID NO: 41 or a variant thereof; CDR-H2 having the amino acid sequence set forth in SEQ ID NO: 42 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(1c) a heavy chain variable region (VH) comprising the following 3 CDRs: CDR-H1 having the amino acid sequence set forth in SEQ ID NO: 55 or a variant thereof; CDR-H2 having the amino acid sequence set forth in SEQ ID NO: 56 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof;
(1d) a heavy chain variable region (VH) comprising the following 3 CDRs: CDR-H1 having the amino acid sequence set forth in SEQ ID NO: 68 or a variant thereof; CDR-H2 having the amino acid sequence set forth in SEQ ID NO: 69 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(1e) a heavy chain variable region (VH) comprising the following 3 CDRs: CDR-H1 having the amino acid sequence set forth in SEQ ID NO: 74 or a variant thereof; CDR-H2 having the amino acid sequence set forth in SEQ ID NO: 69 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(1f) a heavy chain variable region (VH) comprising the following 3 CDRs: CDR-H1 having the amino acid sequence set forth in SEQ ID NO: 92 or a variant thereof; CDR-H2 having the amino acid sequence set forth in SEQ ID NO: 93 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(1g) a heavy chain variable region (VH) comprising the following 3 CDRs: CDR-H1 having the amino acid sequence set forth in SEQ ID NO: 27 or a variant thereof; CDR-H2 having the amino acid sequence set forth in SEQ ID NO: 28 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(1h) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1i) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1j) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(1k) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(1l) a heavy chain variable region (VH) comprising the following 3 CDRs: CDR-H1 having the amino acid sequence set forth in SEQ ID NO: 74 or a variant thereof; CDR-H2 having the amino acid sequence set forth in SEQ ID NO: 69 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof; and
the variant in any one of (1a)-(1h) and (1l) is subjected to replacement, deletion or addition of one or several amino acids (such as replacement, deletion or addition of 1, 2 or 3 amino acids) as compared to the sequence from which the variant is derived. In certain embodiments, the replacement is conservative replacement.

In certain embodiments, the antibody or antigen-binding fragment thereof of the present invention comprises the heavy chain variable region (VH) and/or the light chain variable region (VL), and the CDR as follows:
(1a) a heavy chain variable region (VH) comprising the following 3 CDRs: CDR-H1 having the amino acid sequence set forth in SEQ ID NO: 27 or a variant thereof; CDR-H2 having the amino acid sequence set forth in SEQ ID NO: 28 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(1b) a heavy chain variable region (VH) comprising the following 3 CDRs: CDR-H1 having the amino acid sequence set forth in SEQ ID NO: 41 or a variant thereof; CDR-H2 having the amino acid sequence set forth in SEQ ID NO: 42 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(1c) a heavy chain variable region (VH) comprising the following 3 CDRs: CDR-H1 having the amino acid sequence set forth in SEQ ID NO: 55 or a variant thereof; CDR-H2 having the amino acid sequence set forth in SEQ ID NO: 56 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof;
(1d) a heavy chain variable region (VH) comprising the following 3 CDRs: CDR-H1 having the amino acid sequence set forth in SEQ ID NO: 68 or a variant thereof; CDR-H2 having the amino acid sequence set forth in SEQ ID NO: 69 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(1e) a heavy chain variable region (VH) comprising the following 3 CDRs: CDR-H1 having the amino acid sequence set forth in SEQ ID NO: 74 or a variant thereof; CDR-H2 having the amino acid sequence set forth in SEQ ID NO: 69 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(1f) a heavy chain variable region (VH) comprising the following 3 CDRs: CDR-H1 having the amino acid sequence set forth in SEQ ID NO: 92 or a variant thereof; CDR-H2 having the amino acid sequence set forth in SEQ ID NO: 93 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(1g) a heavy chain variable region (VH) comprising the following 3 CDRs: CDR-H1 having the amino acid sequence set forth in SEQ ID NO: 27 or a variant thereof; CDR-H2 having the amino acid sequence set forth in SEQ ID NO: 28 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(1h) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1i) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1j) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(1k) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(1l) a heavy chain variable region (VH) comprising the following 3 CDRs: CDR-H1 having the amino acid sequence set forth in SEQ ID NO: 74 or a variant thereof; CDR-H2 having the amino acid sequence set forth in SEQ ID NO: 69 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof; and
the variant in any one of (1a)-(1h) and (1l) is subjected to replacement, deletion or addition of one or several amino acids (such as replacement, deletion or addition of 1, 2 or 3 amino acids) as compared to the sequence from which the variant is derived. In certain embodiments, the replacement is conservative replacement.

In certain embodiments, the antibody or antigen-binding fragment thereof of the present invention comprises the heavy chain variable region (VH) and/or the light chain variable region (VL), and the CDR as follows:
(1a) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(1b) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1c) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60;
(1d) a heavy chain variable region (VH) comprising the following 3 CDRs: CDR-H1 having the amino acid sequence set forth in SEQ ID NO: 68; CDR-H2 having the amino acid sequence set forth in SEQ ID NO: 69; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73;
(1e) a heavy chain variable region (VH) comprising the following 3 CDRs: CDR-H1 having the amino acid sequence set forth in SEQ ID NO: 74; CDR-H2 having the amino acid sequence set forth in SEQ ID NO: 69; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86;
(1f) a heavy chain variable region (VH) comprising the following 3 CDRs: CDR-H1 having the amino acid sequence set forth in SEQ ID NO: 92; CDR-H2 having the amino acid sequence set forth in SEQ ID NO: 93; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97;
(1g) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107;
(1h) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1i) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(1j) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(1k) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(1l) a heavy chain variable region (VH) comprising the following 3 CDRs: CDR-H1 having the amino acid sequence set forth in SEQ ID NO: 74; CDR-H2 having the amino acid sequence set forth in SEQ ID NO: 69; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof of the present invention comprises the heavy chain variable region (VH) and/or the light chain variable region (VL), and the CDR as follows: (1a) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof of the present invention comprises the heavy chain variable region (VH) and/or the light chain variable region (VL), and the CDR as follows: (1b) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof of the present invention comprises the heavy chain variable region (VH) and/or the light chain variable region (VL), and the CDR as follows: (1c) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof of the present invention comprises the heavy chain variable region (VH) and/or the light chain variable region (VL), and the CDR as follows: (1d) a heavy chain variable region (VH) comprising the following 3 CDRs: CDR-H1 having the amino acid sequence set forth in SEQ ID NO: 68; CDR-H2 having the amino acid sequence set forth in SEQ ID NO: 69; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof of the present invention comprises the heavy chain variable region (VH) and/or the light chain variable region (VL), and the CDR as follows: (1e) a heavy chain variable region (VH) comprising the following 3 CDRs: CDR-H1 having the amino acid sequence set forth in SEQ ID NO: 74; CDR-H2 having the amino acid sequence set forth in SEQ ID NO: 69; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86.

In certain embodiments, the antibody or antigen-binding fragment thereof of the present invention comprises the heavy chain variable region (VH) and/or the light chain variable region (VL), and the CDR as follows: (1f) a heavy chain variable region (VH) comprising the following 3 CDRs: CDR-H1 having the amino acid sequence set forth in SEQ ID NO: 92; CDR-H2 having the amino acid sequence set forth in SEQ ID NO: 93; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97.

In certain embodiments, the antibody or antigen-binding fragment thereof of the present invention comprises the heavy chain variable region (VH) and/or the light chain variable region (VL), and the CDR as follows: (1g) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107.

In certain embodiments, the antibody or antigen-binding fragment thereof of the present invention comprises the heavy chain variable region (VH) and/or the light chain variable region (VL), and the CDR as follows: (1h) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof of the present invention comprises the heavy chain variable region (VH) and/or the light chain variable region (VL), and the CDR as follows: (1i) a heavy chain variable region (VH) comprising the following 3 CDRs: CDR-H1 having the amino acid sequence set forth in SEQ ID NO: 41; CDR-H2 having the amino acid sequence set forth in SEQ ID NO: 42; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof of the present invention comprises the heavy chain variable region (VH) and/or the light chain variable region (VL), and the CDR as follows: (1j) a heavy chain variable region (VH) comprising the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof of the present invention comprises the heavy chain variable region (VH) and/or the light chain variable region (VL), and the CDR as follows: (1k) a heavy chain variable region (VH) comprising the following 3 CDRs: CDR-H1 having the amino acid sequence set forth in SEQ ID NO: 55; CDR-H2 having the amino acid sequence set forth in SEQ ID NO: 56; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof of the present invention comprises the heavy chain variable region (VH) and/or the light chain variable region (VL), and the CDR as follows: (1l) a heavy chain variable region (VH) comprising the following 3 CDRs: CDR-H1 having the amino acid sequence set forth in SEQ ID NO: 74; CDR-H2 having the amino acid sequence set forth in SEQ ID NO: 69; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR is defined according to the Kabat numbering system as follows:
(2a) a heavy chain variable region (VH) comprising the following 3 CDRs: CDR-H1 having a sequence shown as SEQ ID NO: 33 or a variant thereof; CDR-H2 having a sequence shown as SEQ ID NO: 34 or a variant thereof; and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof; CDR-L2 having a sequence shown as SEQ ID NO: 31 or a variant thereof; and CDR-L3 having a sequence shown as SEQ ID NO: 32 or a variant thereof;
(2b) a heavy chain variable region (VH) comprising the following 3 CDRs: CDR-H1 having the amino acid sequence set forth in SEQ ID NO: 33 or a variant thereof; CDR-H2 having the amino acid sequence set forth in SEQ ID NO: 35 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(2c) a heavy chain variable region (VH) comprising the following 3 CDRs: CDR-H1 having the amino acid sequence set forth in SEQ ID NO: 47 or a variant thereof; CDR-H2 having the amino acid sequence set forth in SEQ ID NO: 49 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(2d) a heavy chain variable region (VH) comprising the following 3 CDRs: CDR-H1 having the amino acid sequence set forth in SEQ ID NO: 61 or a variant thereof; CDR-H2 having the amino acid sequence set forth in SEQ ID NO: 62 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof; or
(2e) a heavy chain variable region (VH) comprising the following 3 CDRs: CDR-H1 having the amino acid sequence set forth in SEQ ID NO: 75 or a variant thereof; CDR-H2 having the amino acid sequence set forth in SEQ ID NO: 76 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(2f) a heavy chain variable region (VH) comprising the following 3 CDRs: CDR-H1 having the amino acid sequence set forth in SEQ ID NO: 87 or a variant thereof; CDR-H2 having the amino acid sequence set forth in SEQ ID NO: 88 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(2g) a heavy chain variable region (VH) comprising the following 3 CDRs: CDR-H1 having the amino acid sequence set forth in SEQ ID NO: 98 or a variant thereof; CDR-H2 having the amino acid sequence set forth in SEQ ID NO: 99 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(2h) a heavy chain variable region (VH) comprising the following 3 CDRs: CDR-H1 having the amino acid sequence set forth in SEQ ID NO: 108 or a variant thereof; CDR-H2 having the amino acid sequence set forth in SEQ ID NO: 35 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(2i) a heavy chain variable region (VH) comprising the following 3 CDRs: CDR-H1 having the amino acid sequence set forth in SEQ ID NO: 47 or a variant thereof; CDR-H2 having the amino acid sequence set forth in SEQ ID NO: 48 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(2j) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(2k) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(2l) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73;
the variant in any one of (2a)-(2i) is subjected to replacement, deletion or addition of one or several amino acids (such as replacement, deletion or addition of 1, 2 or 3 amino acids) compared with the sequence from which the variant is derived; and in certain embodiments, the replacement is conservative replacement.

In certain embodiments, the antibody or antigen-binding fragment thereof according to the present invention comprises the heavy chain variable region (VH) and/or the light chain variable region (VL), and the CDR as follows: (2a) a heavy chain variable region (VH) comprising the following 3 CDRs: CDR-H1 having the amino acid sequence set forth in SEQ ID NO: 33 or a variant thereof; CDR-H2 having the amino acid sequence set forth in SEQ ID NO: 34 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(2b) a heavy chain variable region (VH) comprising the following 3 CDRs: CDR-H1 having the amino acid sequence set forth in SEQ ID NO: 33 or a variant thereof; CDR-H2 having the amino acid sequence set forth in SEQ ID NO: 35 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(2c) a heavy chain variable region (VH) comprising the following 3 CDRs: CDR-H1 having the amino acid sequence set forth in SEQ ID NO: 47 or a variant thereof; CDR-H2 having the amino acid sequence set forth in SEQ ID NO: 49 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(2d) a heavy chain variable region (VH) comprising the following 3 CDRs: CDR-H1 having the amino acid sequence set forth in SEQ ID NO: 61 or a variant thereof; CDR-H2 having the amino acid sequence set forth in SEQ ID NO: 62 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof; or
(2e) a heavy chain variable region (VH) comprising the following 3 CDRs: CDR-H1 having the amino acid sequence set forth in SEQ ID NO: 75 or a variant thereof; CDR-H2 having the amino acid sequence set forth in SEQ ID NO: 76 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(2f) a heavy chain variable region (VH) comprising the following 3 CDRs: CDR-H1 having the amino acid sequence set forth in SEQ ID NO: 87 or a variant thereof; CDR-H2 having the amino acid sequence set forth in SEQ ID NO: 88 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(2g) a heavy chain variable region (VH) comprising the following 3 CDRs: CDR-H1 having the amino acid sequence set forth in SEQ ID NO: 98 or a variant thereof; CDR-H2 having the amino acid sequence set forth in SEQ ID NO: 99 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(2h) a heavy chain variable region (VH) comprising the following 3 CDRs: CDR-H1 having the amino acid sequence set forth in SEQ ID NO: 108 or a variant thereof; CDR-H2 having the amino acid sequence set forth in SEQ ID NO: 35 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(2i) a heavy chain variable region (VH) comprising the following 3 CDRs: CDR-H1 having the amino acid sequence set forth in SEQ ID NO: 47 or a variant thereof; CDR-H2 having the amino acid sequence set forth in SEQ ID NO: 48 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(2j) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(2k) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(2l) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73;
the variant in any one of (2a)-(2i) is subjected to replacement, deletion or addition of one or several amino acids (such as replacement, deletion or addition of 1, 2 or 3 amino acids) compared with the sequence from which the variant is derived; and in certain embodiments, the replacement is conservative replacement.

In certain embodiments, the antibody or antigen-binding fragment thereof according to the present invention comprises the heavy chain variable region (VH) and/or the light chain variable region (VL), and the CDR as follows:
(2a) a heavy chain variable region (VH) comprising the following 3 CDRs: CDR-H1 having the amino acid sequence set forth in SEQ ID NO: 33; CDR-H2 having the amino acid sequence set forth in SEQ ID NO: 34; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(2b) a heavy chain variable region (VH) comprising the following 3 CDRs: CDR-H1 having the amino acid sequence set forth in SEQ ID NO: 33; CDR-H2 having the amino acid sequence set forth in SEQ ID NO: 35; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(2c) a heavy chain variable region (VH) comprising the following 3 CDRs: CDR-H1 having the amino acid sequence set forth in SEQ ID NO: 47; CDR-H2 having the amino acid sequence set forth in SEQ ID NO: 49; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(2d) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(2e) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73;
(2f) a heavy chain variable region (VH) comprising the following 3 CDRs: CDR-H1 having the amino acid sequence set forth in SEQ ID NO: 87; CDR-H2 having the amino acid sequence set forth in SEQ ID NO: 88; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86;
(2g) a heavy chain variable region (VH) comprising the following 3 CDRs: CDR-H1 having the amino acid sequence set forth in SEQ ID NO: 98; CDR-H2 having the amino acid sequence set forth in SEQ ID NO: 99; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97;
(2h) a heavy chain variable region (VH) comprising the following 3 CDRs: CDR-H1 having the amino acid sequence set forth in SEQ ID NO: 108; CDR-H2 having the amino acid sequence set forth in SEQ ID NO: 35; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107;
(2i) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(2j) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(2k) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(2l) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (2a) a heavy chain variable region (VH) comprising the following 3 CDRs: CDR-H1 having the amino acid sequence set forth in SEQ ID NO: 33; CDR-H2 having the amino acid sequence set forth in SEQ ID NO: 34; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (2b) a heavy chain variable region (VH) comprising the following 3 CDRs: CDR-H1 having the amino acid sequence set forth in SEQ ID NO: 33; CDR-H2 having the amino acid sequence set forth in SEQ ID NO: 35; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (2c) a heavy chain variable region (VH) comprising the following 3 CDRs: CDR-H1 having the amino acid sequence set forth in SEQ ID NO: 47; CDR-H2 having the amino acid sequence set forth in SEQ ID NO: 49; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (2d) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (2e) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (2f) a heavy chain variable region (VH) comprising the following 3 CDRs: CDR-H1 having the amino acid sequence set forth in SEQ ID NO: 87; CDR-H2 having the amino acid sequence set forth in SEQ ID NO: 88; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86.

In certain embodiments, the antibody or antigen-binding fragment thereof according to the present invention comprises the heavy chain variable region (VH) and/or the light chain variable region (VL), and the CDR as follows: (2g) a heavy chain variable region (VH) comprising the following 3 CDRs: CDR-H1 having the amino acid sequence set forth in SEQ ID NO: 98; CDR-H2 having the amino acid sequence set forth in SEQ ID NO: 99; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97.

In certain embodiments, the antibody or antigen-binding fragment thereof according to the present invention comprises the heavy chain variable region (VH) and/or the light chain variable region (VL), and the CDR as follows: (2h) a heavy chain variable region (VH) comprising the following 3 CDRs: CDR-H1 having the amino acid sequence set forth in SEQ ID NO: 108; CDR-H2 having the amino acid sequence set forth in SEQ ID NO: 35; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107.

In certain embodiments, the antibody or antigen-binding fragment thereof according to the present invention comprises the heavy chain variable region (VH) and/or the light chain variable region (VL), and the CDR as follows: (2i) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (2j) a heavy chain variable region (VH) comprising the following 3 CDRs: CDR-H1 having the amino acid sequence set forth in SEQ ID NO: 47; CDR-H2 having the amino acid sequence set forth in SEQ ID NO: 48; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (2k) a heavy chain variable region (VH) comprising the following 3 CDRs: CDR-H1 having the amino acid sequence set forth in SEQ ID NO: 61; CDR-H2 having the amino acid sequence set forth in SEQ ID NO: 62; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (2l) a heavy chain variable region (VH) comprising the following 3 CDRs: CDR-H1 having the amino acid sequence set forth in SEQ ID NO: 75; CDR-H2 having the amino acid sequence set forth in SEQ ID NO: 76; and CDR-H3 having the amino acid sequence set forth in SEQ ID NO: 70; and, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR is defined according to the IMGT numbering system as follows:
(3a) a heavy chain variable region (VH) comprising the following 3 CDRs: CDR-H1 having the amino acid sequence set forth in SEQ ID NO: 36 or a variant thereof; CDR-H2 having the amino acid sequence set forth in SEQ ID NO: 37 or a variant thereof; and CDR-H3 having the amino acid sequence set forth in SEQ ID NO: 38 or a variant thereof; and/or, a light chain variable region (VL) comprising the following 3 CDRs: CDR-L1 having the amino acid sequence set forth in SEQ ID NO: 39 or a variant thereof; CDR-L2 having the amino acid sequence set forth in SEQ ID NO: 40 or a variant thereof; and CDR-L3 having the amino acid sequence set forth in SEQ ID NO: 32 or a variant thereof;
(3b) a heavy chain variable region (VH) comprising the following 3 CDRs: CDR-H1 having the amino acid sequence set forth in SEQ ID NO: 50 or a variant thereof; CDR-H2 having the amino acid sequence set forth in SEQ ID NO: 51 or a variant thereof; and CDR-H3 having the amino acid sequence set forth in SEQ ID NO: 52 or a variant thereof; and/or, a light chain variable region (VL) comprising the following 3 CDRs: CDR-L1 having the amino acid sequence set forth in SEQ ID NO: 53 or a variant thereof; CDR-L2 having the amino acid sequence set forth in SEQ ID NO: 54 or a variant thereof; and CDR-L3 having the amino acid sequence set forth in SEQ ID NO: 46 or a variant thereof;
(3c) a heavy chain variable region (VH) comprising the following 3 CDRs: CDR-H1 having the amino acid sequence set forth in SEQ ID NO: 63 or a variant thereof; CDR-H2 having the amino acid sequence set forth in SEQ ID NO: 64 or a variant thereof; and CDR-H3 having the amino acid sequence set forth in SEQ ID NO: 65 or a variant thereof; and/or, a light chain variable region (VL) comprising the following 3 CDRs: CDR-L1 having the amino acid sequence set forth in SEQ ID NO: 66 or a variant thereof; CDR-L2 having the amino acid sequence set forth in SEQ ID NO: 67 or a variant thereof; and CDR-L3 having the amino acid sequence set forth in SEQ ID NO: 60 or a variant thereof;
(3d) a heavy chain variable region (VH) comprising the following 3 CDRs: CDR-H1 having the amino acid sequence set forth in SEQ ID NO: 77 or a variant thereof; CDR-H2 having the amino acid sequence set forth in SEQ ID NO: 78 or a variant thereof; and CDR-H3 having the amino acid sequence set forth in SEQ ID NO: 79 or a variant thereof; and/or, a light chain variable region (VL) comprising the following 3 CDRs: CDR-L1 having the amino acid sequence set forth in SEQ ID NO: 81 or a variant thereof; CDR-L2 having the amino acid sequence set forth in SEQ ID NO: 82 or a variant thereof; and CDR-L3 having the amino acid sequence set forth in SEQ ID NO: 73 or a variant thereof;
(3e) a heavy chain variable region (VH) comprising the following 3 CDRs: CDR-H1 having the amino acid sequence set forth in SEQ ID NO: 80 or a variant thereof; CDR-H2 having the amino acid sequence set forth in SEQ ID NO: 78 or a variant thereof; and CDR-H3 having the amino acid sequence set forth in SEQ ID NO: 89 or a variant thereof; and/or, a light chain variable region (VL) comprising the following 3 CDRs: CDR-L1 having the amino acid sequence set forth in SEQ ID NO: 90 or a variant thereof; CDR-L2 having the amino acid sequence set forth in SEQ ID NO: 91 or a variant thereof; and CDR-L3 having the amino acid sequence set forth in SEQ ID NO: 86 or a variant thereof;
(3f) a heavy chain variable region (VH) comprising the following 3 CDRs: CDR-H1 having the amino acid sequence set forth in SEQ ID NO: 100 or a variant thereof; CDR-H2 having the amino acid sequence set forth in SEQ ID NO: 101 or a variant thereof; and CDR-H3 having the amino acid sequence set forth in SEQ ID NO: 102 or a variant thereof; and/or, a light chain variable region (VL) comprising the following 3 CDRs: CDR-L1 having the amino acid sequence set forth in SEQ ID NO: 103 or a variant thereof; CDR-L2 having the amino acid sequence set forth in SEQ ID NO: 104 or a variant thereof; and CDR-L3 having the amino acid sequence set forth in SEQ ID NO: 97 or a variant thereof;
(3g) a heavy chain variable region (VH) comprising the following 3 CDRs: CDR-H1 having the amino acid sequence set forth in SEQ ID NO: 36 or a variant thereof; CDR-H2 having the amino acid sequence set forth in SEQ ID NO: 37 or a variant thereof; and CDR-H3 having the amino acid sequence set forth in SEQ ID NO: 109 or a variant thereof; and/or, a light chain variable region (VL) comprising the following 3 CDRs: CDR-L1 having the amino acid sequence set forth in SEQ ID NO: 110 or a variant thereof; CDR-L2 having the amino acid sequence set forth in SEQ ID NO: 40 or a variant thereof; and CDR-L3 having the amino acid sequence set forth in SEQ ID NO: 107 or a variant thereof;
(3h) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3i) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3j) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32;
(3k) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60; or
(3l) a heavy chain variable region (VH) comprising the following 3 CDRs: CDR-H1 having the amino acid sequence set forth in SEQ ID NO: 80 or a variant thereof; CDR-H2 having the amino acid sequence set forth in SEQ ID NO: 78 or a variant thereof; and CDR-H3 having the amino acid sequence set forth in SEQ ID NO: 79 or a variant thereof; and/or, a light chain variable region (VL) comprising the following 3 CDRs: CDR-L1 having the amino acid sequence set forth in SEQ ID NO: 81 or a variant thereof; CDR-L2 having the amino acid sequence set forth in SEQ ID NO: 82 or a variant thereof; and CDR-L3 having the amino acid sequence set forth in SEQ ID NO: 73 or a variant thereof;
the variant in any one of (3a)-(3g) and (3l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR as follows:
(3a) a heavy chain variable region (VH) comprising the following 3 CDRs: CDR-H1 having the amino acid sequence set forth in SEQ ID NO: 36 or a variant thereof; CDR-H2 having the amino acid sequence set forth in SEQ ID NO: 37 or a variant thereof; and CDR-H3 having the amino acid sequence set forth in SEQ ID NO: 38 or a variant thereof; and/or, a light chain variable region (VL) comprising the following 3 CDRs: CDR-L1 having the amino acid sequence set forth in SEQ ID NO: 39 or a variant thereof; CDR-L2 having the amino acid sequence set forth in SEQ ID NO: 40 or a variant thereof; and CDR-L3 having the amino acid sequence set forth in SEQ ID NO: 32 or a variant thereof;
(3b) a heavy chain variable region (VH) comprising the following 3 CDRs: CDR-H1 having the amino acid sequence set forth in SEQ ID NO: 50 or a variant thereof; CDR-H2 having the amino acid sequence set forth in SEQ ID NO: 51 or a variant thereof; and CDR-H3 having the amino acid sequence set forth in SEQ ID NO: 52 or a variant thereof; and/or, a light chain variable region (VL) comprising the following 3 CDRs: CDR-L1 having the amino acid sequence set forth in SEQ ID NO: 53 or a variant thereof; CDR-L2 having the amino acid sequence set forth in SEQ ID NO: 54 or a variant thereof; and CDR-L3 having the amino acid sequence set forth in SEQ ID NO: 46 or a variant thereof;
(3c) a heavy chain variable region (VH) comprising the following 3 CDRs: CDR-H1 having the amino acid sequence set forth in SEQ ID NO: 63 or a variant thereof; CDR-H2 having the amino acid sequence set forth in SEQ ID NO: 64 or a variant thereof; and CDR-H3 having the amino acid sequence set forth in SEQ ID NO: 65 or a variant thereof; and/or, a light chain variable region (VL) comprising the following 3 CDRs: CDR-L1 having the amino acid sequence set forth in SEQ ID NO: 66 or a variant thereof; CDR-L2 having the amino acid sequence set forth in SEQ ID NO: 67 or a variant thereof; and CDR-L3 having the amino acid sequence set forth in SEQ ID NO: 60 or a variant thereof; or
(3d) a heavy chain variable region (VH) comprising the following 3 CDRs: CDR-H1 having the amino acid sequence set forth in SEQ ID NO: 77 or a variant thereof; CDR-H2 having the amino acid sequence set forth in SEQ ID NO: 78 or a variant thereof; and CDR-H3 having the amino acid sequence set forth in SEQ ID NO: 79 or a variant thereof; and/or, a light chain variable region (VL) comprising the following 3 CDRs: CDR-L1 having the amino acid sequence set forth in SEQ ID NO: 81 or a variant thereof; CDR-L2 having the amino acid sequence set forth in SEQ ID NO: 82 or a variant thereof; and CDR-L3 having the amino acid sequence set forth in SEQ ID NO: 73 or a variant thereof;
(3e) a heavy chain variable region (VH) comprising the following 3 CDRs: CDR-H1 having the amino acid sequence set forth in SEQ ID NO: 80 or a variant thereof; CDR-H2 having the amino acid sequence set forth in SEQ ID NO: 78 or a variant thereof; and CDR-H3 having the amino acid sequence set forth in SEQ ID NO: 89 or a variant thereof; and/or, a light chain variable region (VL) comprising the following 3 CDRs: CDR-L1 having the amino acid sequence set forth in SEQ ID NO: 90 or a variant thereof; CDR-L2 having the amino acid sequence set forth in SEQ ID NO: 91 or a variant thereof; and CDR-L3 having the amino acid sequence set forth in SEQ ID NO: 86 or a variant thereof;
(3f) a heavy chain variable region (VH) comprising the following 3 CDRs: CDR-H1 having the amino acid sequence set forth in SEQ ID NO: 100 or a variant thereof; CDR-H2 having the amino acid sequence set forth in SEQ ID NO: 101 or a variant thereof; and CDR-H3 having the amino acid sequence set forth in SEQ ID NO: 102 or a variant thereof; and/or, a light chain variable region (VL) comprising the following 3 CDRs: CDR-L1 having the amino acid sequence set forth in SEQ ID NO: 103 or a variant thereof; CDR-L2 having the amino acid sequence set forth in SEQ ID NO: 104 or a variant thereof; and CDR-L3 having the amino acid sequence set forth in SEQ ID NO: 97 or a variant thereof;
(3g) a heavy chain variable region (VH) comprising the following 3 CDRs: CDR-H1 having the amino acid sequence set forth in SEQ ID NO: 36 or a variant thereof; CDR-H2 having the amino acid sequence set forth in SEQ ID NO: 37 or a variant thereof; and CDR-H3 having the amino acid sequence set forth in SEQ ID NO: 109 or a variant thereof; and/or, a light chain variable region (VL) comprising the following 3 CDRs: CDR-L1 having the amino acid sequence set forth in SEQ ID NO: 110 or a variant thereof; CDR-L2 having the amino acid sequence set forth in SEQ ID NO: 40 or a variant thereof; and CDR-L3 having the amino acid sequence set forth in SEQ ID NO: 107 or a variant thereof;
(3h) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3i) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3j) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32;
(3k) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60; or
(3l) a heavy chain variable region (VH) comprising the following 3 CDRs: CDR-H1 having the amino acid sequence set forth in SEQ ID NO: 80 or a variant thereof; CDR-H2 having the amino acid sequence set forth in SEQ ID NO: 78 or a variant thereof; and CDR-H3 having the amino acid sequence set forth in SEQ ID NO: 79 or a variant thereof; and/or, a light chain variable region (VL) comprising the following 3 CDRs: CDR-L1 having the amino acid sequence set forth in SEQ ID NO: 81 or a variant thereof; CDR-L2 having the amino acid sequence set forth in SEQ ID NO: 82 or a variant thereof; and CDR-L3 having the amino acid sequence set forth in SEQ ID NO: 73 or a variant thereof;
the variant in any one of (3a)-(3g) and (3l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR as follows:
(3a) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and/or, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32;
(3b) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and/or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3c) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and/or, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60;
(3d) a heavy chain variable region (VH) comprising the following 3 CDRs: CDR-H1 having the amino acid sequence set forth in SEQ ID NO: 77; CDR-H2 having the amino acid sequence set forth in SEQ ID NO: 78; and CDR-H3 having the amino acid sequence set forth in SEQ ID NO: 79; and/or, a light chain variable region (VL) comprising the following 3 CDRs: CDR-L1 having the amino acid sequence set forth in SEQ ID NO: 81; CDR-L2 having the amino acid sequence set forth in SEQ ID NO: 82; and CDR-L3 having the amino acid sequence set forth in SEQ ID NO: 73;
(3e) a heavy chain variable region (VH) comprising the following 3 CDRs: CDR-H1 having the amino acid sequence set forth in SEQ ID NO: 80; CDR-H2 having the amino acid sequence set forth in SEQ ID NO: 78; and CDR-H3 having the amino acid sequence set forth in SEQ ID NO: 89; and/or, a light chain variable region (VL) comprising the following 3 CDRs: CDR-L1 having the amino acid sequence set forth in SEQ ID NO: 90; CDR-L2 having the amino acid sequence set forth in SEQ ID NO: 91; and CDR-L3 having the amino acid sequence set forth in SEQ ID NO: 86;
(3f) a heavy chain variable region (VH) comprising the following 3 CDRs: CDR-H1 having the amino acid sequence set forth in SEQ ID NO: 100; CDR-H2 having the amino acid sequence set forth in SEQ ID NO: 101; and CDR-H3 having the amino acid sequence set forth in SEQ ID NO: 102; and/or, a light chain variable region (VL) comprising the following 3 CDRs: CDR-L1 having the amino acid sequence set forth in SEQ ID NO: 103; CDR-L2 having the amino acid sequence set forth in SEQ ID NO: 104; and CDR-L3 having the amino acid sequence set forth in SEQ ID NO: 97;
(3g) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 109; and, a light chain variable region (VL) comprising the following 3 CDRs: CDR-L1 having the amino acid sequence set forth in SEQ ID NO: 110; CDR-L2 having the amino acid sequence set forth in SEQ ID NO: 40; and CDR-L3 having the amino acid sequence set forth in SEQ ID NO: 107;
(3h) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3i) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46;
(3j) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32;
(3k) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60; or
(3l) a heavy chain variable region (VH) comprising the following 3 CDRs: CDR-H1 having the amino acid sequence set forth in SEQ ID NO: 80; CDR-H2 having the amino acid sequence set forth in SEQ ID NO: 78; and CDR-H3 having the amino acid sequence set forth in SEQ ID NO: 79; and/or, a light chain variable region (VL) comprising the following 3 CDRs: CDR-L1 having the amino acid sequence set forth in SEQ ID NO: 81; CDR-L2 having the amino acid sequence set forth in SEQ ID NO: 82; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (3a) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and/or, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (3b) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and/or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (3c) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and/or, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (3d) a heavy chain variable region (VH) comprising the following 3 CDRs: CDR-H1 having the amino acid sequence set forth in SEQ ID NO: 77; CDR-H2 having the amino acid sequence set forth in SEQ ID NO: 78; and CDR-H3 having the amino acid sequence set forth in SEQ ID NO: 79; and/or, a light chain variable region (VL) comprising the following 3 CDRs: CDR-L1 having the amino acid sequence set forth in SEQ ID NO: 81; CDR-L2 having the amino acid sequence set forth in SEQ ID NO: 82; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (3e) a heavy chain variable region (VH) comprising the following 3 CDRs: CDR-H1 having the amino acid sequence set forth in SEQ ID NO: 80; CDR-H2 having the amino acid sequence set forth in SEQ ID NO: 78; and CDR-H3 having the amino acid sequence set forth in SEQ ID NO: 89; and/or, a light chain variable region (VL) comprising the following 3 CDRs: CDR-L1 having the amino acid sequence set forth in SEQ ID NO: 90; CDR-L2 having the amino acid sequence set forth in SEQ ID NO: 91; and CDR-L3 having the amino acid sequence set forth in SEQ ID NO: 86.

In certain embodiments, the antibody or antigen-binding fragment thereof according to the present invention comprises the heavy chain variable region (VH) and/or the light chain variable region (VL), and the CDR as follows: (3f) a heavy chain variable region (VH) comprising the following 3 CDRs: CDR-H1 having the amino acid sequence set forth in SEQ ID NO: 100; CDR-H2 having the amino acid sequence set forth in SEQ ID NO: 101; and CDR-H3 having the amino acid sequence set forth in SEQ ID NO: 102; and/or, a light chain variable region (VL) comprising the following 3 CDRs: CDR-L1 having the amino acid sequence set forth in SEQ ID NO: 103; CDR-L2 having the amino acid sequence set forth in SEQ ID NO: 104; and CDR-L3 having the amino acid sequence set forth in SEQ ID NO: 97.

In certain embodiments, the antibody or antigen-binding fragment thereof according to the present invention comprises the heavy chain variable region (VH) and/or the light chain variable region (VL), and the CDR as follows: (3g) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 109; and/or, a light chain variable region (VL) comprising the following 3 CDRs: CDR-L1 having the amino acid sequence set forth in SEQ ID NO: 110; CDR-L2 having the amino acid sequence set forth in SEQ ID NO: 40; and CDR-L3 having the amino acid sequence set forth in SEQ ID NO: 107.

In certain embodiments, the antibody or antigen-binding fragment thereof according to the present invention comprises the heavy chain variable region (VH) and/or the light chain variable region (VL), and the CDR as follows: (3h) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; and, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (3i) a heavy chain variable region (VH) comprising the following 3 CDRs: CDR-H1 having the amino acid sequence set forth in SEQ ID NO: 50; CDR-H2 having the amino acid sequence set forth in SEQ ID NO: 51; and CDR-H3 having the amino acid sequence set forth in SEQ ID NO: 52; or, a light chain variable region (VL) comprising the following 3 CDRs: CDR-L1 having the amino acid sequence set forth in SEQ ID NO: 53; CDR-L2 having the amino acid sequence set forth in SEQ ID NO: 54; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (3j) a heavy chain variable region (VH) comprising the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38; and, a light chain variable region (VL) comprising the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (3k) a heavy chain variable region (VH) comprising the following 3 CDRs: CDR-H1 having the amino acid sequence set forth in SEQ ID NO: 63; CDR-H2 having the amino acid sequence set forth in SEQ ID NO: 64; and CDR-H3 having the amino acid sequence set forth in SEQ ID NO: 65; and, a light chain variable region (VL) comprising the following 3 CDRs: CDR-L1 having the amino acid sequence set forth in SEQ ID NO: 66; CDR-L2 having the amino acid sequence set forth in SEQ ID NO: 67; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (3l) a heavy chain variable region (VH) comprising the following 3 CDRs: CDR-H1 having the amino acid sequence set forth in SEQ ID NO: 80; CDR-H2 having the amino acid sequence set forth in SEQ ID NO: 78; and CDR-H3 having the amino acid sequence set forth in SEQ ID NO: 79; and/or, a light chain variable region (VL) comprising the following 3 CDRs: CDR-L1 having the amino acid sequence set forth in SEQ ID NO: 81; CDR-L2 having the amino acid sequence set forth in SEQ ID NO: 82; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR is defined according to the AbM numbering system as follows:
(4a) a heavy chain variable region (VH) comprising the following 3 CDRs: CDR-H1 having the amino acid sequence set forth in SEQ ID NO: 113 or a variant thereof; CDR-H2 having the amino acid sequence set forth in SEQ ID NO: 114 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(4b) a heavy chain variable region (VH) comprising the following 3 CDRs: CDR-H1 having the amino acid sequence set forth in SEQ ID NO: 115 or a variant thereof; CDR-H2 having the amino acid sequence set forth in SEQ ID NO: 116 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(4c) a heavy chain variable region (VH) comprising the following 3 CDRs: CDR-H1 having the amino acid sequence set forth in SEQ ID NO: 117 or a variant thereof; CDR-H2 having the amino acid sequence set forth in SEQ ID NO: 118 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof; or
(4d) a heavy chain variable region (VH) comprising the following 3 CDRs: CDR-H1 having the amino acid sequence set forth in SEQ ID NO: 119 or a variant thereof; CDR-H2 having the amino acid sequence set forth in SEQ ID NO: 120 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(4e) a heavy chain variable region (VH) comprising the following 3 CDRs: CDR-H1 having the amino acid sequence set forth in SEQ ID NO: 122 or a variant thereof; CDR-H2 having the amino acid sequence set forth in SEQ ID NO: 123 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(4f) a heavy chain variable region (VH) comprising the following 3 CDRs: CDR-H1 having the amino acid sequence set forth in SEQ ID NO: 124 or a variant thereof; CDR-H2 having the amino acid sequence set forth in SEQ ID NO: 125 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(4g) a heavy chain variable region (VH) comprising the following 3 CDRs: CDR-H1 having the amino acid sequence set forth in SEQ ID NO: 126 or a variant thereof; CDR-H2 having the amino acid sequence set forth in SEQ ID NO: 114 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(4h) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4i) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4j) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(4k) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(4l) a heavy chain variable region (VH) comprising the following 3 CDRs: CDR-H1 having the amino acid sequence set forth in SEQ ID NO: 121 or a variant thereof; CDR-H2 having the amino acid sequence set forth in SEQ ID NO: 120 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
the variant in any one of (4a)-(4g) and (4l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR as follows:
(4a) a heavy chain variable region (VH) comprising the following 3 CDRs: CDR-H1 having the amino acid sequence set forth in SEQ ID NO: 113 or a variant thereof; CDR-H2 having the amino acid sequence set forth in SEQ ID NO: 114 or a variant thereof; and CDR-H3 having the amino acid sequence set forth in SEQ ID NO: 29 or a variant thereof; and/or, a light chain variable region (VL) comprising the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof;
(4b) a heavy chain variable region (VH) comprising the following 3 CDRs: CDR-H1 having the amino acid sequence set forth in SEQ ID NO: 115 or a variant thereof; CDR-H2 having the amino acid sequence set forth in SEQ ID NO: 116 or a variant thereof; and CDR-H3 having the amino acid sequence set forth in SEQ ID NO: 43 or a variant thereof; and/or, a light chain variable region (VL) comprising the following 3 CDRs: CDR-L1 having the amino acid sequence set forth in SEQ ID NO: 44 or a variant thereof; CDR-L2 having the amino acid sequence set forth in SEQ ID NO: 45 or a variant thereof; and CDR-L3 having the amino acid sequence set forth in SEQ ID NO: 46 or a variant thereof;
(4c) a heavy chain variable region (VH) comprising the following 3 CDRs: CDR-H1 having the amino acid sequence set forth in SEQ ID NO: 117 or a variant thereof; CDR-H2 having the amino acid sequence set forth in SEQ ID NO: 118 or a variant thereof; and CDR-H3 having the amino acid sequence set forth in SEQ ID NO: 57 or a variant thereof; and/or, a light chain variable region (VL) comprising the following 3 CDRs: CDR-L1 having the amino acid sequence set forth in SEQ ID NO: 58 or a variant thereof; CDR-L2 having the amino acid sequence set forth in SEQ ID NO: 59 or a variant thereof; and CDR-L3 having the amino acid sequence set forth in SEQ ID NO: 60 or a variant thereof; or
(4d) a heavy chain variable region (VH) comprising the following 3 CDRs: CDR-H1 having the amino acid sequence set forth in SEQ ID NO: 119 or a variant thereof; CDR-H2 having the amino acid sequence set forth in SEQ ID NO: 120 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
(4e) a heavy chain variable region (VH) comprising the following 3 CDRs: CDR-H1 having the amino acid sequence set forth in SEQ ID NO: 122 or a variant thereof; CDR-H2 having the amino acid sequence set forth in SEQ ID NO: 123 or a variant thereof; and CDR-H3 having the amino acid sequence set forth in SEQ ID NO: 83 or a variant thereof; and/or, a light chain variable region (VL) comprising the following 3 CDRs: CDR-L1 having the amino acid sequence set forth in SEQ ID NO: 84 or a variant thereof; CDR-L2 having the amino acid sequence set forth in SEQ ID NO: 85 or a variant thereof; and CDR-L3 having the amino acid sequence set forth in SEQ ID NO: 86 or a variant thereof;
(4f) a heavy chain variable region (VH) comprising the following 3 CDRs: CDR-H1 having the amino acid sequence set forth in SEQ ID NO: 124 or a variant thereof; CDR-H2 having the amino acid sequence set forth in SEQ ID NO: 125 or a variant thereof; and CDR-H3 having the amino acid sequence set forth in SEQ ID NO: 94 or a variant thereof; and/or, a light chain variable region (VL) comprising the following 3 CDRs: CDR-L1 having the amino acid sequence set forth in SEQ ID NO: 95 or a variant thereof; CDR-L2 having the amino acid sequence set forth in SEQ ID NO: 96 or a variant thereof; and CDR-L3 having the amino acid sequence set forth in SEQ ID NO: 97 or a variant thereof;
(4g) a heavy chain variable region (VH) comprising the following 3 CDRs: CDR-H1 having the amino acid sequence set forth in SEQ ID NO: 126 or a variant thereof; CDR-H2 having the amino acid sequence set forth in SEQ ID NO: 114 or a variant thereof; and CDR-H3 having the amino acid sequence set forth in SEQ ID NO: 105 or a variant thereof; and/or, a light chain variable region (VL) comprising the following 3 CDRs: CDR-L1 having the amino acid sequence set forth in SEQ ID NO: 106 or a variant thereof; CDR-L2 having the amino acid sequence set forth in SEQ ID NO: 31 or a variant thereof; and CDR-L3 having the amino acid sequence set forth in SEQ ID NO: 107 or a variant thereof;
(4h) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4i) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4j) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(4k) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(4l) a heavy chain variable region (VH) comprising the following 3 CDRs: CDR-H1 having the amino acid sequence set forth in SEQ ID NO: 121 or a variant thereof; CDR-H2 having the amino acid sequence set forth in SEQ ID NO: 120 or a variant thereof; and CDR-H3 having the amino acid sequence set forth in SEQ ID NO: 70 or a variant thereof; and/or, a light chain variable region (VL) comprising the following 3 CDRs: CDR-L1 having the amino acid sequence set forth in SEQ ID NO: 71 or a variant thereof; CDR-L2 having the amino acid sequence set forth in SEQ ID NO: 72 or a variant thereof; and CDR-L3 having the amino acid sequence set forth in SEQ ID NO: 73 or a variant thereof;
the variant in any one of (4a)-(4g) and (4l) is subjected to replacement, deletion or addition of one or several amino acids (such as replacement, deletion or addition of 1, 2 or 3 amino acids) compared with the sequence from which the variant is derived. In certain embodiments, the replacement is conservative replacement.

In certain embodiments, the antibody or antigen-binding fragment thereof according to the present invention comprises the heavy chain variable region (VH) and/or the light chain variable region (VL), and the CDR as follows:
(4a) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(4b) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4c) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60;
(4d) a heavy chain variable region (VH) comprising the following 3 CDRs: CDR-H1 having the amino acid sequence set forth in SEQ ID NO: 119; CDR-H2 having the amino acid sequence set forth in SEQ ID NO: 120; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73;
(4e) a heavy chain variable region (VH) comprising the following 3 CDRs: CDR-H1 having the amino acid sequence set forth in SEQ ID NO: 122; CDR-H2 having the amino acid sequence set forth in SEQ ID NO: 123; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86;
(4f) a heavy chain variable region (VH) comprising the following 3 CDRs: CDR-H1 having the amino acid sequence set forth in SEQ ID NO: 124; CDR-H2 having the amino acid sequence set forth in SEQ ID NO: 125; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97;
(4g) a heavy chain variable region (VH) comprising the following 3 CDRs: CDR-H1 having the amino acid sequence set forth in SEQ ID NO: 126; CDR-H2 having the amino acid sequence set forth in SEQ ID NO: 114; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107;
(4h) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4i) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46;
(4j) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32;
(4k) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60; or
(4l) a heavy chain variable region (VH) comprising the following 3 CDRs: CDR-H1 having the amino acid sequence set forth in SEQ ID NO: 121; CDR-H2 having the amino acid sequence set forth in SEQ ID NO: 120; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (4a) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and/or, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (4b) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and/or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (4c) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and/or, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (4d) a heavy chain variable region (VH) comprising the following 3 CDRs: CDR-H1 having the amino acid sequence set forth in SEQ ID NO: 119; CDR-H2 having the amino acid sequence set forth in SEQ ID NO: 120; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof according to the present invention comprises the heavy chain variable region (VH) and/or the light chain variable region (VL), and the CDR as follows: (4e) a heavy chain variable region (VH) comprising the following 3 CDRs: CDR-H1 having the amino acid sequence set forth in SEQ ID NO: 122; CDR-H2 having the amino acid sequence set forth in SEQ ID NO: 123; and CDR-H3 having the amino acid sequence set forth in SEQ ID NO: 83; and/or, a light chain variable region (VL) comprising the following 3 CDRs: CDR-L1 having the amino acid sequence set forth in SEQ ID NO: 84; CDR-L2 having the amino acid sequence set forth in SEQ ID NO: 85; and CDR-L3 having the amino acid sequence set forth in SEQ ID NO: 86.

In certain embodiments, the antibody or antigen-binding fragment thereof according to the present invention comprises the heavy chain variable region (VH) and/or the light chain variable region (VL), and the CDR as follows: (4f) a heavy chain variable region (VH) comprising the following 3 CDRs: CDR-H1 having the amino acid sequence set forth in SEQ ID NO: 124; CDR-H2 having the amino acid sequence set forth in SEQ ID NO: 125; and CDR-H3 having the amino acid sequence set forth in SEQ ID NO: 94; and/or, a light chain variable region (VL) comprising the following 3 CDRs: CDR-L1 having the amino acid sequence set forth in SEQ ID NO: 95; CDR-L2 having the amino acid sequence set forth in SEQ ID NO: 96; and CDR-L3 having the amino acid sequence set forth in SEQ ID NO: 97.

In certain embodiments, the antibody or antigen-binding fragment thereof according to the present invention comprises the heavy chain variable region (VH) and/or the light chain variable region (VL), and the CDR as follows: (4g) a heavy chain variable region (VH) comprising the following 3 CDRs: CDR-H1 having the amino acid sequence set forth in SEQ ID NO: 126; CDR-H2 having the amino acid sequence set forth in SEQ ID NO: 114; and CDR-H3 having the amino acid sequence set forth in SEQ ID NO: 105; and/or, a light chain variable region (VL) comprising the following 3 CDRs: CDR-L1 having the amino acid sequence set forth in SEQ ID NO: 106; CDR-L2 having the amino acid sequence set forth in SEQ ID NO: 31; and CDR-L3 having the amino acid sequence set forth in SEQ ID NO: 107.

In certain embodiments, the antibody or antigen-binding fragment thereof according to the present invention comprises the heavy chain variable region (VH) and/or the light chain variable region (VL), and the CDR as follows: (4h) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; and, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (4i) a heavy chain variable region (VH) comprising the following 3 CDRs: CDR-H1 having the amino acid sequence set forth in SEQ ID NO: 115; CDR-H2 having the amino acid sequence set forth in SEQ ID NO: 116; and CDR-H3 having the amino acid sequence set forth in SEQ ID NO: 43; or, a light chain variable region (VL) comprising the following 3 CDRs: CDR-L1 having the amino acid sequence set forth in SEQ ID NO: 44; CDR-L2 having the amino acid sequence set forth in SEQ ID NO: 45; and CDR-L3 having the amino acid sequence set forth in SEQ ID NO: 46.

In certain embodiments, the antibody or antigen-binding fragment thereof according to the present invention comprises the heavy chain variable region (VH) and/or the light chain variable region (VL), and the CDR as follows: (4j) a heavy chain variable region (VH) comprising the following 3 CDRs: CDR-H1 having the amino acid sequence set forth in SEQ ID NO: 113; CDR-H2 having the amino acid sequence set forth in SEQ ID NO: 114; and CDR-H3 having the amino acid sequence set forth in SEQ ID NO: 29; and, a light chain variable region (VL) comprising the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof according to the present invention comprises the heavy chain variable region (VH) and/or the light chain variable region (VL), and the CDR as follows: (4k) a heavy chain variable region (VH) comprising the following 3 CDRs: CDR-H1 having the amino acid sequence set forth in SEQ ID NO: 117; CDR-H2 having the amino acid sequence set forth in SEQ ID NO: 118; and CDR-H3 having the amino acid sequence set forth in SEQ ID NO: 57; and, a light chain variable region (VL) comprising the following 3 CDRs: CDR-L1 having the amino acid sequence set forth in SEQ ID NO: 58; CDR-L2 having the amino acid sequence set forth in SEQ ID NO: 59; and CDR-L3 having the amino acid sequence set forth in SEQ ID NO: 60.

In certain embodiments, the antibody or antigen-binding fragment thereof according to the present invention comprises the heavy chain variable region (VH) and/or the light chain variable region (VL), and the CDR as follows: (4l) a heavy chain variable region (VH) comprising the following 3 CDRs: CDR-H1 having the amino acid sequence set forth in SEQ ID NO: 121; CDR-H2 having the amino acid sequence set forth in SEQ ID NO: 120; and CDR-H3 having the amino acid sequence set forth in SEQ ID NO: 70; and/or, a light chain variable region (VL) comprising the following 3 CDRs: CDR-L1 having the amino acid sequence set forth in SEQ ID NO: 71; CDR-L2 having the amino acid sequence set forth in SEQ ID NO: 72; and CDR-L3 having the amino acid sequence set forth in SEQ ID NO: 73.

In certain embodiments, the antibody or antigen-binding fragment thereof includes framework regions (FRs) from human immunoglobulin.

In certain embodiments, the antibody or antigen-binding fragment thereof according to the present invention comprises:
(a) a VH having a sequence shown as SEQ ID NO: 19 or a variant thereof and/or a VL having a sequence shown as SEQ ID NO: 20 or a variant thereof;
(b) a VH having the amino acid sequence set forth in SEQ ID NO: 1 or a variant thereof and/or a VL having the amino acid sequence set forth in SEQ ID NO: 2 or a variant thereof;
(c) a VH having the amino acid sequence set forth in SEQ ID NO: 3 or a variant thereof and/or a VL having the amino acid sequence set forth in SEQ ID NO: 4 or a variant thereof;
(d) a VH having the amino acid sequence set forth in SEQ ID NO: 5 or a variant thereof and/or a VL having the amino acid sequence set forth in SEQ ID NO: 6 or a variant thereof;
(e) a VH having the amino acid sequence set forth in SEQ ID NO: 7 or a variant thereof and/or a VL having the amino acid sequence set forth in SEQ ID NO: 8 or a variant thereof;
(f) a VH having the amino acid sequence set forth in SEQ ID NO: 9 or a variant thereof and/or a VL having the amino acid sequence set forth in SEQ ID NO: 10 or a variant thereof;
(g) a VH having the amino acid sequence set forth in SEQ ID NO: 11 or a variant thereof and/or a VL having the amino acid sequence set forth in SEQ ID NO: 12 or a variant thereof;
(h) a VH having the amino acid sequence set forth in SEQ ID NO: 13 or a variant thereof and/or a VL having the amino acid sequence set forth in SEQ ID NO: 14 or a variant thereof;
(i) a VH having the amino acid sequence set forth in SEQ ID NO: 15 or a variant thereof and/or a VL having the amino acid sequence set forth in SEQ ID NO: 16 or a variant thereof;
(j) a VH having the amino acid sequence set forth in SEQ ID NO: 17 or a variant thereof and/or a VL having the amino acid sequence set forth in SEQ ID NO: 18 or a variant thereof;
(k) a VH having the amino acid sequence set forth in SEQ ID NO: 21 or a variant thereof and/or a VL having the amino acid sequence set forth in SEQ ID NO: 22 or a variant thereof; or
(l) a VH having the amino acid sequence set forth in SEQ ID NO: 23 or a variant thereof and/or a VL having the amino acid sequence set forth in SEQ ID NO: 24 or a variant thereof;
the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants in (a)-(l) have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises:
(a) a VH having the amino acid sequence set forth in SEQ ID NO: 19 and/or a VL having the amino acid sequence set forth in SEQ ID NO: 20;
(b) a VH having the amino acid sequence set forth in SEQ ID NO: 1 and/or a VL having the amino acid sequence set forth in SEQ ID NO: 2;
(c) a VH having the amino acid sequence set forth in SEQ ID NO: 3 and/or a VL having the amino acid sequence set forth in SEQ ID NO: 4;
(d) a VH having the amino acid sequence set forth in SEQ ID NO: 5 and/or a VL having the amino acid sequence set forth in SEQ ID NO: 6;
(e) a VH having the amino acid sequence set forth in SEQ ID NO: 7 and/or a VL having the amino acid sequence set forth in SEQ ID NO: 8;
(f) a VH having the amino acid sequence set forth in SEQ ID NO: 9 and/or a VL having the amino acid sequence set forth in SEQ ID NO: 10;
(g) a VH having the amino acid sequence set forth in SEQ ID NO: 11 and/or a VL having the amino acid sequence set forth in SEQ ID NO: 12;
(h) a VH having the amino acid sequence set forth in SEQ ID NO: 13 and/or a VL having the amino acid sequence set forth in SEQ ID NO: 14;
(i) a VH having the amino acid sequence set forth in SEQ ID NO: 15 and/or a VL having the amino acid sequence set forth in SEQ ID NO: 16;
(j) a VH having the amino acid sequence set forth in SEQ ID NO: 17 and/or a VL having the amino acid sequence set forth in SEQ ID NO: 18;
(k) a VH having the amino acid sequence set forth in SEQ ID NO: 21 and/or a VL having the amino acid sequence set forth in SEQ ID NO: 22;
(l) a VH having the amino acid sequence set forth in SEQ ID NO: 23 and/or a VL having the amino acid sequence set forth in SEQ ID NO: 24.

In certain embodiments, the antibody or antigen-binding fragment thereof according to the present invention comprises: (j) a VH having the amino acid sequence set forth in SEQ ID NO: 19 and/or a VL having the amino acid sequence set forth in SEQ ID NO: 20.

In certain embodiments, the antibody or antigen-binding fragment thereof according to the present invention comprises: (b) a VH having the amino acid sequence set forth in SEQ ID NO: 1 and/or a VL having the amino acid sequence set forth in SEQ ID NO: 2.

In certain embodiments, the antibody or antigen-binding fragment thereof according to the present invention comprises: (c) a VH having the amino acid sequence set forth in SEQ ID NO: 3 and/or a VL having the amino acid sequence set forth in SEQ ID NO: 4.

In certain embodiments, the antibody or antigen-binding fragment thereof according to the present invention comprises: (d) a VH having the amino acid sequence set forth in SEQ ID NO: 5 and/or a VL having the amino acid sequence set forth in SEQ ID NO: 6.

In certain embodiments, the antibody or antigen-binding fragment thereof according to the present invention comprises: (e) a VH having the amino acid sequence set forth in SEQ ID NO: 7 and/or a VL having the amino acid sequence set forth in SEQ ID NO: 8.

In certain embodiments, the antibody or antigen-binding fragment thereof according to the present invention comprises: (f) a VH having the amino acid sequence set forth in SEQ ID NO: 9 and/or a VL having the amino acid sequence set forth in SEQ ID NO: 10.

In certain embodiments, the antibody or antigen-binding fragment thereof according to the present invention comprises: (g) a VH having the amino acid sequence set forth in SEQ ID NO: 11 and/or a VL having the amino acid sequence set forth in SEQ ID NO: 12.

In certain embodiments, the antibody or antigen-binding fragment thereof according to the present invention comprises: (h) a VH having the amino acid sequence set forth in SEQ ID NO: 13 and/or a VL having the amino acid sequence set forth in SEQ ID NO: 14.

In certain embodiments, the antibody or antigen-binding fragment thereof according to the present invention comprises: (i) a VH having the amino acid sequence set forth in SEQ ID NO: 15 and/or a VL having the amino acid sequence set forth in SEQ ID NO: 16;

In certain embodiments, the antibody or antigen-binding fragment thereof according to the present invention comprises: (j) a VH having the amino acid sequence set forth in SEQ ID NO: 17 and/or a VL having the amino acid sequence set forth in SEQ ID NO: 18.

In certain embodiments, the antibody or antigen-binding fragment thereof according to the present invention comprises: (k) a VH having the amino acid sequence set forth in SEQ ID NO: 21 and/or a VL having the amino acid sequence set forth in SEQ ID NO: 22.

In certain embodiments, the antibody or antigen-binding fragment thereof according to the present invention comprises: (1) a VH having the amino acid sequence set forth in SEQ ID NO: 23 and/or a VL having the amino acid sequence set forth in SEQ ID NO: 24.

In certain embodiments, the antibody or antigen-binding fragment according to any one of the above embodiments further has characteristics selected from the following:
(1) binding PTK7 (such as human or monkey PTK7) with EC₅₀ of less than about 50 ng/mL, such as less than about 40 ng/mL, 30 ng/mL, 25 ng/mL, 24 ng/mL, 23 ng/mL, 22 ng/mL, 21 ng/mL, 20 ng/mL, 19 ng/mL, 18 ng/mL, 17 ng/mL, 16 ng/mL, 15 ng/mL, 14 ng/mL, 13 ng/mL, 12 ng/mL, 11 ng/mL, 10 ng/mL, 9 ng/mL, 8 ng/mL, 7 ng/mL, 6 ng/mL or less; preferably, the EC₅₀ is measured by ELISA;
(2) binding PTK7 (such as human or monkey PTK7) with KD of less than about 100 nM, such as less than about 90 nM, 80 nM, 70 nM, 60 nM, 50 nM, 40 nM, 30 nM, 20 nM, 15 nM, 10 nM, 5 nM, 4 nM, 3 nM, 2 nM, 1 nM or less; preferably, the KD is measured by bio-layer interferometry (BLI) (such as ForteBio Octet®);
(3) having ADCC and CDC activities, such as inducing killing of cells (like tumor cells) expressing PTK7 by ADCC and CDC; and in certain embodiments, the antibody or antigen-binding fragment comprises a wild-type Fc region. In certain embodiments, the antibody or antigen-binding fragment comprises the heavy chain constant region (CH) having the amino acid sequence set forth in SEQ ID NO: 25.
or, not having ADCC and CDC activities; in certain embodiments, the antibody or antigen-binding fragment comprises a mutation-containing or chemically modified Fc region. In certain embodiments, the antibody or antigen-binding fragment comprises the heavy chain constant region (CH) having the amino acid sequence set forth in SEQ ID NO: 131;
(4) inducing PTK7 internalization, such as, measuring by flow cytometry;
(5) inhibiting cell proliferation; and/or
(6) inhibiting tumor growth.

In certain embodiments, the antibody or antigen-binding fragment according to any one of the above embodiments may comprise a constant region from or deviated from human immunoglobulin.

In certain embodiments, a heavy chain of the antibody or antigen-binding fragment thereof comprises a heavy chain constant region from or derived from human immunoglobulin (such as IgG1, IgG2, IgG3 or IgG4). In certain embodiments, the antibody or antigen-binding fragment thereof comprises a wild-type Fc region, or comprises a mutation-containing or chemically modified Fc region which has a changed effector function (such as improved ADCC activity) as compared to the wild-type Fc region. In certain embodiments, the heavy chain of the antibody or antigen-binding fragment thereof comprises the amino acid sequence set forth in SEQ ID NO: 25 or a variant thereof, and the variant is subjected to replacement of up to 20 amino acids (such as replacement of up to 15, up to 10 or up to 5 amino acids, like replacement of 1, 2, 3, 4 or 5 amino acids) as compared to the amino acid sequence set forth in SEQ ID NO: 25. In certain embodiments, the replacement is conservative replacement.

In certain embodiments, the heavy chain of the antibody or antigen-binding fragment thereof comprises the sequence as set forth in SEQ ID NO: 25.

In certain embodiments, the variant is subjected to replacement of 3 amino acids as compared to the amino acid sequence set forth in SEQ ID NO: 25. In certain embodiments, the variant is subjected to replacement of amino acids at positions corresponding to positions 117, 118 and 120 in the amino acid sequence set forth in SEQ ID NO: 25. In certain embodiments, the variant is subjected to replacement of alanine at positions corresponding to positions 117, 118 and 120 in the amino acid sequence set forth in SEQ ID NO: 25. In certain embodiments, the variant has the heavy chain constant region (CH) having the amino acid sequence set forth in SEQ ID NO: 131.

In certain embodiments, a light chain of the antibody or antigen-binding fragment thereof comprises a light chain constant region from or derived from human immunoglobulin (such as κ or λ). In certain embodiments, the light chain of the antibody or antigen-binding fragment thereof comprises having the amino acid sequence set forth in SEQ ID NO: 26 and a variant thereof, and the variant is subjected to replacement of up to 20 amino acids (such as replacement of up to 15, up to 10 or up to 5 amino acids, like replacement of 1, 2, 3, 4 or 5 amino acids) as compared to the amino acid sequence set forth in SEQ ID NO: 26. In certain embodiments, the replacement is conservative replacement.

In certain embodiments, the light chain of the antibody or antigen-binding fragment thereof comprises a sequence set forth in SEQ ID NO: 26.

In certain embodiments, the antibody or an antigen-binding fragment thereof comprises the heavy chain constant region (CH) having the amino acid sequence set forth in SEQ ID NO: 25 and the light chain constant region (CL) having the amino acid sequence set forth in SEQ ID NO: 26.

In certain embodiments, the antibody or an antigen-binding fragment thereof comprises the heavy chain constant region (CH) having the amino acid sequence set forth in SEQ ID NO: 131 and the light chain constant region (CL) having the amino acid sequence set forth in SEQ ID NO: 26.

In certain embodiments, the antibody or antigen-binding fragment thereof having the heavy chain constant region is not bound to an Fc receptor (like CD16a protein, CD32a protein, CD32b protein and CD64 protein). Therefore, in such embodiments, the antibody or antigen-binding fragment thereof may reduce Fc receptor-mediated nonspecific cytotoxicity and improve the safety of the antibody or antigen-binding fragment thereof in the subject. In certain embodiments, the antibody or antigen-binding fragment thereof having the heavy chain constant region may be bound to FcRn. Therefore, in such embodiments, the antibody or antigen-binding fragment thereof has a long half-life in the subject. In certain embodiments, the antibody or antigen-binding fragment thereof having the heavy chain constant region may induce PTK7-mediated endocytosis. Therefore, in such embodiments, the antibody or antigen-binding fragment thereof has the potential as a vector for targeted delivery of drugs, toxins, enzymes or DNA for treatment.

In certain embodiments, the antibody or antigen-binding fragment thereof according to the present invention comprises:
(a) a heavy chain comprising the VH having the amino acid sequence set forth in SEQ ID NO: 19 and the heavy chain constant region (CH) having the amino acid sequence set forth in SEQ ID NO: 131, and, a light chain comprising VL having the amino acid sequence set forth in SEQ ID NO: 20 and the light chain constant region (CL) having the amino acid sequence set forth in SEQ ID NO: 26;
(b) a heavy chain comprising the VH having the amino acid sequence set forth in SEQ ID NO: 19 and the heavy chain constant region (CH) having the amino acid sequence set forth in SEQ ID NO: 25, and, a light chain comprising VL having the amino acid sequence set forth in SEQ ID NO: 20 and the light chain constant region (CL) having the amino acid sequence set forth in SEQ ID NO: 26;
(c) a heavy chain comprising the VH having the amino acid sequence set forth in SEQ ID NO: 1 and the heavy chain constant region (CH) having the amino acid sequence set forth in SEQ ID NO: 25, and, a light chain comprising VL having the amino acid sequence set forth in SEQ ID NO: 2 and the light chain constant region (CL) having the amino acid sequence set forth in SEQ ID NO: 26;
(d) a heavy chain comprising the VH having the amino acid sequence set forth in SEQ ID NO: 1 and the heavy chain constant region (CH) having the amino acid sequence set forth in SEQ ID NO: 131, and, a light chain comprising VL having the amino acid sequence set forth in SEQ ID NO: 2 and the light chain constant region (CL) having the amino acid sequence set forth in SEQ ID NO: 26;
(e) a heavy chain comprising the VH having the amino acid sequence set forth in SEQ ID NO: 3 and the heavy chain constant region (CH) having the amino acid sequence set forth in SEQ ID NO: 25, and, a light chain comprising VL having the amino acid sequence set forth in SEQ ID NO: 4 and the light chain constant region (CL) having the amino acid sequence set forth in SEQ ID NO: 26;
(f) a heavy chain comprising the VH having the amino acid sequence set forth in SEQ ID NO: 5 and the heavy chain constant region (CH) having the amino acid sequence set forth in SEQ ID NO: 25, and, a light chain comprising VL having the amino acid sequence set forth in SEQ ID NO: 6 and the light chain constant region (CL) having the amino acid sequence set forth in SEQ ID NO: 26;
(g) a heavy chain comprising the VH having the amino acid sequence set forth in SEQ ID NO: 7 and the heavy chain constant region (CH) having the amino acid sequence set forth in SEQ ID NO: 25, and, a light chain comprising VL having the amino acid sequence set forth in SEQ ID NO: 8 and the light chain constant region (CL) having the amino acid sequence set forth in SEQ ID NO: 26;
(h) a heavy chain comprising the VH having the amino acid sequence set forth in SEQ ID NO: 9 and the heavy chain constant region (CH) having the amino acid sequence set forth in SEQ ID NO: 25, and, a light chain comprising VL having the amino acid sequence set forth in SEQ ID NO: 10 and the light chain constant region (CL) having the amino acid sequence set forth in SEQ ID NO: 26;
(i) a heavy chain comprising the VH having the amino acid sequence set forth in SEQ ID NO: 11 and the heavy chain constant region (CH) having the amino acid sequence set forth in SEQ ID NO: 25, and, a light chain comprising VL having the amino acid sequence set forth in SEQ ID NO: 12 and the light chain constant region (CL) having the amino acid sequence set forth in SEQ ID NO: 26;
(j) a heavy chain comprising the VH having the amino acid sequence set forth in SEQ ID NO: 13 and the heavy chain constant region (CH) having the amino acid sequence set forth in SEQ ID NO: 25, and, a light chain comprising VL having the amino acid sequence set forth in SEQ ID NO: 14 and the light chain constant region (CL) having the amino acid sequence set forth in SEQ ID NO: 26;
(k) a heavy chain comprising the VH having the amino acid sequence set forth in SEQ ID NO: 15 and the heavy chain constant region (CH) having the amino acid sequence set forth in SEQ ID NO: 25, and, a light chain comprising VL having the amino acid sequence set forth in SEQ ID NO: 16 and the light chain constant region (CL) having the amino acid sequence set forth in SEQ ID NO: 26;
(l) a heavy chain comprising the VH having the amino acid sequence set forth in SEQ ID NO: 17 and the heavy chain constant region (CH) having the amino acid sequence set forth in SEQ ID NO: 25, and, a light chain comprising VL having the amino acid sequence set forth in SEQ ID NO: 18 and the light chain constant region (CL) having the amino acid sequence set forth in SEQ ID NO: 26;
(m) a heavy chain comprising the VH having the amino acid sequence set forth in SEQ ID NO: 21 and the heavy chain constant region (CH) having the amino acid sequence set forth in SEQ ID NO: 25, and, a light chain comprising VL having the amino acid sequence set forth in SEQ ID NO: 22 and the light chain constant region (CL) having the amino acid sequence set forth in SEQ ID NO: 26;
(n) a heavy chain comprising the VH having the amino acid sequence set forth in SEQ ID NO: 23 and the heavy chain constant region (CH) having the amino acid sequence set forth in SEQ ID NO: 25, and, a light chain comprising VL having the amino acid sequence set forth in SEQ ID NO: 24 and the light chain constant region (CL) having the amino acid sequence set forth in SEQ ID NO: 26;
(o) a heavy chain comprising the VH having the amino acid sequence set forth in SEQ ID NO: 3 and the heavy chain constant region (CH) having the amino acid sequence set forth in SEQ ID NO: 131, and, a light chain comprising VL having the amino acid sequence set forth in SEQ ID NO: 4 and the light chain constant region (CL) having the amino acid sequence set forth in SEQ ID NO: 26;
(p) a heavy chain comprising the VH having the amino acid sequence set forth in SEQ ID NO: 5 and the heavy chain constant region (CH) having the amino acid sequence set forth in SEQ ID NO: 131, and, a light chain comprising VL having the amino acid sequence set forth in SEQ ID NO: 6 and the light chain constant region (CL) having the amino acid sequence set forth in SEQ ID NO: 26;
(q) a heavy chain comprising the VH having the amino acid sequence set forth in SEQ ID NO: 7 and the heavy chain constant region (CH) having the amino acid sequence set forth in SEQ ID NO: 131, and, a light chain comprising VL having the amino acid sequence set forth in SEQ ID NO: 8 and the light chain constant region (CL) having the amino acid sequence set forth in SEQ ID NO: 26;
(r) a heavy chain comprising the VH having the amino acid sequence set forth in SEQ ID NO: 9 and the heavy chain constant region (CH) having the amino acid sequence set forth in SEQ ID NO: 131, and, a light chain comprising VL having the amino acid sequence set forth in SEQ ID NO: 10 and the light chain constant region (CL) having the amino acid sequence set forth in SEQ ID NO: 26;
(s) a heavy chain comprising the VH having the amino acid sequence set forth in SEQ ID NO: 11 and the heavy chain constant region (CH) having the amino acid sequence set forth in SEQ ID NO: 131, and, a light chain comprising VL having the amino acid sequence set forth in SEQ ID NO: 12 and the light chain constant region (CL) having the amino acid sequence set forth in SEQ ID NO: 26;
(t) a heavy chain comprising the VH having the amino acid sequence set forth in SEQ ID NO: 13 and the heavy chain constant region (CH) having the amino acid sequence set forth in SEQ ID NO: 131, and, a light chain comprising VL having the amino acid sequence set forth in SEQ ID NO: 14 and the light chain constant region (CL) having the amino acid sequence set forth in SEQ ID NO: 26;
(u) a heavy chain comprising the VH having the amino acid sequence set forth in SEQ ID NO: 15 and the heavy chain constant region (CH) having the amino acid sequence set forth in SEQ ID NO: 131, and, a light chain comprising VL having the amino acid sequence set forth in SEQ ID NO: 16 and the light chain constant region (CL) having the amino acid sequence set forth in SEQ ID NO: 26;
(v) a heavy chain comprising the VH having the amino acid sequence set forth in SEQ ID NO: 17 and the heavy chain constant region (CH) having the amino acid sequence set forth in SEQ ID NO: 131, and, a light chain comprising VL having the amino acid sequence set forth in SEQ ID NO: 18 and the light chain constant region (CL) having the amino acid sequence set forth in SEQ ID NO: 26;
(w) a heavy chain comprising the VH having the amino acid sequence set forth in SEQ ID NO: 21 and the heavy chain constant region (CH) having the amino acid sequence set forth in SEQ ID NO: 131, and, a light chain comprising VL having the amino acid sequence set forth in SEQ ID NO: 22 and the light chain constant region (CL) having the amino acid sequence set forth in SEQ ID NO: 26; or
(x) a heavy chain comprising the VH having the amino acid sequence set forth in SEQ ID NO: 23 and the heavy chain constant region (CH) having the amino acid sequence set forth in SEQ ID NO: 131, and, a light chain comprising VL having the amino acid sequence set forth in SEQ ID NO: 24 and the light chain constant region (CL) having the amino acid sequence set forth in SEQ ID NO: 26.

In certain embodiments, the antibody or antigen-binding fragment thereof according to the present invention comprises: (a) a heavy chain comprising the VH having the amino acid sequence set forth in SEQ ID NO: 19 and the heavy chain constant region (CH) having the amino acid sequence set forth in SEQ ID NO: 131, and, a light chain comprising VL having the amino acid sequence set forth in SEQ ID NO: 20 and the light chain constant region (CL) having the amino acid sequence set forth in SEQ ID NO: 26.

In certain embodiments, the antibody or antigen-binding fragment thereof according to the present invention comprises: (b) a heavy chain comprising the VH having the amino acid sequence set forth in SEQ ID NO: 19 and the heavy chain constant region (CH) having the amino acid sequence set forth in SEQ ID NO: 25, and, a light chain comprising VL having the amino acid sequence set forth in SEQ ID NO: 20 and the light chain constant region (CL) having the amino acid sequence set forth in SEQ ID NO: 26.

In certain embodiments, the antibody or antigen-binding fragment thereof according to the present invention comprises: (c) a heavy chain comprising the VH having the amino acid sequence set forth in SEQ ID NO: 1 and the heavy chain constant region (CH) having the amino acid sequence set forth in SEQ ID NO: 25, and, a light chain comprising VL having the amino acid sequence set forth in SEQ ID NO: 2 and the light chain constant region (CL) having the amino acid sequence set forth in SEQ ID NO: 26.

In certain embodiments, the antibody or antigen-binding fragment thereof according to the present invention comprises: (d) a heavy chain comprising the VH having the amino acid sequence set forth in SEQ ID NO: 1 and the heavy chain constant region (CH) having the amino acid sequence set forth in SEQ ID NO: 131, and, a light chain comprising VL having the amino acid sequence set forth in SEQ ID NO: 2 and the light chain constant region (CL) having the amino acid sequence set forth in SEQ ID NO: 26.

In certain embodiments, the antibody or antigen-binding fragment thereof according to the present invention comprises: (e) a heavy chain comprising the VH having the amino acid sequence set forth in SEQ ID NO: 3 and the heavy chain constant region (CH) having the amino acid sequence set forth in SEQ ID NO: 25, and, a light chain comprising VL having the amino acid sequence set forth in SEQ ID NO: 4 and the light chain constant region (CL) having the amino acid sequence set forth in SEQ ID NO: 26.

In certain embodiments, the antibody or antigen-binding fragment thereof according to the present invention comprises: (f) a heavy chain comprising the VH having the amino acid sequence set forth in SEQ ID NO: 5 and the heavy chain constant region (CH) having the amino acid sequence set forth in SEQ ID NO: 25, and, a light chain comprising VL having the amino acid sequence set forth in SEQ ID NO: 6 and the light chain constant region (CL) having the amino acid sequence set forth in SEQ ID NO: 26.

In certain embodiments, the antibody or antigen-binding fragment thereof according to the present invention comprises: (g) a heavy chain comprising the VH having the amino acid sequence set forth in SEQ ID NO: 7 and the heavy chain constant region (CH) having the amino acid sequence set forth in SEQ ID NO: 25, and, a light chain comprising VL having the amino acid sequence set forth in SEQ ID NO: 8 and the light chain constant region (CL) having the amino acid sequence set forth in SEQ ID NO: 26.

In certain embodiments, the antibody or antigen-binding fragment thereof according to the present invention comprises: (h) a heavy chain comprising the VH having the amino acid sequence set forth in SEQ ID NO: 9 and the heavy chain constant region (CH) having the amino acid sequence set forth in SEQ ID NO: 25, and, a light chain comprising VL having the amino acid sequence set forth in SEQ ID NO: 10 and the light chain constant region (CL) having the amino acid sequence set forth in SEQ ID NO: 26.

In certain embodiments, the antibody or antigen-binding fragment thereof according to the present invention comprises: (i) a heavy chain comprising the VH having the amino acid sequence set forth in SEQ ID NO: 11 and the heavy chain constant region (CH) having the amino acid sequence set forth in SEQ ID NO: 25, and, a light chain comprising VL having the amino acid sequence set forth in SEQ ID NO: 12 and the light chain constant region (CL) having the amino acid sequence set forth in SEQ ID NO: 26.

In certain embodiments, the antibody or antigen-binding fragment thereof according to the present invention comprises: (j) a heavy chain comprising the VH having the amino acid sequence set forth in SEQ ID NO: 13 and the heavy chain constant region (CH) having the amino acid sequence set forth in SEQ ID NO: 25, and, a light chain comprising VL having the amino acid sequence set forth in SEQ ID NO: 14 and the light chain constant region (CL) having the amino acid sequence set forth in SEQ ID NO: 26.

In certain embodiments, the antibody or antigen-binding fragment thereof according to the present invention comprises: (k) a heavy chain comprising the VH having the amino acid sequence set forth in SEQ ID NO: 15 and the heavy chain constant region (CH) having the amino acid sequence set forth in SEQ ID NO: 25, and, a light chain comprising VL having the amino acid sequence set forth in SEQ ID NO: 16 and the light chain constant region (CL) having the amino acid sequence set forth in SEQ ID NO: 26.

In certain embodiments, the antibody or antigen-binding fragment thereof according to the present invention comprises: (1) a heavy chain comprising the VH having the amino acid sequence set forth in SEQ ID NO: 17 and the heavy chain constant region (CH) having the amino acid sequence set forth in SEQ ID NO: 25, and, a light chain comprising VL having the amino acid sequence set forth in SEQ ID NO: 18 and the light chain constant region (CL) having the amino acid sequence set forth in SEQ ID NO: 26.

In certain embodiments, the antibody or antigen-binding fragment thereof according to the present invention comprises: (m) a heavy chain comprising the VH having the amino acid sequence set forth in SEQ ID NO: 21 and the heavy chain constant region (CH) having the amino acid sequence set forth in SEQ ID NO: 25, and, a light chain comprising VL having the amino acid sequence set forth in SEQ ID NO: 22 and the light chain constant region (CL) having the amino acid sequence set forth in SEQ ID NO: 26.

In certain embodiments, the antibody or antigen-binding fragment thereof according to the present invention comprises: (n) a heavy chain comprising the VH having the amino acid sequence set forth in SEQ ID NO: 23 and the heavy chain constant region (CH) having the amino acid sequence set forth in SEQ ID NO: 25, and, a light chain comprising VL having the amino acid sequence set forth in SEQ ID NO: 24 and the light chain constant region (CL) having the amino acid sequence set forth in SEQ ID NO: 26.

In certain embodiments, the antibody or antigen-binding fragment thereof according to the present invention comprises: (o) a heavy chain comprising the VH having the amino acid sequence set forth in SEQ ID NO: 3 and the heavy chain constant region (CH) having the amino acid sequence set forth in SEQ ID NO: 131, and, a light chain comprising VL having the amino acid sequence set forth in SEQ ID NO: 4 and the light chain constant region (CL) having the amino acid sequence set forth in SEQ ID NO: 26.

In certain embodiments, the antibody or antigen-binding fragment thereof according to the present invention comprises: (p) a heavy chain comprising the VH having the amino acid sequence set forth in SEQ ID NO: 5 and the heavy chain constant region (CH) having the amino acid sequence set forth in SEQ ID NO: 131, and, a light chain comprising VL having the amino acid sequence set forth in SEQ ID NO: 6 and the light chain constant region (CL) having the amino acid sequence set forth in SEQ ID NO: 26.

In certain embodiments, the antibody or antigen-binding fragment thereof according to the present invention comprises: (q) a heavy chain comprising the VH having the amino acid sequence set forth in SEQ ID NO: 7 and the heavy chain constant region (CH) having the amino acid sequence set forth in SEQ ID NO: 131, and, a light chain comprising VL having the amino acid sequence set forth in SEQ ID NO: 8 and the light chain constant region (CL) having the amino acid sequence set forth in SEQ ID NO: 26.

In certain embodiments, the antibody or antigen-binding fragment thereof according to the present invention comprises: (r) a heavy chain comprising the VH having the amino acid sequence set forth in SEQ ID NO: 9 and the heavy chain constant region (CH) having the amino acid sequence set forth in SEQ ID NO: 131, and, a light chain comprising VL having the amino acid sequence set forth in SEQ ID NO: 10 and the light chain constant region (CL) having the amino acid sequence set forth in SEQ ID NO: 26.

In certain embodiments, the antibody or antigen-binding fragment thereof according to the present invention comprises: (s) a heavy chain comprising the VH having the amino acid sequence set forth in SEQ ID NO: 11 and the heavy chain constant region (CH) having the amino acid sequence set forth in SEQ ID NO: 131, and, a light chain comprising VL having the amino acid sequence set forth in SEQ ID NO: 12 and the light chain constant region (CL) having the amino acid sequence set forth in SEQ ID NO: 26.

In certain embodiments, the antibody or antigen-binding fragment thereof according to the present invention comprises: (t) a heavy chain comprising the VH having the amino acid sequence set forth in SEQ ID NO: 13 and the heavy chain constant region (CH) having the amino acid sequence set forth in SEQ ID NO: 131, and, a light chain comprising VL having the amino acid sequence set forth in SEQ ID NO: 14 and the light chain constant region (CL) having the amino acid sequence set forth in SEQ ID NO: 26.

In certain embodiments, the antibody or antigen-binding fragment thereof according to the present invention comprises: (u) a heavy chain comprising the VH having the amino acid sequence set forth in SEQ ID NO: 15 and the heavy chain constant region (CH) having the amino acid sequence set forth in SEQ ID NO: 131, and, a light chain comprising VL having the amino acid sequence set forth in SEQ ID NO: 16 and the light chain constant region (CL) having the amino acid sequence set forth in SEQ ID NO: 26.

In certain embodiments, the antibody or antigen-binding fragment thereof according to the present invention comprises: (v) a heavy chain comprising the VH having the amino acid sequence set forth in SEQ ID NO: 17 and the heavy chain constant region (CH) having the amino acid sequence set forth in SEQ ID NO: 131, and, a light chain comprising VL having the amino acid sequence set forth in SEQ ID NO: 18 and the light chain constant region (CL) having the amino acid sequence set forth in SEQ ID NO: 26.

In certain embodiments, the antibody or antigen-binding fragment thereof according to the present invention comprises: (w) a heavy chain comprising the VH having the amino acid sequence set forth in SEQ ID NO: 21 and the heavy chain constant region (CH) having the amino acid sequence set forth in SEQ ID NO: 131, and, a light chain comprising VL having the amino acid sequence set forth in SEQ ID NO: 22 and the light chain constant region (CL) having the amino acid sequence set forth in SEQ ID NO: 26.

In certain embodiments, the antibody or antigen-binding fragment thereof according to the present invention comprises: (x) a heavy chain comprising the VH having the amino acid sequence set forth in SEQ ID NO: 23 and the heavy chain constant region (CH) having the amino acid sequence set forth in SEQ ID NO: 131, and, a light chain comprising VL having the amino acid sequence set forth in SEQ ID NO: 24 and the light chain constant region (CL) having the amino acid sequence set forth in SEQ ID NO: 26.

In certain embodiments, the antibody or antigen-binding fragment thereof according to the present invention comprises a heavy chain comprising a VH having the amino acid sequence selected from the group consisting of SEQ ID NOs:1, 3, 5, 7, 9, 11, 13, 15, 17, 19, 21 and 23.

In certain embodiments, the antibody or antigen-binding fragment thereof according to the present invention comprises a heavy chain comprising a VH having the amino acid sequence set forth in SEQ ID NO: 1.

In certain embodiments, the antibody or antigen-binding fragment thereof according to the present invention comprises a heavy chain comprising a VH having the amino acid sequence set forth in SEQ ID NO: 3.

In certain embodiments, the antibody or antigen-binding fragment thereof according to the present invention comprises a heavy chain comprising a VH having the amino acid sequence set forth in SEQ ID NO: 5.

In certain embodiments, the antibody or antigen-binding fragment thereof according to the present invention comprises a heavy chain comprising a VH having the amino acid sequence set forth in SEQ ID NO: 7.

In certain embodiments, the antibody or antigen-binding fragment thereof according to the present invention comprises a heavy chain comprising a VH having the amino acid sequence set forth in SEQ ID NO: 9.

In certain embodiments, the antibody or antigen-binding fragment thereof according to the present invention comprises a heavy chain comprising a VH having the amino acid sequence set forth in SEQ ID NO: 11.

In certain embodiments, the antibody or antigen-binding fragment thereof according to the present invention comprises a heavy chain comprising a VH having the amino acid sequence set forth in SEQ ID NO: 13.

In certain embodiments, the antibody or antigen-binding fragment thereof according to the present invention comprises a heavy chain comprising a VH having the amino acid sequence set forth in SEQ ID NO: 15.

In certain embodiments, the antibody or antigen-binding fragment thereof according to the present invention comprises a heavy chain comprising a VH having the amino acid sequence set forth in SEQ ID NO: 17.

In certain embodiments, the antibody or antigen-binding fragment thereof according to the present invention comprises a heavy chain comprising a VH having the amino acid sequence set forth in SEQ ID NO: 19.

In certain embodiments, the antibody or antigen-binding fragment thereof according to the present invention comprises a heavy chain comprising a VH having the amino acid sequence set forth in SEQ ID NO: 21.

In certain embodiments, the antibody or antigen-binding fragment thereof according to the present invention comprises a heavy chain comprising a VH having the amino acid sequence set forth in SEQ ID NO: 23.

In certain embodiments, the antibody or an antigen-binding fragment thereof comprises a heavy chain comprising a heavy chain variable region (VH) having the amino acid sequence set forth in SEQ ID NO: 19 or a variant thereof and the heavy chain constant region (CH) having the amino acid sequence set forth in SEQ ID NO: 131 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or an antigen-binding fragment thereof comprises a heavy chain comprising a heavy chain variable region (VH) having the amino acid sequence set forth in SEQ ID NO: 19 and the heavy chain constant region (CH) having the amino acid sequence set forth in SEQ ID NO: 131.

In certain embodiments, the antibody or an antigen-binding fragment thereof comprises a heavy chain having the amino acid sequence set forth in SEQ ID NO: 111 or a variant thereof;
wherein the variant has at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variant is derived, or are subjected to replacement, deletion or addition of one or several amino acids (such as replacement, deletion or addition of 1, 2, 3, 4 or 5 amino acids) as compared to the sequence from which the variant is derived. In certain embodiments, the replacement is conservative replacement. In certain embodiments, the variant has variations in the antibody framework region of the sequence from which the variant is derived. In certain embodiments, the variations are replacement, deletion or addition of one or several amino acids (such as replacement, deletion or addition of 1, 2, 3, 4 or 5 amino acids) in the antibody framework region of the sequence from which the variant is derived. In certain embodiments, the variations are replacement of one or several amino acids in the antibody framework region of the sequence from which the variant is derived. In certain embodiments, the replacement is conservative replacement in the antibody framework region of the sequence from which the variant is derived.

In certain embodiments, the antibody or an antigen-binding fragment thereof comprises a heavy chain having the amino acid sequence set forth in SEQ ID NO: 111.

In certain embodiments, the antibody or an antigen-binding fragment thereof comprises a light chain having the amino acid sequence set forth in SEQ ID NO: 112 or a variant thereof;
wherein the variant has at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variant is derived, or is subjected to replacement, deletion or addition of one or several amino acids (such as replacement, deletion or addition of 1, 2, 3, 4 or 5 amino acids) as compared to the sequence from which the variant is derived. In certain embodiments, the replacement is conservative replacement. In certain embodiments, the variant has variations in the antibody framework region of the sequence from which the variant is derived. In certain embodiments, the variations are replacement, deletion or addition of one or several amino acids (such as replacement, deletion or addition of 1, 2, 3, 4 or 5 amino acids) in the antibody framework region of the sequence from which the variant is derived. In certain embodiments, the variations are replacement of one or several amino acids in the antibody framework region of the sequence from which the variant is derived. In certain embodiments, the replacement is conservative replacement in the antibody framework region of the sequence from which the variant is derived.

In certain embodiments, the antibody or an antigen-binding fragment thereof comprises a light chain having the amino acid sequence set forth in SEQ ID NO: 112.

In certain embodiments, the antibody or antigen-binding fragment thereof comprises:
(a) a heavy chain having the amino acid sequence set forth in SEQ ID NO: 135 or a variant thereof and/or a light chain having the amino acid sequence set forth in SEQ ID NO: 112; or
(b) a heavy chain having the amino acid sequence set forth in SEQ ID NO: 111 or a variant thereof and/or a light chain having the amino acid sequence set forth in SEQ ID NO: 112.

In certain embodiments, the antibody or antigen-binding fragment thereof according to the present invention comprises a heavy chain variable region (VH) having the amino acid sequence set forth in SEQ ID NO: 19 or a variant thereof, and the following 3 CDRs: CDR-H1 having the amino acid sequence set forth in SEQ ID NO: 27 or a variant thereof; CDR-H2 having the amino acid sequence set forth in SEQ ID NO: 28 or a variant thereof; and CDR-H3 having the amino acid sequence set forth in SEQ ID NO: 29 or a variant thereof, and a light chain variable region (VL) having the amino acid sequence set forth in SEQ ID NO: 20 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and the following 3 CDRs: CDR-H1 having the amino acid sequence set forth in SEQ ID NO: 27; CDR-H2 having the amino acid sequence set forth in SEQ ID NO: 28; and CDR-H3 having the amino acid sequence set forth in SEQ ID NO: 29, and a light chain variable region (VL) having the amino acid sequence set forth in SEQ ID NO: 20.

In certain embodiments, the antibody or antigen-binding fragment thereof according to the present invention comprises a heavy chain variable region (VH) having the amino acid sequence set forth in SEQ ID NO: 19 or a variant thereof, and the following 3 CDRs: CDR-H1 having the amino acid sequence set forth in SEQ ID NO: 33 or a variant thereof; CDR-H2 having the amino acid sequence set forth in SEQ ID NO: 34 or a variant thereof; and CDR-H3 having the amino acid sequence set forth in SEQ ID NO: 29 or a variant thereof, and a light chain variable region (VL) having the amino acid sequence set forth in SEQ ID NO: 20 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and the following 3 CDRs: CDR-H1 having the amino acid sequence set forth in SEQ ID NO: 33; CDR-H2 having the amino acid sequence set forth in SEQ ID NO: 34; and CDR-H3 having the amino acid sequence set forth in SEQ ID NO: 29, and a light chain variable region (VL) having the amino acid sequence set forth in SEQ ID NO: 20.

In certain embodiments, the antibody or antigen-binding fragment thereof according to the present invention comprises a heavy chain variable region (VH) having the amino acid sequence set forth in SEQ ID NO: 19 or a variant thereof, and the following 3 CDRs: CDR-H1 having the amino acid sequence set forth in SEQ ID NO: 36 or a variant thereof; CDR-H2 having the amino acid sequence set forth in SEQ ID NO: 37 or a variant thereof; and CDR-H3 having the amino acid sequence set forth in SEQ ID NO: 38 or a variant thereof, and a light chain variable region (VL) having the amino acid sequence set forth in SEQ ID NO: 20 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and the following 3 CDRs: CDR-H1 having the amino acid sequence set forth in SEQ ID NO: 36; CDR-H2 having the amino acid sequence set forth in SEQ ID NO: 37; and CDR-H3 having the amino acid sequence set forth in SEQ ID NO: 38, and a light chain variable region (VL) having the amino acid sequence set forth in SEQ ID NO: 20.

In certain embodiments, the antibody or antigen-binding fragment thereof according to the present invention comprises a heavy chain variable region (VH) having the amino acid sequence set forth in SEQ ID NO: 19 or a variant thereof, and the following 3 CDRs: CDR-H1 having the amino acid sequence set forth in SEQ ID NO: 113 or a variant thereof; CDR-H2 having the amino acid sequence set forth in SEQ ID NO: 114 or a variant thereof; and CDR-H3 having the amino acid sequence set forth in SEQ ID NO: 29 or a variant thereof, and a light chain variable region (VL) having the amino acid sequence set forth in SEQ ID NO: 20 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and the following 3 CDRs: CDR-H1 having the amino acid sequence set forth in SEQ ID NO:113; CDR-H2 having the amino acid sequence set forth in SEQ ID NO: 114; and CDR-H3 having the amino acid sequence set forth in SEQ ID NO: 29, and a light chain variable region (VL) having the amino acid sequence set forth in SEQ ID NO: 20.

In certain embodiments, the antibody or antigen-binding fragment thereof according to the present invention comprises a heavy chain variable region (VH) having the amino acid sequence set forth in SEQ ID NO: 19 or a variant thereof, and a light chain variable region (VL) having the amino acid sequence set forth in SEQ ID NO: 20 or a variant thereof, and the following 3 CDRs: CDR-L1 having the amino acid sequence set forth in SEQ ID NO: 30 or a variant thereof; CDR-L2 having the amino acid sequence set forth in SEQ ID NO: 31 or a variant thereof; and CDR-L3 having the amino acid sequence set forth in SEQ ID NO: 32 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and a light chain variable region (VL) having the amino acid sequence set forth in SEQ ID NO: 20, and the following 3 CDRs: CDR-L1 having the amino acid sequence set forth in SEQ ID NO: 30; CDR-L2 having the amino acid sequence set forth in SEQ ID NO: 31; and CDR-L3 having the amino acid sequence set forth in SEQ ID NO: 32.

In certain embodiments, the antibody or antigen-binding fragment thereof according to the present invention comprises a heavy chain variable region (VH) having the amino acid sequence set forth in SEQ ID NO: 19 or a variant thereof, and a light chain variable region (VL) having the amino acid sequence set forth in SEQ ID NO: 20 or a variant thereof, and the following 3 CDRs: CDR-L1 having the amino acid sequence set forth in SEQ ID NO: 39 or a variant thereof; CDR-L2 having the amino acid sequence set forth in SEQ ID NO: 40 or a variant thereof; and CDR-L3 having the amino acid sequence set forth in SEQ ID NO: 32 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention comprises a heavy chain variable region (VH) having the amino acid sequence set forth in SEQ ID NO: 19, and a light chain variable region (VL) having the amino acid sequence set forth in SEQ ID NO: 20, and the following 3 CDRs: CDR-L1 having the amino acid sequence set forth in SEQ ID NO: 39; CDR-L2 having the amino acid sequence set forth in SEQ ID NO: 40; and CDR-L3 having the amino acid sequence set forth in SEQ ID NO: 32.

In certain embodiments of the antibody or antigen-binding fragment disclosed herein, the heavy chain constant domains may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof may undergo cyclization to pyroglutamate. In some embodiments of the antibody or antigen binding fragment thereof, the N-terminal amino acid of the antibody or antigen binding fragment thereof may undergo cyclization to pyroglutamic acid.

As known to those skilled in the art, pyroglutamic acid is the conjugate acid of pyroglutamate., and is in equilibrium with pyroglutamate in solution.

In certain embodiments, provided herein is a composition comprising antibodies or antigen-binding fragments as disclosed herein, various species of the antibodies or antigen-binding fragments therein may independently comprise a C-terminal lysine, lack a C-terminal lysine, lack a C-terminal glycine-lysine and/or comprise an N-terminal glutamine or glutamic acid, cyclization of the N-terminal amino acid to pyroglutamate or cyclization of the N-terminal amino acid to pyroglutamic acid.

In certain embodiments, the antibody or antigen-binding fragment disclosed herein includes the antibody or antigen-binding fragment that specifically binds the antigen, and may include post-translational modifications thereof (e.g., C-terminal Lysine clipping in the heavy chain, conversion of the N-terminal glutamine or glutamic acid to pyroglutamate in the heavy or light chain) which may occur when recombinantly expressed in host cells (e.g., CHO cells), or during purification/storage.

In certain embodiments, the antibody or antigen-binding fragment thereof according to the present invention comprises a heavy chain comprising a VH having the amino acid sequence selected from the group consisting of SEQ ID NOs: 1, 3, 5, 7, 9, 11, 13, 15, 17, 19, 21 and 23, wherein the N-terminal glutamine or glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence selected from the group consisting of SEQ ID NOs: 1, 3, 5, 7, 9, 11, 13, 15, 17, 19, 21 and 23, wherein the N-terminal glutamine or glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 1, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 1, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 3, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 3, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 5, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 5, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 7, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 7, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 9, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 9, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 11, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 11, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 13, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 13, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 15, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 15, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 17, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 17, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 19, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 19, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 21, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 21, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 23, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 23, wherein the N-terminal glutamine of the antibody heavy chain variable region has undergone cyclization to pyroglutamic acid.

In certain embodiments, the antibody or antigen-binding fragment thereof according to the present invention comprises a light chain comprising the VL having the amino acid sequence set forth in SEQ ID NO: 12, wherein the N-terminal glutamine of the light chain variable region has undergone cyclization to pyroglutamate, or comprises a light chain comprising the VL having the amino acid sequence set forth in SEQ ID NO: 16, wherein N-terminal glutamic acid of the light chain variable region has undergone cyclization to pyroglutamate.

In certain embodiments, the antibody or antigen-binding fragment thereof according to the present invention comprises a light chain comprising the VL having the amino acid sequence set forth in SEQ ID NO: 12, wherein the N-terminal glutamine of the light chain variable region has undergone cyclization to pyroglutamic acid, or comprises a light chain comprising the VL having the amino acid sequence set forth in SEQ ID NO: 16, wherein N-terminal glutamic acid of the light chain variable region has undergone cyclization to pyroglutamic acid.

In certain embodiments, the antibody or antigen-binding fragment thereof according to the present invention comprises a light chain comprising a VL having the amino acid sequence set forth in SEQ ID NO: 12, wherein the N-terminal glutamine of the light chain variable region has undergone cyclization to pyroglutamate.

In certain embodiments, the antibody or antigen-binding fragment thereof according to the present invention comprises a light chain comprising a VL having the amino acid sequence set forth in SEQ ID NO: 12, wherein the N-terminal glutamine of the light chain variable region has undergone cyclization to pyroglutamic acid.

In certain embodiments, the antibody or antigen-binding fragment thereof according to the present invention comprises a light chain comprising the VL having the amino acid sequence set forth in SEQ ID NO: 16, wherein the N-terminal glutamic acid of the light chain variable region has undergone cyclization to pyroglutamate.

In certain embodiments, the antibody or antigen-binding fragment thereof according to the present invention comprises a light chain comprising the VL having the amino acid sequence set forth in SEQ ID NO: 16, wherein the N-terminal glutamic acid of the light chain variable region has undergone cyclization to pyroglutamic acid.

In certain embodiments, the antibody or antigen-binding fragment thereof according to the present invention comprises: a VH having the amino acid sequence set forth in SEQ ID NO: 11 and/or a VL having the amino acid sequence set forth in SEQ ID NO: 12, and wherein the N-terminal glutamic acid of SEQ ID NO: 11 and/or the N-terminal glutamine of SEQ ID NO: 12 has undergone cyclization to pyroglutamate.

In certain embodiments, the antibody or antigen-binding fragment thereof according to the present invention comprises: a VH having the amino acid sequence set forth in SEQ ID NO: 11 and/or a VL having the amino acid sequence set forth in SEQ ID NO: 12, and wherein and the N-terminal glutamic acid of SEQ ID NO: 11 and/or the N-terminal glutamine of SEQ ID NO: 12 has undergone cyclization to pyroglutamic acid.

In certain embodiments, the antibody or antigen-binding fragment thereof according to the present invention comprises: a VH having the amino acid sequence set forth in SEQ ID NO: 15 and/or a VL having the amino acid sequence set forth in SEQ ID NO: 16, and wherein the N-terminal glutamine of SEQ ID NO: 15 and/or the N-terminal glutamic acid of SEQ ID NO: 16 has undergone cyclization to pyroglutamate.

In certain embodiments, the antibody or antigen-binding fragment thereof according to the present invention comprises: a VH having the amino acid sequence set forth in SEQ ID NO: 15 and/or a VL having the amino acid sequence set forth in SEQ ID NO: 16, and wherein the N-terminal glutamine of SEQ ID NO: 15 and/or the N-terminal glutamic acid of SEQ ID NO: 16 has undergone cyclization to pyroglutamic acid.

In certain embodiments, the antibody or antigen-binding fragment thereof according to the present invention comprises: (a) a heavy chain comprising the amino acid sequence set forth in SEQ ID NO: 135 or a variant thereof and/or a light chain comprising the amino acid sequence set forth in SEQ ID NO: 112; (b) a heavy chain consisting of the amino acid sequence set forth in SEQ ID NO: 135 and a light chain consisting of the amino acid sequence set forth in SEQ ID NO: 112; (c) a heavy chain comprising the amino acid sequence set forth in SEQ ID NO: 135 and a light chain comprising the amino acid sequence set forth in SEQ ID NO: 112.

In certain embodiments, the antibody or antigen-binding fragment thereof according to the present invention comprises: (a) a heavy chain consisting of the amino acid sequence set forth in SEQ ID NO: 136 and a light chain consisting of the amino acid sequence set forth in SEQ ID NO: 112; (b) a heavy chain consisting of the amino acid sequence set forth in SEQ ID NO: 137 and a light chain consisting of the amino acid sequence set forth in SEQ ID NO: 112; or (c) a heavy chain consisting of the amino acid sequence set forth in SEQ ID NO: 138 and a light chain consisting of the amino acid sequence set forth in SEQ ID NO: 112.

In certain embodiments, the antibody or antigen-binding fragment thereof according to the present invention comprises: (a) a heavy chain comprising the amino acid sequence set forth in SEQ ID NO: 111 or a variant thereof and/or a light chain comprising the amino acid sequence set forth in SEQ ID NO: 112; (b) a heavy chain consisting of the amino acid sequence set forth in SEQ ID NO: 111 and a light chain consisting of the amino acid sequence set forth in SEQ ID NO: 112; or (c) a heavy chain comprising the amino acid sequence set forth in SEQ ID NO: 111 and a light chain comprising the amino acid sequence set forth in SEQ ID NO: 112.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 or a variant thereof and/or a VL having the amino acid sequence set forth in SEQ ID NO: 2 or a variant thereof;
(b) a VH having the amino acid sequence set forth in SEQ ID NO: 3 or a variant thereof and/or a VL having the amino acid sequence set forth in SEQ ID NO: 4 or a variant thereof;
(c) a VH having the amino acid sequence set forth in SEQ ID NO: 5 or a variant thereof and/or a VL having the amino acid sequence set forth in SEQ ID NO: 6 or a variant thereof;
(d) a VH having the amino acid sequence set forth in SEQ ID NO: 7 or a variant thereof and/or a VL having the amino acid sequence set forth in SEQ ID NO: 8 or a variant thereof;
(e) a VH having the amino acid sequence set forth in SEQ ID NO: 9 or a variant thereof and/or a VL having the amino acid sequence set forth in SEQ ID NO: 10 or a variant thereof;
(f) a VH having the amino acid sequence set forth in SEQ ID NO: 11 or a variant thereof and/or a VL having the amino acid sequence set forth in SEQ ID NO: 12 or a variant thereof;
(g) a VH having the amino acid sequence set forth in SEQ ID NO: 13 or a variant thereof and/or a VL having the amino acid sequence set forth in SEQ ID NO: 14 or a variant thereof;
(h) a VH having the amino acid sequence set forth in SEQ ID NO: 15 or a variant thereof and/or a VL having the amino acid sequence set forth in SEQ ID NO: 16 or a variant thereof;
(i) a VH having the amino acid sequence set forth in SEQ ID NO: 17 or a variant thereof and/or a VL having the amino acid sequence set forth in SEQ ID NO: 18 or a variant thereof;
(j) a VH having the amino acid sequence set forth in SEQ ID NO: 19 or a variant thereof and/or a VL having the amino acid sequence set forth in SEQ ID NO: 20 or a variant thereof;
(k) a VH having the amino acid sequence set forth in SEQ ID NO: 21 or a variant thereof and/or a VL having the amino acid sequence set forth in SEQ ID NO: 22 or a variant thereof; or
(l) a VH having the amino acid sequence set forth in SEQ ID NO: 23 or a variant thereof and/or a VL having the amino acid sequence set forth in SEQ ID NO: 24 or a variant thereof;
   wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived. In certain embodiments, the replacement is conservative replacement. In certain embodiments, the variants in (a)-(l) have variations in the antibody framework region of the sequence from which the variants are derived. In certain embodiments, the variations are replacement, deletion or addition of one or several amino acids (such as replacement, deletion or addition of 1, 2, 3, 4 or 5 amino acids) in the antibody framework region of the sequence from which the variants are derived. In certain embodiments, the variations are replacement of one or several amino acids in the antibody framework region of the sequence from which the variants are derived. In certain embodiments, the replacement is conservative replacement in the antibody framework region of the sequence from which the variants are derived.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 and/or a VL having the amino acid sequence set forth in SEQ ID NO: 2;
(b) a VH having the amino acid sequence set forth in SEQ ID NO: 3 and/or a VL having the amino acid sequence set forth in SEQ ID NO: 4;
(c) a VH having the amino acid sequence set forth in SEQ ID NO: 5 and/or a VL having the amino acid sequence set forth in SEQ ID NO: 6;
(d) a VH having the amino acid sequence set forth in SEQ ID NO: 7 and/or a VL having the amino acid sequence set forth in SEQ ID NO: 8;
(e) a VH having the amino acid sequence set forth in SEQ ID NO: 9 and/or a VL having the amino acid sequence set forth in SEQ ID NO: 10;
(f) a VH having the amino acid sequence set forth in SEQ ID NO: 11 and/or a VL having the amino acid sequence set forth in SEQ ID NO: 12;
(g) a VH having the amino acid sequence set forth in SEQ ID NO: 13 and/or a VL having the amino acid sequence set forth in SEQ ID NO: 14;
(h) a VH having the amino acid sequence set forth in SEQ ID NO: 15 and/or a VL having the amino acid sequence set forth in SEQ ID NO: 16;
(i) a VH having the amino acid sequence set forth in SEQ ID NO: 17 and/or a VL having the amino acid sequence set forth in SEQ ID NO: 18;
(j) a VH having the amino acid sequence set forth in SEQ ID NO: 19 and/or a VL having the amino acid sequence set forth in SEQ ID NO: 20;
(k) a VH having the amino acid sequence set forth in SEQ ID NO: 21 and/or a VL having the amino acid sequence set forth in SEQ ID NO: 22; or
(l) a VH having the amino acid sequence set forth in SEQ ID NO: 23 and/or a VL having the amino acid sequence set forth in SEQ ID NO: 24.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (a) a VH having the amino acid sequence set forth in SEQ ID NO: 1 or a variant thereof and/or a VL having the amino acid sequence set forth in SEQ ID NO: 2 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (b) a VH having the amino acid sequence set forth in SEQ ID NO: 3 or a variant thereof and/or a VL having the amino acid sequence set forth in SEQ ID NO: 4 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (c) a VH having the amino acid sequence set forth in SEQ ID NO: 5 or a variant thereof and/or a VL having the amino acid sequence set forth in SEQ ID NO: 6 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (d) a VH having the amino acid sequence set forth in SEQ ID NO: 7 or a variant thereof and/or a VL having the amino acid sequence set forth in SEQ ID NO: 8 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (e) a VH having the amino acid sequence set forth in SEQ ID NO: 9 or a variant thereof and/or a VL having the amino acid sequence set forth in SEQ ID NO: 10 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (f) a VH having the amino acid sequence set forth in SEQ ID NO: 11 or a variant thereof and/or a VL having the amino acid sequence set forth in SEQ ID NO: 12 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (g) a VH having the amino acid sequence set forth in SEQ ID NO: 13 or a variant thereof and/or a VL having the amino acid sequence set forth in SEQ ID NO: 14 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (h) a VH having the amino acid sequence set forth in SEQ ID NO: 15 or a variant thereof and/or a VL having the amino acid sequence set forth in SEQ ID NO: 16 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (i) a VH having the amino acid sequence set forth in SEQ ID NO: 17 or a variant thereof and/or a VL having the amino acid sequence set forth in SEQ ID NO: 18 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (j) a VH having the amino acid sequence set forth in SEQ ID NO: 19 or a variant thereof and/or a VL having the amino acid sequence set forth in SEQ ID NO: 20 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (k) a VH having the amino acid sequence set forth in SEQ ID NO: 21 or a variant thereof and/or a VL having the amino acid sequence set forth in SEQ ID NO: 22 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (1) a VH having the amino acid sequence set forth in SEQ ID NO: 23 or a variant thereof and/or a VL having the amino acid sequence set forth in SEQ ID NO: 24 or a variant thereof.

In certain embodiments, the antibody or antigen-binding fragment thereof according to the present invention is a murine antibody, a chimeric antibody, a humanized antibody or a fully humanized antibody.

In certain embodiments, the antibody or antigen-binding fragment thereof according to the present invention is a murine antibody.

In certain embodiments, the antibody or antigen-binding fragment thereof according to the present invention is a chimeric antibody.

In certain embodiments, the antibody or antigen-binding fragment thereof according to the present invention is a humanized antibody.

In certain embodiments, the antibody or antigen-binding fragment thereof according to the present invention is a fully humanized antibody.

In certain embodiments, the antibody or antigen-binding fragment thereof according to any one of the above embodiments is selected from scFv, Fab, Fab′, (Fab′)2, Fab′-SH, an Fv fragment, disulfide bond linked Fv (dsFv), a diabody, a bispecific antibody and a multispecific antibody.

### Derived Antibody

The antibody or the antigen-binding fragment thereof according to the present invention may be derivatized, e.g., the antibody or antigen-binding fragment thereof may be linked to another molecule (e.g., another polypeptide or protein). Typically, derivatization (like marking) of the antibody or antigen-binding fragment thereof will not adversely affect its binding to PTK7 (particularly human PTK7). Therefore, the antibody or antigen-binding fragment thereof according to the present invention are also contemplated to include such derivatized forms. For example, the antibody or antigen-binding fragment thereof according to the present invention may be functionally linked (by chemical coupling, gene fusion, non-covalent linkage, or otherwise) to one or more other molecule groups, e.g., another antibody (like forming a bispecific antibody), a detection reagent, a pharmaceutical reagent, and/or a protein or polypeptide (like an avidin or a polyhistidine tag) capable of mediating binding of an antibody or antigen-binding fragment to another molecule.

As one of derivatives of the antibody, a conjugate according to the present invention includes the antibody or antigen-binding fragment thereof and the coupling part according to the present invention.

In certain embodiments, the coupling part is selected from a detectable marker. The detectable marker according to the present invention may be any substance which can be detected by means of fluorescence, spectrum, photochemistry, biochemistry, immunology, electricity, optics or chemistry. Such marker is well-known in the art, and the examples of the marker include, but are not limited to, enzymes (like horseradish peroxidase, alkaline phosphatase, (3-galactosidase, urease, glucose oxidase), radionuclides (like 3H, 1251, 35S, 14C or 32P), fluorochrome (like fluorescein isothiocyanate (FITC), fluorescein, tetramethylrhodamine isothiocyanate (TRITC), phycoerythrin (PE), texas red, rhodamine, quantum dots or cyanine dye derivatives (like Cy7 and Alexa 750)), acridinium ester compounds, magnetic beads (like Dynabeads®), thermal measurement markers such as colloidal gold or colored glass or plastic (like polystyrene, polypropylene and latex) beads, and biotin for binding the above marker-modified avidins (like streptavidin). In certain embodiments, such marker can be used for immunological detection (like enzyme linked immunoassay, radioimmunoassay, fluorescence immunoassay and chemiluminescence immunoassay). In certain embodiments, the detectable marker is selected from radioisotopes, fluorescent substances, luminescent substances, colored substances or enzymes. In certain embodiments, the detectable marker described above may be linked to the antibody or antigen-binding fragment thereof according to the present invention by linkers of different lengths so as to reduce potential steric hindrance.

In certain embodiments, the coupling part is selected from a therapeutic reagent. In certain embodiments, the therapeutic reagent is preferably an anti-tumor reagent, such as a cytotoxic reagent, cytokine, toxin or radionuclide.

In certain embodiments, the coupling part is selected from a substance which can improve the biological properties of the antibody (e.g., increasing serum half-life), such as a chemical group like polyethylene glycol (PEG), methyl or ethyl, or a glycosyl group.

As one of the derivatives of the antibody, a multispecific antibody according to the present invention comprises the antibody or antigen-binding fragment thereof according to the present invention.

In certain embodiments, the multispecific antibody comprises the antibody or antigen-binding fragment thereof according to the present invention as a first antigen-binding domain, and further comprises at least one second antigen-binding domain for other targets.

In certain embodiments, each antigen-binding domain of the multispecific antibody retains a respective original binding specificity.

In certain embodiments, the multispecific antibody is a bispecific antibody or a trispecific antibody or a tetraspecific antibody.

### Preparation of Antibody

The antibody according to the present invention may be prepared by various methods known in the art, such as by genetic engineering recombination technology. For example, DNA molecules encoding the heavy chain and light chain genes of the antibody according to the present invention may be obtained by chemical synthesis or PCR amplification. The obtained DNA molecules are inserted into an expression vector and then transfected into a host cell. Then, the transfected host cell is cultured under specific conditions and then the antibody according to the present invention is expressed.

The antigen-binding fragments according to the present invention may be obtained by hydrolyzing complete antibody molecules (see Morimoto et al., J. Biochem. Biophys. Methods 24:107-117 (1992) and Brennan et al., Science 229:81 (1985)). In addition, these antigen-binding fragments may also be directly generated by recombinant host cells (reviewed in Hudson, Curr. Opin. Immunol. 11: 548-557 (1999); Little et al., Immunol. Today, 21: 364-370 (2000)). For example, a Fab′ fragment may be directly obtained from the host cell; the Fab′ fragment may be chemically coupled to form an F(ab′)2 fragment (Carter et al., Bio/Technology, 10: 163-167 (1992)). In addition, Fv, Fab or F(ab′)₂ fragments may also be directly separated from a recombinant host cell culture solution. Ordinary technicians in the art are fully aware of other techniques for preparing these antigen-binding fragments.

Therefore, in another aspect, the present invention provides isolated nucleic acid molecules comprising a nucleotide sequence encoding the antibody or antigen-binding fragment thereof according to the present invention, or the heavy chain variable region and/or light chain variable region thereof. In certain embodiments, the nucleotide sequence is substitutable according to the codon degeneracy in the art. In certain embodiments, the nucleotide sequence is codon-optimized.

In certain embodiments, the isolated nucleic acid molecules comprise nucleic acid molecules encoding the heavy chain variable region of the antibody, and/or nucleic acid molecules encoding the light chain variable region of the antibody;
the nucleic acid molecules encoding the heavy chain variable region of the antibody comprise: (i) a nucleotide sequence set forth in SEQ ID NO: 127, (ii) a sequence substantially identical to SEQ ID NO: 127 (e.g., a sequence having at least about 85%, 90%, 95%, 99% or higher sequence identity, or a sequence subjected to substitution of one or more nucleotides, as compared to SEQ ID NO: 127), or (iii) a degenerate sequence of (i) or (ii); and/or, the nucleic acid molecules encoding the light chain variable region of the antibody comprise: (iv) a nucleotide sequence set forth in SEQ ID NO: 128, (v) a sequence substantially identical to SEQ ID NO: 128 (e.g., a sequence having at least about 85%, 90%, 95%, 99% or higher sequence identity, or a sequence subjected to substitution of one or more nucleotides, as compared to SEQ ID NO: 128), or (vi) a degenerate sequence of (iv) or (v).

In certain embodiments, the isolated nucleic acid molecules comprise nucleic acid molecules encoding the heavy chain of the antibody, and/or nucleic acid molecules encoding the light chain of the antibody, wherein
the nucleic acid molecules encoding the heavy chain of the antibody comprise: (i) a nucleotide sequence shown as SEQ ID NO: 132, (ii) a sequence substantially identical to SEQ ID NO: 132, or (iii) a degenerate sequence of (i) or (ii); and/or, the nucleic acid molecules encoding the light chain of the antibody comprise: (iv) a nucleotide sequence shown as SEQ ID NO: 130, (v) a sequence substantially identical to SEQ ID NO: 130, or (vi) a degenerate sequence of (iv) or (v).

In certain embodiments, the isolated nucleic acid molecules comprise nucleic acid molecules encoding the heavy chain of the antibody, and/or nucleic acid molecules encoding the light chain of the antibody, wherein
the nucleic acid molecules encoding the heavy chain of the antibody comprise: (i) a nucleotide sequence shown as SEQ ID NO: 129, (ii) a sequence substantially identical to SEQ ID NO: 129, or (iii) a degenerate sequence of (i) or (ii); and/or, the nucleic acid molecules encoding the light chain of the antibody comprise: (iv) a nucleotide sequence shown as SEQ ID NO: 130, (v) a sequence substantially identical to SEQ ID NO: 130, or (vi) a degenerate sequence of (iv) or (v).

In another aspect, the present invention provides a vector (like a cloning vector or an expression vector) comprising the isolated nucleic acid molecules according to the present invention. In certain embodiments, the vector according to the present invention is, for example, a plasmid, a cosmid, a bacteriophage and a lentivirus. In certain embodiments, the vector is capable of expressing the antibody or antigen-binding fragment thereof according to the present invention in a subject (e.g., mammal like human).

In certain embodiments, the vector comprises a first nucleotide sequence encoding the heavy chain or heavy chain variable region of the antibody or antigen-binding fragment thereof according to the present invention and a second nucleotide sequence encoding the light chain or light chain variable region thereof; the first nucleotide sequence and the second nucleotide sequence are in the same or different vectors. When the first nucleotide sequence and the second nucleotide sequence are in different vectors, the vector according to the present invention comprises a first vector having the first nucleotide sequence and a second vector having the second nucleotide sequence.

In certain embodiments, the antibody or antigen-binding fragment thereof according to the present invention can be used for constructing a chimeric antigen receptor (CAR) comprising an extracellular antigen-binding domain (like scFv) that specifically binds PTK7, a transmembrane domain, and one or more intracellular T cell signaling domains. In such embodiments, the isolated nucleic acid molecules according to the present invention may comprise a nucleotide sequence encoding the chimeric antigen receptor, and the nucleotide sequence encoding the chimeric antigen receptor further comprises a nucleotide sequence encoding the antibody or antigen-binding fragment thereof (like scFv) according to the present invention. In certain embodiments, the isolated nucleic acid molecules according to the present invention encodes chimeric antigen receptor comprising the antigen-binding fragment of the antibody according to the present invention (like scFv).

In certain embodiments, the antibody or antigen-binding fragment thereof according to the present invention can be used for constructing chimeric antigen receptor modified immune cells, and the chimeric antigen receptor modified immune cells comprise the CAR and immune cells (like T lymphocytes and NK cells).

In another aspect, the present invention provides a host cell comprising the isolated nucleic acid molecules according to the present invention or the vector according to the present invention. The host cell may be eukaryotic cell (like mammalian cell, insect cell or yeast cell) or prokaryotic cell (like Escherichia coli). Suitable eukaryotic cells include but are not limited to NS0 cell, Vero cell, Hela cell, COS cell, CHO cell, ExpiCHO cell, HEK293 cell, Expi293 cell, BHK cell and MDCKII cell. Suitable insect cells include but are not limited to Sf9 cell. In certain embodiments, the host cell according to the present invention is mammalian cell, such as CHO (like CHO-K1, CHO-S, CHO DXB11, ExpiCHO and CHO DG44).

In certain embodiments, the host cell according to the present invention may be chimeric antigen receptor T cell (CAR-T). In such embodiments, the isolated nucleic acid molecules in the host cell may comprise a nucleotide sequence encoding the chimeric antigen receptor, and the nucleotide sequence encoding the chimeric antigen receptor further comprises a nucleotide sequence encoding the antibody or antigen-binding fragment thereof (like scFv) according to the present invention. In certain embodiments, the isolated nucleic acid molecules in the host cell encode the chimeric antigen receptor comprising the antigen-binding fragment (like scFv) of the antibody according to the present invention.

In another aspect, the present invention provides a method for preparing the antibody or antigen-binding fragment thereof according to the present invention, and the method includes: culturing the host cell according to the present invention under a condition that the expression of the antibody or antigen-binding fragment thereof is allowed, and recovering the antibody or antigen-binding fragment thereof from the cultured host cell culture.

### Therapeutic Use

In another aspect, the present invention provides a pharmaceutical composition comprising the antibody or antigen-binding fragment thereof, the nucleic acid molecules, the vector, the host cell, the conjugate, or the multispecific antibody according to the present invention, and a pharmaceutically acceptable carrier and/or excipient.

In certain embodiments, the pharmaceutical composition according to the present invention comprises the antibody or antigen-binding fragment thereof, and the pharmaceutically acceptable carrier and/or excipient according to the present invention.

In certain embodiments, the pharmaceutical composition may also comprise an additional pharmaceutical active agent. In certain embodiments, the additional pharmaceutical active agent is a drug having anti-tumor activity. In certain embodiments, the additional pharmaceutical active agent is selected from an EGFR inhibitor, a BCR-ABL, FLT3, KIT or RET inhibitor, an HER2 inhibitor, an HER3 inhibitor, an HER4 inhibitor, an IGFR-1 inhibitor, an mTOR inhibitor, a PI3 kinase inhibitor, a c-met or VEGF inhibitor, a PARP inhibitor, a chemotherapeutic drug or any combination thereof. In certain embodiments, the antibody or antigen-binding fragment thereof and the additional pharmaceutical active agent according to the present invention are provided as independent components or mixed components. Therefore, the antibody or antigen-binding fragment thereof according to the present invention may be simultaneously, separately or sequentially administrated with the additional pharmaceutical active agent.

In certain embodiments, the antibody or antigen-binding fragment thereof, the nucleic acid molecules, the vector, the host cell, the conjugate, or the multispecific antibody in the pharmaceutical composition according to the present invention are sufficient to (e.g., in the subject):
(a) inhibit cell proliferation;
(b) inhibit tumor growth;
(c) induce and/or improve antibody-dependent cytotoxic activity;
(d) inhibit PTK7-mediated signaling;
(e) prevent and/or treat PTK7-mediated disease/disorder; or
(f) any combination of (a)-(e).

In certain embodiments, the PTK7-mediated disease/disorder is tumor, such as tumor expressing PTK7. In certain embodiments, the tumor is selected from uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

In another aspect, the present invention provides use of the antibody or antigen-binding fragment thereof, the nucleic acid molecules, the vector, the host cell, the conjugate, the multispecific antibody or the pharmaceutical composition according to the present invention in preparation of drugs. The drugs are used for inhibiting cell proliferation, or preventing and/or treating and/or assisting in tumor treatment.

In certain embodiments, the drugs are used for inhibiting the proliferation of cell (such as tumor cell) expressing the PTK7.

In another aspect, the present invention provides a method for inhibiting cell proliferation, and the method includes: making the cell in contact with the antibody or antigen-binding fragment thereof, the nucleic acid molecules, the vector, the host cell, the conjugate, the multispecific antibody or the pharmaceutical composition according to the present invention. In certain embodiments, the cell is the cell expressing PTK7, such as tumor cell.

In another, the present invention provides a method for preventing and/or treating/or assisting in tumor treatment in a subject, and the method includes: administrating an effective amount of the antibody or antigen-binding fragment thereof, the nucleic acid molecules, the vector, the host cell, the conjugate, the multispecific antibody or the pharmaceutical composition according to the present invention to the subject in need thereof.

In certain embodiments, the method further includes: performing a second therapy to the subject, the second therapy being selected from surgery, chemotherapy, radiotherapy, immunotherapy, gene therapy, DNA therapy, RNA therapy, nano therapy, virus therapy, adjuvant therapy, and any combination thereof. In certain embodiments, the second therapy can be applied simultaneously, separately, or sequentially to the above method.

In any one of the above embodiments, the tumor involved in the antibody or antigen-binding fragment thereof, the nucleic acid molecules, the vector, the host cell, the conjugate, the multispecific antibody or the pharmaceutical composition according to the present invention may be of any tumor form. In certain embodiments, the tumor involved in the antibody or antigen-binding fragment thereof, the nucleic acid molecules, the vector, the host cell, the conjugate, the multispecific antibody or the pharmaceutical composition according to the present invention is a PTK7 positive tumor. In certain embodiments, the tumor involved in the antibody or antigen-binding fragment thereof, the nucleic acid molecules, the vector, the host cell, the conjugate, the multispecific antibody or the pharmaceutical composition according to the present invention is selected from uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

The antibody or the antigen-binding fragment thereof according to the present invention and the pharmaceutical composition according to the present invention may be prepared into any dosage form known in the medical field, such as tablets, pills, suspensions, emulsions, solutions, gels, capsules, powder, granules, elixirs, lozenges, suppositories, injections (including injections, sterile powder for injection and concentrated solutions for injection), inhalers, sprays and the like. The preferable dosage form depends on the expected administration mode and treatment use. The pharmaceutical composition according to the present invention is to be sterile and is to be stable under production and storage conditions. One preferred dosage form is the injection. Such injection may be a sterile injection solution. For example, the sterile injection solution may be prepared by the following method: doping an essential dose of the antibody according to the present invention in a suitable solvent, and optionally simultaneously doping other desirable ingredients (including, but not limited to, a pH adjuster, a surfactant, an adjuvant, an ionic strength enhancer, an isotonic reagent, a preservative, a diluent, or any combination thereof), and then performing filtration sterilization. In addition, the sterile injection solution may be prepared as sterile lyophilized powder (e.g., by vacuum drying or freeze drying) facilitating storage and use. Such sterile lyophilized powder may be dispersed in a suitable vector, e.g., sterile pyrogen-free water, prior to use.

In addition, the antibody or antigen-binding fragment thereof according to the present invention may be present in the pharmaceutical composition in a unit dose form for ease of administration.

The antibody or antigen-binding fragment thereof, and the pharmaceutical composition according to the present invention may be administered by any suitable method known in the art, including, but not limited to, oral, buccal, sublingual, eyeball, local, parenteral, rectal, intrathecal, intraalveolar, inguinal, intravesical, topical (e.g., powder, ointment, or drops), or nasal administration. However, for many therapeutic uses, the preferred route/manner of administration is parenteral administration (e.g., intravenous injection, subcutaneous injection, intraperitoneal injection, intramuscular injection). The technician needs to understand that the route and/or manner of administration will vary according to the intended purpose. In a preferred embodiment, the antibody or antigen-binding fragment thereof, and the pharmaceutical compositions according to the present invention are administered by intravenous infusion or injection.

The pharmaceutical composition according to the present invention may include a “therapeutically effective amount” or a “prophylactically effective amount” of the antibody or antigen-binding fragment thereof according to the present invention. The “prophylactically effective amount” refers to the amount sufficient to prevent, or delay the occurrence of a disease. The “therapeutically effective amount” refers to the amount sufficient to achieve a desired clinical effect in an individual being treated. For instance, this may be the amount necessary to alleviate any particular disease symptom or inhibit or reduce the severity of a disease in an individual. The therapeutically effective amount of the antibody or antigen-binding fragment thereof according to the present invention may vary according to the following factors: the severity of the disease to be treated, the overall state of the immune system of the patient, the general conditions of the patient such as age, weight, and gender, the manner of administration of the drug, and other treatments performed simultaneously, and the like.

According to the present invention, the dosing regimen may be adjusted to achieve an optimal response for purposes (e.g., treatment or prevention response). For example, the drug may be administrated by a single dose, or the drug may be administrated by multiple doses over a period of time, or the dose may be reduced or increased in proportion to the degree of urgency of the treatment.

According to the present invention, the subject may be mammal, such as human.

### Detection Use

The antibody or the antigen-binding fragment according to the present invention is capable of being specifically bound to PTK7 so as to detect the presence or level of the PTK7 in the sample.

Therefore, in another aspect, the present invention provides a kit comprising the antibody or antigen-binding fragment according to the present invention. In certain embodiments, the antibody or antigen-binding fragment according to the present invention has a detectable marker. In a preferred embodiment, the kit further includes a secondary antibody for specifically recognizing the antibody or antigen-binding fragment thereof according to the present invention. Preferably, the secondary antibody further includes a detectable marker.

According to the present invention, the detectable marker may be any substance which can be detected by means of fluorescence, spectrum, photochemistry, biochemistry, immunology, electricity, optics or chemistry. Particularly preferably, such marker can be used for immunological detection (like enzyme linked immunoassay, radioimmunoassay, fluorescence immunoassay and chemiluminescence immunoassay). Such marker is well-known in the art, and the examples of the marker include, but are not limited to, enzymes (like horseradish peroxidase, alkaline phosphatase, β-galactosidase, urease, glucose oxidase), radionuclides (like 3H, 125I, 35S, 14C or 32P), fluorochrome (like fluorescein isothiocyanate (FITC), fluorescein, tetramethylrhodamine isothiocyanate (TRITC), phycoerythrin (PE), texas red, rhodamine, quantum dots or cyanine dye derivatives (like Cy7 and Alexa 750)), acridinium ester compounds, magnetic beads (like Dynabeads®), thermal measurement markers such as colloidal gold or colored glass or plastic (like polystyrene, polypropylene, latex) beads, and biotin for binding to the above marker-modified avidins (like streptavidin). In certain embodiments, the detectable marker described above may be linked to the antibody according to the present invention by linkers of different lengths so as to reduce potential steric hindrance.

In another aspect, the present invention provides a method for detecting the presence or level of PTK7 in a sample, and the method includes a step of using the antibody or antigen-binding fragment thereof according to the present invention. In a preferred embodiment, the antibody or antigen-binding fragment thereof according to the present invention further has a detectable marker. In another preferred embodiment, the method further includes: detecting the antibody or antigen-binding fragment thereof according to the present invention by a reagent having the detectable marker. The method may be suitable for diagnosis or non-diagnostic purposes (for example, the sample is a cell sample, rather than a sample from the patient).

In certain embodiments, the method includes: making the sample in contact with the antibody or antigen-binding fragment thereof according to the present invention under a condition that a complex is allowed to be formed between the antibody or antigen-binding fragment thereof and PTK7, and detecting the formation of the complex.

Because of low expression or no expression of PTK7 in normal tissue, as well as expression or high expression in some cancers, the tumor may be diagnosed by detecting the presence or level of the PTK7 in the sample. Therefore, in certain embodiments, the method is used for diagnosing the tumor, such as PTK7 positive tumor, like uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer (such as small cell lung cancer, non-small cell lung cancer, lung adenocarcinoma and lung squamous cell carcinoma), esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

In certain embodiments, the method includes: detecting the expression level of the PTK7 in a to-be-detected sample from the subject, and comparing the expression level with a reference value (such as health control); and the increase in the expression level compared with the reference value is an indication of the tumor.

In another aspect, the present invention provides use of the antibody or antigen-binding fragment thereof according to the present invention; and the kit is used for detecting the presence or level of the PTK7 in the sample, and/or diagnosing tumor.

In another aspect, the present invention provides a diagnostic or therapeutic kit comprising the antibody or antigen-binding fragment thereof, the nucleic acid molecules, the vector, the host cell, the conjugate or the multispecific antibody according to the present invention, and an instruction for use.

The antibody according to the invention has high binding affinity with PTK7, can induce efficient endocytosis after being bound to cells expressing PTK7, and has good hydrophilicity, and may have advantages of being better in quality and in vivo metabolic properties, and suitable for coupling. Therefore, the antibody according to the present invention has a potential for preventing and/or treating tumors. In addition, the antibody according to the present invention is the humanized antibody, which can be safely administered to human subjects, and can reduce the risk of causing immunogenic reaction. Therefore, the antibody according to the invention has great clinical value.

The embodiments of the present invention will be described in detail below with reference to the accompanying drawings and examples, but those skilled in the art will understand that the following drawings and examples are only used for illustrating the present invention, and are not intended to limit the scope of the present invention. According to the accompanying drawings and the following detailed description of preferred embodiments, various objects and advantageous aspects of the present invention will become practical for those skilled in the art.

### EXAMPLES

The following descriptions of specific embodiments will further illustrate the present invention, but this is not a limitation to the present invention. Those skilled in the art can make various modifications or improvements according to the teaching of the present invention without departing from the basic idea and scope of the present invention.

### Sequence Information

Information about the sequences involved in the present invention is described in the following table:

| SEQ ID | | |
|---|---|---|
| NO: | Description | Sequence |
| 1 | 101A6 VH | EVKLVESGGGLVQPGGSLSLSCAASGFTFTDYYLSWVR |
| | amino acid | QPPGKALEWLALIRNKANGYTTEYSASVKGRFTISRDS |
| | sequence | SQSILYLQMDALRAEDSATYYCARDTLAAYWGQGTSV |
| | | TVSS |
| 2 | 101A6 VL | DIVMTQSHKFMSTSVGDRVSFTCKASQDVGSAVIWYQ |
| | amino acid | QKPGQSPKLLIYWASTRHTGVPDRFTGSGSGTDFTLTIS |
| | sequence | NVQSEDLADYFCQQYSSYPLTFGAGTKLEIK |
| 3 | 64A10 VH | QVQLKESGPVLVAPSQSLSITCTVSGFSLTSYGVYWVRQ |
| | amino acid | PPGKGPEWLGVIWTSGNTNYNSALMSRLSISKDNSKSQ |
| | sequence | VFLKMNSLQTDDTAMYYCVKHDGGNYVGVMNYWGQ |
| | | GTSVTVSS |
| 4 | 64A10 VL | DIQMTQSPATLSVTPGDRVSLSCRASQSISDYLHWYQQ |
| | amino acid | KSQESPRLLIKYASQSISGIPSRFSGSGSGSDFTLSINSVEP |
| | sequence | EDVGVYYCQNGHSFPPTFGGGTKLEIK |
| 5 | 4E12 VH amino | QVQLQQSGAELMRPGVSVKISCKGSGYTFTDYAMHW |
| | acid sequence | VKQNHAKSLEWIGVISTYYGNVSYNQKFKGKATMTVD |
| | | KSSSTAYMELARLTSEDSAIYYCARGFAYWGQGTLVTV |
| | | SA |
| 6 | 4E12 VL amino | DIVITQDELSNLVTSGESVSISCRSSKSLQKKDGKTYLN |
| | acid sequence | WFLQRPGQSPQLLIYLMSTRASGVSDRFSGSGSGTDFT |
| | | LEISRVKAEDVGVYYCQQLVEYPFTFGSGTKLEIK |
| 7 | 19C2 VH amino | QVTLKESGPGILKPSQTLSLTCSFSGFSLTTSGMGINWIR |
| | acid sequence | QPSGKGLEWLALIWWDDDKYYNPSLKNRLTVSKDPSR |
| | | NQVFLTITSVDTTDSATYYCARSLITTAPSDYWGQGTSV |
| | | TVSS |
| 8 | 19C2 VL amino | DIVMTQSQKFMSTSVGDRVSITCKASQNVGTAITWYQQ |
| | acid sequence | RPGQSPQLLIYSASNRYTGVPDRFTGSGSGTDFTLTISNL |
| | | QSEDLADYFCLQYSDYPLTFGAGTNLELK |
| 9 | 4B12 VH amino | QVTLKESGPGILKPAQTLSLTCSFSGFSLSTSGMGVGWI |
| | acid sequence | RQPSGKGLEWLAHIWWDDDKHYNPSLKSRLTISKDAS |
| | | RNQVFLRISSVDTADTATYYCVRSNYGYDWFAYWGQG |
| | | TLVTVSA |
| 10 | 4B12 VL amino | DIKMTQSPSSMYVSLGERVTITCKASQDINTYLGWFQQ |
| | acid sequence | KPGKSPKTLIYRANRLVDGVPSRFSGSGSGQDYSLTISSL |
| | | EYEDMEIYYCLQYDEFPLTFGAGTKLEIK |
| 11 | 57F4 VH amino | EVQLVESGGGLVKPGGSRKLSCAASGFTFSDYGMHWV |
| | acid sequence | RQAPEKGLEWVAYISSGSSALYYADTVKGRFTISRDNV |
| | | KNTLFLQMTSLRSEDTAMYYCARRSYYSYFAYWGQGT |
| | | LVTVSA |
| 12 | 57F4 VL amino | QIVLTQSPALMAASPGEKVTITCRVSSSISSSTLHWYQQ |
| | acid sequence | KSETSPKPWIYGTSNLASGVPVRFSGSGSGTSYSLTISSM |
| | | EAEDAATYYCQQWSSFPFTFGSGTKLEIK |
| 13 | 8H3 VH amino | EVKLVESGGGLVQPGGSLSLSCAASGFTFTDYYMSWV |
| | acid sequence | RQPPGKALEWLALIRNKANGYTTEYSASVKGRFTISRD |
| | | NSQSILYLQMNALRAEDSATYYCARDSFEVYWGQGTL |
| | | VTVSA |
| 14 | 8H3 VL amino | DIVLTQSHKFMSTSVGDRVNITCKASQDVGTAVAWYQQ |
| | acid sequence | KPGQSPKLLIYWASTRHTGVPDRFTGSGSGTDFTLTIRN |
| | | VQSEDLADYFCQQFSSFPFTFGGGTKLEIK |
| 15 | 64A10HZ VH | QVQLQESGPGLVKPSETLSLTCTVSGFSLTSYGVYWVR |
| | amino acid | QPPGKGPEWLGVIWTSGNTNYNPSLKSRLTISKDNSKN |
| | sequence | QVSLKLSSVTAADTAVYYCVKHDGGNYVGVMNYWGQ |
| | | GTLVTVSS |
| 16 | 64A10HZ VL | EIVMTQSPDFQSVTPKEKVTITCRASQSISDYLHWYQQ |
| | amino acid | KPDQSPKLLIKYASQSISGVPSRFSGSGSGSDFTLTINSLE |
| | sequence | AEDAATYYCQNGHSFPPTFGQGTKLEIK |
| 17 | 64A10HZ05 VH | QVQLQESGPGLVKPSETLSLTCTVSGFSLTSYGVYWVR |
| | amino acid | QPPGKGLEWLGVIWTSGNTNYNPSLKSRLTISKDNSKN |
| | sequence | QVSLKLSSVTAADTAVYYCVKHDGGNYVGVMNYWGQ |
| | | GTLVTVSS |
| 18 | 64A10HZ05 VL | DIQMTQSPSSLSASVGDRVTITCRASQSISDYLHWYQQK |
| | amino acid | PGKAPKLLIKYASQSISGVPSRFSGSGSGSDFTLTISSLQP |
| | sequence | EDFATYYCQNGHSFPPTFGGGTKVEIK |
| 19 | 101A6HZ VH | EVQLVESGGGLVQPGGSLRLSCAASGFTFTDYYLSWVR |
| | amino acid | QAPGKGLEWLALIRNKANGYTTEYAASVKGRFTISRDS |
| | sequence | SKNSLYLQMNSLKTEDTAVYYCARDTLAAYWGQGTLV |
| | | TVSS |
| 20 | 101A6HZ VL | DIVMTQSPDSLAVSLGERATINCKASQDVGSAVIWYQQ |
| | amino acid | KPGQSPKLLIYWASTRHTGVPDRFSGSGSGTDFTLTISSL |
| | sequence | QAEDVAVYYCQQYSSYPLTFGQGTKLEIK |
| 21 | 4E12HZ VH | QVQLVQSGAEVKRPGASVKVSCKTSGYTFTDYAMHW |
| | amino acid | VRQAPGKGLEWIGVISTYYGNVSYNQKFKGRVTMTRD |
| | sequence | TSTSTVYMELSSLTSEDTAIYYCARGFAYWGQGTLVTV |
| | | SS |
| 22 | 4E12HZ VL | DIVITQTELSLPVTPGERASISCRSSKSLQKKDGKTYLN |
| | amino acid | WFLQKPGQSPQLLIYLMSTRASGVSDRFSGSGSGTDFT |
| | sequence | LKISRVEAEDVGVYYCQQLVEYPFTFGQGTKLEIK |
| 23 | 19C2HZ VH | QVTLKESGPTLVKPTQTLTLTCTFSGFSLSTSGMGINWIR |
| | amino acid | QPPGKALEWLALIWWDDDKYYNPSLKNRLTITKDTSK |
| | sequence | NQVVLTMTNMDPVDTATYYCARSLITTAPSDYWGQGT |
| | | LVTVSS |
| 24 | 19C2HZ VL | DIQMTQSPSSLSASVGDRVTITCKASQNVGTAITWYQQ |
| | amino acid | RPGKAPKLLIYSASNRYTGVPSRFSGSGSGTDFTLTISNL |
| | sequence | QPEDLATYFCLQYSDYPLTFGQGTRLEIK |
| 25 | Amino acid | ASTKGPSVFPLAPSSKSTSGGTAALGCLVKDYFPEPVTV |
| | sequence of | SWNSGALTSGVHTFPAVLQSSGLYSLSSVVTVPSSSLGT |
| | human heavy | QTYICNVNHKPSNTKVDKKVEPKSCDKTHTCPPCPAPE |
| | chain constant | LLGGPSVFLFPPKPKDTLMISRTPEVTCVVVDVSHEDPE |
| | region IgG1 | VKFNWYVDGVEVHNAKTKPREEQYNSTYRVVSVLTVL |
| | | HQDWLNGKEYKCKVSNKALPAPIEKTISKAKGQPREPQ |
| | | VYTLPPSRDELTKNQVSLTCLVKGFYPSDIAVEWESNGQ |
| | | PENNYKTTPPVLDSDGSFFLYSKLTVDKSRWQQGNVFS |
| | | CSVMHEALHNHYTQKSLSLSPGK |
| 26 | Amino acid | RTVAAPSVFIFPPSDEQLKSGTASVVCLLNNFYPREAKV |
| | sequence of | QWKVDNALQSGNSQESVTEQDSKDSTYSLSSTLTLSKA |
| | human light | DYEKHKVYACEVTHQGLSSPVTKSFNRGEC |
| | chain constant | |
| | region CL | |
| 27 | 101A6/101A6HZ/ | GFTFTDY |
| | 8H3 Chothia | |
| | CDR H1 | |
| 28 | 101A6/101A6HZ/ | RNKANGYT |
| | 8H3 Chothia | |
| | CDR H2 | |
| 29 | 101A6/101A6HZ | DTLAAY |
| | Chothia/kabat/ | |
| | AbM CDR H3 | |
| 30 | 101A6/101A6HZ | KASQDVGSAVI |
| | Chothia/Kabat/ | |
| | AbM CDR L1 | |
| 31 | 101A6/101A6HZ/ | WASTRHT |
| | 8H3 | |
| | Chothia/Kabat/ | |
| | AbM CDR L2 | |
| 32 | 101A6/101A6HZ | QQYSSYPLT |
| | Chothia/Kabat/ | |
| | IMGT/AbM | |
| | CDR L3 | |
| 33 | 101A6/101A6HZ | DYYLS |
| | Kabat CDR | |
| | H1 | |
| 34 | 101A6HZ Kabat | LIRNKANGYTTEYAASVKG |
| | CDR H2 | |
| 35 | 101A6/8H3 | LIRNKANGYTTEYSASVKG |
| | Kabat CDR H2 | |
| 36 | 101A6/101A6HZ/ | GFTFTDYY |
| | 8H3 IMGT | |
| | CDR H1 | |
| 37 | 101A6/101A6HZ/ | IRNKANGYTT |
| | 8H3 IMGT | |
| | CDR H2 | |
| 38 | 101A6/101A6HZ | ARDTLAAY |
| | IMGT CDR | |
| | H3 | |
| 39 | 101A6/101A6HZ | QDVGSA |
| | IMGT CDR | |
| | L1 | |
| 40 | 101A6/101A6HZ/ | WAS |
| | 8H3 IMGT | |
| | CDR L2 | |
| 41 | 64A10/64A10HZ/ | GFSLTSY |
| | 64A10HZ05 | |
| | Chothia CDR | |
| | H1 | |
| 42 | 64A10/64A10HZ/ | WTSGN |
| | 64A10HZ05 | |
| | Chothia CDR | |
| | H2 | |
| 43 | 64A10/64A10HZ/ | HDGGNYVGVMNY |
| | 64A10HZ05 | |
| | Chothia/Kabat/ | |
| | AbM CDR H3 | |
| 44 | 64A10/64A10HZ/ | RASQSISDYLH |
| | 64A10HZ05 | |
| | Chothia/Kabat/ | |
| | AbM CDR L1 | |
| 45 | 64A10/64A10HZ/ | YASQSIS |
| | 64A10HZ05 | |
| | Chothia/Kabat/ | |
| | AbM CDR L2 | |
| 46 | 64A10/64A10HZ/ | QNGHSFPPT |
| | 64A10HZ05 | |
| | Chothia/Kabat/ | |
| | IMGT/AbM | |
| | CDR L3 | |
| 47 | 64A10/64A10H | SYGVY |
| | Z/64A10HZ05 | |
| | Kabat CDR H1 | |
| 48 | 64A10HZ/64A1 | VIWTSGNTNYNPSLKS |
| | 0HZ05 Kabat | |
| | CDR H2 | |
| 49 | 64A10 Kabat | VIWTSGNTNYNSALMS |
| | CDR H2 | |
| 50 | 64A10/64A10HZ/ | GFSLTSYG |
| | 64A10HZ05 | |
| | IMGT CDR H1 | |
| 51 | 64A10/64A10HZ/ | IWTSGNT |
| | 64A10HZ05 | |
| | IMGT CDR H2 | |
| 52 | 64A10/64A10HZ/ | VKHDGGNYVGVMNY |
| | 64A10HZ05 | |
| | IMGT CDR H3 | |
| 53 | 64A10/64A10HZ/ | QSISDY |
| | 64A10HZ05 | |
| | IMGT CDR L1 | |
| 54 | 64A10/64A10HZ/ | YAS |
| | 64A10HZ05 | |
| | IMGT CDR L2 | |
| 55 | 4E12/4E12HZ | GYTFTDY |
| | Chothia CDR | |
| | H1 | |
| 56 | 4E12/4E12HZ | STYYGN |
| | Chothia CDR | |
| | H2 | |
| 57 | 4E12/4E12HZ | GFAY |
| | Chothia/Kabat/ | |
| | AbM CDR H3 | |
| 58 | 4E12/4E12HZ | RSSKSLQKKDGKTYLN |
| | Chothia/Kabat/ | |
| | AbM CDR L1 | |
| 59 | 4E12/4E12HZ | LMSTRAS |
| | Chothia/Kabat/ | |
| | AbM CDR L2 | |
| 60 | 4E12/4E12HZ | QQLVEYPFT |
| | Chothia/Kabat/ | |
| | IMGT/AbM | |
| | CDR L3 | |
| 61 | 4E12/4E12HZ | DYAMH |
| | Kabat CDR H1 | |
| 62 | 4E12/4E12HZ | VISTYYGNVSYNQKFKG |
| | Kabat CDR H2 | |
| 63 | 4E12/4E12HZ | GYTFTDYA |
| | IMGT CDR H1 | |
| 64 | 4E12/4E12HZ | ISTYYGNV |
| | IMGT CDR H2 | |
| 65 | 4E12/4E12HZ | ARGFAY |
| | IMGT CDR H3 | |
| 66 | 4E12/4E12HZ | KSLQKKDGKTY |
| | IMGT CDR L1 | |
| 67 | 4E12/4E12HZ | LMS |
| | IMGT CDR L2 | |
| 68 | 19C2 Chothia | GFSLTTSGM |
| | CDR H1 | |
| 69 | 19C2/19C2HZ/ | WWDDD |
| | 4B12 Chothia | |
| | CDR H2 | |
| 70 | 19C2/19C2HZ | SLITTAPSDY |
| | Chothia/Kabat/ | |
| | AbM CDR H3 | |
| 71 | 19C2/19C2HZ | KASQNVGTAIT |
| | Chothia/Kabat/ | |
| | AbM CDR L1 | |
| 72 | 19C2/19C2HZ | SASNRYT |
| | Chothia/Kabat/ | |
| | AbM CDR L2 | |
| 73 | 19C2/19C2HZ | LQYSDYPLT |
| | Chothia/Kabat/I | |
| | MGT/AbM | |
| | CDR L3 | |
| 74 | 19C2HZ/4B12 | GFSLSTSGM |
| | Chothia CDR | |
| | H1 | |
| 75 | 19C2/19C2HZ | TSGMGIN |
| | Kabat CDR H1 | |
| 76 | 19C2/19C2HZ | LIWWDDDKYYNPSLKN |
| | Kabat CDR H2 | |
| 77 | 19C2 IMGT | GFSLTTSGMG |
| | CDR H1 | |
| 78 | 19C2/19C2HZ/ | IWWDDDK |
| | 4B12 IMGT CDR | |
| | H2 | |
| 79 | 19C2/19C2HZ | ARSLITTAPSDY |
| | IMGT CDR H3 | |
| 80 | 19C2HZ/4B12 | GFSLSTSGMG |
| | IMGT CDR H1 | |
| 81 | 19C2/19C2HZ | QNVGTA |
| | IMGT CDR L1 | |
| 82 | 19C2/19C2HZ | SAS |
| | IMGT CDR L2 | |
| 83 | 4B12 | SNYGYDWFAY |
| | Chothia/Kabat/ | |
| | AbM CDR H3 | |
| 84 | 4B12 | KASQDINTYLG |
| | Chothia/Kabat/ | |
| | AbM CDR L1 | |
| 85 | 4B12 | RANRLVD |
| | Chothia/Kabat/ | |
| | AbM CDR L2 | |
| 86 | 4B12 | LQYDEFPLT |
| | Chothia/Kabat/I | |
| | MGT/AbM | |
| | CDR L3 | |
| 87 | 4B12 Kabat | TSGMGVG |
| | CDR H1 | |
| 88 | 4B12 Kabat | HIWWDDDKHYNPSLKS |
| | CDR H2 | |
| 89 | 4B12 IMGT | VRSNYGYDWFAY |
| | CDR H3 | |
| 90 | 4B12 IMGT | QDINTY |
| | CDR L1 | |
| 91 | 4B12 IMGT | RAN |
| | CDR L2 | |
| 92 | 57F4 Chothia | GFTFSDY |
| | CDR H1 | |
| 93 | 57F4 Chothia | SSGSSA |
| | CDR H2 | |
| 94 | 57F4 | RSYYSYFAY |
| | Chothia/Kabat/ | |
| | AbM CDR H3 | |
| 95 | 57F4 | RVSSSISSSTLH |
| | Chothia/Kabat/ | |
| | AbM CDR L1 | |
| 96 | 57F4 | GTSNLAS |
| | Chothia/Kabat/ | |
| | AbM CDR L2 | |
| 97 | 57F4Chothia/ | QQWSSFPFT |
| | Kabat/IMGT/AbM | |
| | CDR L3 | |
| 98 | 57F4 Kabat | DYGMH |
| | CDR H1 | |
| 99 | 57F4 Kabat | YISSGSSALYYADTVKG |
| | CDR H2 | |
| 100 | 57F4 IMGT | GFTFSDYG |
| | CDR H1 | |
| 101 | 57F4 IMGT | ISSGSSAL |
| | CDR H2 | |
| 102 | 57F4 IMGT | ARRSYYSYFAY |
| | CDR H3 | |
| 103 | 57F4 IMGT | SSISSST |
| | CDR L1 | |
| 104 | 57F4 IMGT | GTS |
| | CDR L2 | |
| 105 | 8H3 | DSFEVY |
| | Chothia/Kabat/ | |
| | AbM CDR H3 | |
| 106 | 8H3 | KASQDVGTAVA |
| | Chothia/Kabat/ | |
| | AbM CDR L1 | |
| 107 | 8H3 | QQFSSFPFT |
| | Chothia/Kabat/ | |
| | I | |
| | CDR L3 | |
| 108 | 8H3 Kabat CDR | DYYMS |
| | H1 | |
| 109 | 8H3 IMGT | ARDSFEVY |
| | CDR H3 | |
| 110 | 8H3 IMGT | QDVGTA |
| | CDR L1 | |
| 111 | 101A6HZ heavy | EVQLVESGGGLVQPGGSLRLSCAASGFTFTDYYLSWVR |
| | chain amino | QAPGKGLEWLALIRNKANGYTTEYAASVKGRFTISRDS |
| | acid sequence | SKNSLYLQMNSLKTEDTAVYYCARDTLAAYWGQGTLV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPELLGGPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| 112 | 101A6HZ light | DIVMTQSPDSLAVSLGERATINCKASQDVGSAVIWYQQ |
| | chain amino | KPGQSPKLLIYWASTRHTGVPDRFSGSGSGTDFTLTISSL |
| | acid sequence | QAEDVAVYYCQQYSSYPLTFGQGTKLEIKRTVAAPSVFI |
| | | FPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| 113 | 101A6/101A6HZ | GFTFTDYYLS |
| | AbM CDR H1 | |
| 114 | 101A6/101A6HZ/ | LIRNKANGYTTE |
| | 8H3 AbM | |
| | CDR H2 | |
| 115 | 64A10/64A10HZ/ | GFSLTSYGVY |
| | 64A10HZ05 | |
| | AbM CDR H1 | |
| 116 | 64A10/64A10HZ/ | VIWTSGNTN |
| | 64A10HZ05 | |
| | AbM CDR H2 | |
| 117 | 4E12/4E12HZ | GYTFTDYAMH |
| | AbM CDR H1 | |
| 118 | 4E12/4E12HZ | VISTYYGNVS |
| | AbM CDR H2 | |
| 119 | 19C2 AbM | GFSLTTSGMGIN |
| | CDR H1 | |
| 120 | 19C2/19C2HZ | LIWWDDDKY |
| | AbM CDR H2 | |
| 121 | 19C2HZ AbM | GFSLSTSGMGIN |
| | CDR H1 | |
| 122 | 4B12 AbM | GFSLSTSGMGVG |
| | CDR H1 | |
| 123 | 4B12 AbM | HIWWDDDKH |
| | CDR H2 | |
| 124 | 57F4 AbM CDR | GFTFSDYGMH |
| | H1 | |
| 125 | 57F4 AbM CDR | YISSGSSALY |
| | H2 | |
| 126 | 8H3 AbM CDR | GFTFTDYYMS |
| | H1 | |
| 127 | 101A6HZ VH | GAGGTGCAGCTGGTGGAATCCGGAGGAGGCCTGGTC |
| | nucleotide | CAACCCGGCGGCTCCCTGAGACTGTCTTGTGCCGCCT |
| | sequence | CCGGCTTTACATTCACCGACTACTACCTGTCTTGGGTG |
| | | CGGCAGGCCCCTGGCAAGGGCCTGGAATGGCTGGCT |
| | | CTGATCCGGAACAAGGCCAACGGCTACACAACCGAG |
| | | TACGCCGCTTCTGTGAAGGGCAGATTCACCATCTCTC |
| | | GGGATTCCTCTAAGAACTCGCTGTACCTGCAGATGAA |
| | | CTCTCTGAAGACCGAGGACACAGCCGTGTACTACTG |
| | | CGCCAGAGATACCCTGGCCGCCTACTGGGGCCAGGG |
| | | CACCCTGGTGACCGTGTCCTCC |
| 128 | 101A6HZ VL | GACATCGTGATGACCCAGTCTCCTGATTCCCTGGCCG |
| | nucleotide | TGAGCCTGGGCGAGAGAGCCACCATCAACTGCAAGG |
| | sequence | CTTCTCAGGATGTGGGATCTGCCGTGATCTGGTACCA |
| | | GCAGAAGCCTGGCCAGAGCCCCAAGCTGCTGATCTA |
| | | CTGGGCCTCCACCAGGCATACCGGCGTGCCTGACCG |
| | | GTTCAGCGGCTCTGGCTCCGGCACAGACTTCACCCTG |
| | | ACAATCTCCAGTCTGCAGGCCGAGGACGTCGCTGTG |
| | | TACTACTGCCAGCAGTACTCCTCCTATCCTCTGACCTT |
| | | TGGACAAGGTACCAAGCTGGAGATCAAG |
| 129 | 101A6HZ heavy | GAAGTGCAACTAGTGGAAAGTGGTGGTGGTCTCGTG |
| | chain nucleotide | CAGCCTGGCGGAAGCCTGAGACTGAGCTGTGCTGCT |
| | sequence | TCTGGCTTCACCTTCACCGACTACTATCTGTCTTGGGT |
| | | CCGCCAGGCCCCCGGCAAGGGCCTGGAATGGCTGGC |
| | | TCTGATCCGGAACAAGGCCAACGGCTACACCACAGA |
| | | GTACGCCGCTTCCGTGAAAGGCAGATTTACCATCTCC |
| | | CGGGACTCCTCCAAGAACTCCCTGTACCTGCAGATGA |
| | | ACTCCCTGAAGACCGAGGACACCGCCGTGTACTACT |
| | | GCGCCAGAGATACACTGGCCGCCTACTGGGGCCAAG |
| | | GCACCCTGGTGACCGTGTCCTCTGCCTCTACAAAGGG |
| | | CCCCTCCGTGTTTCCACTGGCTCCCTCCTCTAAGAGC |
| | | ACCTCTGGCGGCACAGCCGCTCTGGGCTGTCTGGTG |
| | | AAGGATTACTTCCCAGAGCCCGTGACAGTGAGCTGG |
| | | AACTCTGGCGCCCTGACCTCTGGAGTGCACACATTTC |
| | | CAGCTGTGCTGCAGTCCAGCGGCCTGTACTCCCTGTC |
| | | TTCCGTGGTGACCGTGCCCAGCTCTTCCCTGGGCACC |
| | | CAGACATATATCTGCAACGTGAACCACAAGCCATCCA |
| | | ATACAAAGGTGGACAAGAAGGTGGAGCCCAAGTCCT |
| | | GTGATAAGACCCATACATGCCCCCCTTGTCCTGCTCC |
| | | AGAGCTGCTGGGAGGACCTTCCGTGTTCCTGTTTCCA |
| | | CCCAAACCTAAGGACACCCTGATGATCAGCAGGACC |
| | | CCCGAGGTGACATGCGTGGTGGTGGACGTGTCTCAC |
| | | GAGGATCCCGAGGTGAAGTTCAACTGGTATGTCGATG |
| | | GCGTGGAGGTGCATAATGCTAAGACCAAGCCTCGGG |
| | | AGGAGCAGTACAACTCCACCTATAGAGTGGTGAGCG |
| | | TGCTGACAGTGCTGCATCAGGACTGGCTGAACGGCA |
| | | AGGAGTATAAGTGCAAGGTGTCCAATAAGGCCCTGCC |
| | | CGCTCCCATCGAGAAGACCATCAGCAAGGCCAAGGG |
| | | CCAGCCTAGGGAGCCACAGGTGTACACACTGCCTCC |
| | | ATCCCGGGACGAGCTGACCAAGAACCAGGTGAGCCT |
| | | GACATGTCTGGTGAAGGGCTTCTATCCTAGCGATATCG |
| | | CTGTGGAGTGGGAGTCCAATGGCCAGCCAGAGAACA |
| | | ATTACAAGACCACACCCCCTGTGCTGGACTCCGATGG |
| | | CAGCTTCTTTCTGTATTCTAAGCTGACCGTGGATAAGT |
| | | CCCGGTGGCAGCAGGGCAACGTGTTCTCCTGTAGCG |
| | | TGATGCACGAAGCCCTGCATAATCACTATACTCAGAA |
| | | AAGTCTGTCACTGTCACCTGGTAAA |
| 130 | 101A6HZ light | GACATCGTGATGACCCAGTCTCCTGATTCCCTGGCCG |
| | chain nucleotide | TGAGCCTGGGCGAGAGAGCCACCATCAACTGCAAGG |
| | sequence | CTTCTCAGGATGTGGGATCTGCCGTGATCTGGTACCA |
| | | GCAGAAGCCTGGCCAGAGCCCCAAGCTGCTGATCTA |
| | | CTGGGCCTCCACCAGGCATACCGGCGTGCCTGACCG |
| | | GTTCAGCGGCTCTGGCTCCGGCACAGACTTCACCCTG |
| | | ACAATCTCCAGTCTGCAGGCCGAGGACGTCGCTGTG |
| | | TACTACTGCCAGCAGTACTCCTCCTATCCTCTGACCTT |
| | | TGGACAAGGTACCAAGCTGGAGATCAAGCGGACCGT |
| | | GGCTGCTCCTTCCGTGTTCATCTTCCCTCCATCTGATG |
| | | AACAGCTGAAGTCTGGCACCGCTTCTGTGGTGTGCCT |
| | | GCTGAACAACTTCTACCCCAGAGAAGCCAAGGTCCA |
| | | GTGGAAGGTGGACAACGCCCTGCAGTCCGGCAATAG |
| | | CCAAGAGTCTGTGACCGAGCAGGACTCCAAGGACTC |
| | | TACCTACTCCCTGTCCTCTACCCTGACACTCAGCAAG |
| | | GCCGACTACGAGAAGCACAAAGTGTACGCCTGCGAG |
| | | GTTACCCACCAGGGCCTGTCCTCTCCTGTGACCAAAT |
| | | CCTTCAACAGAGGCGAATGT |
| 131 | Mutant human | ASTKGPSVFPLAPSSKSTSGGTAALGCLVKDYFPEPVTV |
| | heavy chain | SWNSGALTSGVHTFPAVLQSSGLYSLSSVVTVPSSSLGT |
| | constant region | QTYICNVNHKPSNTKVDKKVEPKSCDKTHTCPPCPAPE |
| | IgGlm | AAGAPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALPAPIEKTISKAKGQPREPQ |
| | | VYTLPPSRDELTKNQVSLTCLVKGFYPSDIAVEWESNGQ |
| | | PENNYKTTPPVLDSDGSFFLYSKLTVDKSRWQQGNVFS |
| | | CSVMHEALHNHYTQKSLSLSPGK |
| 132 | 101A6HZm | GAGGTGCAGCTGGTGGAATCTGGCGGAGGACTCGTG |
| | heavy chain | CAGCCTGGCGGATCTCTGAGACTGTCTTGCGCTGCCT |
| | nucleotide | CTGGCTTTACCTTCACCGACTACTACCTGTCTTGGGTG |
| | sequence | CGGCAGGCCCCAGGCAAGGGCCTGGAATGGCTGGCA |
| | | CTGATCCGGAACAAGGCCAACGGCTACACCACCGAG |
| | | TACGCCGCCTCCGTCAAAGGCAGATTCACCATCTCCC |
| | | GCGACTCCAGCAAGAACTCTCTGTACCTGCAGATGA |
| | | ACAGCCTGAAGACCGAGGACACCGCCGTGTACTACT |
| | | GCGCCAGAGATACCCTGGCCGCTTACTGGGGCCAGG |
| | | GCACACTGGTGACCGTCTCCTCTGCTTCTACCAAGGG |
| | | CCCTTCTGTGTTTCCTCTGGCCCCTTCTAGCAAGTCTA |
| | | CCTCTGGCGGCACCGCTGCCCTGGGATGTCTTGTGAA |
| | | GGATTACTTCCCCGAACCTGTGACCGTGTCCTGGAAC |
| | | TCCGGCGCCCTGACATCTGGAGTGCATACCTTTCCCG |
| | | CTGTGCTGCAGTCTAGCGGCCTGTACTCCCTGTCCTC |
| | | CGTCGTTACCGTGCCTTCCAGCAGCCTGGGCACCCAG |
| | | ACCTACATCTGCAACGTGAACCACAAGCCCTCCAAC |
| | | ACCAAAGTGGATAAGAAGGTGGAACCCAAGTCCTGC |
| | | GACAAGACCCACACCTGTCCTCCTTGCCCTGCCCCAG |
| | | AGGCCGCTGGCGCTCCATCAGTGTTCCTGTTCCCTCC |
| | | TAAACCTAAGGACACCCTGATGATCTCTCGGACACCT |
| | | GAGGTGACCTGCGTGGTGGTCGACGTGTCTCACGAG |
| | | GACCCTGAAGTGAAGTTCAACTGGTACGTGGATGGC |
| | | GTGGAAGTGCACAACGCCAAGACCAAGCCTAGAGAG |
| | | GAACAGTACAACTCCACCTATAGAGTGGTGTCCGTAC |
| | | TGACAGTGCTGCATCAGGACTGGCTGAACGGCAAAG |
| | | AGTACAAGTGCAAGGTGTCCAACAAAGCTCTGCCTG |
| | | CTCCTATCGAGAAGACCATCTCCAAGGCCAAGGGCC |
| | | AGCCTCGGGAGCCTCAAGTGTACACCCTGCCCCCTAG |
| | | CAGGGACGAGCTGACCAAGAATCAGGTGTCCCTGAC |
| | | TTGTCTGGTCAAAGGCTTCTACCCCTCCGACATCGCC |
| | | GTGGAGTGGGAGTCCAATGGCCAACCCGAGAACAAC |
| | | TACAAAACCACACCTCCAGTGCTGGACTCTGACGGC |
| | | TCCTTCTTCCTGTATTCCAAGCTGACCGTGGACAAGT |
| | | CCAGATGGCAGCAGGGCAACGTGTTCTCCTGCTCCGT |
| | | GATGCACGAGGCTCTGCACAATCACTACACACAGAA |
| | | GTCCCTGAGCTTGTCTCCTGGCAAG |
| 133 | Linker | GGGGS |
| 134 | Linker | GGGGSGGGGSGGGGSGGGGS |
| 135 | 101A6HZm | EVQLVESGGGLVQPGGSLRLSCAASGFTFTDYYLSWVR |
| | heavy chain | QAPGKGLEWLALIRNKANGYTTEYAASVKGRFTISRDS |
| | amino acid | SKNSLYLQMNSLKTEDTAVYYCARDTLAAYWGQGTLV |
| | sequence | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKKVEPKSCDKTHTCPPC |
| | | PAPEAAGAPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| 136 | 101A6HZm | XVQLVESGGGLVQPGGSLRLSCAASGFTFTDYYLSWVR |
| | heavy chain | QAPGKGLEWLALIRNKANGYTTEYAASVKGRFTISRDS |
| | amino acid | SKNSLYLQMNSLKTEDTAVYYCARDTLAAYWGQGTLV |
| | sequence | TVSSASTKGPSVFPLAPSSKSTSGGTAALGCLVKDYFPE |
| | (Pyroglutamate | PVTVSWNSGALTSGVHTFPAVLQSSGLYSLSSVVTVPSS |
| | at N-terminus; | SLGTQTYICNVNHKPSNTKVDKKVEPKSCDKTHTCPPC |
| | no C-terminal | PAPEAAGAPSVFLFPPKPKDTLMISRTPEVTCVVVDVSH |
| | lysine) | EDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| | | X is pyroglutamate or pyroglutamic acid |
| 137 | 101A6HZm | EVQLVESGGGLVQPGGSLRLSCAASGFTFTDYYLSWVR |
| | heavy chain | QAPGKGLEWLALIRNKANGYTTEYAASVKGRFTISRDS |
| | amino acid | SKNSLYLQMNSLKTEDTAVYYCARDTLAAYWGQGTLV |
| | sequence (no C- | TVSSASTKGPSVFPLAPSSKSTSGGTAALGCLVKDYFPE |
| | terminal lysine) | PVTVSWNSGALTSGVHTFPAVLQSSGLYSLSSVVTVPSS |
| | | SLGTQTYICNVNHKPSNTKVDKKVEPKSCDKTHTCPPC |
| | | PAPEAAGAPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| 138 | 101A6HZm | XVQLVESGGGLVQPGGSLRLSCAASGFTFTDYYLSWVR |
| | heavy chain | QAPGKGLEWLALIRNKANGYTTEYAASVKGRFTISRDS |
| | amino acid | SKNSLYLQMNSLKTEDTAVYYCARDTLAAYWGQGTLV |
| | sequence | TVSSASTKGPSVFPLAPSSKSTSGGTAALGCLVKDYFPE |
| | (Pyroglutamate | PVTVSWNSGALTSGVHTFPAVLQSSGLYSLSSVVTVPSS |
| | at N-terminus) | SLGTQTYICNVNHKPSNTKVDKKVEPKSCDKTHTCPPC |
| | | PAPEAAGAPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSRDELTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| | | X is pyroglutamate or pyroglutamic acid |

Abbreviations herein have the following meanings:

| | |
|---|---|
| CDR | Complementarity determining region in immunoglobulin variable region |
| FR | Antibody framework region: amino acid residue excepting CDR residue in |
| | variable region of antibody |
| VH | Heavy chain variable region of antibody |
| VL | Light chain variable region of antibody |
| IgG | Immunoglobulin G |
| IMGT | Numbering system based on The international ImMunoGeneTics information |
| | system ® (IMGT) initiated by Lefranc et al., referring to Lefranc et al., Dev. |
| | Comparat. Immunol. 27: 55-77, 2003. |
| Kabat | Immunoglobulin comparing and numbering system proposed by Elvin A. Kabat (see, |
| | for example, Kabat et al., Sequences of Proteins of Immunological Interest, 5th |
| | Ed. Public Health Service, National Institutes of Health, Bethesda, Md., 1991). |
| Chothia | Immunoglobulin numbering system proposed by Chothia et al. It is a classical |
| | rule for identifying the boundary of CDR region based on the position of |
| | structural loop region (see, for example, Chothia & Lesk (1987) J. Mol. Biol. |
| | 196: 901-917; Chothia et al. (1989) Nature 342: 878-883). |
| AbM | The definition mode of AbM CDR is originated from related research of Martin |
| | (Martin A C R, Cheetham J C, Rees A R (1989) Modelling antibody hypervariable |
| | loops: A combined algorithm. Proc Natl Acad Sci USA 86: 9268-9272). |
| mAb | Monoclonal antibody |
| EC50 | Concentration achieving 50% efficiency or binding |
| IC50 | Concentration achieving 50% inhibiting |
| ELISA | Enzyme-linked immunosorbent assay |
| PCR | Polymerase chain reaction |
| HRP | Horseradish peroxidase |
| K_{D} | Equilibrium dissociation constant |
| Ka | Binding rate constant |
| Kd | Dissociation rate constant |
| ADCC | Antibody-dependent cellular cytotoxicity |
| FACS | Flow cytometry |
| CDR-H1 | Complementarity determining region in immunoglobulin heavy chain variable |
| | region 1 |
| CDR-H2 | Complementarity determining region in immunoglobulin heavy chain variable |
| | region 2 |
| CDR-H3 | Complementarity determining region in immunoglobulin heavy chain variable |
| | region 3 |
| CDR-L1 | Complementarity determining region in immunoglobulin light chain variable |
| | region 1 |
| CDR-L2 | Complementarity determining region in immunoglobulin light chain variable |
| | region 2 |
| CDR-L3 | Complementarity determining region in immunoglobulin light chain variable |
| | region 3 |
| PyroE | Pyroglutamate |

Unless otherwise specified, molecular biology experimental methods and immune detection methods used in the present invention are basically carried out with reference to methods proposed by J. Sambrook et al. in Molecular Cloning: A Laboratory Manual, Version 2, The Cold Spring Harbor Laboratory Press, 1989, and methods proposed by F. M. Ausubel et al. in Short Protocols in Molecular Biology, Version 3, John Wiley & Sons, Inc., 1995. Those skilled in the art are aware that the examples describe the present invention by way of example and are not intended to limit the scope claimed by the present invention.

### Example 1: Preparation of Anti-Human PTK7 Monoclonal Antibody

### 1.1 Immunization of Mice

6×His tags were added to C-terminals of extracellular sequences of human PTK7 (NP_002812.2), monkey PTK7 (NP_001248508.1), rat PTK7 (NP_001382670.1) and mouse PTK7 (NP_780377.1), and GenScript Biotech Corporation was entrusted to perform codon optimization and gene synthesis, and construct the extracellular sequences to a pTT5 expression vector; recombinant plasmids were extracted, sequencing was carried out to ensure that no error existed, then HEK293 cells were transfected for transient expression, and cell supernatants were collected after 7 days and respectively purified to obtain human PTK7 protein, monkey PTK7 protein, rat PTK7 protein and mouse PTK7 protein. Wild type mice were immunized by using the human PTK7 protein, and a Freund's complete adjuvant and a Freund's incomplete adjuvant were respectively adopted for immunization. During immunization, the serum titer of an anti-PTK7 antibody was monitored by protein ELISA and flow cytometry every two weeks; specifically, ELISA: human PTK7-His was diluted with CBS coating liquid, and coated overnight at 4° C.; washed once, and blocked for 2 h with BSA at 37° C.; the blocking liquid was removed, and the serum was diluted in a triple gradient manner, and then incubated for 2 h at 37° C.; the serum was washed for 3 times, and incubated for 1 h at 37° C. with HRP-Goat-Anti-mouse IgG (1:10000); washing was performed for 5 times; and TMB (Huzhou INNOREAGENTS) was added for developing and terminating, and reading was performed at 450 nm by a microplate reader. Flow cytometry: HCT116 cells (Cell Bank, Chinese Academy of Sciences) were collected, and washed for 3 times; the mice serum was subjected to triple dilution, with 11 concentration points in total, and meanwhile, blank cell wells and negative mouse serum control wells were simultaneously arranged; the HCT116 cells (1×10⁵ cells/well) were re-suspended, respectively added into each well, uniformly mixed, and then incubated for 1 h at 4° C.; washing was carried out for 3 times, and the cells were re-suspended by an APC anti-mouse IgG Fc antibody and then incubated for 0.5 h at 4° C.; and the cells were washed for 3 times, then re-suspended, and detected by a machine. Optimal mice were selected for hybridoma fusion according to the binding protein and the cell titer.

### 1.2 Hybridoma Fusion Screening

ELISA was adopted for supernatant screening 7 days after hybridoma fusion. Specifically, the monkey PTK7 protein was diluted with the coating liquid and coated overnight at 4° C. Washing was performed once, blocking was performed with BSA for 1 h at 37° C., and hybridoma supernatant was directly taken and added into a microplate reader, and incubated for 2 h at 37° C. The solution was removed, and washing was performed three times. A microplate reader was drained, and incubation was performed for 1 h at 37° C. with HRP-Goat anti-mouse IgG (Thermo Fisher) diluted in 1:10000. The solution was removed, washing was performed five times, the microplate reader was drained, TMB (Huzhou INNOREAGENTS) was added, developed in dark place, then the reaction was terminated, and the absorbance at 450 nm was read by the microplate reader. A clone with the signal value 5 times larger than that of a negative control was further subjected to flow cytometry validation by human PTK7 protein ELISA and SKOV-3 cells (NANJING COBIOER BIOSCIENCES CO., LTD). Positive clones were subjected to subcloning, monoclones were screened by the above method, and then optimal monoclones were selected for amplification culture.

### Example 2: Evaluation of Anti-Human PTK7 Murine Antibody

### 2.1 Evaluation of Binding Activity of Anti-Human PTK7 Murine Antibody

All preferred monoclones were amplified for serum-free culture; 5-10 ml of culture supernatant was subjected to affinity purification by using Protein-A beads; an elution product was neutralized by a Tris solution; and the concentration of the antibody protein was quantified by using Nanodrop and then subjected to candidate evaluation.

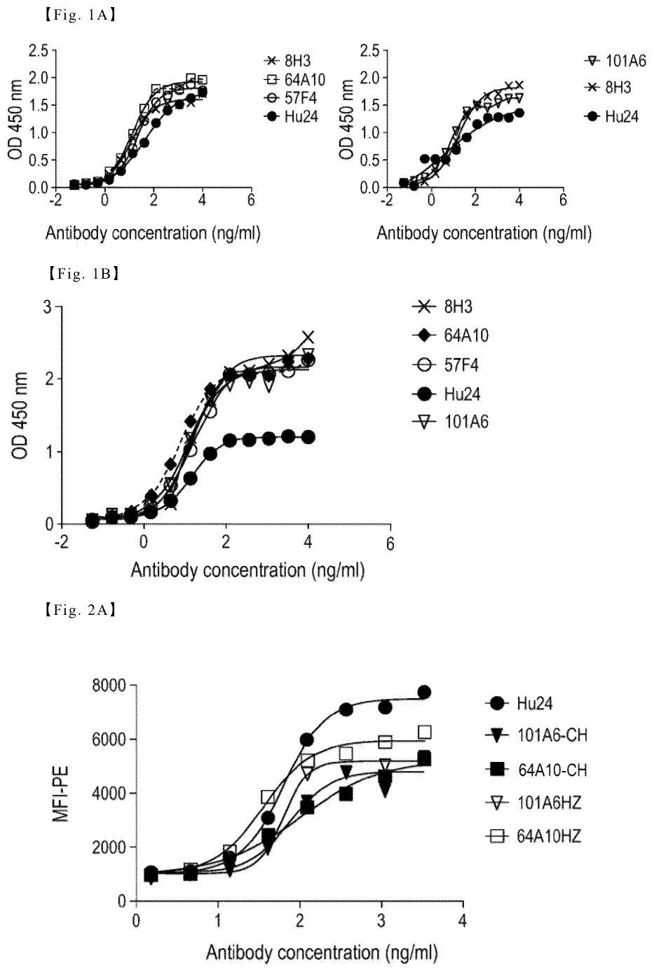

ELISA affinity determination of the PTK7 murine antibody: the human and monkey PTK7 proteins were diluted to reach 1 μg/ml by using the CBS coating liquid, and stood overnight at 4° C.; gradient dilution was performed on the purified murine antibody and a control antibody by using 2% BSA, respectively; the gradient dilution was started from 10 μg/ml, with triple gradient and 12 concentration points; and the rest steps were the same as 1.1. Original data was imported into software for nonlinear curve fitting, and EC₅₀ of binding of each antibody to human PTK7 and monkey PTK7 was calculated, respectively. As shown in Table 1 and FIGS. 1A-1B, the screened antibodies could be bound to the human and monkey PTK7 proteins with affinity higher than that of the control antibody Hu24.

TABLE 1
Detection of binding of murine antibody to PTK7 protein

| | Human PTK7 protein | Monkey PTK7 protein |
|---|---|---|
| Antibody | EC₅₀(ng/mL) | EC₅₀(ng/mL) |
| Hu24 | 45.72 | 13.30 |
| 64A10 | 13.21 | 7.65 |
| 101A6 | 13.10 | 18.7 |
| 8H3 | 11.27 | 16.76 |
| 57F4 | 21.79 | 15.84 |

Flow cytometry determination of affinity of the PTK7 murine antibody: the cell binding capacity of the murine antibody was subjected to flow cytometry detection by using H1299 and H358 cells (COBIOER BIOSCIENCES CO., LTD), and the detection was started from 10 μg/ml of antibody, with triple gradient and 11 concentration points; the signal value was imported into software to calculate EC₅₀ of the antibody; and the result is shown as Table 2.

TABLE 2
Detection of binding of murine antibody to
cell tumor endogenously expressing PTK7

| Antibody | Cell | EC₅₀(ng/mL) |
|---|---|---|
| Hu24 | H1299 | 292.6 |
| 101A6 | H1299 | 245.5 |
| 8H3 | H1299 | 209.8 |
| 57F4 | H1299 | 87.51 |
| 4B12 | H358 | 44.06 |
| 4E12 | H358 | 34.99 |
| 19C2 | H358 | 58.26 |

### 2.2 Sequencing of Anti-Human PTK7 Murine Antibody and Construction of Chimeric Antibody

Hybridoma cells were cultured to reach 8000 cells; the cells were lysed, and a first chain cDNA was synthesized by adopting a cDNA reverse transcription kit (Thermo Fisher). VH and VK genes were amplified from the cDNA by adopting a primer in a PCR mode; the PCR product was purified by a DNA purification kit (MACHEREY-NAGEL), and was homologously recombined into a pTT5 vector expressing a human heavy chain constant region IgG1 (SEQ ID NO: 27) and light chain constant region CL (SEQ ID NO: 28) so as to construct a chimeric antibody expression vector. Positive clones were picked for sequencing after PCR validation. The sequence was analyzed through IMGT and Abysis websites. An anti-human PTK7 antibody variable region sequence and a CDR sequence were obtained shown as Table 3.

TABLE 3
Anti-human PTK7 antibody variable region and CDR amino acid sequence

| | SEQ ID NO: | | | | | | | | |
|---|---|---|---|---|---|---|---|---|---|
| | Heavy | Light | | SEQ ID NO: | | | | | |
| | chain | chain | | Heavy | Heavy | Heavy | Light | Light | Light |
| | variable | variable | Definition | chain | chain | chain | chain | chain | chain |
| Antibody | region | region | mode | CDR1 | CDR2 | CDR3 | CDR1 | CDR2 | CDR3 |
| 101A6 | 1 | 2 | Chothia | 27 | 28 | 29 | 30 | 31 | 32 |
| | | | Kabat | 33 | 35 | 29 | 30 | 31 | 32 |
| | | | IMGT | 36 | 37 | 38 | 39 | 40 | 32 |
| | | | AbM | 113 | 114 | 29 | 30 | 31 | 32 |
| 64A10 | 3 | 4 | Chothia | 41 | 42 | 43 | 44 | 45 | 46 |
| | | | Kabat | 47 | 49 | 43 | 44 | 45 | 46 |
| | | | IMGT | 50 | 51 | 52 | 53 | 54 | 46 |
| | | | AbM | 115 | 116 | 43 | 44 | 45 | 46 |
| 4E12 | 5 | 6 | Chothia | 55 | 56 | 57 | 58 | 59 | 60 |
| | | | Kabat | 61 | 62 | 57 | 58 | 59 | 60 |
| | | | IMGT | 63 | 64 | 65 | 66 | 67 | 60 |
| | | | AbM | 117 | 118 | 57 | 58 | 59 | 60 |
| 19C2 | 7 | 8 | Chothia | 68 | 69 | 70 | 71 | 72 | 73 |
| | | | Kabat | 75 | 76 | 70 | 71 | 72 | 73 |
| | | | IMGT | 77 | 78 | 79 | 81 | 82 | 73 |
| | | | AbM | 119 | 120 | 70 | 71 | 72 | 73 |
| 4B12 | 9 | 10 | Chothia | 74 | 69 | 83 | 84 | 85 | 86 |
| | | | Kabat | 87 | 88 | 83 | 84 | 85 | 86 |
| | | | IMGT | 80 | 78 | 89 | 90 | 91 | 86 |
| | | | AbM | 122 | 123 | 83 | 84 | 85 | 86 |
| 57F4 | 11 | 12 | Chothia | 92 | 93 | 94 | 95 | 96 | 97 |
| | | | Kabat | 98 | 99 | 94 | 95 | 96 | 97 |
| | | | IMGT | 100 | 101 | 102 | 103 | 104 | 97 |
| | | | AbM | 124 | 125 | 94 | 95 | 96 | 97 |
| 8H3 | 13 | 14 | Chothia | 27 | 28 | 105 | 106 | 31 | 107 |
| | | | Kabat | 108 | 35 | 105 | 106 | 31 | 107 |
| | | | IMGT | 36 | 37 | 109 | 110 | 40 | 107 |
| | | | AbM | 126 | 114 | 105 | 106 | 31 | 107 |
| 64A10HZ | 15 | 16 | Chothia | 41 | 42 | 43 | 44 | 45 | 46 |
| | | | Kabat | 47 | 48 | 43 | 44 | 45 | 46 |
| | | | IMGT | 50 | 51 | 52 | 53 | 54 | 46 |
| | | | AbM | 115 | 116 | 43 | 44 | 45 | 46 |
| 64A10HZ05 | 17 | 18 | Chothia | 41 | 42 | 43 | 44 | 45 | 46 |
| | | | Kabat | 47 | 48 | 43 | 44 | 45 | 46 |
| | | | IMGT | 50 | 51 | 52 | 53 | 54 | 46 |
| | | | AbM | 115 | 116 | 43 | 44 | 45 | 46 |
| 101A6HZ | 19 | 20 | Chothia | 27 | 28 | 29 | 30 | 31 | 32 |
| | | | Kabat | 33 | 34 | 29 | 30 | 31 | 32 |
| | | | IMGT | 36 | 37 | 38 | 39 | 40 | 32 |
| | | | AbM | 113 | 114 | 29 | 30 | 31 | 32 |
| 4E12HZ | 21 | 22 | Chothia | 55 | 56 | 57 | 58 | 59 | 60 |
| | | | Kabat | 61 | 62 | 57 | 58 | 59 | 60 |
| | | | IMGT | 63 | 64 | 65 | 66 | 67 | 60 |
| | | | AbM | 117 | 118 | 57 | 58 | 59 | 60 |
| 19C2HZ | 23 | 24 | Chothia | 74 | 69 | 70 | 71 | 72 | 73 |
| | | | Kabat | 75 | 76 | 70 | 71 | 72 | 73 |
| | | | IMGT | 80 | 78 | 79 | 81 | 82 | 73 |
| | | | AbM | 121 | 120 | 70 | 71 | 72 | 73 |

### Example 3: Humanization and Evaluation of Anti-Human PTK7 Antibody

### 3.1 Humanization and Expression of Anti-Human PTK7 Antibody

Murine antibodies 64A10, 101A6, 4E12 and 19C2 were subjected to humanization by adopting a CDR-grafted antibody humanization method. In short, the humanization included the following steps: comparing an amino acid sequence of a murine monoclonal antibody with an amino acid sequence of a human embryonic system antibody, and finding out a sequence with high homology and relatively excellent physicochemical property as a human embryonic system framework sequence; analyzing and inspecting HLA-DR affinity, and selecting out a human embryonic system framework sequence with low affinity; and then respectively grafting six CDRs of the murine antibody to selected heavy chain and light chain framework sequences.

A computer simulation technology was further utilized to analyze the variable region and peripheral framework amino acid sequence thereof by molecular docking, so as to investigate a spatial three-dimensional binding mode. By calculating electrostatic force, Van der Waals' force, hydrophilicity and hydrophobicity and entropy, key amino acids which could act with the PTK7 protein and maintain a spatial framework in the murine antibody amino acid sequence were analyzed, and the murine amino acids were remained in the grafted antibody. That is, a series of reverse mutations were performed on FR region amino acid residues of a above humanized template, so that the humanized antibody retained the antigen-binding capability of the murine antibody as much as possible.

According to the above method, humanized antibodies were constructed on the basis of CDRs of the murine antibodies 64A10, 101A6, 4E12 and 19C2, and were respectively named as 64A10HZ, 64A10HZ05 (VH3+VL2), 101A6HZ, 4E12HZ and 19C2HZ; wherein the heavy chain constant region of each antibody was a human IgG1 heavy chain constant region (SEQ ID NO: 25), and the light chain constant region of each antibody was a human Kappa light chain constant region (SEQ ID NO: 26). In addition, according to the above method, the human IgG1 heavy chain constant region (SEQ ID NO: 25) of 101A6HZ was substituted by a mutant human heavy chain constant region IgG1m (SEQ ID NO: 131), and the expressed antibody was named as 101A6HZm.

Chimeric and humanized antibodies: the PTK7 control antibody was taken from Hu24 in the Patent CN201580030255, Nanjing GenScript Biotech Corporation was entrusted to perform codon optimization, and synthesize and link cDNA into an expression plasmid pTT5. The heavy chain and light chain expression plasmids of the humanized antibody were simultaneously transfected into CHO-S cells, the supernatant was centrifugally collected after expression for 7 days, and the recombinant antibody in the supernatant was purified by utilizing Protein A (MabSelect SuRe, GE) to obtain the anti-human PTK7 chimeric and humanized antibodies.

### 3.2 Detection of Binding of Anti-Human PTK7 Chimeric and Humanized Antibodies to Human PTK7 Protein

In order to detect the affinity of the anti-human PTK7 chimeric and humanized antibodies with human PTK7, the human PTK7 protein was diluted to reach 1 μg/ml through the CBS coating liquid, and was coated into a 96-well microtiter plate at a density of 100 μl/well, and then was stood at 4° C. overnight. The original data was imported into software for nonlinear curve fitting, and the EC₅₀ of binding of each antibody to the human PTK7 was calculated, respectively. As shown in Table 4, the screened antibodies could be well bound to the human PTK7 protein, and the binding capability was not obviously affected after humanization.

TABLE 4
Binding of anti-human PTK7 chimeric and humanized
antibodies to human PTK7-His protein

| | Human PTK7 protein |
|---|---|
| Antibody | EC₅₀(ng/mL) |
| Hu24 | 46.57 |
| 64A10HZ | 15.53 |
| 64A10-CH | 25.77 |
| 101A6HZ | 26.57 |
| 101A6-CH | 31.55 |

### 3.3 Detection of Dynamic Affinity of Anti-Human PTK7 Humanized Antibody

The dynamic affinity of the control Hu24 and candidate antibodies 64A10HZ, 101A6HZ, 4E12HZ and 19C2HZ with human PTK7-His was detected by using ForteBio (Pall life sciences). The specific method included: diluting the to-be-detected antibody to reach 5 μg/ml by using PBST; diluting human PTK7-His protein to reach 200 nM, 100 nM, 50 nM, 25 nM, 12.50 nM, 6.25 nM, 3.125 nM and 0 nM in a dilution gradient manner; capturing the antibody to be detected for 60 s by using a Protein A Sensor (Pall life sciences) in a PBST solution, respectively; binding to the human PTK7-His protein for 60 s; then dissociating for 180 s; opening the detection result in Data Analysis 11.0 software; selecting a 1:1 mode and performing global fitting; analyzing the result to obtain the binding rate, dissociation rate and affinity constant; and the result is shown in Table 5.

TABLE 5
Detection of dynamic affinity of anti-human
PTK7 humanized antibody with human PTK7-His

| Antibody | K_{D} (M) | kₒₙ(1/Ms) | k_{dis}(1/s) |
|---|---|---|---|
| Hu24 | 4.96E−09 | 2.11E+05 | 1.05E−03 |
| 64A10HZ | 1.09E−08 | 2.55E+05 | 2.78E−02 |
| 101A6HZ | 4.24E−08 | 1.34E+05 | 5.66E−03 |
| 4E12HZ | 2.20E−08 | 2.43E+05 | 5.34E−03 |
| 19C2HZ | 3.70E−09 | 1.97E+05 | 7.28E−04 |

### 3.4 Cross Species Detection of Anti-Human PTK7 Chimeric and Humanized Antibodies

ELISA and ForteBio were adopted to detect binding of the anti-human PTK7 chimeric and humanized antibodies to the monkey, mouse and rat PTK7 proteins. The specific method refers to 2.1 and 3.3, and the result is shown in Table 6.

TABLE 6
Detection of cross binding of anti-human PTK7
humanized antibody to monkey, mouse and rat

| | ELISA EC₅₀(ng/mL) | | | Dynamic binding K_{D} (M) | | |
|---|---|---|---|---|---|---|
| | Monkey | Mouse | Rat | Monkey | Mouse | Rat |
| Antibody | PTK7 | PTK7 | PTK7 | PTK7 | PTK7 | PTK7 |
| Hu24 | 133.74 | Not | Not | 1.19E−08 | Not | Not |
| | | bound | bound | | bound | bound |
| 64A10HZ | 20.09 | Not | Not | 2.67E−08 | Not | Not |
| | | bound | bound | | bound | bound |
| 101A6HZ | 1227.86 | Not | Not | 1.34E−07 | Not | Not |
| | | bound | bound | | bound | bound |
| 4E12HZ | — | — | — | 9.68E−09 | — | — |
| 19C2HZ | — | — | — | 9.20E−11 | — | — |

### 3.5 Detection of Cell Affinity of Anti-Human PTK7 Humanized Antibody

According to full-length proteins of human PTK7 (NP_002812.2), monkey PTK7 (NP_001248508.1), rat PTK7 (NP_001382670.1) and mouse PTK7 (NP_780377.1), GenScript Biotech Corporation was entrusted to perform codon optimization and gene synthesis, and constructed into a lentiviral vector pLVX-IRES-puro, the viruses were packed and infected into CHO cells, and the pressurized screening and flow cytometry validation were performed to obtain an overexpression cell line stably expressing human, monkey, rat and mouse PTK7.

A flow cytometer (Beckman, Cytoflex) was used for detecting the affinity of the anti-human PTK7 chimeric and humanized antibodies with human non-small cell lung cancer cells H1299, human lung adenocarcinoma cells H1975, human breast cancer cells T47D, human ovarian cancer cells OVCAR-3 (all purchased from NANJING COBIOER BIOSCIENCES CO., LTD) and CHO-hPTK7 overexpression cells.

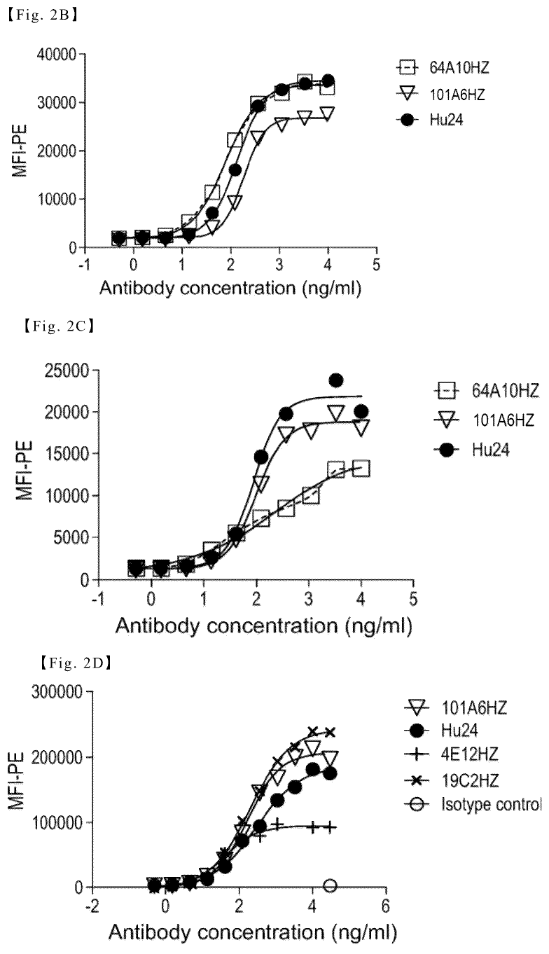

The adherent cells were digested by using a pancreatin (Gibco) solution; a proper amount of cells were counted and taken, washed twice by using 1×PBS, and re-suspended in a 1% BSA solution; and then the cells were transferred into a 96-well deep plate at a density of 50 μl/well; a candidate antibody was diluted by using 1% BSA, then 50 μl of diluted antibody was added into the deep plate containing the cells, and incubated for 40 min at 4° C.; the cells were washed twice by using PBS, then 50 μl of diluted secondary antibody was added into each well, uniformly mixed, and incubated for 30 min at 4° C.; and the cells were washed twice by using PBS, then re-suspended in 400 μl of PBS, and subjected to examination by flow cytometer. Data processing: a Median PE value was exported, and then imported into software to calculate EC₅₀; according to the results shown as FIGS. 2A-2D and Table 7, 64A10HZ, 64A10HZ05, 101A6HZ, 4E12HZ and 19C2HZ were bound to tumor cells or over-expression cells, and EC₅₀ was smaller than that of the control antibody Hu24, which indicated that the affinity of the bound cells was higher than that of Hu24, and the candidate antibody had better activity.

TABLE 7
Detection of affinity of anti-human PTK7 humanized antibody
with H1299, H1975, T47D, OVCAR-3 and CHO-hPTK7 cells

| | H1299 | H1975 | T47D | OVCAR-3 | CHO-hPTK7 |
|---|---|---|---|---|---|
| Antibody | EC₅₀(ng/ml) | EC₅₀(ng/ml) | EC₅₀(ng/ml) | EC₅₀(ng/ml) | EC₅₀(ng/ml) |
| Hu24 | 64.91 | 133.82 | 86.11 | 123.10 | 308.10 |
| 64A10HZ | 47.48 | 76.72 | 216.66 | 61.34 | — |
| 64A10HZ05 | — | — | — | 39.72 | — |
| 64A10-CH | 355.36 | — | — | — | — |
| 101A6HZ | 84.33 | 178.27 | 96.78 | — | 175.60 |
| 101A6-CH | 158.26 | — | — | — | — |
| 4E12HZ | — | — | — | — | 41.44 |
| 19C2HZ | — | — | — | — | 201.20 |

### 3.6 ADCC Activity Detection of Anti-Human PTK7 Humanized Antibody

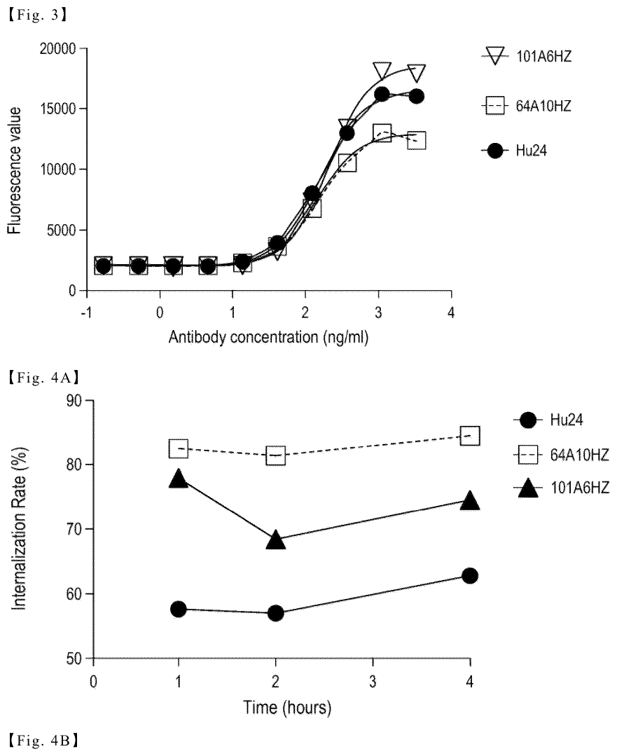

Under a condition that effector cells Jurkat-NFAT/luciferase-CD16a (obtained by lentivirus packaging, infection and pressurized screening), the antibody and target cells coexisted, the expression of luciferase would be activated, and an ONE-Glo reagent (Promega) was used as a substrate, and a fluorescence signal could be emitted. A specific method included as follows: centrifugally collecting Jurkat-NFAT/luciferase-CD16a and CHO-S-hPTK7 cells (obtained by lentivirus packaging, infection and pressurized screening), resuspending an RPMI 1640+1% FBS culture medium, respectively adjusting the densities to be 2.0×10⁶/ml and 1.0×10⁶/ml, and respectively adding into a 96-well plate at a density of 50 μl/well; diluting the antibody (starting from 10 μg/ml, with triple dilution and 11 concentration points) by the RPMI 1640+1% FBS culture medium, adding into the 96-well plate at a density of 50 μl/well, and incubating at 37° C. for 5 h; and adding 20 μl of One-glo (Promega) detection reagent, shaking to uniformly mix, reading a fluorescence signal value by the microplate reader, and importing the result into software to calculate the EC₅₀; and according to the result shown as FIG. 3, the ADCC activity of 64A10HZ and 101A6HZ was equivalent to that of the control antibody Hu24.

### 3.7 Endocytosis Detection of Anti-Human PTK7 Humanized Antibody

A flow cytometer (Beckman, Cytoflex) was used for detecting endocytosis of anti-human PTK7 chimeric and humanized antibodies and human non-small cell lung cancer cells H1299, human lung adenocarcinoma cells H1975 and human breast cancer cells T47D.

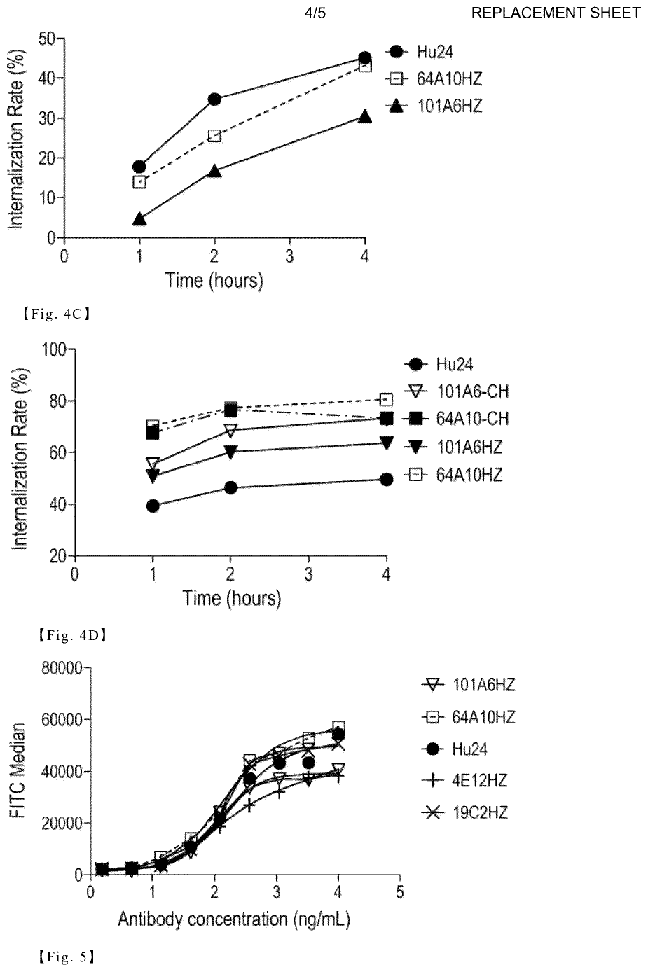

The adherent cells were digested by using a pancreatin (Gibco) solution; a proper amount of cells (2×10⁵ cells/cell) were counted and taken, and washed and suspended; then the cells were transferred into the 96-well deep plate at a density of 100 μl/well; then 100 μl of 10 μg/mL antibody diluted by 1% BSA was added into the deep plate containing the cells, and incubated on ice for 1 h; an irrelevant antibody was used as a control; washing was carried out, then 200 μl of culture medium (DMEM+10% FBS+P/S) was added into each well to re-suspend each group of cells, then distributed into the deep plate of 0 h, 1 h, 2 h and 4 h at a volume of 50 μl/well, and incubated at 4° C. and 37° C. for 0 h, 1 h, 2 h and 4 h respectively; after the incubation at a specific time point was finished, washing was carried out, 50 μL of fluorescent secondary antibody solution prepared by 0.5 μl of PE anti-human IgG Fc (50 μl of 1% BSA) was added into each well to re-suspend the cells, and the wells were continuously incubated on ice for 0.5 h; and the cells were washed, and detected by a machine. Median PE value was exported, and the endocytosis rate was calculated, the endocytosis rate (%)=[1−(MFI treatment group of sample for test in certain time point-MFI control hIgG at this time point)/(MFI treatment group of sample for test at 0 h-MFI control hIgG at 0 h)]×100%; and the result is shown in FIGS. 4A-4C. The antibody could be quickly and efficiently endocytosed in different cell lines, and no obvious difference existed between chimeric and humanized antibodies; the maximum endocytosis rate could be achieved in T47D and H1299 cells within 2 h, the endocytosis rate was continuously increased in H1975 cells along with the time extension, and the endocytosis rate reached about 40% within 4 h. The endocytosis rates of candidate humanized antibodies 64A10HZ and 101A6HZ in the T47D and H1299 cells were obviously higher than those of the control Hu24 antibody, and the endocytosis rate of the 64A10HZ in the H1975 cells was equivalent to that of Hu24.

The pharyngeal squamous cancer cells FADU (NANJING COBIOER BIOSCIENCES CO., LTD) was digested with pancreatin, washed twice with 1% BSA, resuspended and then counted; the cells were collected at a density of 2×10⁵ cells/well, and suspended by 1% of BSA at a density of 50 μl/well, and then plated on the deep plate; a first antibody, a secondary antibody and an anti-AF488 quenching antibody (PLoS ONE 10(4): e0124708) were incubated with reference to the method in the literature, and detected by a machine after incubation. Data analysis: fitting curves at different concentration points were made by using fluorescence signal values quenched at 37° C., EC₅₀ was calculated; and according to the result shown in FIG. 4D and Table 8, the candidate antibody had excellent endocytosis activity.

TABLE 8
FADU cell endocytosis activity detection
of anti-human PTK7 humanized antibody

| Antibody | EC₅₀(ng/ml) |
|---|---|
| Hu24 | 179.9 |
| 64A10HZ | 146.6 |
| 101A6HZ | 114.8 |
| 4E12HZ | 146.8 |
| 19C2HZ | 133.8 |

### 3.8 Antigen-Binding Epitope Analysis of Anti-Human PTK7 Humanized Antibody

The antigen-binding epitope of the anti-human PTK7 humanized antibody was analyzed through ELISA and FACS, respectively.

### 3.8.1 ELISA Detection

Hu24, 64A10HZ and 101A6HZ were labeled by adopting a biotin labeling kit (Thermo), ELISA detection showed that the binding activity to the human PTK7 protein was not obviously changed after labeling was completed, and EC₅₀ of each antibody was determined. The human PTK7 protein was diluted to reach 1 μg/ml by using the CBS coating liquid, coated by a 96-well microtiter plate at a density of 100 μl/well, and stood overnight at 4° C.; and after the plate was washed and blocked, the biotin-labeled antibody and the to-be-detected antibody were added, and incubated for 2 h at room temperature. After the plate was washed, the secondary antibody was added for incubation, and finally was subjected to color development, then the reaction was terminated, and the absorbance at OD₄₅₀ nm was read by the microplate reader. The original data was imported into software for nonlinear curve fitting (four parameters). According to the result shown in FIG. 5: the candidate antibodies 64A10HZ and 101A6HZ did not compete with the control antibody Hu24, and 64A10HZ did not compete with 101A6HZ, so that the three antibodies were different in antigen-binding epitopes.

### 3.8.2 FACS Detection

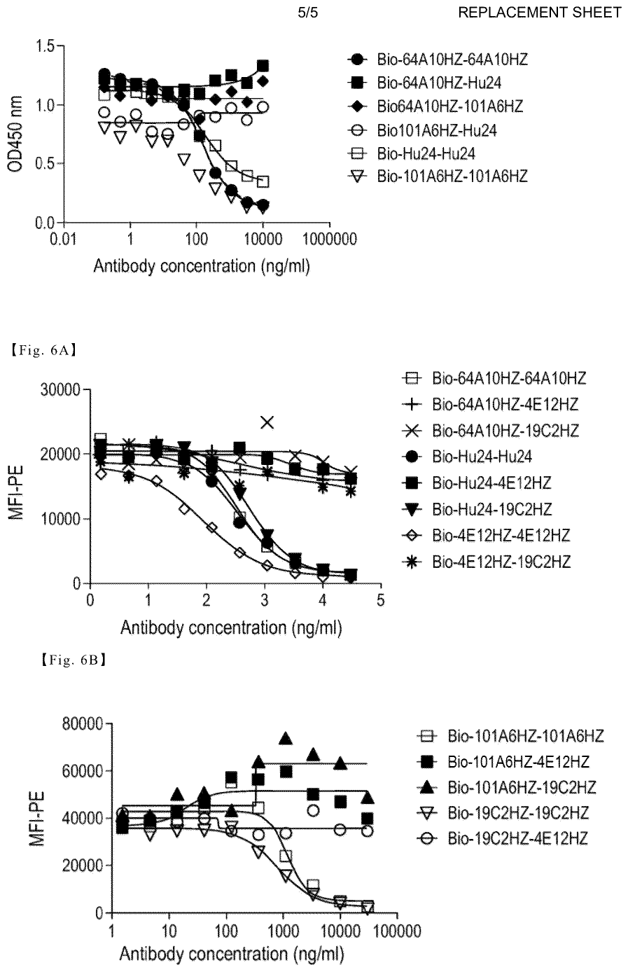

293T-hPTK7 cells were digested by pancreatin and collected; sufficient cells were counted and collected at a density of 2×10⁵ cells/well; the cells were added into a PCR deep plate at a density of 50 μl/well; the gradient-diluted antibody to be detected and the biotin-labeled antibody were added, and incubated at 4° C. for 60 min; the secondary antibody streptavidin PE was added after washing was performed, and incubating was performed at 4° C. in the dark for 30 min; and the cells were subjected to flow cytometry (FACS) by a machine after being washed, and the obtained fluorescence signal value was imported into software for analysis. According to the result shown in FIGS. 6A-6B: 19C2HZ could completely compete with the control antibody Hu24, so that the two antibodies were bound with similar epitopes, and the rest candidate antibodies did not compete with each other, which indicated that the binding epitopes were different.

### 3.9 Hydrophilicity Detection of Anti-Human PTK7 Humanized Antibody

Agilent 1260 and an analytical column TSKgel Butyl-NPR were adopted to detect the hydrophilicity of the antibody; the column temperature was 30° C., the detection wavelength was 280 nm, the flow rate was 0.5 ml/min, a mobile phase A was 1.5 mol/L (NH₄)₂SO₄, a mobile phase B was 25 mmol/L Na₂HPO₄, the pH was 7.0, and 25% IPA was adopted. An appropriate amount of the test sample was taken and diluted with a diluent (0.75 mol/L (NH₄)₂SO₄) to prepare 1.0 mg/ml solution as a test sample solution; and the control antibodies (hydrophilic control, Temelimab; and hydrophobic control, Sacituzumab, the controls were produced by Sichuan Kelun-Biotech Biopharmaceutical Co., Ltd.) were taken respectively and prepared into 1 mg/ml solution as a system applicability solution by using the diluent. About 40 μg of sample was injected, and analytical gradient elution was performed for 0-3 min, the mobile phase A was retained at 95%, and the mobile phase B was retained at 5%; the mobile phase B was raised from 5% to 100% within 3-40 min; and the mobile phase A was retained at 95%, and the mobile phase B was retained at 5% within 40-45 min. After detection, the hydrophobic value of the test sample was calculated according to the control sample, and the calculation formula was: (test sample retention time-hydrophilic control retention time)/(hydrophobic control retention time-hydrophilic control retention time); the smaller the retention time and the hydrophobic value were, the better the hydrophilicity of the antibody was. According to the result shown in Table 9, the candidate humanized antibodies 64A10HZ, 101A6HZ, 4E12HZ and 19C2HZ had better hydrophilicity than that of the control Hu24, and the good hydrophilicity was beneficial to production and quality control of the antibodies or small molecule coupling and the like, and meanwhile the in-vivo efficacy was enhanced and the drug metabolism condition was improved.

TABLE 9
hydrophilicity detection of anti-human PTK7 humanized antibody

| | Retention | Hydrophobic |
|---|---|---|
| Antibody | time | value |
| Hydrophilicity control | 11.479 | 0.00 |
| Hydrophobicity control | 17.846 | 1.00 |
| Hu24 | 16.083 | 0.72 |
| 64A10HZ | 11.65 | 0.03 |
| 101A6HZ | 11.375 | −0.02 |
| 4E12HZ | 12.207 | 0.11 |
| 19C2HZ | 11.888 | 0.06 |

### 3.10 Detection of Binding Activity of Anti-Human PTK7 Humanized Mutant Antibody to Fc Receptor

The humanized 101A6 heavy chain variable region was fused to a mutation-containing human IgG1 heavy chain constant region (SEQ ID NO: 131), and an expressed antibody was named as 101A6HZm. The dynamic affinity of the antibodies 101A6HZ, 101A6HZm and Hu24 with human Fc receptor proteins CD16a, CD32a, CD32b, CD64 and FcRn was detected through ForteBio (Pall life sciences). A specific method included: respectively capturing biotinylated proteins to be detected in a PBST solution by using SA sensors (Pall life sciences); diluting the antibodies to be detected and the conjugate to reach an initial concentration of 5,000 nM by using PBST, and performing double dilution until reaching 7 concentration points; binding, dissociating and opening the detection result in Data Analysis 11.0 software; and analyzing the result by selecting a 1:1 mode and global fitting to obtain the binding rate, dissociation rate and affinity constant; and the result is shown as Table 10.

TABLE 10
Detection of binding activity of PTK7 humanized mutant antibody to Fc receptor

| | KD(M) | | | | |
|---|---|---|---|---|---|
| Antibody | CD16a | CD32a | CD32b | CD64 | FcRn |
| 101A6HZ | 1.01E−6 | 2.27E−6 | 1.78E−6 | 5.08E−10 | 1.18E−8 |
| 101A6HZm | not detected | not detected | not detected | not detected | 1.66E−8 |
| Hu24 | 1.19E−6 | 1.73E−6 | 1.76E−6 | 4.65E−10 | 1.16E−8 |

The result showed that the 101A6HZm subjected to mutation modification was not bound to the CD16a, CD32a, CD32b and CD64 proteins of the Fc receptor, so that the Fc receptor-mediated nonspecific killing could be reduced, and the drug safety was improved; and meanwhile, the 101A6HZm retained the binding activity of FcRn protein, and the half-life of the drug was not affected.

### 3.11 Cell Endocytosis Detection of Anti-Human PTK7 Antibody

HCC1806 and OVCAR3 cells were digested by pancreatin, and were counted after being resuspended; the cells were taken at a density of 2×10⁵ cells/well, resuspended by 1% BSA at a density of 50 μl/well, and then plated to a deep plate; the antibody was added, and incubated at 4° C. for 60 min; the cells were washed twice by using 1% BSA, then the secondary antibody (Anti-human IgG Alexa Fluor 488) was added, and incubated at 4° C. for 30 min; and the endocytosis (PLoS ONE 10(4): e0124708) at different temperatures was detected with reference to the literature; 150 μl of 1% BSA was added for resuspension after incubation, and then the cells were detected by a machine. Data analysis: a fitting curve at different concentration points was made by using fluorescence signal values after quenching at 37° C., and EC50 was calculated; and according to the result shown in Table 11, the candidate antibody had excellent endocytosis activity.

TABLE 11
Cell endocytosis detection of antibody

EC₅₀(ng/ml)

| Drug | OVCAR3 | NCI-H358 | HCC1806 | A431 |
|---|---|---|---|---|
| 101A6HZm | 66.53 | 72.94 | 57.60 | 73.19 |
| Hu24 | 101.40 | 70.77 | 65.88 | 74.30 |

Although the specific embodiments of the present invention have been described in detail, those skilled in the art will understand that various modifications and changes can be made in details in light of all the published teachings, and these changes are within the protection scope of the present invention. The full scope of the present invention is given by the appended claims and any equivalents thereof.

1-67. (canceled)

68. A specific PTK7-binding antibody or antigen-binding fragment thereof, comprising the following complementarity determining regions (CDRs):
(a) CDR-H1, CDR-H2 and CDR-H3 contained in a heavy chain variable region (VH) shown as SEQ ID NO: 19; and/or CDR-L1, CDR-L2 and CDR-L3 contained in a light chain variable region (VL) shown as SEQ ID NO: 20;
(b) CDR-H1, CDR-H2 and CDR-H3 contained in a heavy chain variable region (VH) shown as SEQ ID NO: 1; and/or CDR-L1, CDR-L2 and CDR-L3 contained in a light chain variable region (VL) shown as SEQ ID NO: 2;
(c) CDR-H1, CDR-H2 and CDR-H3 contained in a heavy chain variable region (VH) shown as SEQ ID NO: 3; and/or CDR-L1, CDR-L2 and CDR-L3 contained in a light chain variable region (VL) shown as SEQ ID NO: 4;
(d) CDR-H1, CDR-H2 and CDR-H3 contained in a heavy chain variable region (VH) shown as SEQ ID NO: 5; and/or CDR-L1, CDR-L2 and CDR-L3 contained in a light chain variable region (VL) shown as SEQ ID NO: 6;
(e) CDR-H1, CDR-H2 and CDR-H3 contained in a heavy chain variable region (VH) shown as SEQ ID NO: 7; and/or CDR-L1, CDR-L2 and CDR-L3 contained in a light chain variable region (VL) shown as SEQ ID NO: 8;
(f) CDR-H1, CDR-H2 and CDR-H3 contained in a heavy chain variable region (VH) shown as SEQ ID NO: 9; and/or CDR-L1, CDR-L2 and CDR-L3 contained in a light chain variable region (VL) shown as SEQ ID NO: 10;
(g) CDR-H1, CDR-H2 and CDR-H3 contained in a heavy chain variable region (VH) shown as SEQ ID NO: 11; and/or CDR-L1, CDR-L2 and CDR-L3 contained in a light chain variable region (VL) shown as SEQ ID NO: 12;
(h) CDR-H1, CDR-H2 and CDR-H3 contained in a heavy chain variable region (VH) shown as SEQ ID NO: 13; and/or CDR-L1, CDR-L2 and CDR-L3 contained in a light chain variable region (VL) shown as SEQ ID NO: 14;
(i) CDR-H1, CDR-H2 and CDR-H3 contained in a heavy chain variable region (VH) shown as SEQ ID NO: 15; and/or CDR-L1, CDR-L2 and CDR-L3 contained in a light chain variable region (VL) shown as SEQ ID NO: 16;
(j) CDR-H1, CDR-H2 and CDR-H3 contained in a heavy chain variable region (VH) shown as SEQ ID NO: 17; and/or CDR-L1, CDR-L2 and CDR-L3 contained in a light chain variable region (VL) shown as SEQ ID NO: 18;
(k) CDR-H1, CDR-H2 and CDR-H3 contained in a heavy chain variable region (VH) shown as SEQ ID NO: 21; and/or CDR-L1, CDR-L2 and CDR-L3 contained in a light chain variable region (VL) shown as SEQ ID NO: 22;
(l) CDR-H1, CDR-H2 and CDR-H3 contained in a heavy chain variable region (VH) shown as SEQ ID NO: 23; and/or CDR-L1, CDR-L2 and CDR-L3 contained in a light chain variable region (VL) shown as SEQ ID NO: 24; or
(m) CDR-H1, CDR-H2 and CDR-H3 contained in the following heavy chain variable region (VH), and/or CDR-L1, CDR-L2 and CDR-L3 contained in the following light chain variable region (VL), wherein at least one CDR of the heavy chain variable region (VH) and/or the light chain variable region (VL) contains a mutation as compared to the heavy chain variable regions (VH) and/or the light chain variable regions (VL) in any one of (a) to (1), and the mutation refers to replacement, deletion or addition of one or several amino acids.

69. The antibody or antigen-binding fragment thereof according to claim 68, comprising:
(1) the following heavy chain variable regions (VH) and/or light chain variable regions (VL), wherein the CDRs are defined according to the Chothia numbering system:
(1a) a heavy chain variable region (VH) comprising the following 3 CDRs: CDR-H1 having a sequence shown as SEQ ID NO: 27 or a variant thereof, CDR-H2 having a sequence shown as SEQ ID NO: 28 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
(1b) a heavy chain variable region (VH) comprising the following 3 CDRs: CDR-H1 having a sequence shown as SEQ ID NO: 41 or a variant thereof, CDR-H2 having a sequence shown as SEQ ID NO: 42 or a variant thereof, and CDR-H3 having a sequence shown as SEQ ID NO: 43 or a variant thereof, and/or, a light chain variable region (VL) comprising the following 3 CDRs: CDR-L1 having a sequence shown as SEQ ID NO: 44 or a variant thereof, CDR-L2 having a sequence shown as SEQ ID NO: 45 or a variant thereof, and CDR-L3 having a sequence shown as SEQ ID NO: 46 or a variant thereof,
(1c) a heavy chain variable region (VH) comprising the following 3 CDRs: CDR-H1 having a sequence shown as SEQ ID NO: 55 or a variant thereof, CDR-H2 having a sequence shown as SEQ ID NO: 56 or a variant thereof, and CDR-H3 having a sequence shown as SEQ ID NO: 57 or a variant thereof; and/or, a light chain variable region (VL) comprising the following 3 CDRs: CDR-L1 having a sequence shown as SEQ ID NO: 58 or a variant thereof, CDR-L2 having a sequence shown as SEQ ID NO: 59 or a variant thereof, and CDR-L3 having a sequence shown as SEQ ID NO: 60 or a variant thereof;
(1d) a heavy chain variable region (VH) comprising the following 3 CDRs: CDR-H1 having a sequence shown as SEQ ID NO: 68 or a variant thereof, CDR-H2 having a sequence shown as SEQ ID NO: 69 or a variant thereof, and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof, CDR-L2 having a sequence shown as SEQ ID NO: 72 or a variant thereof, and CDR-L3 having a sequence shown as SEQ ID NO: 73 or a variant thereof;
(1e) a heavy chain variable region (VH) comprising the following 3 CDRs: CDR-H1 having a sequence shown as SEQ ID NO: 74 or a variant thereof, CDR-H2 having a sequence shown as SEQ ID NO: 69 or a variant thereof, and CDR-H3 having a sequence shown as SEQ ID NO: 83 or a variant thereof; and/or, a light chain variable region (VL) comprising the following 3 CDRs: CDR-L1 having a sequence shown as SEQ ID NO: 84 or a variant thereof, CDR-L2 having a sequence shown as SEQ ID NO: 85 or a variant thereof, and CDR-L3 having a sequence shown as SEQ ID NO: 86 or a variant thereof;
(1f) a heavy chain variable region (VH) comprising the following 3 CDRs: CDR-H1 having a sequence shown as SEQ ID NO: 92 or a variant thereof, CDR-H2 having a sequence shown as SEQ ID NO: 93 or a variant thereof, and CDR-H3 having a sequence shown as SEQ ID NO: 94 or a variant thereof; and/or, a light chain variable region (VL) comprising the following 3 CDRs: CDR-L1 having a sequence shown as SEQ ID NO: 95 or a variant thereof, CDR-L2 having a sequence shown as SEQ ID NO: 96 or a variant thereof, and CDR-L3 having a sequence shown as SEQ ID NO: 97 or a variant thereof,
(1g) a heavy chain variable region (VH) comprising the following 3 CDRs: CDR-H1 having a sequence shown as SEQ ID NO: 27 or a variant thereof, CDR-H2 having a sequence shown as SEQ ID NO: 28 or a variant thereof, and CDR-H3 having a sequence shown as SEQ ID NO: 105 or a variant thereof; and/or, a light chain variable region (VL) comprising the following 3 CDRs: CDR-L1 having a sequence shown as SEQ ID NO: 106 or a variant thereof, CDR-L2 having a sequence shown as SEQ ID NO: 31 or a variant thereof, and CDR-L3 having a sequence shown as SEQ ID NO: 107 or a variant thereof,
(1h) a heavy chain variable region (VH) comprising the following 3 CDRs: CDR-H1 having a sequence shown as SEQ ID NO: 41; CDR-H2 having a sequence shown as SEQ ID NO: 42; and CDR-H3 having a sequence shown as SEQ ID NO: 43; and, a light chain variable region (VL) comprising the following 3 CDRs: CDR-L1 having a sequence shown as SEQ ID NO: 44; CDR-L2 having a sequence shown as SEQ ID NO: 45; and CDR-L3 having a sequence shown as SEQ ID NO: 46;
(1i) a heavy chain variable region (VH) comprising the following 3 CDRs: CDR-H1 having a sequence shown as SEQ ID NO: 41; CDR-H2 having a sequence shown as SEQ ID NO: 42; and CDR-H3 having a sequence shown as SEQ ID NO: 43; or, a light chain variable region (VL) comprising the following 3 CDRs: CDR-L1 having a sequence shown as SEQ ID NO: 44; CDR-L2 having a sequence shown as SEQ ID NO: 45; and CDR-L3 having a sequence shown as SEQ ID NO: 46;
(1j) a heavy chain variable region (VH) comprising the following 3 CDRs: CDR-H1 having a sequence shown as SEQ ID NO: 27; CDR-H2 having a sequence shown as SEQ ID NO: 28; and CDR-H3 having a sequence shown as SEQ ID NO: 29; and, a light chain variable region (VL) comprising the following 3 CDRs: CDR-L1 having a sequence shown as SEQ ID NO: 30; CDR-L2 having a sequence shown as SEQ ID NO: 31; and CDR-L3 having a sequence shown as SEQ ID NO: 32;
(1k) a heavy chain variable region (VH) comprising the following 3 CDRs: CDR-H1 having a sequence shown as SEQ ID NO: 55; CDR-H2 having a sequence shown as SEQ ID NO: 56; and CDR-H3 having a sequence shown as SEQ ID NO: 57; and, a light chain variable region (VL) comprising the following 3 CDRs: CDR-L1 having a sequence shown as SEQ ID NO: 58; CDR-L2 having a sequence shown as SEQ ID NO: 59; and CDR-L3 having a sequence shown as SEQ ID NO: 60; or
(1l) a heavy chain variable region (VH) comprising the following 3 CDRs: CDR-H1 having a sequence shown as SEQ ID NO: 74 or a variant thereof, CDR-H2 having a sequence shown as SEQ ID NO: 69 or a variant thereof, and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof, CDR-L2 having a sequence shown as SEQ ID NO: 72 or a variant thereof, and CDR-L3 having a sequence shown as SEQ ID NO: 73 or a variant thereof;
or,
(2) the following heavy chain variable regions (VH) and/or light chain variable regions (VL), wherein the CDRs are defined according to the Kabat numbering system:
(2a) a heavy chain variable region (VH) comprising the following 3 CDRs: CDR-H1 having a sequence shown as SEQ ID NO: 33 or a variant thereof, CDR-H2 having a sequence shown as SEQ ID NO: 34 or a variant thereof, and CDR-H3 having a sequence shown as SEQ ID NO: 29 or a variant thereof, and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
(2b) a heavy chain variable region (VH) comprising the following 3 CDRs: CDR-H1 having a sequence shown as SEQ ID NO: 33 or a variant thereof, CDR-H2 having a sequence shown as SEQ ID NO: 35 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
(2c) a heavy chain variable region (VH) comprising the following 3 CDRs: CDR-H1 having a sequence shown as SEQ ID NO: 47 or a variant thereof, CDR-H2 having a sequence shown as SEQ ID NO: 49 or a variant thereof, and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof, CDR-L2 having a sequence shown as SEQ ID NO: 45 or a variant thereof, and CDR-L3 having a sequence shown as SEQ ID NO: 46 or a variant thereof,
(2d) a heavy chain variable region (VH) comprising the following 3 CDRs: CDR-H1 having a sequence shown as SEQ ID NO: 61 or a variant thereof, CDR-H2 having a sequence shown as SEQ ID NO: 62 or a variant thereof, and CDR-H3 having a sequence shown as SEQ ID NO: 57 or a variant thereof; and/or, a light chain variable region (VL) comprising the following 3 CDRs: CDR-L1 having a sequence shown as SEQ ID NO: 58 or a variant thereof, CDR-L2 having a sequence shown as SEQ ID NO: 59 or a variant thereof, and CDR-L3 having a sequence shown as SEQ ID NO: 60 or a variant thereof;
(2e) a heavy chain variable region (VH) comprising the following 3 CDRs: CDR-H1 having a sequence shown as SEQ ID NO: 75 or a variant thereof, CDR-H2 having a sequence shown as SEQ ID NO: 76 or a variant thereof, and CDR-H3 having a sequence shown as SEQ ID NO: 70 or a variant thereof; and/or, a light chain variable region (VL) comprising the following 3 CDRs: CDR-L1 having a sequence shown as SEQ ID NO: 71 or a variant thereof, CDR-L2 having a sequence shown as SEQ ID NO: 72 or a variant thereof, and CDR-L3 having a sequence shown as SEQ ID NO: 73 or a variant thereof;
(2f) a heavy chain variable region (VH) comprising the following 3 CDRs: CDR-H1 having a sequence shown as SEQ ID NO: 87 or a variant thereof, CDR-H2 having a sequence shown as SEQ ID NO: 88 or a variant thereof, and CDR-H3 having a sequence shown as SEQ ID NO: 83 or a variant thereof; and/or, a light chain variable region (VL) comprising the following 3 CDRs: CDR-L1 having a sequence shown as SEQ ID NO: 84 or a variant thereof, CDR-L2 having a sequence shown as SEQ ID NO: 85 or a variant thereof, and CDR-L3 having a sequence shown as SEQ ID NO: 86 or a variant thereof;
(2g) a heavy chain variable region (VH) comprising the following 3 CDRs: CDR-H1 having a sequence shown as SEQ ID NO: 98 or a variant thereof, CDR-H2 having a sequence shown as SEQ ID NO: 99 or a variant thereof, and CDR-H3 having a sequence shown as SEQ ID NO: 94 or a variant thereof; and/or, a light chain variable region (VL) comprising the following 3 CDRs: CDR-L1 having a sequence shown as SEQ ID NO: 95 or a variant thereof, CDR-L2 having a sequence shown as SEQ ID NO: 96 or a variant thereof, and CDR-L3 having a sequence shown as SEQ ID NO: 97 or a variant thereof;
(2h) a heavy chain variable region (VH) comprising the following 3 CDRs: CDR-H1 having a sequence shown as SEQ ID NO: 108 or a variant thereof, CDR-H2 having a sequence shown as SEQ ID NO: 35 or a variant thereof, and CDR-H3 having a sequence shown as SEQ ID NO: 105 or a variant thereof, and/or, a light chain variable region (VL) comprising the following 3 CDRs: CDR-L1 having a sequence shown as SEQ ID NO: 106 or a variant thereof, CDR-L2 having a sequence shown as SEQ ID NO: 31 or a variant thereof, and CDR-L3 having a sequence shown as SEQ ID NO: 107 or a variant thereof,
(2i) a heavy chain variable region (VH) comprising the following 3 CDRs: CDR-H1 having a sequence shown as SEQ ID NO: 47 or a variant thereof, CDR-H2 having a sequence shown as SEQ ID NO: 48 or a variant thereof, and CDR-H3 having a sequence shown as SEQ ID NO: 43 or a variant thereof; and/or, a light chain variable region (VL) comprising the following 3 CDRs: CDR-L1 having a sequence shown as SEQ ID NO: 44 or a variant thereof, CDR-L2 having a sequence shown as SEQ ID NO: 45 or a variant thereof, and CDR-L3 having a sequence shown as SEQ ID NO: 46 or a variant thereof;
(2j) a heavy chain variable region (VH) comprising the following 3 CDRs: CDR-H1 having a sequence shown as SEQ ID NO: 47; CDR-H2 having a sequence shown as SEQ ID NO: 48; and CDR-H3 having a sequence shown as SEQ ID NO: 43; and, a light chain variable region (VL) comprising the following 3 CDRs: CDR-L1 having a sequence shown as SEQ ID NO: 44; CDR-L2 having a sequence shown as SEQ ID NO: 45; and CDR-L3 having a sequence shown as SEQ ID NO: 46;
(2k) a heavy chain variable region (VH) comprising the following 3 CDRs: CDR-H1 having a sequence shown as SEQ ID NO: 61; CDR-H2 having a sequence shown as SEQ ID NO: 62; and CDR-H3 having a sequence shown as SEQ ID NO: 57; and, a light chain variable region (VL) comprising the following 3 CDRs: CDR-L1 having a sequence shown as SEQ ID NO: 58; CDR-L2 having a sequence shown as SEQ ID NO: 59; and CDR-L3 having a sequence shown as SEQ ID NO: 60; or
(2l) a heavy chain variable region (VH) comprising the following 3 CDRs: CDR-H1 having a sequence shown as SEQ ID NO: 75; CDR-H2 having a sequence shown as SEQ ID NO: 76; and CDR-H3 having a sequence shown as SEQ ID NO: 70; and, a light chain variable region (VL) comprising the following 3 CDRs: CDR-L1 having a sequence shown as SEQ ID NO: 71; CDR-L2 having a sequence shown as SEQ ID NO: 72; and CDR-L3 having a sequence shown as SEQ ID NO: 73;
or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having a sequence shown as SEQ ID NO: 36 or a variant thereof, CDR-H2 having a sequence shown as SEQ ID NO: 37 or a variant thereof, and CDR-H3 having a sequence shown as SEQ ID NO: 38 or a variant thereof; and/or, a light chain variable region (VL) comprising the following 3 CDRs: CDR-L1 having a sequence shown as SEQ ID NO: 39 or a variant thereof, CDR-L2 having a sequence shown as SEQ ID NO: 40 or a variant thereof, and CDR-L3 having a sequence shown as SEQ ID NO: 32 or a variant thereof;
(3b) a heavy chain variable region (VH) comprising the following 3 CDRs: CDR-H1 having a sequence shown as SEQ ID NO: 50 or a variant thereof, CDR-H2 having a sequence shown as SEQ ID NO: 51 or a variant thereof, and CDR-H3 having a sequence shown as SEQ ID NO: 52 or a variant thereof, and/or, a light chain variable region (VL) comprising the following 3 CDRs: CDR-L1 having a sequence shown as SEQ ID NO: 53 or a variant thereof, CDR-L2 having a sequence shown as SEQ ID NO: 54 or a variant thereof, and CDR-L3 having a sequence shown as SEQ ID NO: 46 or a variant thereof;
(3c) a heavy chain variable region (VH) comprising the following 3 CDRs: CDR-H1 having a sequence shown as SEQ ID NO: 63 or a variant thereof, CDR-H2 having a sequence shown as SEQ ID NO: 64 or a variant thereof, and CDR-H3 having a sequence shown as SEQ ID NO: 65 or a variant thereof; and/or, a light chain variable region (VL) comprising the following 3 CDRs: CDR-L1 having a sequence shown as SEQ ID NO: 66 or a variant thereof, CDR-L2 having a sequence shown as SEQ ID NO: 67 or a variant thereof, and CDR-L3 having a sequence shown as SEQ ID NO: 60 or a variant thereof;
(3d) a heavy chain variable region (VH) comprising the following 3 CDRs: CDR-H1 having a sequence shown as SEQ ID NO: 77 or a variant thereof, CDR-H2 having a sequence shown as SEQ ID NO: 78 or a variant thereof, and CDR-H3 having a sequence shown as SEQ ID NO: 79 or a variant thereof; and/or, a light chain variable region (VL) comprising the following 3 CDRs: CDR-L1 having a sequence shown as SEQ ID NO: 81 or a variant thereof, CDR-L2 having a sequence shown as SEQ ID NO: 82 or a variant thereof, and CDR-L3 having a sequence shown as SEQ ID NO: 73 or a variant thereof;
(3e) a heavy chain variable region (VH) comprising the following 3 CDRs: CDR-H1 having a sequence shown as SEQ ID NO: 80 or a variant thereof, CDR-H2 having a sequence shown as SEQ ID NO: 78 or a variant thereof, and CDR-H3 having a sequence shown as SEQ ID NO: 89 or a variant thereof, and/or, a light chain variable region (VL) comprising the following 3 CDRs: CDR-L1 having a sequence shown as SEQ ID NO: 90 or a variant thereof, CDR-L2 having a sequence shown as SEQ ID NO: 91 or a variant thereof, and CDR-L3 having a sequence shown as SEQ ID NO: 86 or a variant thereof;
(3f) a heavy chain variable region (VH) comprising the following 3 CDRs: CDR-H1 having a sequence shown as SEQ ID NO: 100 or a variant thereof, CDR-H2 having a sequence shown as SEQ ID NO: 101 or a variant thereof, and CDR-H3 having a sequence shown as SEQ ID NO: 102 or a variant thereof, and/or, a light chain variable region (VL) comprising the following 3 CDRs: CDR-L1 having a sequence shown as SEQ ID NO: 103 or a variant thereof, CDR-L2 having a sequence shown as SEQ ID NO: 104 or a variant thereof, and CDR-L3 having a sequence shown as SEQ ID NO: 97 or a variant thereof,
(3g) a heavy chain variable region (VH) comprising the following 3 CDRs: CDR-H1 having a sequence shown as SEQ ID NO: 36 or a variant thereof, CDR-H2 having a sequence shown as SEQ ID NO: 37 or a variant thereof, and CDR-H3 having a sequence shown as SEQ ID NO: 109 or a variant thereof; and/or, a light chain variable region (VL) comprising the following 3 CDRs: CDR-L1 having a sequence shown as SEQ ID NO: 110 or a variant thereof, CDR-L2 having a sequence shown as SEQ ID NO: 40 or a variant thereof, and CDR-L3 having a sequence shown as SEQ ID NO: 107 or a variant thereof,
(3h) a heavy chain variable region (VH) comprising the following 3 CDRs: CDR-H1 having a sequence shown as SEQ ID NO: 50; CDR-H2 having a sequence shown as SEQ ID NO: 51; and CDR-H3 having a sequence shown as SEQ ID NO: 52; and, a light chain variable region (VL) comprising the following 3 CDRs: CDR-L1 having a sequence shown as SEQ ID NO: 53; CDR-L2 having a sequence shown as SEQ ID NO: 54; and CDR-L3 having a sequence shown as SEQ ID NO: 46;
(3i) a heavy chain variable region (VH) comprising the following 3 CDRs: CDR-H1 having a sequence shown as SEQ ID NO: 50; CDR-H2 having a sequence shown as SEQ ID NO: 51; and CDR-H3 having a sequence shown as SEQ ID NO: 52; or, a light chain variable region (VL) comprising the following 3 CDRs: CDR-L1 having a sequence shown as SEQ ID NO: 53; CDR-L2 having a sequence shown as SEQ ID NO: 54; and CDR-L3 having a sequence shown as SEQ ID NO: 46;
(3j) a heavy chain variable region (VH) comprising the following 3 CDRs: CDR-H1 having a sequence shown as SEQ ID NO: 36; CDR-H2 having a sequence shown as SEQ ID NO: 37; and CDR-H3 having a sequence shown as SEQ ID NO: 38; and, a light chain variable region (VL) comprising the following 3 CDRs: CDR-L1 having a sequence shown as SEQ ID NO: 39; CDR-L2 having a sequence shown as SEQ ID NO: 40; and CDR-L3 having a sequence shown as SEQ ID NO: 32;
(3k) a heavy chain variable region (VH) comprising the following 3 CDRs: CDR-H1 having a sequence shown as SEQ ID NO: 63; CDR-H2 having a sequence shown as SEQ ID NO: 64; and CDR-H3 having a sequence shown as SEQ ID NO: 65; and, a light chain variable region (VL) comprising the following 3 CDRs: CDR-L1 having a sequence shown as SEQ ID NO: 66; CDR-L2 having a sequence shown as SEQ ID NO: 67; and CDR-L3 having a sequence shown as SEQ ID NO: 60; or
(3l) a heavy chain variable region (VH) comprising the following 3 CDRs: CDR-H1 having a sequence shown as SEQ ID NO: 80 or a variant thereof, CDR-H2 having a sequence shown as SEQ ID NO: 78 or a variant thereof, and CDR-H3 having a sequence shown as SEQ ID NO: 79 or a variant thereof; and/or, a light chain variable region (VL) comprising the following 3 CDRs: CDR-L1 having a sequence shown as SEQ ID NO: 81 or a variant thereof, CDR-L2 having a sequence shown as SEQ ID NO: 82 or a variant thereof, and CDR-L3 having a sequence shown as SEQ ID NO: 73 or a variant thereof;
or,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having a sequence shown as SEQ ID NO: 113 or a variant thereof, CDR-H2 having a sequence shown as SEQ ID NO: 114 or a variant thereof, and CDR-H3 having a sequence shown as SEQ ID NO: 29 or a variant thereof, and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
(4b) a heavy chain variable region (VH) comprising the following 3 CDRs: CDR-H1 having a sequence shown as SEQ ID NO: 115 or a variant thereof, CDR-H2 having a sequence shown as SEQ ID NO: 116 or a variant thereof, and CDR-H3 having a sequence shown as SEQ ID NO: 43 or a variant thereof, and/or, a light chain variable region (VL) comprising the following 3 CDRs: CDR-L1 having a sequence shown as SEQ ID NO: 44 or a variant thereof, CDR-L2 having a sequence shown as SEQ ID NO: 45 or a variant thereof, and CDR-L3 having a sequence shown as SEQ ID NO: 46 or a variant thereof;
(4c) a heavy chain variable region (VH) comprising the following 3 CDRs: CDR-H1 having a sequence shown as SEQ ID NO: 117 or a variant thereof, CDR-H2 having a sequence shown as SEQ ID NO: 118 or a variant thereof, and CDR-H3 having a sequence shown as SEQ ID NO: 57 or a variant thereof, and/or, a light chain variable region (VL) comprising the following 3 CDRs: CDR-L1 having a sequence shown as SEQ ID NO: 58 or a variant thereof, CDR-L2 having a sequence shown as SEQ ID NO: 59 or a variant thereof, and CDR-L3 having a sequence shown as SEQ ID NO: 60 or a variant thereof;
(4d) a heavy chain variable region (VH) comprising the following 3 CDRs: CDR-H1 having a sequence shown as SEQ ID NO: 119 or a variant thereof, CDR-H2 having a sequence shown as SEQ ID NO: 120 or a variant thereof, and CDR-H3 having a sequence shown as SEQ ID NO: 70 or a variant thereof, and/or, a light chain variable region (VL) comprising the following 3 CDRs: CDR-L1 having a sequence shown as SEQ ID NO: 71 or a variant thereof, CDR-L2 having a sequence shown as SEQ ID NO: 72 or a variant thereof, and CDR-L3 having a sequence shown as SEQ ID NO: 73 or a variant thereof;
(4e) a heavy chain variable region (VH) comprising the following 3 CDRs: CDR-H1 having a sequence shown as SEQ ID NO: 122 or a variant thereof, CDR-H2 having a sequence shown as SEQ ID NO: 123 or a variant thereof, and CDR-H3 having a sequence shown as SEQ ID NO: 83 or a variant thereof, and/or, a light chain variable region (VL) comprising the following 3 CDRs: CDR-L1 having a sequence shown as SEQ ID NO: 84 or a variant thereof, CDR-L2 having a sequence shown as SEQ ID NO: 85 or a variant thereof, and CDR-L3 having a sequence shown as SEQ ID NO: 86 or a variant thereof,
(4f) a heavy chain variable region (VH) comprising the following 3 CDRs: CDR-H1 having a sequence shown as SEQ ID NO: 124 or a variant thereof, CDR-H2 having a sequence shown as SEQ ID NO: 125 or a variant thereof, and CDR-H3 having a sequence shown as SEQ ID NO: 94 or a variant thereof, and/or, a light chain variable region (VL) comprising the following 3 CDRs: CDR-L1 having a sequence shown as SEQ ID NO: 95 or a variant thereof, CDR-L2 having a sequence shown as SEQ ID NO: 96 or a variant thereof, and CDR-L3 having a sequence shown as SEQ ID NO: 97 or a variant thereof;
(4g) a heavy chain variable region (VH) comprising the following 3 CDRs: CDR-H1 having a sequence shown as SEQ ID NO: 126 or a variant thereof, CDR-H2 having a sequence shown as SEQ ID NO: 114 or a variant thereof, and CDR-H3 having a sequence shown as SEQ ID NO: 105 or a variant thereof, and/or, a light chain variable region (VL) comprising the following 3 CDRs: CDR-L1 having a sequence shown as SEQ ID NO: 106 or a variant thereof, CDR-L2 having a sequence shown as SEQ ID NO: 31 or a variant thereof, and CDR-L3 having a sequence shown as SEQ ID NO: 107 or a variant thereof,
(4h) a heavy chain variable region (VH) comprising the following 3 CDRs: CDR-H1 having a sequence shown as SEQ ID NO: 115; CDR-H2 having a sequence shown as SEQ ID NO: 116; and CDR-H3 having a sequence shown as SEQ ID NO: 43; and, a light chain variable region (VL) comprising the following 3 CDRs: CDR-L1 having a sequence shown as SEQ ID NO: 44; CDR-L2 having a sequence shown as SEQ ID NO: 45; and CDR-L3 having a sequence shown as SEQ ID NO: 46;
(4i) a heavy chain variable region (VH) comprising the following 3 CDRs: CDR-H1 having a sequence shown as SEQ ID NO: 115; CDR-H2 having a sequence shown as SEQ ID NO: 116; and CDR-H3 having a sequence shown as SEQ ID NO: 43; or, a light chain variable region (VL) comprising the following 3 CDRs: CDR-L1 having a sequence shown as SEQ ID NO: 44; CDR-L2 having a sequence shown as SEQ ID NO: 45; and CDR-L3 having a sequence shown as SEQ ID NO: 46;
(4j) a heavy chain variable region (VH) comprising the following 3 CDRs: CDR-H1 having a sequence shown as SEQ ID NO: 113; CDR-H2 having a sequence shown as SEQ ID NO: 114; and CDR-H3 having a sequence shown as SEQ ID NO: 29; and, a light chain variable region (VL) comprising the following 3 CDRs: CDR-L1 having a sequence shown as SEQ ID NO: 30; CDR-L2 having a sequence shown as SEQ ID NO: 31; and CDR-L3 having a sequence shown as SEQ ID NO: 32;
(4k) a heavy chain variable region (VH) comprising the following 3 CDRs: CDR-H1 having a sequence shown as SEQ ID NO: 117; CDR-H2 having a sequence shown as SEQ ID NO: 118; and CDR-H3 having a sequence shown as SEQ ID NO: 57; and, a light chain variable region (VL) comprising the following 3 CDRs: CDR-L1 having a sequence shown as SEQ ID NO: 58; CDR-L2 having a sequence shown as SEQ ID NO: 59; and CDR-L3 having a sequence shown as SEQ ID NO: 60; or
(4l) a heavy chain variable region (VH) comprising the following 3 CDRs: CDR-H1 having a sequence shown as SEQ ID NO: 121 or a variant thereof, CDR-H2 having a sequence shown as SEQ ID NO: 120 or a variant thereof, and CDR-H3 having a sequence shown as SEQ ID NO: 70 or a variant thereof, and/or, a light chain variable region (VL) comprising the following 3 CDRs: CDR-L1 having a sequence shown as SEQ ID NO: 71 or a variant thereof, CDR-L2 having a sequence shown as SEQ ID NO: 72 or a variant thereof, and CDR-L3 having a sequence shown as SEQ ID NO: 73 or a variant thereof;
wherein the variant in any one of (1a)-(1g), (1l), (2a)-(2i), (3a)-(3g), (3l), (4a)-(4g) and (4l) is subjected to replacement, deletion or addition of one or several amino acids as compared to the sequence from which the variant is derived.

70. The antibody or antigen-binding fragment thereof according to claim 68, comprising:
(a) a VH having a sequence shown as SEQ ID NO: 19 or a variant thereof and/or a VL having a sequence shown as SEQ ID NO: 20 or a variant thereof,
(b) a VH having a sequence shown as SEQ ID NO: 1 or a variant thereof and/or a VL having a sequence shown as SEQ ID NO: 2 or a variant thereof,
(c) a VH having a sequence shown as SEQ ID NO: 3 or a variant thereof and/or a VL having a sequence shown as SEQ ID NO: 4 or a variant thereof,
(d) a VH having a sequence shown as SEQ ID NO: 5 or a variant thereof and/or a VL having a sequence shown as SEQ ID NO: 6 or a variant thereof,
(e) a VH having a sequence shown as SEQ ID NO: 7 or a variant thereof and/or a VL having a sequence shown as SEQ ID NO: 8 or a variant thereof,
(f) a VH having a sequence shown as SEQ ID NO: 9 or a variant thereof and/or a VL having a sequence shown as SEQ ID NO: 10 or a variant thereof,
(g) a VH having a sequence shown as SEQ ID NO: 11 or a variant thereof and/or a VL having a sequence shown as SEQ ID NO: 12 or a variant thereof,
(h) a VH having a sequence shown as SEQ ID NO: 13 or a variant thereof and/or a VL having a sequence shown as SEQ ID NO: 14 or a variant thereof,
(i) a VH having a sequence shown as SEQ ID NO: 15 or a variant thereof and/or a VL having a sequence shown as SEQ ID NO: 16 or a variant thereof,
(j) a VH having a sequence shown as SEQ ID NO: 17 or a variant thereof and/or a VL having a sequence shown as SEQ ID NO: 18 or a variant thereof,
(k) a VH having a sequence shown as SEQ ID NO: 21 or a variant thereof and/or a VL having a sequence shown as SEQ ID NO: 22 or a variant thereof, or
(l) a VH having a sequence shown as SEQ ID NO: 23 or a variant thereof and/or a VL having a sequence shown as SEQ ID NO: 24 or a variant thereof,
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids as compared to the sequence from which the variants are derived.

71. The antibody or antigen-binding fragment thereof according to claim 68, wherein the antibody or antigen-binding fragment thereof is characterized by one or more of the following:
(1) the antibody or antigen-binding fragment thereof is a murine antibody, a chimeric antibody, a humanized antibody or a fully humanized antibody;
(2) the antibody or antigen-binding fragment thereof further comprising a constant region from or derived from human immunoglobulin;
(3) a heavy chain of the antibody or antigen-binding fragment thereof comprises a heavy chain constant region from or derived from human immunoglobulin;
(4) the antibody or antigen-binding fragment thereof comprises a wild-type Fc region, or a mutation-containing or chemically modified Fc region which has a changed effector function as compared to the wild-type Fc region;
(5) a light chain of the antibody or antigen-binding fragment thereof comprises a light chain constant region from or derived from human immunoglobulin; and
(6) the antibody or antigen-binding fragment thereof comprises a heavy chain constant region (CH) shown as SEQ ID NO: 25 or a variant thereof, and the variant is subjected to replacement of up to 20 amino acids as compared to SEQ ID NO: 25.

72. The antibody or antigen-binding fragment thereof according to claim 71, wherein the antibody or antigen-binding fragment thereof is characterized by one or more of the following:
(1) the variant is subjected to replacement of 3 amino acids as compared to SEQ ID NO: 25;
(2) the variant is subjected to replacement of amino acids at positions corresponding to positions 117, 118 and 120 in SEQ ID NO: 25;
(3) the variant is subjected to replacement of alanine at positions corresponding to the positions 117, 118 and 120 in SEQ ID NO: 25; and
(4) the variant has a heavy chain constant region (CH) shown as SEQ ID NO: 131.

73. The antibody or antigen-binding fragment thereof according to claim 68, wherein the antibody or antigen-binding fragment thereof is characterized by one or more of the following:
(1) the antibody or antigen-binding fragment thereof comprises a light chain constant region (CL) shown as SEQ ID NO: 26 or a variant thereof, and the variant is subjected to replacement of up to 20 amino acids as compared to SEQ ID NO: 26; and
(2) the antibody or antigen-binding fragment thereof comprises the heavy chain constant region (CH) shown as SEQ ID NO: 25 or 131 and the light chain constant region (CL) shown as SEQ ID NO: 26.

74. The antibody or antigen-binding fragment thereof according to claim 68, comprising:
(1) a heavy chain comprising the VH shown as SEQ ID NO: 19 and the heavy chain constant region (CH) shown as SEQ ID NO: 131, and, a light chain comprising VL having the sequence shown as SEQ ID NO: 20 and the light chain constant region (CL) shown as SEQ ID NO: 26;
(2) a heavy chain comprising the VH shown as SEQ ID NO: 19 and the heavy chain constant region (CH) shown as SEQ ID NO: 25, and, a light chain comprising VL having the sequence shown as SEQ ID NO: 20 and the light chain constant region (CL) shown as SEQ ID NO: 26;
(3) a heavy chain comprising the VH shown as SEQ ID NO: 1 and the heavy chain constant region (CH) shown as SEQ ID NO: 25, and, a light chain comprising VL having the sequence shown as SEQ ID NO: 2 and the light chain constant region (CL) shown as SEQ ID NO: 26;
(4) a heavy chain comprising the VH shown as SEQ ID NO: 1 and the heavy chain constant region (CH) shown as SEQ ID NO: 131, and, a light chain comprising VL having the sequence shown as SEQ ID NO: 2 and the light chain constant region (CL) shown as SEQ ID NO: 26;
(5) a heavy chain comprising the VH shown as SEQ ID NO: 3 and the heavy chain constant region (CH) shown as SEQ ID NO: 25, and, a light chain comprising VL having the sequence shown as SEQ ID NO: 4 and the light chain constant region (CL) shown as SEQ ID NO: 26;
(6) a heavy chain comprising the VH shown as SEQ ID NO: 5 and the heavy chain constant region (CH) shown as SEQ ID NO: 25, and, a light chain comprising VL having the sequence shown as SEQ ID NO: 6 and the light chain constant region (CL) shown as SEQ ID NO: 26;
(7) a heavy chain comprising the VH shown as SEQ ID NO: 7 and the heavy chain constant region (CH) shown as SEQ ID NO: 25, and, a light chain comprising VL having the sequence shown as SEQ ID NO: 8 and the light chain constant region (CL) shown as SEQ ID NO: 26;
(8) a heavy chain comprising the VH shown as SEQ ID NO: 9 and the heavy chain constant region (CH) shown as SEQ ID NO: 25, and, a light chain comprising VL having the sequence shown as SEQ ID NO: 10 and the light chain constant region (CL) shown as SEQ ID NO: 26;
(9) a heavy chain comprising the VH shown as SEQ ID NO: 11 and the heavy chain constant region (CH) shown as SEQ ID NO: 25, and, a light chain comprising VL having the sequence shown as SEQ ID NO: 12 and the light chain constant region (CL) shown as SEQ ID NO: 26;
(10) a heavy chain comprising the VH shown as SEQ ID NO: 13 and the heavy chain constant region (CH) shown as SEQ ID NO: 25, and, a light chain comprising VL having the sequence shown as SEQ ID NO: 14 and the light chain constant region (CL) shown as SEQ ID NO: 26;
(11) a heavy chain comprising the VH shown as SEQ ID NO: 15 and the heavy chain constant region (CH) shown as SEQ ID NO: 25, and, a light chain comprising VL having the sequence shown as SEQ ID NO: 16 and the light chain constant region (CL) shown as SEQ ID NO: 26;
(12) a heavy chain comprising the VH shown as SEQ ID NO: 17 and the heavy chain constant region (CH) shown as SEQ ID NO: 25, and, a light chain comprising VL having the sequence shown as SEQ ID NO: 18 and the light chain constant region (CL) shown as SEQ ID NO: 26;
(13) a heavy chain comprising the VH shown as SEQ ID NO: 21 and the heavy chain constant region (CH) shown as SEQ ID NO: 25, and, a light chain comprising VL having the sequence shown as SEQ ID NO: 22 and the light chain constant region (CL) shown as SEQ ID NO: 26;
(14) a heavy chain comprising the VH shown as SEQ ID NO: 23 and the heavy chain constant region (CH) shown as SEQ ID NO: 25, and, a light chain comprising VL having the sequence shown as SEQ ID NO: 24 and the light chain constant region (CL) shown as SEQ ID NO: 26;
(15) a heavy chain comprising the VH shown as SEQ ID NO: 3 and the heavy chain constant region (CH) shown as SEQ ID NO: 131, and, a light chain comprising VL having the sequence shown as SEQ ID NO: 4 and the light chain constant region (CL) shown as SEQ ID NO: 26;
(16) a heavy chain comprising the VH shown as SEQ ID NO: 5 and the heavy chain constant region (CH) shown as SEQ ID NO: 131, and, a light chain comprising VL having the sequence shown as SEQ ID NO: 6 and the light chain constant region (CL) shown as SEQ ID NO: 26;
(17) a heavy chain comprising the VH shown as SEQ ID NO: 7 and the heavy chain constant region (CH) shown as SEQ ID NO: 131, and, a light chain comprising VL having the sequence shown as SEQ ID NO: 8 and the light chain constant region (CL) shown as SEQ ID NO: 26;
(18) a heavy chain comprising the VH shown as SEQ ID NO: 9 and the heavy chain constant region (CH) shown as SEQ ID NO: 131, and, a light chain comprising VL having the sequence shown as SEQ ID NO: 10 and the light chain constant region (CL) shown as SEQ ID NO: 26;
(19) a heavy chain comprising the VH shown as SEQ ID NO: 11 and the heavy chain constant region (CH) shown as SEQ ID NO: 131, and, a light chain comprising VL having the sequence shown as SEQ ID NO: 12 and the light chain constant region (CL) shown as SEQ ID NO: 26;
(20) a heavy chain comprising the VH shown as SEQ ID NO: 13 and the heavy chain constant region (CH) shown as SEQ ID NO: 131, and, a light chain comprising VL having the sequence shown as SEQ ID NO: 14 and the light chain constant region (CL) shown as SEQ ID NO: 26;
(21) a heavy chain comprising the VH shown as SEQ ID NO: 15 and the heavy chain constant region (CH) shown as SEQ ID NO: 131, and, a light chain comprising VL having the sequence shown as SEQ ID NO: 16 and the light chain constant region (CL) shown as SEQ ID NO: 26;
(22) a heavy chain comprising the VH shown as SEQ ID NO: 17 and the heavy chain constant region (CH) shown as SEQ ID NO: 131, and, a light chain comprising VL having the sequence shown as SEQ ID NO: 18 and the light chain constant region (CL) shown as SEQ ID NO: 26;
(23) a heavy chain comprising the VH shown as SEQ ID NO: 21 and the heavy chain constant region (CH) shown as SEQ ID NO: 131, and, a light chain comprising VL having the sequence shown as SEQ ID NO: 22 and the light chain constant region (CL) shown as SEQ ID NO: 26; or
(24) a heavy chain comprising the VH shown as SEQ ID NO: 23 and the heavy chain constant region (CH) shown as SEQ ID NO: 131, and, a light chain comprising VL having the sequence shown as SEQ ID NO: 24 and the light chain constant region (CL) shown as SEQ ID NO: 26.

75. The antibody or antigen-binding fragment thereof according to claim 70, wherein the antibody or antigen-binding fragment thereof is characterized by one or more of the following:
(1) the N-terminal glutamine or glutamic acid of the VH having a sequence shown as SEQ ID NOs: 1, 3, 5, 7, 9, 11, 13, 15, 17, 19, 21 or 23 has undergone cyclization to pyroglutamate or pyroglutamic acid;
(2) the N-terminal glutamine of the VL having a sequence shown as SEQ ID NO: 12 has undergone cyclization to pyroglutamate or pyroglutamic acid, or the N-terminal glutamic acid of the VL having a sequence shown as SEQ ID NO: 16 has undergone cyclization to pyroglutamate or pyroglutamic acid; and
(3) the antibody or antigen-binding fragment thereof comprises: a VH having the amino acid sequence set forth in SEQ ID NO: 19 and N-terminal glutamic acid has undergone cyclization to pyroglutamate or pyroglutamic acid and a VL having the amino acid sequence set forth in SEQ ID NO: 20.

76. The antibody according to claim 68, comprising:
(a) a heavy chain comprising the sequence shown as SEQ ID NO: 135 or a variant thereof and/or a light chain comprising the sequence shown as SEQ ID NO: 112;
(b) a heavy chain consisting of the sequence shown as SEQ ID NO: 135 and a light chain consisting of the sequence shown as SEQ ID NO: 112;
(c) a heavy chain comprising the sequence shown as SEQ ID NO: 135 and a light chain comprising the sequence shown as SEQ ID NO: 112;
(d) a heavy chain consisting of the sequence shown as SEQ ID NO: 136 and a light chain consisting of the sequence shown as SEQ ID NO: 112;
(e) a heavy chain consisting of the sequence shown as SEQ ID NO: 137 and a light chain consisting of the sequence shown as SEQ ID NO: 112;
(f) a heavy chain consisting of the sequence shown as SEQ ID NO: 138 and a light chain consisting of the sequence shown as SEQ ID NO: 112;
(g) a heavy chain comprising the sequence shown as SEQ ID NO: 111 or a variant thereof and/or a light chain comprising the sequence shown as SEQ ID NO: 112;
(h) a heavy chain consisting of the sequence shown as SEQ ID NO: 111 and a light chain consisting of the sequence shown as SEQ ID NO: 112; or
(i) a heavy chain comprising the sequence shown as SEQ ID NO: 111 and a light chain comprising the sequence shown as SEQ ID NO: 112.

77. The antibody or antigen-binding fragment thereof according to claim 68,
wherein the antibody or antigen-binding fragment thereof is characterized by one or more of the following:
(1) the antibody or antigen-binding fragment thereof is selected from scFv, Fab, Fab′, Fab′-SH, (Fab′)2, an Fv fragment, disulfide bond linked Fv (dsFv), a diabody, a bispecific antibody and a multispecific antibody; and
(2) the antibody or antigen-binding fragment thereof is provided with a detectable marker, the detectable marker selected from an enzyme, radionuclide, fluorochrome, a luminescent substance and biotin.

78. The antibody or antigen-binding fragment thereof according to claim 68, wherein the antibody or antigen-binding fragment thereof has characteristics selected from the following:
(1) binding PTK7 with EC₅₀ of less than about 50 ng/mL;
(2) binding PTK7 with KD of less than about 100 nM;
(3) having ADCC and CDC activities or, not having ADCC and CDC activities;
(4) inducing PTK7 internalization;
(5) inhibiting cell proliferation; and/or
(6) inhibiting tumor growth.

79. Isolated nucleic acid molecules encoding the antibody or antigen-binding fragment thereof according to claim 68, the heavy chain and/or light chain thereof, or the heavy chain variable region and/or light chain variable region thereof.

80. The isolated nucleic acid molecules according to claim 79, comprising nucleic acid molecules encoding the heavy chain variable region of the antibody, and/or nucleic acid molecules encoding the light chain variable region of the antibody, wherein
(1) the nucleic acid molecules encoding the heavy chain variable region of the antibody comprise: (i) a nucleotide sequence shown as SEQ ID NO: 127, (ii) a sequence substantially identical to SEQ ID NO: 127, or (iii) a degenerate sequence of (i) or (ii); and/or, the nucleic acid molecules encoding the light chain variable region of the antibody comprise: (iv) a nucleotide sequence shown as SEQ ID NO: 128, (v) a sequence substantially identical to SEQ ID NO: 128, or (vi) a degenerate sequence of (iv) or (v);
(2) the nucleic acid molecules encoding the heavy chain of an antibody comprise: (i) a nucleotide sequence shown as SEQ ID NO: 132, (ii) a sequence substantially identical to SEQ ID NO: 132, or (iii) a degenerate sequence of (i) or (ii); and/or, the nucleic acid molecules encoding the light chain of the antibody comprise: (iv) a nucleotide sequence shown as SEQ ID NO: 130, (v) a sequence substantially identical to SEQ ID NO: 130, or (vi) a degenerate sequence of (iv) or (v); or
(3) the nucleic acid molecules encoding the heavy chain of the antibody comprise: (i) a nucleotide sequence shown as SEQ ID NO: 129, (ii) a sequence substantially identical to SEQ ID NO: 129, or (iii) a degenerate sequence of (i) or (ii); and/or, the nucleic acid molecules encoding the light chain of the antibody comprise: (iv) a nucleotide sequence shown as SEQ ID NO: 130, (v) a sequence substantially identical to SEQ ID NO: 130, or (vi) a degenerate sequence of (iv) or (v).

81. A vector, comprising the nucleic acid molecules according to claim 79.

82. The vector according to claim 81, wherein the vector is a cloning vector or an expression vector.

83. A host cell, comprising the nucleic acid molecules according to claim 79 or a vector comprising the nucleic acid molecules.

84. A method of preparing the antibody or antigen-binding fragment thereof according to claim 68, comprising: culturing a host cell under a condition that the expression of the antibody or antigen-binding fragment thereof is allowed, and recovering the antibody or antigen-binding fragment thereof from the cultured host cell culture.

85. An antibody or antigen-binding fragment thereof obtainable by the method of claim 84.

86. A conjugate, comprising the antibody or antigen-binding fragment thereof according to claim 68 and a coupling part linked thereto; optionally, the coupling part is selected from a detectable marker and a therapeutic reagent.

87. A multispecific antibody, comprising the antibody or antigen-binding fragment thereof according to claim 68;
optionally, the multispecific antibody is characterized by one or more of the following:
(1) the multispecific antibody comprises the antibody or antigen-binding fragment thereof as a first antigen-binding domain, and further comprises at least one second antigen-binding domain for other targets; and
(2) the multispecific antibody is a bispecific antibody or a trispecific antibody or a tetraspecific antibody.

88. A chimeric antigen receptor, comprising the antibody or antigen-binding fragment thereof according to claim 68, a transmembrane domain and one or more intracellular T cell signal domains.

89. A pharmaceutical composition, comprising the antibody or antigen-binding fragment thereof according to claim 68, or an isolated nucleic acid molecules comprising the antigen-binding fragment thereof, or a vector comprising the antigen-binding fragment thereof, or a host cell comprising the antigen-binding fragment thereof, or a conjugate comprising the antigen-binding fragment thereof, or a multispecific antibody comprising the antigen-binding fragment thereof, or a chimeric antigen receptor comprising the antigen-binding fragment thereof or a host cell expressing the chimeric antigen receptor, and a pharmaceutically acceptable carrier and/or excipient;
optionally, the pharmaceutical composition is characterized by one or more of the following:
(1) the pharmaceutical composition further comprising an additional pharmaceutical active agent;
(2) the pharmaceutical composition further comprising a drug having anti-tumor activity;
(3) the pharmaceutical composition further comprising an EGFR inhibitor, a BCR-ABL, FLT3, KIT or RET inhibitor, an HER2 inhibitor, an HER3 inhibitor, an HER4 inhibitor, an IGFR-1 inhibitor, a mTOR inhibitor, a PI3 kinase inhibitor, a c-met or VEGF inhibitor, a PARP inhibitor, a chemotherapeutic drug and any combination thereof; and
(4) the antibody or antigen-binding fragment thereof and the additional pharmaceutical active agent are provided as independent components or mixed components.

90. A diagnostic or therapeutic kit, comprising the antibody or antigen-binding fragment thereof according to claim 68, or an isolated nucleic acid molecules comprising the antigen-binding fragment thereof, or a vector comprising the antigen-binding fragment thereof, or a host cell comprising the antigen-binding fragment thereof, or a conjugate comprising the antigen-binding fragment thereof, or a multispecific antibody comprising the antigen-binding fragment thereof, or a chimeric antigen receptor comprising the antigen-binding fragment thereof or a host cell expressing the chimeric antigen receptor, or a pharmaceutical composition comprising the antigen-binding fragment thereof, and optionally, instruction for use.

91. A method for inhibiting cell proliferation, comprising: making the cells in contact with the antibody or antigen-binding fragment thereof according to claim 68, or an isolated nucleic acid molecules comprising the antigen-binding fragment thereof, or a vector comprising the antigen-binding fragment thereof, or a host cell comprising the antigen-binding fragment thereof, or a conjugate comprising the antigen-binding fragment thereof, or a multispecific antibody comprising the antigen-binding fragment thereof, or a chimeric antigen receptor comprising the antigen-binding fragment thereof or a host cell expressing the chimeric antigen receptor, or a pharmaceutical composition comprising the antigen-binding fragment thereof;
optionally, the method is characterized by one or more of the following:
(1) the cell is a cell expressing PTK7;
(2) the cell is a tumor cell.

92. A method for preventing and/or treating and/or assisting in treatment of tumor in a subject, comprising: administrating an effective amount of the antibody or antigen-binding fragment thereof according to claim 68, or an isolated nucleic acid molecules comprising the antigen-binding fragment thereof, or a vector comprising the antigen-binding fragment thereof, or a host cell comprising the antigen-binding fragment thereof, or a conjugate comprising the antigen-binding fragment thereof, or a multispecific antibody comprising the antigen-binding fragment thereof, or a chimeric antigen receptor comprising the antigen-binding fragment thereof or a host cell expressing the chimeric antigen receptor, or a pharmaceutical composition comprising the antigen-binding fragment thereof to the subject in need thereof;
optionally, the method further comprising: performing a second therapy to the subject, the second therapy being selected from surgery, chemotherapy, radiotherapy, immunotherapy, gene therapy, DNA therapy, RNA therapy, nano therapy, virus therapy, adjuvant therapy, and any combination thereof;

93. The method according to claim 92, the method is characterized by one or more of the following:
(1) the second therapy can be performed simultaneously, separately, or sequentially to the method according to claim 92;
(2) the tumor is PTK7 positive tumor;
(3) the tumor is selected from uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer, esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof.

94. A method of detecting the presence or level of PTK7 in a sample, comprising: making the sample in contact with the antibody or antigen-binding fragment thereof according to claim 68 under a condition that a complex is allowed to be formed between the antibody or antigen-binding fragment thereof and PTK7, and detecting the formation of the complex;
optionally, wherein the method is characterized by one or more of the following:
(1) the method is used for diagnosing PTK7 positive tumor;
(2) the method is used for diagnosing uterine cancer, testicular cancer, thyroid cancer, nasopharyngeal carcinoma, glioblastoma, leukemia, lymphoma, colonic adenocarcinoma, glioblastoma cerebri, hepatic bile duct carcinoma, osteosarcoma, esophageal squamous cancer, intrahepatic cholangiocarcinoma, breast cancer, ovarian cancer, lung cancer, esophageal cancer, colorectal cancer, pancreatic cancer, head and neck squamous cell carcinoma, gastric cancer, melanoma, prostate cancer, liver cancer, kidney cancer, bladder cancer and pharyngeal squamous cell carcinoma or any combination thereof; and
(3) the method comprises: detecting the expression level of PTK7 in a to-be-tested sample from the subject, and comparing the expression level with a reference value, wherein the increase in the expression level compared with the reference value is an indication of the tumor.
